SEQUENCE LISTING (216 sequences)

SEQ ID NO: 1 - protein, 315 aa, Shigella dysenteriae [sequence omitted]

SEQ ID NO: 2 - protein, 89 aa, Shigella dysenteriae
  Shiga Toxin B-chain
MKKTLLIAASLSFFSASALATPDCVTGKVEYTKYNDDDTFTVKVGDKELFTNRWNLQSLLLSAQITGMTVTIKTNACHNGGGFSEVIFR

SEQ ID NO: 3 - protein, 319 aa, Escherichia coli
  Shiga-like Toxin II subunit A Precursor
MKCILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEESQPECQITGDRPVIKINNTLWESNTAAAFLNRKSQFLYTTGK

SEQ ID NO: 4 - protein, 89 aa, Escherichia coli
  Shiga-like Toxin II subunit B Precursor
MKKMFMAVLFALASVNAMAADCAKGKIEFSKYNEDDTFTVKVDGKEYWTSRWNLQPLLQSAQLTGMTVTIKSSTCESGSGFAEVQFNND

SEQ ID NO: 5 - protein, 293 aa, Shigella dysenteriae
  Shiga toxin A-chain Mature
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 6 - protein, 293 aa, Shigella dysenteriae
  VARIANT 45: Xaa= Thr or Ser
KEFTLDFSTAKTYVDSLNVIRSAIGTPLQTISSGGTSLLMIDSGXGDNLFAVDVRGIDPEEGRFNNLRLIVERNNLXVTGFVNRTNNVFYRFADFSHVTFPGTTAVTLSGDSSXTTLQRVAGISRTGMQINRHSLTTSYLDLMSHSGTSLTQSVARAMLRFVTVTAXALXFRQIQRGFRTTLDDLSGRSYVMTAEDVDLTLNXXXLSSVLPDYHGQDSVRVGRISXGSINXXXXSVALIXXXHHHASRVARMXXDEFPSMCPADGRVRGITHNKILXXSSTLGAILMXXTISS

SEQ ID NO: 7 - protein, 293 aa, Escherichia coli
  Stx1c Mature
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSVNAILGSVALILNCHHHASRVARIVPNEFPSMCPVDGRVRGITHNKILWDSSTLGAILIRRAISS

SEQ ID NO: 8 - protein, 293 aa, Escherichia coli
  Stx1d Mature
KEFTLDFSTAKKYVDSLNVIRSAIGTPLQTISSGGTSLLMIDSGTGDNLFAVDIMGLEPEEERFNNLRLIVERNNLYVTGFVNRTNNVFYRFADFSHVTFPGTRAVTLSGDSSYTTLQRVAGISRTGMQINRHSLTTSYLDLMSYSGTSLTQSVARAMLRFVTVTAEALRFRQIQRGFRTTLDDLSGRSYVMTAEDVDLTLNWGRLSSILPDYHGQDSVRVGRISFGSINAILGSVALILNCHHHASRVARMTPDEFPSMCPTDGSGRGITHNKILWDSSTLGAILIRRTISS

SEQ ID NO: 9 - protein, 293 aa, Escherichia coli
  Shiga toxin allelic variant
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCQADGRVRGITHNKILWDSSTLGAILMRRTISS

SEQ ID NO: 10 - protein, 293 aa, Escherichia coli
  Shiga toxin allelic variant
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 11 - protein, 293 aa, Shigella dysenteriae
  Shiga toxin allelic variant
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEPLRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 12 - protein, 293 aa, Escherichia coli
  Shiga toxin allelic variant
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTLLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 13 - protein, 293 aa, Escherichia coli
  Shiga toxin allelic variant
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSSDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 14 - protein, 293 aa, Escherichia coli
  Shiga toxin allelic variant
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFESINAILGSVALILNCHHHASRVARMASDEFPSMCPADGRVRGITHNKILWDSSTLGAILMRRTISS

SEQ ID NO: 15 - protein, 293 aa, Escherichia coli
  Shiga toxin allelic variant
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHRGTSLPQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 16 - protein, 293 aa, Escherichia coli
  Shiga toxin allelic variant
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHRGTSLPQSGARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 17 - protein, 293 aa, Escherichia coli
  Shiga toxin allelic variant
KEFTLDFSTAKTYVDSLNVIRSAIGTPLQTISSGGTSLLMIDSGTGDNLFAVDVRGIDPQEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSVNAILGSVALILNCHHHASRVARMASDEFPSMCPADGRGRGITHNKILWDSSTLGAILMRRTISS

SEQ ID NO: 18 - protein, 293 aa, Escherichia coli
  Shiga toxin allelic variant
KEFTLDFSTAKTYVDSLNVIRSAIGTPLQTISSGGTSLLMIDSGTGGNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSVNAILGSVALILNCHHHASRVARIVPNEFPSMCPVDGRVRGITHNKILWDSSTLGAILIRRAISS

SEQ ID NO: 19 - protein, 293 aa, Escherichia coli
  Shiga toxin allelic variant
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSVNAILGSVALILNCHHHASRVARIVPNELPSMCPVDGRVRGITHNKILWDSSTLGAILIRRAISS

SEQ ID NO: 20 - protein, 293 aa, Escherichia coli
  Shiga toxin allelic variant
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAGTLSGDSSYTTLQRVAGISRTGMQINRHSLTTPYLDLMSHSGTSLTQSVARAMLPFVTVTAEALRFRQIQRGFRTTLDDLSGRSYVMTAEDVDLTLNWGRLSSVLPDYHGQDSVRVGRISFGSVNAILGSVALILNCPHHASRVARIVPNEFPSMCPVDGRVRGITHNKILWDSSTLGAILIRRAISS

SEQ ID NO: 21 - protein, 293 aa, Escherichia coli; identical to SEQ ID NO: 8
  Shiga toxin allelic variant

SEQ ID NO: 22 - protein, 246 aa, Shigella dysenteriae
  SA1 Variant 1
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHA

SEQ ID NO: 23 - DNA, 738 bp, Shigella dysenteriae
  SA1 Variant 1
aaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgtacgtgtaggccgtatctcctttggttccattaacgccattttaggttctgttgcacttattctgaactgccaccaccatgca

SEQ ID NO: 24 - protein, 241 aa, Shigella dysenteriae
  SA1 Variant 2
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILN

SEQ ID NO: 25 - DNA, 723 bp, Shigella dysenteriae
  SA1 Variant 2
aaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgtacgtgtaggccgtatctcctttggttccattaacgccattttaggttctgttgcacttattctgaac

SEQ ID NO: 26 - protein, 246 aa, Shigella dysenteriae
  SA1 Variant 3
KEFTLDFSTAKTYVDSLNVIRSAIGTPLQTISSGGTSRLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHA

SEQ ID NO: 27 - DNA, 738 bp, Shigella dysenteriae
  SA1 Variant 3
aaagaattcacactcgacttcagcaccgcaaaaacttacgtagactccctgaatgtaatccgctccgctatcggcaccccgttacaaactattagctccggcggtacatctcgcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgtacgtgtaggccgtatctcctttggttccattaacgccattttaggttctgttgcacttattctgaactgccaccaccatgca

SEQ ID NO: 28 - protein, 241 aa, Shigella dysenteriae
  SA1 Variant 4
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSARVGRISFGSINAILGSVALILN

SEQ ID NO: 29 - DNA, 723 bp, Shigella dysenteriae
  SA1 Variant 4
aaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaac

SEQ ID NO: 30 - protein, 22 aa, Artificial Sequence
  Description of Artificial Sequence SA1 Peptide for Antibody Production
CLFAVDVDVRGIDPEEGRFNNL

SEQ ID NO: 31 - DNA, 6 bp, Artificial Sequence
  Description of Artificial Sequence NdeI restriction site
catatg

SEQ ID NO: 32 - DNA, 6 bp, Artificial Sequence
  Description of Artificial Sequence EcoRI restriction site
gaattc

SEQ ID NO: 33 - DNA, 6 bp, Artificial Sequence
  Description of Artificial Sequence BamHI restriction site
ggattc

SEQ ID NO: 34 - DNA, 6 bp, Artificial Sequence
  Description of Artificial Sequence Ala-Met linker
gccatg

SEQ ID NO: 35 - DNA, 20 bp, Artificial Sequence
  Description of Artificial Sequence T7 Primer
taatacgactcactataggg

SEQ ID NO: 36 - DNA, 18 bp, Artificial Sequence
  Description of Artificial Sequence T7t Primer
gctagttattgctcagcg

SEQ ID NO: 37 - DNA, 987 bp, Artificial Sequence
  Description of Artificial Sequence Construct encoding chemokine- toxin fusion protein MCP-1-AM-Shiga-A1 Variant 1
catatgcaacctgacgcaatcaacgctcctgtcacctgttgttacaattttaccaatcgcaaaatttctgtccaacgtcttgcatcttatcgccgtattacttcctctaaatgtcctaaagaagccgtcattttcaaaaccattgttgcaaaagaaatctgtgccgacccgaaacaaaaatgggtacaagactccatggaccacctcgataaacaaactcaaaccccaaaaacagccatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgtacgtgtaggccgtatctcctttggttccattaacgccattttaggttctgttgcacttattctgaactgccaccaccatgcataaggatcc

SEQ ID NO: 38 - protein, 325 aa, Artificial Sequence
  Description of Artificial Sequence Chemokine-toxin fusion protein MCP-1-AM-Shiga-A1 Variant 1
MQPDAINAPVTCCYNFTNRKISVQRLASYRRITSSKCPKEAVIFKTIVAKEICADPKQKWVQDSMDHLDKQTQTPKTAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHA

SEQ ID NO: 39 - DNA, 972 bp, Artificial Sequence
  Description of Artificial Sequence Construct encoding chemokine-toxin fusion protein MCP-1-AM-Shiga-A1 Variant 2
catatgcaacctgacgcaatcaacgctcctgtcacctgttgttacaattttaccaatcgcaaaatttctgtccaacgtcttgcatcttatcgccgtattacttcctctaaatgtcctaaagaagccgtcattttcaaaaccattgttgcaaaagaaatctgtgccgacccgaaacaaaaatgggtacaagactccatggaccacctcgataaacaaactcaaaccccaaaaacagccatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgtacgtgtaggccgtatctcctttggttccattaacgccattttaggttctgttgcacttattctgaactaaggatcc

SEQ ID NO: 40 - protein, 320 aa, Artificial Sequence
  Description of Artificial Sequence Chemokine-toxin fusion protein MCP-1-AM-Shiga-A1 Variant 2
MQPDAINAPVTCCYNFTNRKISVQRLASYRRITSSKCPKEAVIFKTIVAKEICADPKQKWVQDSMDHLDKQTQTPKTAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILN

SEQ ID NO: 41 - DNA, 987 bp, Artificial Sequence
  Description of Artificial Sequence Construct encoding chemokine- toxin fusion protein MCP-1-AM-Shiga-A1 Variant 3
catatgcaacctgacgcaatcaacgctcctgtcacctgttgttacaattttaccaatcgcaaaatttctgtccaacgtcttgcatcttatcgccgtattacttcctctaaatgtcctaaagaagccgtcattttcaaaaccattgttgcaaaagaaatctgtgccgacccgaaacaaaaatgggtacaagactccatggaccacctcgataaacaaactcaaaccccaaaaacagccatgaaagaattcacactcgacttcagcaccgcaaaaacttacgtagactccctgaatgtaatccgctccgctatcggcaccccgttacaaactattagctccggcggtacatctcgcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgtacgtgtaggccgtatctcctttggttccattaacgccattttaggttctgttgcacttattctgaactgccaccaccatgcataaggatcc

SEQ ID NO: 42 - protein, 325 aa, Artificial Sequence
  Description of Artificial Sequence Chemokine-toxin fusion protein MCP-1-AM-Shiga-A1 Variant 3
MQPDAINAPVTCCYNFTNRKISVQRLASYRRITSSKCPKEAVIFKTIVAKEICADPKQKWVQDSMDHLDKQTQTPKTAMKEFTLDFSTAKTYVDSLNVIRSAIGTPLQTISSGGTSRLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHA

SEQ ID NO: 43 - DNA, 972 bp, Artificial Sequence
  Description of Artificial Sequence Construct encoding chemokine-toxin fusion protein MCP-1-AM-Shiga-A1 Variant 4
catatgcaacctgacgcaatcaacgctcctgtcacctgttgttacaattttaccaatcgcaaaatttctgtccaacgtcttgcatcttatcgccgtattacttcctctaaatgtcctaaagaagccgtcattttcaaaaccattgttgcaaaagaaatctgtgccgacccgaaacaaaaatgggtacaagactccatggaccacctcgataaacaaactcaaaccccaaaaacagccatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 44 - protein, 320 aa, Artificial Sequence
  Description of Artificial Sequence Chemokine-toxin fusion protein MCP-1-AM-Shiga-A1 Variant 4
MQPDAINAPVTCCYNFTNRKISVQRLASYRRITSSKCPKEAVIFKTIVAKEICADPKQKWVQDSMDHLDKQTQTPKTAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSARVGRISFGSINAILGSVALILN

SEQ ID NO: 45 - DNA, 966 bp, Artificial Sequence
  Description of Artificial Sequence Construct encoding chemokine-toxin fusion protein Eotaxin-1-AM-Shiga-A1 Variant 4
catatgggccccgcatccgttccaactacatgttgttttaatctggcgaaccgcaagattcctctccagcgtcttgaatcatacagacggatcacgtctggtaaatgcccgcaaaaggccgtgatattcaaaaccaaattggcgaaagatatctgcgctgaccctaagaaaaagtgggtacaggactcgatgaagtatctggatcaaaaaagcccaaccccgaaaccggccatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 46 - protein, 318 aa, Artificial Sequence
  Description of Artificial Sequence Chemokine-toxin fusion protein Eotaxin-1-AM-Shiga-A1 Variant 4
MGPASVPTTCCFNLANRKIPLQRLESYRRITSGKCPQKAVIFKTKLAKDICADPKKKWVQDSMKYLDQKSPTPKPAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSARVGRISFGSINAILGSVALILN

SEQ ID NO: 47 - DNA, 960 bp, Artificial Sequence
  Description of Artificial Sequence Construct encoding chemokine-toxin fusion protein SDF-1beta-AM-Shiga-A1 Variant 4
catatgaagccggtgtctctgtcctaccgttgcccatgtagatttttcgagagccatgttgcccgggcaaacgttaaacacctaaagatactcaatacccctaactgtgcgttacagatcgtcgcgaggcttaaaaacaataaccgccaagtatgcatcgaccccaagttgaagtggattcaggaatatctggaaaaagctctgaataaacgattcaaaatggccatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 48 - protein, 316 aa, Artificial Sequence
  Description of Artificial Sequence Chemokine-toxin fusion protein SDF-1beta-AM-Shiga-A1 Variant 4
MKPVSLSYRCPCRFFESHVARANVKHLKILNTPNCALQIVARLKNNNRQVCIDPKLKWIQEYLEKALNKRFKMAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSARVGRISFGSINAILGSVALILN

SEQ ID NO: 49 - DNA, 963 bp, Artificial Sequence
  Description of Artificial Sequence Construct encoding chemokine-toxin fusion protein GRO-alpha-AM-Shiga-A1 Variant 4
catatggcgtccgttgctaccgagctgcgttgtcagtgcctgcaaactctgcagggtatccacccgaaaaacatccagagcgtaaacgtgaaatctccaggtccgcactgcgcgcagaccgaagttattgctaccctgaaaaacggccgtaaagcgtgtctgaacccggcctccccgatcgttaagaaaattatcgaaaagatgctgaactctgacaaaagcaatgcaatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 50 - protein, 317 aa, Artificial Sequence
  Description of Artificial Sequence Chemokine-toxin fusion protein GRO-alpha-AM-Shiga-A1 Variant 4
MASVATELRCQCLQTLQGIHPKNIQSVNVKSPGPHCAQTEVIATLKNGRKACLNPASPIVKKIIEKMLNSDKSNAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSARVGRISFGSINAILGSVALILN

SEQ ID NO: 51 - DNA, 951 bp, Artificial Sequence
  Description of Artificial Sequence Construct encoding chemokine-toxin fusion protein MIP-1beta-AM-Shiga-A1 Variant 4
catatggctccgatgggttctgacccgccgactgcttgctgtttttcttataccgcacgtaaactgccgcgtaacttcgttgttgactactacgagacctcctctctgtgctctcagccagccgtagtcttccagaccaagcgcagcaaacaggtgtgcgcggatccttccgaaagctgggtgcaagaatatgtttacgatctggaactgaacgcgatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 52 - protein, 313 aa, Artificial Sequence
  Description of Artificial Sequence Chemokine-toxin fusion protein MIP-1beta-AM-Shiga-A1 Variant 4
MAPMGSDPPTACCFSYTARKLPRNFVVDYYETSSLCSQPAVVFQTKRSKQVCADPSESWVQEYVYDLELNAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSARVGRISFGSINAILGSVALILN

SEQ ID NO: 53 - DNA, 975 bp, Artificial Sequence
  Description of Artificial Sequence Construct encoding chemokine-toxin fusion protein IL-8-AM-Shiga-A1 Variant 4
catatggcggtcctgccacgttccgcgaaagaactgcgctgccagtgcattaagacctacagcaaaccgtttcacccgaaattcatcaaagaactgcgtgttatcgagtctggtccgcactgtgcaaacaccgaaattatcgttaaactgtctgatggccgtgaactgtgcctggacccgaaagagaactgggtgcagcgtgtggtagaaaagttcctgaaacgcgccgaaaattccgctatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 54 - protein, 321 aa, Artificial Sequence
  Description of Artificial Sequence Chemokine-toxin fusion protein IL-8-AM-Shiga-A1 Variant 4
MAVLPRSAKELRCQCIKTYSKPFHPKFIKELRVIESGPHCANTEIIVKLSDGRELCLDPKENWVQRVVEKFLKRAENSAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSARVGRISFGSINAILGSVALILN

SEQ ID NO: 55 - DNA, 975 bp, Artificial Sequence
  Description of Artificial Sequence Construct encoding chemokine-toxin fusion protein IP-10-AM-Shiga-A1 Variant 4
catatggttccgctgtctcgcaccgttcgttgtacttgcatctctatctctaatcagccggtcaacccgcgcagcctggaaaaactggaaatcatcccggcgtcccagttctgccctcgtgtggaaattatcgctaccatgaagaagaaaggtgagaagcgttgcctgaacccagagtctaaagcaattaaaaacctgctgaaagctgtatccaaagaacggtcgaaacgtagcccggcgatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 56 - protein, 321 aa, Artificial Sequence
  Description of Artificial Sequence Chemokine-toxin fusion protein IP-10-AM-Shiga-A1 Variant 4
MVPLSRTVRCTCISISNQPVNPRSLEKLEIIPASQFCPRVEIIATMKKKGEKRCLNPESKAIKNLLKAVSKERSKRSPAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSARVGRISFGSINAILGSVALILN

SEQ ID NO: 57 - DNA, 972 bp, Artificial Sequence
  Description of Artificial Sequence Construct encoding chemokine-toxin fusion protein MCP-3-AM-Shiga-A1 Variant 4
catatgcagcctgtgggtatcaatacctctaccacttgttgctatcgctttatcaacaaaaagatcccgaagcagcgtctcgaatcgtaccgtcgcacgacttccagccattgcccgcgtgaggctgttattttcaaaaccaaacttgataaagaaatttgcgcggacccaacccagaaatgggtacaggatttcatgaaacacttggacaaaaagactcaaaccccgaaactggccatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 58 - protein, 320 aa, Artificial Sequence
  Description of Artificial Sequence Chemokine-toxin fusion protein MCP-3-AM-Shiga-A1 Variant 4
MQPVGINTSTTCCYRFINKKIPKQRLESYRRTTSSHCPREAVIFKTKLDKEICADPTQKWVQDFMKHLDKKTQTPKLAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSARVGRISFGSINAILGSVALILN

SEQ ID NO: 59 - DNA, 954 bp, Artificial Sequence
  Description of Artificial Sequence Construct encoding chemokine-toxin fusion protein MIP-3alpha-AM-Shiga-A1 Variant 4
catatggcaagcaactttgattgttgtctgggttataccgaccgcattctgcatccgaaattcattgtcggcttcactcgtcagctggctaatgaaggttgcgacatcaacgccatcatcttccacaccaaaaagaaactctccgtatgcgcgaacccaaaacagacgtgggttaaatacatcgttcgtctgctttctaaaaaggtgaagaacatggccatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 60 - protein, 314 aa, Artificial Sequence
  Description of Artificial Sequence Chemokine-toxin fusion protein MIP-3alpha-AM-Shiga-A1 Variant 4
MASNFDCCLGYTDRILHPKFIVGFTRQLANEGCDINAIIFHTKKKLSVCANPKQTWVKYIVRLLSKKVKNMAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSARVGRISFGSINAILGSVALILN

SEQ ID NO: 61 - DNA, 951 bp, Artificial Sequence
  Description of Artificial Sequence Construct encoding chemokine-toxin fusion protein MDC-AM-Shiga-A1 Variant 4
catatgggtccatacggtgcgaatatggaggactccgtgtgctgtcgtgattatgtccgttatcgtctgcctctgcgtgtggttaaacacttttactggacttctgactcttgcccgcgcccgggcgttgttctgctgaccttccgtgacaaagaaatttgcgctgatccgcgcgttccgtgggtaaaaatgatcctgaacaagctgagccaggccatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 62 - protein, 313 aa, Artificial Sequence
  Description of Artificial Sequence Chemokine-toxin fusion protein MDC-AM-Shiga-A1 Variant 4
MGPYGANMEDSVCCRDYVRYRLPLRVVKHFYWTSDSCPRPGVVLLTFRDKEICADPRVPWVKMILNKLSQAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSARVGRISFGSINAILGSVALILN

SEQ ID NO: 63 - DNA, 951 bp, Artificial Sequence
  Description of Artificial Sequence Construct encoding chemokine-toxin fusion protein MIP-1alpha-AM-Shiga-A1 Variant 4
catatgtctctggcggctgataccccgactgcatgttgcttctcttacacgtcccgccagatcccacagaacttcatcgccgattattttgaaacctcctctcaatgcagcaaacctggtgtaattttcctgaccaagcgtagccgtcaggtctgcgctgacccgtccgaggaatgggttcagaaatacgtgtctgacctggaactgagcgcggccatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 64 - protein, 313 aa, Artificial Sequence
  Description of Artificial Sequence Chemokine-toxin fusion protein MIP-1alpha-AM-Shiga-A1 Variant 4
MSLAADTPTACCFSYTSRQIPQNFIADYFETSSQCSKPGVIFLTKRSRQVCADPSEEWVQKYVSDLELSAAMKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSARVGRISFGSINAILGSVALILN

SEQ ID NO: 65 - DNA, 966 bp, Artificial Sequence
  Description of Artificial Sequence Construct encoding chemokine-toxin fusion protein Eotaxin-1-AM-Shiga-A1 Variant 4
catatgggccctgcctccgttccaaccacctgctgttttaatctcgccaatcgtaaaatcccccttcaacgcttagaatcttaccgtcgtattacctctggaaaatgccctcaaaaagccgtaatctttaaaaccaaacttgccaaagacatctgtgccgatccaaaaaaaaaatgggttcaagactcaatgaaatatctcgaccaaaaatctccaactcccaaacctgccatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 66 - DNA, 1005 bp, Artificial Sequence
  Description of Artificial Sequence Construct encoding chemokine-toxin fusion protein BCA-1-AM-Shiga-A1 Variant 4
catatggttctggaagtgtactataccagcctgcgctgccgctgcgtgcaagaatcctctgttttcatccctcgtcgcttcatcgaccgtatccagattctgccgcgtggtaacggctgcccgcgtaaagaaatcatcgtgtggaaaaagaacaaatctatcgtttgtgtagatccgcaggcggagtggattcagcgtatgatggaagttctgcgcaaacgtagctcttccaccctgccagtaccggtctttaaacgtaaaattccggccatgaaagaattcacactcgacttcagcaccgcaaaaacttacgtagactccctgaatgtaatccgctccgctatcggcaccccgttacaaactattagctccggcggtacatctctcttaatgatcgattccggtactggcgacaatttattcgctgtggatgtacgtggcattgacccagaagaaggccgtttcaataacctgcgcttaattgttgaacgtaataacctgtatgtaactggcttcgtaaaccgtaccaacaacgtcttttaccgcttcgctgacttttctcacgtaacctttcccggaacaactgcagtaactctctccggcgacagttcctatacgaccctccaacgtgttgcaggtatttctcgcaccggtatgcaaatcaatcgtcactctcttactacatcgtatctcgatttaatgtcacactccggcacctctttaacccagtccgtcgcacgcgcaatgttacgttttgttactgtcacagcagaggctcttcgctttcgtcagattcaacgtggtttccgcacaactcttgatgatttatctggccgctcttatgtaatgaccgcagaagatgtagatctgaccttgaactggggccgcctgagcagtgtgttacctgattatcacggacaagacagtgcacgtgtaggccgtatctcctttggttccattaacgccattttaggttctgttgcacttattctgaactaaggatcc

SEQ ID NO: 67 - protein, 331 aa, Artificial Sequence [sequence omitted]

SEQ ID NO: 68 - DNA, 228 bp, Homo sapien
  Mature MCP-1
caacctgacgcaatcaacgctcctgtcacctgttgttacaattttaccaatcgcaaaatttctgtccaacgtcttgcatcttatcgccgtattacttcctctaaatgtcctaaagaagccgtcattttcaaaaccattgttgcaaaagaaatctgtgccgacccgaaacaaaaatgggtacaagactccatggaccacctcgataaacaaactcaaaccccaaaaaca

SEQ ID NO: 69 - protein, 76 aa, Homo sapien
  Mature MCP-1
QPDAINAPVTCCYNFTNRKISVQRLASYRRITSSKCPKEAVIFKTIVAKEICADPKQKWVQDSMDHLDKQTQTPKT

SEQ ID NO: 70 - DNA, 231 bp, Homo sapien
  Mature MCP-1 with N-Terminal Methionine
atgcaacctgacgcaatcaacgctcctgtcacctgttgttacaattttaccaatcgcaaaatttctgtccaacgtcttgcatcttatcgccgtattacttcctctaaatgtcctaaagaagccgtcattttcaaaaccattgttgcaaaagaaatctgtgccgacccgaaacaaaaatgggtacaagactccatggaccacctcgataaacaaactcaaaccccaaaaaca

SEQ ID NO: 71 - protein, 77 aa, Homo sapien
  Mature MCP-1 with N-Terminal Methionine
MQPDAINAPVTCCYNFTNRKISVQRLASYRRITSSKCPKEAVIFKTIVAKEICADPKQKWVQDSMDHLDKQTQTPKT

SEQ ID NO: 72 - DNA, 243 bp, Homo sapien
  Eotaxin construct
catatgggccccgcatccgttccaactacatgttgttttaatctggcgaaccgcaagattcctctccagcgtcttgaatcatacagacggatcacgtctggtaaatgcccgcaaaaggccgtgatattcaaaaccaaattggcgaaagatatctgcgctgaccctaagaaaaagtgggtacaggactcgatgaagtatctggatcaaaaaagcccaaccccgaaaccggccatgaaagaattc

SEQ ID NO: 73 - DNA, 237 bp, Homo sapien
  SDF-1beta construct
catatgaagccggtgtctctgtcctaccgttgcccatgtagatttttcgagagccatgttgcccgggcaaacgttaaacacctaaagatactcaatacccctaactgtgcgttacagatcgtcgcgaggcttaaaaacaataaccgccaagtatgcatcgaccccaagttgaagtggattcaggaatatctggaaaaagctctgaataaacgattcaaaatggccatgaaagaattc

SEQ ID NO: 74 - DNA, 240 bp, Homo sapien
  GRO-alpha construct
catatggcgtccgttgctaccgagctgcgttgtcagtgcctgcaaactctgcagggtatccacccgaaaaacatccagagcgtaaacgtgaaatctccaggtccgcactgcgcgcagaccgaagttattgctaccctgaaaaacggccgtaaagcgtgtctgaacccggcctccccgatcgttaagaaaattatcgaaaagatgctgaactctgacaaaagcaatgcaatgaaagaattc

SEQ ID NO: 75 - DNA, 228 bp, Homo sapien
  MIP-1beta construct
catatggctccgatgggttctgacccgccgactgcttgctgtttttcttataccgcacgtaaactgccgcgtaacttcgttgttgactactacgagacctcctctctgtgctctcagccagccgtagtcttccagaccaagcgcagcaaacaggtgtgcgcggatccttccgaaagctgggtgcaagaatatgtttacgatctggaactgaacgcgatgaaagaattc

SEQ ID NO: 76 - DNA, 252 bp, Homo sapien
  IL-8 construct
catatggcggtcctgccacgttccgcgaaagaactgcgctgccagtgcattaagacctacagcaaaccgtttcacccgaaattcatcaaagaactgcgtgttatcgagtctggtccgcactgtgcaaacaccgaaattatcgttaaactgtctgatggccgtgaactgtgcctggacccgaaagagaactgggtgcagcgtgtggtagaaaagttcctgaaacgcgccgaaaattccgctatgaaagaattc

SEQ ID NO: 77 - DNA, 252 bp, Homo sapien
  IP-10 construct
catatggttccgctgtctcgcaccgttcgttgtacttgcatctctatctctaatcagccggtcaacccgcgcagcctggaaaaactggaaatcatcccggcgtcccagttctgccctcgtgtggaaattatcgctaccatgaagaagaaaggtgagaagcgttgcctgaacccagagtctaaagcaattaaaaacctgctgaaagctgtatccaaagaacggtcgaaacgtagcccggcgatgaaagaattc

SEQ ID NO: 78 - DNA, 249 bp, Homo sapien
  MCP-3 construct
catatgcagcctgtgggtatcaatacctctaccacttgttgctatcgctttatcaacaaaaagatcccgaagcagcgtctcgaatcgtaccgtcgcacgacttccagccattgcccgcgtgaggctgttattttcaaaaccaaacttgataaagaaatttgcgcggacccaacccagaaatgggtacaggatttcatgaaacacttggacaaaaagactcaaaccccgaaactggccatgaaagaattc

SEQ ID NO: 79 - DNA, 231 bp, Homo sapien
  MIP-3alpha construct
catatggcaagcaactttgattgttgtctgggttataccgaccgcattctgcatccgaaattcattgtcggcttcactcgtcagctggctaatgaaggttgcgacatcaacgccatcatcttccacaccaaaaagaaactctccgtatgcgcgaacccaaaacagacgtgggttaaatacatcgttcgtctgctttctaaaaaggtgaagaacatggccatgaaagaattc

SEQ ID NO: 80 - DNA, 228 bp, Homo sapien
  MDC construct
catatgggtccatacggtgcgaatatggaggactccgtgtgctgtcgtgattatgtccgttatcgtctgcctctgcgtgtggttaaacacttttactggacttctgactcttgcccgcgcccgggcgttgttctgctgaccttccgtgacaaagaaatttgcgctgatccgcgcgttccgtgggtaaaaatgatcctgaacaagctgagccaggccatgaaagaattc

SEQ ID NO: 81 - DNA, 228 bp, Homo sapien
  MIP-1alpha construct
catatgtctctggcggctgataccccgactgcatgttgcttctcttacacgtcccgccagatcccacagaacttcatcgccgattattttgaaacctcctctcaatgcagcaaacctggtgtaattttcctgaccaagcgtagccgtcaggtctgcgctgacccgtccgaggaatgggttcagaaatacgtgtctgacctggaactgagcgcggccatgaaagaattc

SEQ ID NO: 82 - DNA, 243 bp, Homo sapien
  Eotaxin construct (Blue Heron)
catatgggccctgcctccgttccaaccacctgctgttttaatctcgccaatcgtaaaatcccccttcaacgcttagaatcttaccgtcgtattacctctggaaaatgccctcaaaaagccgtaatctttaaaaccaaacttgccaaagacatctgtgccgatccaaaaaaaaaatgggttcaagactcaatgaaatatctcgaccaaaaatctccaactcccaaacctgccatgaaagaattc

SEQ ID NO: 83 - DNA, 282 bp, Homo sapien
  BCA-1 construct
catatggttctggaagtgtactataccagcctgcgctgccgctgcgtgcaagaatcctctgttttcatccctcgtcgcttcatcgaccgtatccagattctgccgcgtggtaacggctgcccgcgtaaagaaatcatcgtgtggaaaaagaacaaatctatcgtttgtgtagatccgcaggcggagtggattcagcgtatgatggaagttctgcgcaaacgtagctcttccaccctgccagtaccggtctttaaacgtaaaattccggccatgaaagaattc

SEQ ID NO: 84 - DNA, 4339 bp, Artificial Sequence
  Description of Artificial Sequence pET9C Vector (Novagen)
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagttattgctcagcggtggcagcagccaactcagcttcctttcgggctttgttagcagccggatccgacccatttgctgtccaccagtcatgctagccatatgtatatctccttcttaaagttaaacaaaattatttctagagggaaaccgttgtggtctccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaa

SEQ ID NO: 85 - DNA, 2686 bp, Artificial Sequence
  Description of Artificial Sequence pUC19 Vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 86 - DNA, 3171 bp, Artificial Sequence
  Description of Artificial Sequence pUC Plasmid M (minus MCS)
gaaagtcctctccactgactgtagcctccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctccttcctcttccagcccttcctctttc

SEQ ID NO: 87 - DNA, 5675 bp, Artificial Sequence
  Description of Artificial Sequence pET-11c Vector (Novagen)
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatccggatatagttcctcctttcagcaaaaaacccctcaagacccgtttagaggccccaaggggttatgctagttattgctcagcggtggcagcagccaactcagcttcctttcgggctttgttagcagccggatccgacccatttgctgtccaccagtcatgctagccatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 88 - DNA, 4338 bp, Artificial Sequence
  Description of Artificial Sequence pET-9d Plasmid
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagttattgctcagcggtggcagcagccaactcagcttcctttcgggctttgttagcagccggatccgacccatttgctgtccaccagtcatgctagccatggtatatctccttcttaaagttaaacaaaattatttctagagggaaaccgttgtggtctccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaa

SEQ ID NO: 89 - protein, 299 aa, Saponaria officinalis
  Ribosome-inactivating protein Saporin-6 Precursor
MKIYVVATIAWILLQFSAWTTTDAVTSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAESTALFPEATTANQKALEYTEDYQSIEKNAQITQGDQSRKELGLGIDLLSTSMEAVNKKARVVKDEARFLLIAIQMTAEAARFRYIQNLVIKNFPNKFNSENKVIQFEVNWKKISTAIYGDAKNGVFNKDYDFGFGKVRQVKDLQMGLLMYLGKPKSSNEANSTVRHYGPLKPTLLIT

SEQ ID NO: 90 - protein, 280 aa, Hordeum vulgare
  Ribosome-inactivating protein I (Barley)
AAKMAKNVDKPLFTATFNVQASSADYATFIAGIRNKLRNPAHFSHNRPVLPPVEPNVPPSRWFHVVLKASPTSAGLTLAIRADNIYLEGFKSSDGTWWELTPGLIPGATYVGFGGTYRDLLGDTDKLTNVALGRQQLADAVTALHGRTKADKPSGPKQQQAREAVTTLLLMVNEATRFQTVSGFVAGLLHPKAVEKKSGKIGNEMKAQVNGWQDLSAALLKTDVKPPPGKSPAKFAPIEKMGVRTAVQAANTLGILLFVEVPGGLTVAKALELFHASGGK

SEQ ID NO: 91 - protein, 280 aa, Hordeum vulgare
  Ribosome-inactivating protein II (Barley)
AAKMAKNVDKPLFTATFNVQASSADYATFIAGIRNKLRNPAHFSHNEPVLPPVEPNVPPSRWFHVVLKASPTSAGLTLAIRADNIYLEGFKSSDGTWWELTPGLIPGATYVGFGGTYRDLLGDTDKLTNVALGRQQLEDAVTALHGRTKADKASGPKQQQAREAVTTLLLMVNEATRFQTVSGFVAGLLHPKAVEKKSGKIGNEMKAQVNGWQDLSAALLKTDVKPPPGKSPAKFTPIEKMGVRTAEQAAATLGILLFVEVPGGLTVAKALELFHASGGK

SEQ ID NO: 92 - protein, 316 aa, Gelonium multiflorum
  Ribosome-inactivating protein Gelonin Precursor
MKGNMKVYWIKIAVATWFCCTTIVLGSTARIFSLPTNDEEETSKTLGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPKTSLAAELIIQNYESLVGFD

SEQ ID NO: 93 - protein, 576 aa, Ricinus communis
  Ricin Precursor
MKPGGNTIVIWMYAVATWLCFGSTSGWSFTLEDNNIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQFSLLIRPVVPNFNADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 94 - protein, 286 aa, Momordica charantia
  Ribosome-inactivating protein Momordin I Precursor
MSRFSVLSFLILAIFLGGSIVKGDVSFRLSGADPRSYGMFIKDLRNALPFREKVYNIPLLLPSVSGAGRYLLMHLFNYDGKTITVAVDVTNVYIMGYLADTTSYFFNEPAAELASQYVFRDARRKITLPYSGNYERLQIAAGKPREKIPIGLPALDSAISTLLHYDSTAAAGALLVLIQTTAEAARFKYIEQQIQERAYRDEVPSLATISLENSWSGLSKQIQLAQGNNGIFRTPIVLVDNKGNRVQITNVTSKVVTSNIQLLLNTRNIAEGDNGDVSTTHGFSSY

SEQ ID NO: 95 - protein, 286 aa, Momordica balsamina
  Ribosome-inactivating protein Momordin II Precursor
MVKCLLLSFLIIAIFIGVPTAKGDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVN

SEQ ID NO: 96 - protein, 290 aa, Bryonia dioica
  Ribosome-inactivating protein Bryodin I Precursor
MIKLLVLWLLILTIFLKSPTVEGDVSFRLSGATTTSYGVFIKNLREALPYERKVYNIPLLRSSISGSGRYTLLHLTNYADETISVAVDVTNVYIMGYLAGDVSYFFNEASATEAAKFVFKDAKKKVTLPYSGNYERLQTAAGKIRENIPLGLPALDSAITTLYYYTASSAASALLVLIQSTAESARYKFIEQQIGKRVDKTFLPSLATISLENNWSALSKQIQIASTNNGQFESPVVLIDGNNQRVSITNASARVVTSNIALLLNRNNIAAIGEDISMTLIGFEHGLYGI

SEQ ID NO: 97 - protein, 282 aa, Bryonia dioica
  Ribosome-inactivating protein Bryodin II Precursor
MRSIGFYSVLALYVGAHVTEDVDINFSLIGATGATYKTFIRNLRTKLTVGTPRVYDIPVLRNAAAGLARFQLVTLTNYNGESVTVALDVVNVYVVAYRAGNTAYFLADASTEANNVLFAGINHVRLPYGGNYDGLETAAGRISRENIELGFSEISSAIGNMFRHNPGTSVPRAFIVIIQTVSEAARFKYIEQRVSENVGTKFKPDPAFLSLQNAWGSLSEQIQIAQTRGGEFARPVELRTVSNTPTFVTNVNSPVVKGIALLLYFRVNVGTDNVFAMSLSTY

SEQ ID NO: 98 - protein, 261 aa, Phytolacca americana
  Ribosome-inactivating protein PAP-S
INTITFDAGNATINKYATFMESLRNEAKDPSLKCYGIPMLPNTNSTIKYLLVKLQGASLKTITLMLRRNNLYVMGYSDPYDNKCRYHIFNDIKGTEYSDVENTLCPSSNPRVAKPINYNGLYPTLEKKAGVTSRNEVQLGIQILSSDIGKISGQGSFTEKIEAKFLLVAIQMVSEAARFKYIENQVKTNFNRDFSPNDKVLDLEENWGKISTAIHNSKNGALPKPLELKNADGTKWIVLRVDEIKPDVGLLNYVNGTCQAT

SEQ ID NO: 99 - protein, 277 aa, Luffa cylindrica
  Ribosome-inactivating protein Luffin-alpha Precursor
MKRFTVLILAIFVAASTVEADVRFSLSGSSSTSYSKFIGDLRKALPSNGTVYNITLLLSSASGASRYTLMTLSNYDGKAITVAVDVTNVYIMGYLVNSTSYFFNESDAKLASQYVFKGSTIVTLPYSGNYEKLQTAAGKIREKIPLGFPALDSAITTLFHYDSTAAAAAFLVIIQTTAEASRFKYIEGQIIERISKNQVPSLATISLENEWSALSKQIQLAQTNNGTFKTPVVITDDKGQRVEITNVTSKVVTKNIQLLLNYKQNVAAFDEDVSAKH

SEQ ID NO: 100 - protein, 289 aa, Trichosanthes kirilowii
  Ribosome-inactivating protein alpha-trichosanthin Precursor
MIRFLVLSLLILTLFLTTPAVEGDVSFRLSGATSSSYGVFISNLRKALPNERKLYDIPLLRSSLPGSQRYALIHLTNYADETISVAIDVTNVYIMGYRAGDTSYFFNEASATEAAKYVFKDAMRKVTLPYSGNYERLQTAAGKIRENIPLGLPALDSAITTLFYYNANSAASALMVLIQSTSEAARYKFIEQQIGKRVDKTFLPSLAIISLENSWSALSKQIQIASTNNGQFESPVVLINAQNQRVTITNVDAGVVTSNIALLLNRNNMAAMDDDVPMTQSFGCGSYAI

SEQ ID NO: 101 - protein, 177 aa, Aspergillus clavatus
  Ribonuclease Clavin Precursor
MVAIKNLVLVALTAVTALAMPSPLEERAATWTCMNEQKNPKTNKYENKRLLYNQNNAESNAHHAPLSDGKTGSSYPHWFTNGYDGDGKILKGRTPIKWGNSDCDRPPKHSKNGDGKNDHYLLEFPTFPDGHQYNFDSKKPKEDPGPARVIYTYPNKVFCGIVAHTRENQGDLKLCSH

SEQ ID NO: 102 - protein, 528 aa, Abrus precatorius
  Abrin-a
QDRPIKFSTEGATSQSYKQFIEALRERLRGGLIHDIPVLPDPTTLQERNRYITVELSNSDTESIEVGIDVTNAYVVAYRAGTQSYFLRDAPSSASDYLFTGTDQHSLPFYGTYGDLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDSLSHPTVAVLALMLFVCNPPNANQSPLLIRSIVEKSKICSSRYEPTVRIGGRDGMCVDVYDNGYHNGNRIIMWKCKDRLEENQLWTLKSDKTIRSNGKCLTTYGYAPGSYVMIYDCTSAVAEATYWEIWDNGTIINPKSALVLSAESSSMGGTLTVQTNEYLMRQGWRTGNNTSPFVTSISGYSDLCMQAQGSNVWMADCDSNKKEQQWALYTDGSIRSVQNTNNCLTSKDHKQGSTILLMGCSNGWASQRWVFKNDGSIYSLYDDMVMDVKGSDPSLKQIILWPYTGKPNQIWLTLF

SEQ ID NO: 103 - protein, 300 aa, Zea mays
  Ribosome-inactivating protein 3 (Maize) Precursor
MAEITLEPSDLMAQTNKRIVPKFTEIFPVEDANYPYSAFIASVRKDVIKHCTDHKGIFQPVLPPEKKVPELWLYTELKTRTSSITLAIRMDNLYLVGFRTPGGVWWEFGKDGDTHLLGDNPRWLGFGGRYQDLIGNKGLETVTMGRAEMTRAVNDLAKKKKMATLEEEEVQMQMQMPEAADLAAAAAADPQADTKSKLVKLVVMVCEGLRFNTVSRTVDAGFNSQHGVTLTVTQGKQVQKWDRISKAAFEWADHPTAVIPDMQKLGIKDKNEAARIVALVKNQTTACATAASADNDDDEA

SEQ ID NO: 104 - protein, 304 aa, Zea mays
  Ribosome-inactivating protein 9 (Maize) Precursor
MAETNPELSDLMAQTNKKIVPKFTEIFPVEDVNYPYSAFIASVRKDVIKHCTDHKGIFQPVLPPEKKVPELWFYTELKTRTSSITLAIRMDNLYLVGFRTPGGVWWEFGKAGDTHLLGDNPRWLGFGGRYQDLIGNKGLETVTMGRAEMTRAVNDLAKKKKMATLEEEEVQMQMQMPEAAELAAAAAAADPQADTKSKLVKLVVMVCEGLRFNTVSRTVDAGFNSQHGVTLTVTQGKQVQKWDRISKAAFEWADHPTAVIPDMQKLGIKDKNEAARIVALVKNQTTAAAAAATAASADNDDDEA

SEQ ID NO: 105 - protein, 301 aa, Zea mays
  Ribosome-inactivating protein X (Maize) Precursor
MAEITLEPSDLMAQTNKRIVPKFTEIFPVEDANYPYSAFIASVRKDVIKHCTDHKGIFQPVLPPEKKVPELWFYTELKTRTSSITLAIRMDNLYLVGFRTPGGVWWEFGKDGDTHLLGDNPRWLGFGGRYQDLIGNKGLETVTMGRAEMTRAVNDLAKKKKMATLEEEEVKMQMQMPEAADLAAAAAADPQADTKSKLVKLVVMVCEGLRFNTVSRTVDAGFNSQHGVTLTVTQGKQVQKWDRISKAAFEWADHPTAVIPDMQKLGIKDKNEAARIVALVKNQTTAAAATAASADNDDDEA

SEQ ID NO: 106 - protein, 275 aa, Triticum aestivum
  Tritin Precursor
MAKNVDKPLFTATFNVQASSADYVTFINGIRNKLRNPGHSSHNRPVLPPIEPNVPPSRWFHIVLKTSPASTGLTLATRADNLYWEGFKSSDGTWWELTPGLIPGATHVGFGGTYRDLLGDTDKLTNVALGRQQMADAVTALYGRTKADKTSGPKQQQAREAVTTLLLMVHEATRFQTVSGFVAGVLHPKEKKSGKIGNEMKAQVNGWQDLSEALLKTDANAPPGKAPAKFTPIEKMGVRTAEQAAATLGILLFVQVPGGMTVAQALELFHKSGGK

SEQ ID NO: 107 - protein, 278 aa, Mirabilis jalapa
  Ribosome-inactivating protein MAP Precursor
MLTTTKVFFLLLTTWITWYAIVNPQSRAAPTLETIASLDLNNPTTYLSFITNIRTKVADKTEQCTIQKISKTFTQRYSYIDLIVSSTQKITLAIDMADLYVLGYSDIANNKGRAFFFKDVTEAVANNFFPGATGTNRIKLTFTGSYGDLEKNGGLRKDNPLGIFRLENSIVNIYGKAGDVKKQAKFFLLAIQMVSEAARFKYISDKIPSEKYEEVTVDEYMTALENNWAKLSTAVYNSKPSTTTATKCQLATSPVTISPWIFKTVEEIKLVMGLLKSS

SEQ ID NO: 108 - protein, 293 aa, Dianthus caryophyllus
  Ribosome-inactivating protein Dianthin 30 Precursor
MKIYLVAAIAWILFQSSSWTTDAATAYTLNLANPSASQYSSFLDQIRNNVRDTSLIYGGTDVAVIGAPSTTDKFLRLNFQGPRGTVSLGLRRENLYVVAYLAMDNANVNRAYYFKNQITSAELTALFPEVVVANQKQLEYGEDYQAIEKNAKITTGDQSRKELGLGINLLITMIDGVNKKVRVVKDEARFLLIAIQMTAEAARFRYIQNLVTKNFPNKFDSENKVIQFQVSWSKISTAIFGDCKNGVFNKDYDFGFGKVRQAKDLQMGLLKYLGRPKSSSIEANSTDDTADVL

SEQ ID NO: 109 - protein, 563 aa, Sambucus nigra
  Nigrin b Precursor
MRVVAAAMLYFYIVVLAICSVGIQGIDYPSVSFNLDGAKSATYRDFLSNLRKTVATGTYEVNGLPVLRRESEVQVKSRFVLVPLTNYNGNTVTLAVDVTNLYVVAFSGNANSYFFKDATEVQKSNLFVGTKQNTLSFTGNYDNLETAANTRRESIELGPSPLDGAITSLYHGDSVARSLLVVIQMVSEAARFRYIEQEVRRSLQQATSFTPNALMLSMENNWSSMSLEIQQAGNNVSPFFGTVQLLNYDHTHRLVDNFEELYKITGIAILLFRCSSPSNDNAIRMPLDLAGEDNKYNDGETCTLRTSFTRNIVGRDGLCVDVRNGYDTDGTPLQLWPCGTQRNQRWTFDSDDTIRSMGKCMTANGLNNGSNIVIFNCSTAAENAIKWEVPIDGSIINPSSGLVMTAPRAASRTILLLEDNIYAASQGWTVTNNVKPIVASIVGYKEMCLQSNGENNGVWMEDCEATSLQQQWALYGDRTIRVNSTRGLCVTTNGYNSKDLIIILKCQGLPSQRWFFNSDGAIVNPKSRHVMDVRASNVSLREIIIFPATGNPNQQWVTQVLPS

SEQ ID NO: 110 - protein, 563 aa, Sambucus nigra
  Ribosome-inactivating protein Nigrin I Precursor
MRVVAAAMLYFYIVVLAICSVGIQGIDYPSVSFNLDGAKSATYRDFLSNLRKTVATGTYEVNGLPVLRRESEVQVKSRFVLVPLTNYNGNTVTLAVDVTNLYVVAFSGNANSYFFKDATEVQKSNLFVGTKQNTLSFTGNYDNLETAANTRRESIELGPSPLDGAITSLYHGDSVARSLLVVIQMVSEAARFRYIEQEVRRSLQQATSFTPNASMLSMENNWSSMSLEIQQAGNNVSPFSGTVQLLNYDHTHRLVDNFEELYKITGIAILLFRCSSPSNDNAIRMPLDLAGEDNKYNDGETCTLRTSFTRNIVGRDGLCVDVRNGYDTDGTPLQLWPCGTQRNQRWTFNTDDTIRSMGKCMTANGLNNGSNIVIFNCSTAVENAIKWEVPIDGSIINPSSGRVVTAPSAASRTILLLEDNIYAASQGWTVTNNVKPIVASIVGYKEMCLQSNGENNGVWMEDCEATSLQQQWALYGDRTIRVNSTRGLCVTTNGYNSKDLIIILKCQGLPSQRWFFNSDGAIVNPKSRLVMDVRASNVSLREIIIFPATGNPNQQWVTQVLPS

SEQ ID NO: 111 - protein, 564 aa, Sambucus ebulus
  Ribosome-inactivating protein Ebulin 1 Precursor
MRVVKAAMLYLHIVVLAIYSVGIQGIDYPSVSFNLAGAKSTTYRDFLKNLRDRVATGTYEVNGLPVLRRESEVQVKNRFVLVRLTNYNGDTVTSAVDVTNLYLVAFSANGNSYFFKDATELQKSNLFLGTTQHTLSFTGNYDNLETAAGTRRESIELGPNPLDGAITSLWYDGGVARSLLVLIQMVPEAARFRYIEQEVRRSLQQLTSFTPNALMLSMENNWSSMSLEVQLSGDNVSPFSGTVQLQNYDHTPRLVDNFEELYKITGIAILLFRCVATKTTHNAIRMPHVLVGEDNKFNDGETCAIPAPFTRRIVGRDGLCVDVRNGYDTDGTPIQLWPCGTQRNQQWTFYNDKTIRSMGKCMTANGLNSGSYIMITDCSTAAEDATKWEVLIDGSIINPSSGLVMTAPSGASRTTLLLENNIHAASQGWTVSNDVQPIATLIVGYNEMCLQANGENNNVWMEDCDVTSVQQQWALFDDRTIRVNNSRGLCVTSNGYVSKDLIVIRKCQGLATQRWFFNSDGSVVNLKSTRVMDVKESDVSLQEVIIFPATGNPNQQWRTQVPQI

SEQ ID NO: 112 - protein, 99 aa, Homo sapien
  MCP-1 Precursor
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 113 - protein, 97 aa, Homo sapien
  Eotaxin Precursor
MKVSAALLWLLLIAAAFSPQGLAGPASVPTTCCFNLANRKIPLQRLESYRRITSGKCPQKAVIFKTKLAKDICADPKKKWVQDSMKYLDQKSPTPKP

SEQ ID NO: 114 - protein, 93 aa, Homo sapien
  SDF-1 Precursor
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 115 - protein, 107 aa, Homo sapien
  GRO-alpha Precursor
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 116 - protein, 92 aa, Homo sapien
  MIP-1-beta Precursor
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 117 - protein, 99 aa, Homo sapien
  IL-8 Precursor
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 118 - protein, 98 aa, Homo sapien
  IP-10 Precursor
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 119 - protein, 99 aa, Homo sapien
  MCP-3 Precursor
MKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 120 - protein, 96 aa, Homo sapien
  MIP-3-alpha Precursor
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 121 - protein, 93 aa, Homo sapien
  MDC Precursor
MARLQTALLVVLVLLAVALQATEAGPYGANMEDSVCCRDYVRYRLPLRVVKHFYWTSDSCPRPGVVLLTFRDKEICADPRVPWVKMILNKLSQ

SEQ ID NO: 122 - protein, 92 aa, Homo sapien
  MIP-1-alpha Precursor
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 123 - protein, 109 aa, Homo sapien
  BCA-1 Precursor
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 124 - protein, 114 aa, Homo sapien
  GCP-2 Precursor
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLKKVIQKILDSGNKKN

SEQ ID NO: 125 - protein, 114 aa, Homo sapien
  ENA-78 Precursor
MSLLSSRAARVPGPSSSLCALLVLLLLLTQPGPIASAGPAAAVLRELRCVCLQTTQGVHPKMISNLQVFAIGPQCSKVEVVASLKNGKEICLDPEAPFLKKVIQKILDGGNKEN

SEQ ID NO: 126 - protein, 128 aa, Homo sapien
  PBP Precursor
MSLRLDTTPSCNSARPLHALQVLLLLSLLLTALASSTKGQTKRNLAKGKEESLDSDLYAELRCMCIKTTSGIHPKNIQSLEVIGKGTHCNQVEVIATLKDGRKICLDPDAPRIKKIVQKKLAGDESAD

SEQ ID NO: 127 - protein, 125 aa, Homo sapien
  MIG Precursor
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 128 - protein, 101 aa, Homo sapien
  PF-4 Precursor
MSSAAGFCASRPGLLFLGLLLLPLVVAFASAEAEEDGDLQCLCVKTTSQVRPRHITSLEVIKAGPHCPTAQLIATLKNGRKICLDLQAPLYKKIIKKLLES

SEQ ID NO: 129 - protein, 104 aa, Homo sapien
  PF4 Variant Precursor
MSSAARSRLTRATRQEMLFLALLLLPVVVAFARAEAEEDGDLQCLCVKTTSQVRPRHITSLEVIKAGPHCPTAQLIATLKNGRKICLDLQALLYKKIIKEHLES

SEQ ID NO: 130 - protein, 211 aa, Homo sapien
  SDF-2 Precursor
MAVVPLLLLGGLWSAVGASSLGVVTCGSVVKLLNTRHNVRLHSHDVRYGSSSGQQSVTGVTSVDDSNSYWRIRRKSATVCERGTPIKCGQPIRLTHVNTGRNLHSHHFTSPLSGNQEVTAFGEEGEGDYLDDWTVLCNGPYWVRDGEVRFKHSSTEVLLSVTGEQYGRPISGQKEVHGMAQPSQNNYWKAMEGIFMKPSELLKAEAHHAEL

SEQ ID NO: 131 - protein, 99 aa, Homo sapien
  MCP-2 Precursor
MKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

SEQ ID NO: 132 - protein, 98 aa, Homo sapien
  MCP-4 Precursor
MKVSAVLLCLLLMTAAFNPQGLAQPDALNVPSTCCFTFSSKKISLQRLKSYVITTSRCPQKAVIFRTKLGKEICADPKEKWVQNYMKHLGRKAHTLKT

SEQ ID NO: 133 - protein, 89 aa, Homo sapien
  MIP-4 Precursor
MKGLAAALLVLVCTMALCSCAQVGTNKELCCLVYTSWQIPQKFIVDYSETSPQCPKPGVILLTKRGRQICADPNKKWVQKYISDLKLNA

SEQ ID NO: 134 - protein, 98 aa, Homo sapien
  MIP-3-beta Precursor
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 135 - protein, 107 aa, Homo sapien
  MIP2-alpha Precursor
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 136 - protein, 107 aa, Homo sapien
  MIP2-beta Precursor
MAHATLSAAPSNPRLLRVALLLLLLVAASRRAAGASVVTELRCQCLQTLQGIHLKNIQSVNVRSPGPHCAQTEVIATLKNGKKACLNPASPMVQKIIEKILNKGSTN

SEQ ID NO: 137 - protein, 113 aa, Homo sapien
  MIP-5 Precursor
MKVSVAALSCLMLVAVLGSQAQFINDAETELMMSKLPLENPVVLNSFHFAADCCTSYISQSIPCSLMKSYFETSSECSKPGVIFLTKKGRQVCAKPSGPGVQDCMKKLKPYSI

SEQ ID NO: 138 - protein, 93 aa, Homo sapien
  HCC-1 Precursor
MKISVAAIPFFLLITIALGTKTESSSRGPYHPSECCFTYTTYKIPRQRIMDYYETNSQCSKPGIVFITKRGHSVCTNPSDKWVQDYIKDMKEN

SEQ ID NO: 139 - protein, 91 aa, Homo sapien
  RANTES Precursor
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 140 - protein, 119 aa, Homo sapien
  Eotaxin-2 Precursor
MAGLMTIVTSLLFLGVCAHHIIPTGSVVIPSPCCMFFVSKRIPENRVVSYQLSSRSTCLKAGVIFTTKKGQQFCGDPKQEWVQRYMKNLDAKQKKASPRARAVAVKGPVQRYPGNQTTC

SEQ ID NO: 141 - protein, 94 aa, Homo sapien
  TARC Precursor
MAPLKMLALVTLLLGASLQHIHAARGTNVGRECCLEYFKGAIPLRKLKTWYQTSEDCSRDAIVFVTVQGRAICSDPNNKRVKNAVKYLQSLERS

SEQ ID NO: 142 - protein, 96 aa, Homo sapien
  I-309 Precursor
MQIITTALVCLLLAGMWPEDVDSKSMQVPFSRCCFSFAEQEIPLRAILCYRNTSSICSNEGLIFKLKRGKEACALDTVGWVQRHRKMLRHCPSKRK

SEQ ID NO: 143 - protein, 114 aa, Homo sapien
  Lymphotactin Precursor
MRLLILALLGICSLTAYIVEGVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 144 - protein, 167 aa, Homo sapien
  Lungkine Precursor
MAAQGWSMLLLAVLNLGIFVRPCDTQELRCLCIQEHSEFIPLKLIKNIMVIFETIYCNRKEVIAVPKNGSMICLDPDAPWVKATVGPITNRFLPEDLKQKEFPPAMKLLYSVEHEKPLYLSFGRPENKRIFPFPIRETSRHFADLAHNSDRNFLRDSSEVSLTGSDA

SEQ ID NO: 145 - protein, 116 aa, Mus musculus
  C10 Precursor
MRNSKTAISFFILVAVLGSQAGLIQEMEKEDRRYNPPIIHQGFQDTSSDCCFSYATQIPCKRFIYYFPTSGGCIKPGIIFISRRGTQVCADPSDRRVQRCLSTLKQGPRSGNKVIA

SEQ ID NO: 146 - protein, 122 aa, Mus musculus
  MIP-1-gamma Precursor
MKPFHTALSFLILTTALGIWAQITHATETKEVQSSLKAQQGLEIEMFHMGFQDSSDCCLSYNSRIQCSRFIGYFPTSGGCTRPGIIFISKRGFQVCANPSDRRVQRCIERLEQNSQPRTYKQ

SEQ ID NO: 147 - protein, 104 aa, Mus musculus
  MCP-5 Precursor
MKISTLLCLLLIATTISPQVLAGPDAVSTPVTCCYNVVKQKIHVRKLKSYRRITSSQCPREAVIFRTILDKEICADPKEKWVKNSINHLDKTSQTFILEPSCLG

SEQ ID NO: 148 - protein, 120 aa, Homo sapien
  LEC Precursor
MKVSEAALSLLVLILIITSASRSQPKVPEWVNTPSTCCLKYYEKVLPRRLVVGYRKALNCHLPAIIFVTKRNREVCTNPNDDWVQEYIKDPNLPLLPTRNLSTVKIITAKNGQPQLLNSQ

SEQ ID NO: 149 - protein, 134 aa, Homo sapien
  Exodus-2 Precursor
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 150 - protein, 120 aa, Homo sapien
  MIP-3 Precursor
MKVSVAALSCLMLVTALGSQARVTKDAETEFMMSKLPLENPVLLDRFHATSADCCISYTPRSIPCSLLESYFETNSECSKPGVIFLTKKGRRFCANPSDKQVQVCMRMLKLDTRIKTRKN

SEQ ID NO: 151 - protein, 150 aa, Homo sapien [sequence omitted]

SEQ ID NO: 152 - protein, 94 aa, Homo sapien
  Eotaxin-3 Precursor
MMGLSLASAVLLASLLSLHLGTATRGSDISKTCCFQYSHKPLPWTWVRSYEFTSNSCSQRAVIFTTKRGKKVCTHPRKKWVQKYISLLKTPKQL

SEQ ID NO: 153 - protein, 112 aa, Homo sapien
  CTACK Precursor
MKGPPTFCSLLLLSLLLSPDPTAAFLLPPSTACCTQLYRKPLSDKLLRKVIQVELQEADGDCHLQAFVLHLAQRSICIHPQNPSLSQWFEHQERKLHGTLPKLNFGMLRKMG

SEQ ID NO: 154 - protein, 127 aa, Homo sapien
  MEC Precursor
MQQRGLAIVALAVCAALHASEAILPIASSCCTEVSHHISRRLLERVNMCRIQRADGDCDLAAVILHVKRRRICVSPHNHTVKQWMKVQAAKKNGKGNVCHRKKHHGKRNSNRAHQGKHETYGHKTPY

SEQ ID NO: 155 - protein, 114 aa, Homo sapien
  SCM-1 beta Precursor
MRLLILALLGICSLTAYIVEGVGSEVSHRRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 156 - protein, 94 aa, Homo sapien
  I-TAC Precursor
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 157 - protein, 99 aa, Homo sapien
  BRAK Precursor
MRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 158 - protein, 254 aa, Homo sapien [sequence omitted]

SEQ ID NO: 159 - protein, 395 aa, Homo sapien
  Fractalkine Precursor
MAPSPLAWLLRLAAFFHLCTLLPGQHLGMTKCEIMCGKMTSRIPVALLIRYQLNQESCGKRAIVLETTQHRRFCADPKEKWVQDAMKHLDHQAAALTKNGGKFEKRVDNVTPGITLATRGLSPSALTKPESATLEDLALELTTISQEARGTMGTSQEPPAAVTGSSLSTSEAQDAGLTAKPQSIGSFEAADISTTVWPSPAVYQSGSSSWAEEKATESPSTTAPSPQVSTTSPSTPEENVGSEGQPPWVQGQDLSPEKSLGSEEINPVHTDNFQERGPGNTVHPSVAPISSEETPSPELVASGSQAPKIEEPIHATADPQKLSVLITPVPDTQAATRRQAVGLLAFLGLLFCLGVAMFAYQSLQGCPRKMAGEMVEGLRYVPRSCGSNSYVLVPV

SEQ ID NO: 160 - protein, 93 aa, Homo sapien
  LD78-beta Precursor
MQVSTAALAVLLCTMALCNQVLSAPLAADTPTACCFSYTSRQIPQNFIADYFETSSQCSKPSVIFLTKRGRQVCADPSEEWVQKYVSDLELSA

SEQ ID NO: 161 - protein, 92 aa, Homo sapien
  MIP-1b2 Precursor
MKLCVTVLSLLVLVAAFCSLALSAPMGSDPPTACCFSYTARKLPRNFVVDYYETSSLCSQPAVVFQTKRGKQVCADPSESWVQEYVYDLELN

SEQ ID NO: 162 - protein, 299 aa, Saponaria officinalis
  Saporin Variants
MKIYVVATIAWILLQFSAWTTTDAVTSITLDLVNPTAGQYSSFVDKIRNNVKDPNLKYGXTDIAVIGPPSKXKFLRINFQSSRGTVSLGLKRDNLYVVAYLAMDNTNVNRAYYFXSEITSAEXTALFPEATTANQKALEYTEDYQSIEKNAQITQGDQSRKELGLGIDLLSTSMEAVNKKARVVKDEARFLLIAIQMTAEAARFRYIQNLVXKNFPNKFNSENKVIQFEVNWKKISTAIYGDAKNGVFNKDYDFGFGKVRQVKDLQMGLLMYLGKPKSSNEANSTVRHYGPLKPTLLIT

SEQ ID NO: 163 - protein, 316 aa, Gelonium multiflorum
  Gelonin Variants
MKGNMKVYWIKIAVATWFCCTTIVLGSTARIFSLPTNDEEETSKTLGLDTVSFSTKGATYITYVNFLNELRVKLKPEGNSHGIPLLRKKXDDXGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPKTSLAAELIIQNYESLVGFD

SEQ ID NO: 164 - protein, 576 aa, Ricinus communis
  Ricin A Variants
MKPGGNTIVIWMYAVATWLCFGSTSGWSFTLEDNNIFPKQYPIINFTTAGATVQSYTNFIRAVRGRLTTGADVRHXIPVLPNXVGLPINQRFILVELSNHAELSVTLAXXXTNAYVVGYRAGNSAYFFHPDXQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQFSLLIRPVVPNFNADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRXSDPSLKQIILYPLHGDPNQIWLPLF

SEQ ID NO: 165 - protein, 290 aa, Bryonia dioica
  Bryodin I Variants
MIKLLVLWLLILTIFLKSPTVEGDVSFRLSGATTTSYGVFIKNLREALPYERKVYNIPLLRSSISGSGRYTLLHLTNYADETISVAVDVTNVYIMGYLAGDVSYFFNEASATEAAKFVFKDAKKKVTLPYSGNYERLQTAAGKIRENIPLGLPALDSAITTLYYYTASSAASALLVLIQSTAESARYKFIEQQIGKRVDKTFLPSLATISLXNNWSALSKQIQIASTNNGQFESPVVLIDGNNQRVSITNASARVVTSNIALLLNRNNIAAIGEDISMTLIGFEHGLYGI

SEQ ID NO: 166 - protein, 289 aa, Thrichosanthes kirilowii
  Trichosanthin Variants
MIRFLVLSLLILTLFLTTPAVEGDVSFRLSGATSSSYGVFISNLRKALPNERKLYDIPLLRSSLPGSQRYALIHLTNYADETISVAXDVTNVYIMGYRAGDTSYFFNEASATEAAKYVFKDAMRKVTLPYSGNYERLQTAAGKIRENIPLGLPALDSAITTLFYYNANSAASALMVLIQSTSEAARYKFIEQQIGXRVDKTFLPSLAIISLENSWSALSKQIQIASTNNGXFEXPVVLINAQNQRVXITNVDAGVVTSNIALLLNRNNMAAMDDDVPMTQSFGCGSYAI

SEQ ID NO: 167 - protein, 528 aa, Abrus precatorius
  Abrin Variants
XDRPIKFSTEGATSQSYKQFIEALRERLRGGLIHDIPVLPDPTTLQERNRYITVELSNSDTESIEVGIDVTNAYVVAYRAGTQSYFLRDAPSSASDYLFTGTDQHSLPFYGTYGDLERWAHQSRQQIPLGLQALTHGISFFRSGGNDNEEKARTLIVIIQMVAEAARFRYISNRVRVSIQTGTAFQPDAAMISLENNWDXLSRGVQESVQDTFPNQVTLTNIRNEPVIVDSLSHPTVAVLALMLFVCNPPNANQSPLLIRSIVEKSKICSSRYEPTVRIGGRDGMCVDVYDNGYHNGXRIIMWKCKDRLEENQLWTLKSDKTIRSNGKCLTTYGYAPGSYVMIYDCTSAVAEATYWEIWDNGTIINPKSALVLSAESSSMGGTLTVQTNEYLMRQGWRTGNNTSPFVTSISGYSDLCMQAQGSNVWXADCDSNKKEQQWALYTDGSIRSVQNTNNCLTSKDHKQGSXILLMGCSNGWASQRWXFKNDGSIYSLYDDMVMDVKGSDPSLKQIILWPYTGKPNQIWLTLF

SEQ ID NO: 168 - protein, 278 aa, Mirabilis jalapa
  MAP Variants
MLTTTKVFFLLLTTWITWYAIVNPQSRAAPTLETXASLDLNNPTTYLSFITNIRTKVXDKTEQCTIQKISKTFTQRYSYIDLIVSSTQKITLAIDMADLYVLGYSDIANNKGRAFFFKDVTEAVANNFFPGATGTNRIKLTFTGSYGDLEKNGGLRKDNPLGIFRLENSIVNIYGKAGDXKKQAKFFLLAIQMVSEAARFKYISDKIPSEKYEEVTVXEYMTALENNWAKLSTAVYNSKPSTTTATKCQLATSPVTISPWIFKTVEEIKLVMGLLKSS

SEQ ID NO: 169 - protein, 563 aa, Sambucus nigra
  Nigrin b Variants
MRVVAAAMLYFYIVVLAICSVGIQGIDYPSVSFNLDGAXSATYRDFLSNLRKTVATGTYEVNGLPVLRRESEVQVKSRFVLVPLTNYNGNTVTLAVDVTNLYVVAFSGNANSYFFKDATEVQKSNLFVGTKQNTLSFTGNYDNLETAANTRRESIELGPSPLDGAITSLYHGDSVARSLLVVIQMVSEAARFRYIEQEVRRSLQQATSFTPNALMLSMENNWSSMSLEIQQAGNNVSPFFGTVQLLNYDHTHRLVDNFEELYKITGIAILLFRCSSPSNDNAIRMPLDLAGEDNKYNDGETCTLRTSFTRNIVGRDGLCVDVRNGYDTDGTPLQLWPCGTQRNQRWTFDSDDTIRSMGKCMTANGLNNGSNIVIFNCSTAAENAIKWEVPIDGSIINPSSGLVMTAPRAASRTILLLEDNIYAASQGWTVTNNVKPIVASIVGYKEMCLQSNGENNGVWMEDCEATSLQQQWALYGDRTIRVNSTRGLCVTTNGYNSKDLIIILKCQGLPSQRWFFNSDGAIVNPKSRHVMDVRASNVSLREIIIFPATGNPNQQWVTQVLPS

SEQ ID NO: 170 - protein, 99 aa, Homo sapien
  MCP-1 Variants
MKVSAALLCLLLIAATFIPQGLAQPXAXXAXXXCCXNFTNRKISVQXLAXXRXITSSKCPKEAVIFKTIVAKEICADPXQXWVQDSMDXLXKQTQTPKT

SEQ ID NO: 171 - protein, 97 aa, Homo sapien
  Eotaxin Variants
MKVSAAXLWLLLIAAAFSPQGLXGPASVPTTCCFNLANRKIPLQRLESYRXITSGKCPQKAVIFKTKLAKDICADPKKXWVQDSMKYLDQKSPTPKP

SEQ ID NO: 172 - protein, 92 aa, Homo sapien
  MIP-1-beta Variants
MKLCVXVLSLLMLVXAFCSPALSAPMGSDPPTACCFSYTARKLPRNFVVDYYETSXLCSQPAVVFQTKRXKQVCADPSEXWVQEYVYDLELN

SEQ ID NO: 173 - protein, 99 aa, Homo sapien
  IL-8 Variants
MTSKLAVALLAAFLISAALCEGAVLPRSAKELRCQCIKTYSKPFHPKFIKELXVIESGPHCANTEIIVKLSDGRELCLDPKENWVQRVVEKFLKRAENS

SEQ ID NO: 174 - protein, 98 aa, Homo sapien
  IP-10 Variants
MNQTAILICCLIFLTLSGIQGVPLSRTVRCTCISISNQPVNPRSLEKLEIIPASQFCPRVEIIATMKKKGEKRCLNPESKAIKNLLKAVSKEXSKRSP

SEQ ID NO: 175 - protein, 99 aa, Homo sapien
  MCP-3 Variants
MKASAALLCLLLTAAAFSPQGLAQPVGINXSTTCCYRFINKKIPKQRLESYRRTTSSHCPREAVIFKTKLDKEICADPTQKWVQDFMKHLDKKTQTPKL

SEQ ID NO: 176 - protein, 96 aa, Homo sapien
  MIP-3-alpha Variants
MCCTKSLLLAALMSVLLLHLCGESEAASNFDCCLGYTDRILHPKFIXGFTRQLANEGCDINAIIFHTKKKLSVCANPKQTWVKYIVRLLSKKVKNM

SEQ ID NO: 177 - protein, 92 aa, Homo sapien
  MIP-1-alpha Variants
MQVSTAALAVLLCTMALCNQFSASLAADTPTACCFSYTSXQIPQNFIAXYFETSSQCSKPGVIFLTKXSXQVCADPSEEWVQKYVSDLXLSA

SEQ ID NO: 178 - protein, 101 aa, Homo sapien
  PF-4 Variants
MSSAAGFCASRPGLLFLGLLLLPLVVAFASAEAEEDGDLQCLCVKTTSQVRPRHITSLEVIKAGPHCPTAQLIATLKXGRKICLDLQAPLYKKIIKKLLES

SEQ ID NO: 179 - protein, 104 aa, Homo sapien
  PF-4 var1 Variants
MSSAARSRLTRATRQEMLFLALLLLPVVVAFARAEAEEDGDLQCLCVKTTSQVRPRHITSLEVIKAGPHCPTAQLIXTLKNGRKICLDLQALLYKKIIKEHLES

SEQ ID NO: 180 - protein, 211 aa, Homo sapien
  SDF-2 Variants
MAVVPLLLLGGLWSAVGASSLGVVTCGSVVKLLNTRHNVRLHSHDVRYGSXSGQQSVTGVTSVDDSNSYWRIRXKSATVCERGTPIKCGQPIRLTHVNTGRNLHSHHFTSPLSGNQEVXAFGEEGEGDYLDDWTVLCNGPYWVRDGEVRFKHSSTEVLLSVTGEQYGRPISGQKEVHGMAQPSQNNYWKAMEGIFMKPSELLKAEAHHAEL

SEQ ID NO: 181 - protein, 99 aa, Homo sapien
  MCP-2 Variants
MKVSAALLCLLLMAATFSPQGLAQPDSVSIPITCCFNVINRKIPIQRLESYTRITNIQCPKEAVIFKTXRGKEVCADPKERWVRDSMKHLDQIFQNLKP

SEQ ID NO: 182 - protein, 113 aa, Homo sapien
  MIP-5 Variants
MKVSVAALSCLMLXAVLGSQAQFXNDAETELMMSKLPLENPVVLNSFHFAADCCTSYISQSIPCSLMKSYFETSSECSKPGVIFLTKKGRQVCAKPSGPGVQDCMKKLKPYSI

SEQ ID NO: 183 - protein, 119 aa, Homo sapien
  Eotaxin-2 Variants
MAGLMTIVTSLLFLGVCAHHIIPTGSVVXPSPCCMFFVSKRIPENRVVSYQLSSRSTCLKXGVIFTTKKGQQXCGDPKQEWVQRYMKNLDAKQKKASPRARAVAVKGPVQRYPGNQTTC

SEQ ID NO: 184 - protein, 116 aa, Mus musculus
  C10 Variants
MRNSKTAISFFILVAVLGSQAGLIQEMEKEDRRYNPPIIHQGXQDTSSDCCFSYATQIPCKRFIYYFPTSGGCIKPGIIFISRRGTQVCADPXDRRVQRCLSTLKQGPRSXNKVIA

SEQ ID NO: 185 - protein, 122 aa, Mus musculus
  MIP-1-gamma Variants
MKPFHTALSFLILTTALGIWAQITHATETKEVQSSLKAQQGLEIEMFHMGFQDSSDCCLSYNSRIQCSRFIGYFPTSGGCTRPGIIFISKRGFQVCANPSDRRVQRCIERLEXNSQPRTYKQ

SEQ ID NO: 186 - protein, 120 aa, Homo sapien
  MIP-3 Variants
MKVSVAALSCLMLVTALGSQARVTKDAETEFMMSKLPLENPVLLDRFHATSADCCISYTPRSIPCSLLESYFETNSECSKPGVIFLTKKGRRFCANPSDKQVQVCXRMLKLDTRIKTRKN

SEQ ID NO: 187 - protein, 112 aa, Homo sapien
  CTACK Variants
MKGPPTFCSLLLLSLLLSPDPTAAFLLPPSTACCTQLYRKPLSDKLLRKVIQVELQEADGDCHLQAFVLHLAQRSICXHPQNPSLSQWFEHQERKXHGTLPKLNFGMLRKMG

SEQ ID NO: 188 - protein, 94 aa, Homo sapien
  I-TAC Variants
MSVKGMAIALAVILCATVVQGFPMFKRGRCLCIGPGVKAVKVADIEKASIMYPSNNCDKIEVIITLKENKGQRCLNPKSKQARLIIKKXERKNF

SEQ ID NO: 189 - protein, 254 aa, Homo sapien
  SR-PSOX Variants
MGRDLRPGSRVLLLLLLLLLVYLTQPGNGNEGSVTGSCYCGKRISSDSPPSVQFMNRLRKHLRAYHRCLYYTRFQLLSWSVCGGNKDPWVQELMSCLDLKECGHAYSGIVAHQKHLLPTSPPXSQASEGASSDIXTPAQMLLSTLQSTQRPTLPVGSLSSDKELTRPNETTIHTAGHSLAXGPEAGENQKQPEKNAGPTARTSATVPVLCLLAIIFILTAAXSYVLCKRRRGQSPQSSPDLPVHYIPVAPDSNT

SEQ ID NO: 190 - protein, 395 aa, Homo sapien
  Fractalkine Variants
MAPSPLAWLLRLAAFFHLCTLLPGQHLGMTKCEIMCXKMTSRIPVALLIRYQLNQESCGKRAIVLETTQHRRFCADPKEKWVQDAMKHLDHQAAALTKNGGKFEKRVDNVTPGITLATRGLSPSALTKPESATLEDLALELTTISQEARGTMGTSQEPPAAVTGSSLSTSEAQDAGLTAKPQSIGSFEAADISTTVWPSPAVYQSGSSSWAEEKATESPSTTAPSPQVSTTSPSTPEENVGSEGQPPWVQGQDLSPEKSLGSEEINPVHTDNFQERGPGNTVHPSVAPISSEETPSPELVASGSQAPKIEEPIHATADPQKLSVLITPVPDTQAATRRQAVGLLAFLGLLFCLGVAMFAYQSLQGCPRKMAGEMVEGLRYVPRSCGSNSYVLVPV

SEQ ID NO: 191 - protein, 93 aa, Homo sapien
  LD78-beta Variants
MQVSTAALAVLLCTMALCNQVLSAPLAADTPTACCFSYTSRQIPQNFIADYFETSSQCSKPSVIFLTKRGRQVCADPSEEWVQKYVSDLEXSA

SEQ ID NO: 192 - protein, 5 aa, Artificial Sequence
  Description of Artificial Sequence Linker peptide
GGGGS

SEQ ID NO: 193 - protein, 5 aa, Artificial Sequence
  Description of Artificial Sequence Linker peptide
GGGGS

SEQ ID NO: 194 - protein, 12 aa, Artificial Sequence
  Description of Artificial Sequence Linker peptide
GKSSGSGSESKS

SEQ ID NO: 195 - protein, 14 aa, Artificial Sequence
  Description of Artificial Sequence Linker peptide
GSTSGSGKSSEGKG

SEQ ID NO: 196 - protein, 18 aa, Artificial Sequence
  Description of Artificial Sequence Linker peptide
GSTSGSGKSSEGSGSTKG

SEQ ID NO: 197 - protein, 14 aa, Artificial Sequence
  Description of Artificial Sequence Linker peptide
GSTSGSGKSSEGKG

SEQ ID NO: 198 - protein, 18 aa, Artificial Sequence
  Description of Artificial Sequence Linker peptide
GSTSGSGKPGSGEGSTKG

SEQ ID NO: 199 - protein, 14 aa, Artificial Sequence
  Description of Artificial Sequence Linker peptide
EGKSSGSGSESKEF

SEQ ID NO: 200 - protein, 5 aa, Artificial Sequence
  Description of Artificial Sequence Linker peptide
SRSSG

SEQ ID NO: 201 - protein, 5 aa, Artificial Sequence
  Description of Artificial Sequence Linker peptide
SGSSC

SEQ ID NO: 202 - protein, 28 aa, Corynebacterium diphtheriae
  Diphtheria toxin trypsin sensitive peptide linker
AMGRSGGGCAGNRVGSSLSCGGLNLQAM

SEQ ID NO: 203 - protein, 6 aa, Artificial Sequence
  Description of Artificial Sequence Linker peptide
AMGSAM

SEQ ID NO: 204 - protein, 232 aa, Homo sapien
  VEGF-A Precursor (splice variant 206)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 205 - protein, 215 aa, Homo sapien
  VEGF-A Precursor (splice variant 189)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 206 - protein, 209 aa, Homo sapien
  VEGF-A Precursor (splice variant 183)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 207 - protein, 191 aa, Homo sapien
  VEGF-A (splice variant 165)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 208 - protein, 174 aa, Homo sapien
  VEGF-A Precursor (splice variant 148)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 209 - protein, 171 aa, Homo sapien
  VEGF-A Precursor (splice variant 145)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 210 - protein, 147 aa, Homo sapien
  VEGF-A Precursor (splice variant 121)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENCDKPRR

SEQ ID NO: 211 - protein, 207 aa, Homo sapien
  VEGF-B Precursor (splice variant 186)
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 212 - protein, 188 aa, Homo sapien
  VEGF-B Precursor (splice variant 167)
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 213 - protein, 419 aa, Homo sapien
  VEGF-C Precursor
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 214 - protein, 354 aa, Homo sapien
  VEGF-D Precursor
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 215 - protein, 170 aa, Homo sapien
  Placental Growth Factor Precursor
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 216 - DNA, 963 bp, Artificial Sequence
  Description of Artificial Sequence Construct encoding chemokine-toxin fusion protein SDF-1beta-AM-truncated Shiga-A1 Subunit
aagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatggcgatgaaagaattcaccctggacttttccactgcaaaaacttacgtcgatagcctgaatgtgattcgttccgcgatcggtacgccgctgcaaacgatttccagcggtggtacttccctcctgatgattgattccggtacgggtgataacttgtttgctgttgatgtgcgcggcattgacccggaagaaggccgttttaataatctgcgtctgatcgtcgaacgcaacaacctgtatgtgacgggttttgtgaaccgtacgaacaacgtcttctatcgtttcgctgatttctcccacgtaacgtttccgggcaccactgctgttactctgagcggcgattcttcttatactacgttacagcgtgtggctggtatcagccgcactggtatgcaaatcaatcgccattctctgacgaccagctatctggacttaatgagccattctggcaccagcctgacccagtctgttgcccgtgcgatgctgcgcttcgtgacggtcaccgccgaagccctgcgtttccgtcaaatccaacgcggcttccgcaccactttagacgatctgtctggccgcagctatgtgatgactgccgaagatgtcgatctgaccctgaactggggtcgcttgtcttccgttctgccggattatcacggtcaggattctgtccgtgttggccgtatcagctttggctctattaatgccatcctaggctccgtcgcactgattctcaattgccaccaccacgcttaa

(19) United States
(12) Patent Application Publication
(10) Pub. No.: US 2009/0092578 A1
(43) Pub. Date: Apr. 9, 2009

(54) Methods of selecting and producing modified toxins, conjugates containing modified toxins, and uses thereof

(76) Inventors: Hongsheng Su, Westmount (CA); Philip J. Coggins, Pointe Claire (CA); John R. McDonald, Kirkland (CA); Laura M. Mcintosh, Pincourt (CA)

Correspondence Address: Bell, Boyd & Lloyd LLP, 3580 Carmel Mountain Road, Suite 200, San Diego, CA, 92130, US

(21) Appl. No.: 12/004,025

(22) Filed: Dec. 18, 2007

Related U.S. Application Data
(60) Provisional application No. 60/965,977, filed on Aug. 22, 2007.
(60) Provisional application No. 60/878,166, filed on Dec. 29, 2006.

(30) Foreign Application Priority Data
Dec. 17, 2007 (CA) PCT/CA2007/002278

(51) Int. Cl. A61K 38/20; C12P 21/02; A61K 38/16; C12N 15/74; C12N 15/70; A61K 39/395; C12N 15/31; C07K 14/195

(52) U.S. Cl. 424/85.2; 435/69.1; 530/350; 514/12; 536/23.7; 435/320.1; 424/130.1

(57) ABSTRACT

Methods for selecting and identifying modified toxins and conjugates thereof are provided. The methods are select for toxins that exhibit reduced toxicity to the host cell in which they are expressed. Methods of increasing production of toxins, such as the modified toxins, or conjugates thereof, also are provided. In particular, in the methods the toxins, or conjugates thereof, are produced in the presence of an inhibitor molecule. Also provided, are modified toxins and conjugates thereof. Such conjugates can be used in the treatment of various disease or disorders associated with proliferation, migration, and physiological activity of cells involved in immune or inflammatory responses.

### PRIORITY CLAIM AND RELATED APPLICATIONS

Benefit of priority is claimed under 35 U.S.C. § 119(e) to U.S. provisional application Ser. No. 60/965,977, filed Aug. 22, 2007, to Hongesheng Su, Philip J. Coggins, John R. McDonald and Laura M. McIntosh, entitled “METHODS OF SELECTING AND PRODUCING MODIFIED TOXINS, CONJUGATES CONTAINING MODIFIED TOXINS, AND USES THEREOF,” and to U.S. provisional application Ser. No. 60/878,166, filed Dec. 29, 2006, to Hongesheng Su, Philip J. Coggins, John R. McDonald and Laura M. McIntosh, entitled “METHODS OF SELECTING AND PRODUCING MODIFIED TOXINS, CONJUGATES CONTAINING MODIFIED TOXINS, AND USES THEREOF.” Benefit of priority also is claimed under 35 U.S.C. §120 to International PCT application No (attorney dkt. No. 17080-010WO1/609PC), filed in the RO/CA on Dec. 17, 2007, to Osprey Pharmaceuticals, Hongesheng Su, Philip J. Coggins, John R. McDonald and Laura M. McIntosh, entitled “METHODS OF SELECTING AND PRODUCING MODIFIED TOXINS, CONJUGATES CONTAINING MODIFIED TOXINS, AND USES THEREOF.”

This application also is related to U.S. application Ser. No. 09/360,242, filed Jul. 22, 1999, now U.S. Pat. No. 7,157,418, entitled “METHODS AND COMPOSITIONS FOR TREATING SECONDARY TISSUE DAMAGE AND OTHER INFLAMMATORY CONDITIONS AND DISORDERS,” which claims the benefit of priority under 35 U.S.C. §120 as a continuation-in-part of International PCT application No. PCT/CA99/00659, filed Jul. 21, 1999, by Osprey Pharmaceuticals Limited, McDONALD, John R. and COGGINS, Philip J. entitled “METHODS AND COMPOSITIONS FOR TREATING SECONDARY TISSUE DAMAGE AND OTHER INFLAMMATORY CONDITIONS AND DISORDERS,” and claims the benefit of priority under 35 U.S.C. §119(e) to U.S. provisional application Ser. No. 60/155,186, filed on Jul. 22, 1998, to John R. McDonald and Philip J. Coggins, entitled “METHODS AND COMPOSITIONS FOR TREATING SECONDARY TISSUE DAMAGE.”

This application also is related to U.S. application Ser. No. 09/453,851, now U.S. Pat. No. 7,166,702, filed Dec. 2, 1999, to John R. McDonald and Philip J. Coggins, entitled “CYTOTOXIC CONJUGATES COMPRISING A CHEMOKINE RECEPTOR TARGETING AGENT,” which is a divisional of U.S. application Ser. No. 09/360,242, now U.S. Pat. No. 7,157,418. This application also is related to U.S. application Ser. No. 09/792,793, now U.S. Pat. No. 7,192,736, filed Feb. 22, 2001, entitled “NUCLEIC ACID MOLECULES ENCODING CYTOTOXIC CONJUGATES THAT CONTAIN A CHEMOKINE RECEPTOR TARGETING AGENT,” which is a divisional of U.S. application Ser. No. 09/360,242 and U.S. application Ser. No. 09/453,851.

This application also is related to U.S. application Ser. No. 10/375,209, now abandoned, filed Feb. 24, 2003, entitled “METHODS AND COMPOSITIONS FOR TREATING SECONDARY TISSUE DAMAGE AND OTHER INFLAMMATORY CONDITIONS AND DISORDERS,” which is a continuation of U.S. application Ser. No. 09/792,793, U.S. application Ser. No. 09/453,851, and U.S. application Ser. No. 09/360,242.

This application also is related to U.S. application Ser. No. 11/361,977, filed Feb. 24, 2006, entitled “METHODS AND COMPOSITIONS FOR TREATING SECONDARY TISSUE DAMAGE AND OTHER INFLAMMATORY CONDITIONS AND DISORDERS,” which is a continuation of U.S. application Ser. No. 10/375,209, U.S. application Ser. No. 09/792,793, U.S. application Ser. No. 09/453,851, and U.S. application Ser. No. 09/360,242.

The subject matter of each of the above noted applications and patents is incorporated by reference in its entirety.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy # 1 and Copy # 2), the contents of which are incorporated by reference in their entirety. The computer-readable file on each of the aforementioned compact discs, created on Dec. 18, 2007, is identical, 370 kilobytes in size, and titled 609SEQ.001.txt.

### FIELD OF THE INVENTION

Provided are methods for selecting for and identifying modified toxins that have reduced toxicity. Also provided are modified toxins that have reduced toxicity, and conjugates containing such modified toxin. Methods for producing such modified toxins, or conjugates thereof, are provided. The conjugates are used in methods for treating diseases associated with proliferation, migration, and physiological activity of cells involved in inflammatory responses.

### BACKGROUND

Inflammatory responses are mediated by immune defense cells and associated tissue residential cells that accumulate at the site of tissue injury or trauma to rid the body of unwanted exogenous agents (e.g., microbes) and endogenous agents (e.g., cancer cell clones); to clean up cellular debris, and to participate in tissue and wound healing. Unfortunately, the molecular mechanisms involved in these reparatory (inflammatory) processes due to, for example, the inappropriate activation of leukocytes can initiate secondary tissue damage, which, in turn, contributes to the pathogenesis and persistent pathology of several inflammatory and immunomodulatory diseases.

The molecular mechanisms and the cellular and chemical mediators involved in secondary tissue damage, are similar, if not identical, in most inflammatory diseases of man. Hence, various therapeutics have been developed to treat such inflammatory and immunomodulatory diseases by targeting these molecular mechanisms and/or other common mediators. For example, therapeutics have been developed that target specific single biochemical events that occur at the cellular level (e.g., cytotoxic actions of excitatory amino acids or reactive oxygen species) involved with the pathophysiological process of such inflammatory and immunomodulatory diseases. Included among such therapeutics are steroids such as, but not limited to, methylprednisolone and its synthetic 21 aminosteroid (lazaroid) derivative (e.g., trisilazad), which act as oxygen free radical scavengers. Beneficial side effects of steroids are hindered by debilitating side effects, so that long term steroid treatment is not a viable clinical option.

Therapeutics also have been developed to treat inflammatory diseases by targeting specific inflammatory mediators (i.e. cytokines, growth factors, or their receptors) induced and/or involved in the pathophysiological process. Included among such therapeutics are Remicade® (infliximab, a neutralizing antibody to tumor necrosis factor (TNF)-α), Enbrel® (etanercept, a soluble TNFα receptor), and neutralizing antibodies to various growth factors including basic fibroblast and vascular endothelial growth factors (McDonald et al. (2001) IDrugs, 4:427-442). While being specific, such therapeutics all focus on a single component involved in the pathology of the disease. Hence, such therapeutics typically only provide partial or temporary benefits, due to the compensatory nature of the inflammatory response and the existence of other inflammatory cytokines and growth factors that are left to participate in the pathological process.

Therapeutics that provide a more comprehensive approach to treat inflammatory disease and other conditions having an immunomodulatory component by targeting the cellular mediators of the disease have been developed. Included among such cell-targeted therapeutics are those that contain a toxin moiety and that are able to gain entry into one or more cells by various mechanisms resulting in elimination of the cell(s). Exemplary of such molecules are any set forth in U.S. application Ser. Nos. 09/360,242, 09/453,851, and 09/792,793, now U.S. Pat. Nos. 7,166,702, 7,157,418 and 7,192,736. Such conjugates can be designed to specifically and predictably target cell types associated with disease pathology, and hence are useful for disease treatment. Fusion protein conjugates are produced in host cells. The toxin moiety in the conjugates, however, limits efficient production of these molecules. While such molecules are known and available, a need exists to efficiently produce large quantities for widespread dissemination and use thereof. Accordingly, among the objects herein, it is an object to provide methods for more efficient production of toxins and conjugates containing the toxins.

### SUMMARY

Provided herein are methods for production of therapeutic molecules, and the use of modified toxin conjugates to target cellular mediators associated with the pathology of inflammatory or immunomodulatory diseases or conditions. In particular, provided are modified toxin polypeptides, conjugates containing the modified toxin polypeptides and methods for generating, and preparing modified toxin polypeptides. The modified toxin polypeptides (and/or conjugates containing them) exhibit reduced toxicity in host cells in which they are expressed, permitting expression of higher levels compared to toxin polypeptides not so-modified. The modifications occur in the primary amino acid sequence of the polypeptide.

Provided are methods of selection or identification of a modified ribosome inactivation protein (RIP), or active portion or fragment thereof, that are identified by virtue of expression in a host cell or cells. In particular, the methods select for RIPs that have reduced toxicity to the host cell compared to the starting RIP protein used in the selection methods herein. In practicing the methods provided herein, a nucleic acid encoding a RIP, or active portion thereof, is introducing into a host cell(s), the cells are grown, cells that grow are isolated, and from among the cells that grow a cell expressing a RIP is isolated. The methods provided herein can be performed such that the cells are grown in medium that does not contain a selective modulator, for example, an adenine analog such as 4-aminopyrazolo[3,4-d]pyrimidine (4-APP). The methods provided herein can further include the step of expanding the cells that expresses a RIP. In one example, the RIP expressed in the isolated cell is identified, isolated or purified. The RIP can be identified by its sequence or its molecular weight. In some cases, the RIP can be identified by sequencing. Also provided are the RIPs produced by the methods.

In some examples of the method, the cells with nucleic acid encoding a RIP are grown in the presence of a selective modulator. The selective modulator can be a RIP inhibitor, for example, an adenine analog. The adenine analog can be 4-aminopyrazolo[3,4-d]pyrimidine (4-APP). In using a selective modulator, such as a RIP inhibitor, for example, an adenine analog such as 4-APP, the concentration is chosen such that it is not toxic to the host cells. In some aspects, the concentration is chosen to inhibit toxicity of the RIP on the host cell. In one example, the inhibition of toxicity is sufficient to increase the amount of RIP expressed compared to the absence of the RIP inhibitor, adenine analog, or 4-APP. For example, the toxicity of the RIP is inhibited by 0.1%, 0.5% 1%, 5%, 10%, 15%, 20%, 30%, 40%, 50%, 60%, 70%, 80%, 90%, 95%, or 100%. Where the RIP inhibitor is 4-APP, the concentration of inhibitor used in the methods herein is about or is 0.1 mM to about or 5.0 mM. For other inhibitors, suitable concentrations can be determined empirically or by reference to 4-APP. In some examples, the concentration of 4-APP is between about or is 0.1 to 2, 3, or 4 mM, or is 0.2, 0.3, 0.4, 0.5, 0.6, 0.7, 0.9, or 1 mM. In other examples, the concentration of 4-APP is about or is 0.5 mM.

Hence provided are methods for selection of toxins with reduced toxicity and also methods for production of toxins with reduced toxicity. The selection method identifies toxins that have a reduced toxicity. Such toxins, normally, when nucleic acids encoding them are introduced into bacteria for expression, are not expressed, but generally are expressed a low levels. The selection method looks for the toxins that are expressed, and then expands the cells that express toxins with reduced toxicity to permit expression. Also provided are production methods, which is another way to select for mutants by growing the cells in the presence of an inhibitor, such as 4-APP, typically a low dose form of 4-APP, which further inhibits the high level of toxicity, and results production of toxin and also mutants that retain a good deal of toxicity, but not as much as the wildtype.

Thus the method of selection permits the identification of toxins with reduced toxicity. Such modified toxins can be produced at higher levels than the wildtype. In the methods of production, the toxins, wildtype or modified, can be expressed in the presence of 4-APP (generally higher concentrations than used in the selection methods) to render the toxins less toxic. If a modified toxin identified in the selection method already is less toxic than wild type, the presence of 4-APP will further limit the toxicity, so that more can be produced compared to wildtype or the mutant in the absence of 4-APP or that a lower concentration of 4-APP could be used. In all instances, sufficient toxicity is retained to render them cytotoxic for use in the methods. The toxins are so toxic, that even with a large reduction in the their toxicity, such as reduction to 1% toxicity, they are sufficiently toxic for the methods herein.

In one aspect, the methods provided herein are such that the host cell is a eukaryotic cell. In another aspect, the host cell used in the methods herein is a prokaryotic cell, for example, E. coli.

In the methods provided herein, the RIP encoded by the introduced nucleic acid molecule can be a type I RIP, or an active fragment thereof. For example, the RIP used in the methods herein include, but are not limited to, dianthin 30, dianthin 32, lychnin, saporin-1, saporin-2, saporin-3, saporin-4, saporin-5, saporin-6, saporin-7, saporin-8, saporin-9, PAP, PAP II, PAP-R, PAP-S, PAP-C, mapalmin, dodecandrin, bryodin-L, bryodin, bryodin II, clavin, colicin-1, colicin-2, luffin-A, luffin-B, luffin-S, 19K-PSI, 15K-PSI, 9K-PSI, alpha-kirilowin, beta-kirilowin, gelonin, momordin, momordin-II, momordin-Ic, Mirabilis Antiviral Protein (MAP), MAP-30, alpha-momorcharin, beta-momorcharin, trichosanthin, TAP-29, trichokirin, barley RIP I, barley RIP II, tritin, flax RIP, maize RIP 3, maize RIP 9, maize RIP X, asparin-1, or asparin 2.

In other examples of the method provided herein, the RIP encoded by the introduced nucleic acid molecule is a type II RIP, the catalytic subunit thereof or an active fragment thereof. For example, the RIP used in the methods herein include, but are not limited to, Shiga toxin (Stx), Shiga-like toxin II (Stx2), volkensin, ricin, nigrin-CIP-29, abrin, vircumin, modeccin, ebulitin-α, ebulitin-β, ebulitin-γ, or porrectin. In one aspect, the introduced nucleic acid encodes REP subunit A, or an active fragment thereof. In another aspect, the introduced nucleic acid encodes subunit A1 (SA1) of the RIP Shiga Toxin. The SA1 can be truncated. For example, the SA1 can be truncated by deletion of 1, 2, 3, 4, 5, 6, 7, 8, 9, 10, 11 or 12 contiguous amino acids at the N- or C-terminus. In another example, the SA1 can be modified by replacement of Cys with another amino acid such as Ser. Exemplary of nucleic acids introduced into host cells in the methods provided herein are nucleic acid molecules that encode an SA1 having a sequence of amino acids set forth in SEQ ID NO: 22 or SEQ ID NO:24. For example, the SA1 can be encoded by a nucleic acid molecule containing bases whose sequence is set forth in SEQ ID NO: 21 or SEQ ID NO:23.

In the methods provided herein, the RIP encoded by the introduced nucleic acid molecule can be conjugated to a ligand to form a ligand-toxin conjugate. The RIP and ligand in the conjugate can be linked directly via a covalent or ionic linkage. For example, the RIP and ligand can be joined via a linker such as a peptide, polypeptide or an amino acid. Exemplary of a linker is an Ala-Met linker. Typically, the ligand-toxin conjugate is a fusion protein.

The ligand in the ligand-toxin conjugate can be a chemokine receptor targeting agent, a non-chemokine cytokine, a hormone, a growth factor, an antibody specific for a cell surface receptor, a TNF superfamily ligand, and a pattern recognition receptor (PRR) ligand. In one example, the ligand is a growth factor such as vascular endothelial growth factor (VEGF). In another example, the ligand is a chemokine receptor targeting agent such as a chemokine, or a fragment of the chemokine, or an antibody that specifically binds to a chemokine receptor, or a fragment of an antibody, wherein the fragment binds to the chemokine receptor. Where the ligand is an antibody, it can be a monoclonal antibody, or an antigen-specific fragment thereof. Exemplary of monoclonal antibodies are those that are specific for an antigen including, but not limited to, (DARC), D6, CXCR-1, CXCR-2, CXCR-3A, CXCR3B, CXCR-4, CXCR-5, CXCR6, CXCR7, CCR-1, CCR-2A, CCR-2B, CCR-3, CCR-4, CCR-5, CCR-6, CCR-7, CCR-8, CCR-9, CCR10, CX3CR-1, and XCR1.

In an additional example, the ligand is a chemokine. Exemplary of chemokines in the ligand-toxin conjugates used in the methods herein include, but are not limited to, monocytes chemotactic protein-1 (MCP-1), MCP-2, MCP-3, MCP-4, MCP-5, eosinophils chemotactic protein 1 (Eotaxin-1), Eotaxin-2, Eotaxin-3, stromal derived factor-1β, SDF-1α, SDF-2, macrophage inhibitory protein 1α (MIP-1α), MIP-1β, MIP-1γ, MIP-2, MIP-2α, MIP-2β, MIP-3, MIP-3β, MIP-3α, MIP-4, MIP-5, Regulated on Activation, Normal T cell Expressed and Secreted (RANTES) protein, interleukin-8 (IL-8), growth regulated protein α (GRO-α), interferon-inducible protein 10 (IP-10), macrophage-derived chemokine (MDC), granulocyte chemotactic protein 2 (GCP-2), epithelial-derived neutrophil-activating protein 78 (ENA-78), platelet basic protein (PBP), gamma interferon-induced monokine (MIG), platelet factor 4 (PF-4), hemofiltrate CC chemokine 1 (HCC-1), thymus and activation-regulated chemokine (TARC), lymphotactin, lungkine, C10, liver-expressed chemokine (LEC), exodus-2 (SLC), thymus expressed chemokine (TECK), cutaneous T-cell attracting chemokine (CTACK), mucosae-associated epithelial chemokine (MEC), single C motif 1-β (SCM-1β), interferon-inducible T-cell alpha chemoattractant (I-TAC), breast and kidney-expressed chemokine (BRAK), fractalkine, and B cell-attracting chemokine 1 (BCA-1), and allelic or species variants thereof. In one example, the chemokine is any of MCP-1, Eotaxin-1, SDF-1β, GRO-α, MIP-1β, IL-8, IP-10, MCP-3, MIP-3α, MDC, MIP-1α, and BCA-1, and allelic or species variants thereof. In another example, the chemokine is MCP-1. Exemplary of a nucleic acid encoding a ligand toxin conjugate is a nucleic acid molecule encoding a ligand-toxin conjugate having the sequence of amino acid residues set forth in SEQ ID NO: 38 or SEQ ID NO:40. Among such a nucleic acid molecule are those having the sequence set forth as in SEQ ID NO: 37 or SEQ ID NO:39.

In one aspect of the methods herein, the identified RIP contains a mutation compared to the RIP encoded by the introduced nucleic acid molecule. In the methods provided herein, the identified RIP is assessed for its toxicity. The toxicity can be assessed by assays including, but not limited to, a protein synthesis assay, a depurination assay, and a cell growth/viability assay. Typically, the identified RIP retains toxicity compared to the RIP encoded by the introduced nucleic acid molecule. The identified RIP retains 0.5%, 1%, 2%, 3%, 4%, 5%, 6%, 7%, 8%, 9%, 10%, 20%, 30%, 40%, 50%, 60%, 70%, 80%, 90% or more toxicity.

Also provided in the methods herein are additional steps to produce the identified RIP. Such methods include introducing a nucleic acid molecule encoding the identified RIP, or active fragment thereof into a host cell(s), incubating the cells in the presence of a RIP inhibitor, wherein the amount of REP inhibitor is selected to decrease the toxicity of the RIP polypeptide; and growing the cells under conditions, whereby the RIP or active fragment thereof is produced. The RIP can be purified such that, generally, the amount of RIP expressed or purified or both is greater than in the absence of the RIP inhibitor.

The methods provided herein also can include a further step of preparing a conjugate containing the identified RIP. In the methods provided herein, the methods also include synthesizing the identified RIP, or conjugate containing the identified RIP.

Also provided herein is a method for increasing production of a ribosome inactivating protein (RIP), or active fragment thereof. Such methods allow for efficient production of RIPs, or conjugates containing RIPs, for example, to provide for a viable source of such conjugates for use as therapeutics. In the methods of production herein, nucleic acid encoding a REP, or active fragment thereof, is introduced into a host cell. The cells are incubated in the presence of a RIP inhibitor, such that the amount of RIP inhibitor is selected to decrease the toxicity of the RIP. In the methods of increased production herein, the cells are grown under conditions where a RIP or active fragment thereof is produced. In one aspect, the method of production includes a step where the RIP is purified. Typically, the amount of RIP expressed or purified or both is greater than in the absence of the REP inhibitor.

In the methods of production provided herein, the RIP encoded by the introduced nucleic acid molecule can be a type I RIP, or an active fragment thereof. For example, the REP used in the methods herein include, but are not limited to, dianthin 30, dianthin 32, lychnin, saporin-1, saporin-2, saporin-3, saporin-4, saporin-5, saporin-6, saporin-7, saporin-8, saporin-9, PAP, PAP II, PAP-R, PAP-S, PAP-C, mapalmin, dodecandrin, bryodin-L, bryodin, bryodin II, clavin, colicin-1, colicin-2, luffin-A, luffin-B, luffin-S, 19K-PSI, 15K-PSI, 9K-PSI, alpha-kirilowin, beta-kirilowin, gelonin, momordin, momordin-II, momordin-Ic, Mirabilis Antiviral Protein (MAP), MAP-30, alpha-momorcharin, beta-momorcharin, trichosanthin, TAP-29, trichokirin, barley RIP I, barley REP II, tritin, flax RIP, maize RIP 3, maize RIP 9, maize RIP X, asparin-1, or asparin 2.

In other examples of the method of production provided herein, the RIP encoded by the introduced nucleic acid molecule is a type II RIP, the catalytic subunit thereof or an active fragment thereof. For example, the RIP used in the methods herein include, but are not limited to, Shiga toxin (Stx), Shiga-like toxin II (Stx2), volkensin, ricin, nigrin-CIP-29, abrin, vircumin, modeccin, ebulitin-α, ebulitin-β, ebulitin-γ, or porrectin. In one aspect, the introduced nucleic acid encodes RIP subunit A, or an active fragment thereof. In another aspect, the introduced nucleic acid encodes subunit A1 (SA1) of the RIP Shiga Toxin. The SA1 can be truncated. For example, the SA1 can be truncated by deletion of 1, 2, 3, 4, 5, 6, 7, 8, 9, 10, 11 or 12 contiguous amino acids at the N- or C-terminus.

In one aspect, the REP, for example an SA1, encoded by the introduced nucleic acid is modified. In one example, the SA1 can be modified by replacement of Cys with another amino acid such as Ser. In another example, the SA1 is modified by replacement of one or both of positions 38 or position 219 with reference to amino acid positions in an SA1 having a sequence of amino acids set forth in SEQ ID NO:22. For example, the amino acid replacement can correspond to L38R and/or V219A. In one example, the amino acid replacement corresponds to V219A. Exemplary of nucleic acids introduced into host cells in the methods provided herein are nucleic acid molecules that encode an SA1 having a sequence of amino acids set forth in SEQ ID NO: 26 or SEQ ID NO:28. For example, the SA1 can be encoded by a nucleic acid molecule containing nucleotides whose sequence is set forth in SEQ ID NO: 27 or SEQ ID NO:29.

In the methods of production provided herein, the RIP inhibitor is an adenine analog. For example, the adenine analog is 4-aminopyrazolo[3,4-d]pyrimidine (4-APP). Generally, in the methods of production herein, the concentration of the RIP inhibitor, adenine analog or 4-APP is chosen such that it is effective to decrease the toxicity of the RIP by at or about 10%, 20%, 30%, 40%, 50%, 60%, 70%, 80%, 90%, 91%, 92%, 93%, 94%, 95%, 96%, 97%, 98%, 99%, or 100%. Where the RIP inhibitor is 4-APP, the concentration used in the methods herein is about or is 1 mM to about or 40.0 mM. In one example, the concentration of 4-APP is between about or is 2.0 mM, 3.0 mM, 4.0 mM, 5.0 mM, 6.0 mM, 7.0 mM, 8.0 mM, 9.0 mM, 10.0 mM, 15.0 mM, or 20.0 mM.

In one aspect in the methods of production provided herein production is in eukaryotic host cells. In another aspect, production is in prokaryotic host cells, for example, E. coli.

In the methods of production herein, an induction agent can be used in the methods of production such that the RIP polypeptide is expressed after induction with an induction agent. The induction agent can be isopropyl-β-D-1-thiogalactopyranoside (IPTG). The REP inhibitor used in the methods of production herein can be added before, during and/or after the addition of the induction agent.

In some aspects, the methods of production herein are used to increase production of a conjugate containing a RIP. In such methods, the nucleic acid molecule that encodes the RIP includes a sequence of nucleotides encoding a ligand, whereby the molecule encodes a ligand-toxin conjugate. The RIP and ligand in the conjugate can be linked directly via a covalent or ionic linkage. For example, the RIP and ligand can be joined via a linker such as a peptide, polypeptide or an amino acid. Exemplary of a linker is an Ala-Met linker; the Met can be included as the start codon in the linked polypeptide. Typically, the ligand-toxin conjugate is a fusion protein.

Conjugates that contain one or more receptor targeting agents, such as chemokine-receptor targeting linked, either directly or via a linker, to one or more targeted agents are provided. In particular, conjugates provided herein contain the following components: (receptor targeting agent)ₙ, (L)_{q}, and (targeted agent)ₘ in which at least one receptor targeting agent, such as a receptor ligand or receptor-specific antibody, or an effective portion of the ligand or antibody, is(are) linked directly or via one or more linkers (L) to at least one targeted agent. L refers to a linker. Any suitable association among the elements of the conjugate is contemplated as long as the resulting conjugates interacts with a targeted receptor such that internalization of an associated targeted agent is effected. In the conjugates provided herein, the targeted agent is a modified toxin, such as a modified RIP, or a toxic fragment thereof. The toxin or fragment is modified in its primary amino acid sequence such that it is less toxic to host cells in which it is expressed for production thereof than the unmodified form thereof. The toxins or conjugates are modified by the methods provided herein.

The variables n and m are integers of 1 or greater and q is 0 or any integer. The variables n, q and m are selected such that the resulting conjugate interacts with the targeted receptor and a targeted agent is internalized by a cell to which it has been targeted. Typically n is between 1 and 3; q is 0 or more, depending upon the number of linked targeting and targeted agents and/or functions of the linker, q is generally 1 to 4; m is 1 or more, generally 1 or 2. When more than one targeted agent is present in a conjugate the targeted agents may be the same or different. Similarly, when more than one receptor targeting agent is present in the conjugates they can be the same or different.

The conjugates provided herein can be produced as fusion proteins, can be chemically coupled or include a fusion protein portion and a chemically linked portion or any combination thereof. For purposes herein, the receptor targeting agent is any agent, typically a polypeptide, that specifically interacts with a receptor, such as chemokine receptors on activated leukocytes, and that, upon interacting with the receptor, internalizes a linked or otherwise associated targeted agent, such as a toxin, intended to be internalized by the targeted cell.

The ligand in the ligand-toxin conjugate can be a chemokine receptor targeting agent, a non-chemokine cytokine, a hormone, a growth factor, an antibody specific for a cell surface receptor, a TNF superfamily ligand, and a pattern recognition receptor (PRR) ligand. In one example, the ligand is a growth factor such as VEGF. In another example, the ligand is a chemokine receptor targeting agent such as a chemokine, or a fragment of the chemokine, or an antibody that specifically binds to a chemokine receptor, or a fragment of an antibody, wherein the fragment binds to the chemokine receptor. Where the ligand is an antibody, it can be a monoclonal antibody, or an antigen-specific fragment thereof. Exemplary of monoclonal antibodies are those that are specific for an antigen selected including, but not limited to, (DARC), D6, CXCR-1, CXCR-2, CXCR-3A, CXCR3B, CXCR-4, CXCR-5, CCR-1, CCR-2A, CCR-2B, CCR-3, CCR-4, CCR-5, CCR-6, CCR-7, CCR-8, CCR-9, CCR10, CX3CR-1, and XCR1.

In an additional example, the ligand is a chemokine. Exemplary of chemokines in the ligand-toxin conjugates used in the methods herein include, but are not limited to, monocytes chemotactic protein-1 (MCP-1), MCP-2, MCP-3, MCP-4, MCP-5, eosinophils chemotactic protein 1 (Eotaxin-1), Eotaxin-2, Eotaxin-3, stromal derived factor-1β, SDF-1α, SDF-2, macrophage inhibitory protein 1α (MIP-1α), MIP-1β, MIP-1γ, MIP-2, MIP-2α, MIP-2β, MIP-3, MIP-3β, MIP-3α, MIP-4, MIP-5, Regulated on Activation, Normal T cell Expressed and Secreted (RANTES) protein, interleukin-8 (IL-8), growth regulated protein α (GRO-α), interferon-inducible protein 10 (IP-10), macrophage-derived chemokine (MDC), granulocyte chemotactic protein 2 (GCP-2), epithelial-derived neutrophil-activating protein 78 (ENA-78), platelet basic protein (PBP), gamma interferon-induced monokine (MIG), platelet factor 4 (PF-4), hemofiltrate CC chemokine 1 (HCC-1), thymus and activation-regulated chemokine (TARC), lymphotactin, lungkine, C10, liver-expressed chemokine (LEC), exodus-2 (SLC), thymus expressed chemokine (TECK), cutaneous T-cell attracting chemokine (CTACK), mucosae-associated epithelial chemokine (MEC), single C motif 1-β (SCM-1β), interferon-inducible T-cell alpha chemoattractant (I-TAC), breast and kidney-expressed chemokine (BRAK), fractalkine, and B cell-attracting chemokine 1 (BCA-1), and allelic or species variants thereof. In one example, the chemokine is any of MCP-1, Eotaxin-1, SDF-1β, GRO-α, MIP-1β, IL-8, IP-1β, MCP-3, MIP-3α, MDC, MIP-1α, and BCA-1, and allelic or species variants thereof. In another example, the chemokine is MCP-1.

The toxin moiety in the ligand-toxin conjugate can be a Shiga toxin, catalytically active fragment thereof, or active fragment thereof. For example, the toxin moiety in the ligand-toxin conjugate produced in the methods herein can be SA1. The toxin moiety, such as SA1, in the ligand-toxin conjugate can be a modified toxin. Exemplary of a nucleic acid encoding a ligand-toxin conjugate produced in the methods herein is a nucleic acid molecule encoding a ligand-toxin conjugate having the sequence of amino acid residues set forth any of SEQ ID NOS: 42, 44, 46, 48, 50, 52, 54, 56, 58, 60, 62, 64, or 67. Among such a nucleic acid molecule are those having the sequence set forth in any of SEQ ID NO:41, 43, 45, 47, 49, 50, 53, 55, 57, 59, 61, 63, 65 or 66.

Provided are modified toxins, particularly, modified RIPs, that exhibit reduced toxicity compared to the starting materials, which are RIPs, which include wildtype and variant RIPs. Included among such modified RIP toxins, or conjugates thereof, are any identified in the methods herein.

Among the modified toxins provided herein are modified Shiga Toxin polypeptide, or active fragment thereof, that has one or more amino acid modifications in a Shiga Toxin, allelic or species variant thereof, catalytically active portion thereof, or active fragment thereof, such that the modification confers reduced toxicity. In one example, the one or more amino acid modifications are replacements of one or both of positions corresponding to positions 38 and/or 219 with reference to amino acid positions in Shiga Toxin A1 subunit (SA1) having a sequence of amino acids set forth in SEQ ID NO:22. The modified Shiga toxins provided herein have at least about 40%, 50%, 60%, 70%, 80%, 90%, 95%, 96%, 97%, 98%, 99% sequence identity to the polypeptide having the sequence of amino acids set forth set forth in SEQ ID NO: 22 and that includes modifications at loci corresponding to amino acid positions 38 and/or 219. Among modifications at positions 38 and/or 219 are those that correspond to L38R and/or V219A. The modified Shiga toxins include subunit A. For example, the modified Shiga toxins can include only the SA1 of Shiga toxin, or an active fragment thereof. The SA1 can be truncated. For example, the truncated SA1 can be truncated by deletion of 1, 2, 3, 4, 5, 6, 7, 8, 9, 10, 11 or 12 contiguous amino acids at the N- or C-terminus. Exemplary of modified Shiga Toxins are those having a sequence of amino acids set forth in SEQ ID NOS: 26 or 28, or is an allelic or species variant thereof.

Also provided herein, are conjugates containing a targeted agent that is a modified ribosome inactivating protein (RIP), such as any modified RIP identified in the methods herein. Also provided are conjugates containing a targeted agent that is a modified Shiga Toxin, or active fragment thereof, such as any modified Shiga Toxin as noted above. The conjugates also contain a targeting agent, or a portion thereof, that facilitates binding of the conjugate to a cell surface receptor resulting in internalization of the targeted agent in cells bearing the receptor.

Among such conjugates are those having the following components: (targeting agent)ₙ, (L)_{q}, and (targeted agent)ₘ, where L is a linker for linking the targeting agent to the targeted agent, the targeting agent is any moiety that selectively binds to a cell surface receptor, m and n, which are selected independently, are at least 1, and q is 0 or more as long as the resulting conjugate binds to the targeted receptor, is internalized and delivers the targeted agent. Typically, the resulting conjugate binds to a receptor that interacts with and internalizes a targeting agent, whereby the targeted agent(s) is internalized in a cell bearing the receptor. In some cases where the conjugate contains a plurality of targeted agents, the targeted agents are the same or different. Typically, the targeted agents are all modified forms of a RIP toxin. Also, when the conjugate contains a plurality of targeting agents, the targeting agents are the same or different. In one example, m and n, which are selected independently, are 1-6. In another example, q is 1, n is 2 and m is 1.

In conjugates provided herein, the targeting agent includes a receptor targeting agent, such as but not limited to, a chemokine receptor targeting agent, a non-chemokine cytokine, a hormone, a growth factor, an antibody specific for a cell surface receptor, a TNF superfamily ligand, and a pattern recognition receptor (PRR) ligand. In one example, the ligand is a growth factor such as VEGF. In another example, the ligand is a chemokine receptor targeting agent such as a chemokine, or a fragment of the chemokine, or an antibody that specifically binds to a chemokine receptor, or a fragment of an antibody, wherein the fragment binds to the chemokine receptor. Where the ligand is an antibody, it can be a monoclonal antibody, or an antigen-specific fragment thereof. Exemplary of monoclonal antibodies are those that are specific for an antigen including, but not limited to, (DARC), D6, CXCR-1, CXCR-2, CXCR-3A, CXCR3B, CXCR-4, CXCR-5, CXCR6, CXCR7, CCR-1, CCR-2A, CCR-2B, CCR-3, CCR-4, CCR-5, CCR-6, CCR-7, CCR-8, CCR-9, CCR10, CX3CR-1, and XCR1.

In an additional example, the ligand is a chemokine. Exemplary of chemokines in the ligand-toxin conjugates provided herein include, but are not limited to, monocytes chemotactic protein-1 (MCP-1), MCP-2, MCP-3, MCP-4, MCP-5, eosinophils chemotactic protein 1 (Eotaxin-1), Eotaxin-2, Eotaxin-3, stromal derived factor-1β, SDF-1α, SDF-2, macrophage inhibitory protein 1α (MIP-1α), MIP-1β, MIP-1γ, MIP-2, MIP-2α, MIP-2β, MIP-3, MIP-3β, MIP-3α, MIP-4, MIP-5, Regulated on Activation, Normal T cell Expressed and Secreted (RANTES) protein, interleukin-8 (IL-8), growth regulated protein α (GRO-α), interferon-inducible protein 10 (IP-10), macrophage-derived chemokine (MDC), granulocyte chemotactic protein 2 (GCP-2), epithelial-derived neutrophil-activating protein 78 (ENA-78), platelet basic protein (PBP), gamma interferon-induced monokine (MIG), platelet factor 4 (PF-4), hemofiltrate CC chemokine 1 (HCC-1), thymus and activation-regulated chemokine (TARC), lymphotactin, lungkine, C10, liver-expressed chemokine (LEC), exodus-2 (SLC), thymus expressed chemokine (TECK), cutaneous T-cell attracting chemokine (CTACK), mucosae-associated epithelial chemokine (MEC), single C motif 1-β (SCM-1β), interferon-inducible T-cell alpha chemoattractant (1-TAC), breast and kidney-expressed chemokine (BRAK), fractalkine, and B cell-attracting chemokine 1 (BCA-1), and allelic or species variants thereof. In one example, the chemokine is any of MCP-1, Eotaxin-1, SDF-1β, GRO-α, MIP-1β, IL-8, IP-1β, MCP-3, MIP-3α, MDC, MIP-1α, and BCA-1, and allelic or species variants thereof. In another example, the chemokine is MCP-1.

The targeting agent in the conjugates provided herein specifically bind to one or more cell surface receptors on one or more immune effector cells, or other cells associated with an immune or inflammatory response. In one example, the immune effector cell or cells is a leukocyte. In another example, the other cells associated with an immune or inflammatory response are tissue residential cells (TRC). The cells targeted by the conjugates provided herein include, but are not limited to, monocytes, macrophages, dendritic cells, T cells, B cells, eosinophils, basophils, mast cells, natural killer (NK) cells, and neutrophils. Included among macrophages are tissue macrophages such as alveolar macrophages, microglia, and kupfer cells. Included among dendritic cells are immature dendritic cells, mature dendritic cells, and langerhans cells. Included among T cells are CD4+ (such as Th1, Th2 or Th17 cells) and CD8+ T cells. Included among TRC are mesangial cells, glial cells, endothelial cells, epithelial cells, tumor cells, fibroblasts, and synoviocytes. The cells targeted by the conjugates can be activated. For example, cell activation can induce the expression of one or more cell surface receptors targeted by the conjugates.

Among the conjugates provided herein, are those that target cell surface receptors that bind to one or more chemokines. Such chemokine receptors include, but are not limited to, CXCR1, CXCR2, CXCR3A, CXCR3B, CXCR4, CXCR5, CXCR6, CXCR7, CCR1, CCR2A, CCR2B, CCR3, CCR4, CCR5, CCR6, CCR7, CCR8, CCR9, XCR1 and CX3CR-1. Binding of the conjugate to the chemokine receptor promotes internalization of the conjugate into a cell bearing the receptor.

In the conjugates provided herein, the targeting agent and targeted agent, or active fragment thereof, are linked via a covalent or ionic linkage. For example, a modified RIP and ligand in the conjugate can be linked directly via a covalent or ionic linkage. In some cases, the RIP and ligand can be joined via a linker such as a peptide, polypeptide, amino acid or chemical linker. Exemplary of a linker is an Ala-Met linker. Exemplary of a linker also includes, but is not limited to, N-succinimidyl (4-iodoacetyl)-aminobenzoate, sulfosuccinimydil (4-iodoacetyl)-aminobenzoate, 4-succinimidyl-oxycarbonyl-α-(2-pyridyldithio)toluene, sulfosuccinimidyl-6-(α-methyl-α-(pyridyldithiol)-toluamido) hexanoate, N-succinimidyl-3-(−2-pyridyldithio)-proprionate, succinimidyl 6(3(-(-2-pyridyldithio)-proprionamido) hexanoate, sulfosuccinimidyl 6(3(-(-2-pyridyldithio)-propionamido) hexanoate, 3-(2-pyridyldithio)-propionyl hydrazide, Ellman's reagent, dichlorotriazinic acid, and S-(2-thiopyridyl)-L-cysteine.

Provided herein are conjugates having a sequence of amino acids set forth in any of SEQ ID NOS: 44, 46, 48, 50, 52, 54, 56, 58, 60, 62, 64 or 67.

Also provided are pharmaceutical compositions containing any of the modified toxin conjugates provided herein, such as any having a modified SA1. The pharmaceutical compositions contain pharmaceutically acceptable excipients, and can be formulated for any suitable route of administration, including, but not limited to, systemic, oral, nasal, pulmonary, local and topical administration. Also provided are kits containing any of the pharmaceutical compositions a device for administration of the composition and, optionally, instructions for administration.

Nucleic acid molecules encoding any of the conjugates provided herein also are provided. Also provided are plasmids containing the nucleic acid molecules and cells containing the nucleic acid molecules or plasmids.

Also provided herein is a method of targeting a toxin to a cell. Such a method includes administering a conjugate, such as to a sample of subject. The conjugate that is administered contains a modified toxin, such as any provided herein, and a cell surface receptor targeting agent, such as a ligand. The targeted cell expresses the cell surface receptor for the targeting agent.

Provided herein is a method of treatment of subjects having an immune or inflammatory disease or disorder. In the method of treatment, a pharmaceutical composition containing any of the conjugates provided herein is administered to a subject and such that the composition inhibits the proliferation, migration or physiological activity of secondary tissue damage-promoting inflammatory cells.

Also provided herein is a method of inhibiting a disease or disorder in an animal or subject or treating an animal or subject having a disease or disorder, such as, a disease or disorder that is an immune or inflammatory condition associated with inflammatory responses and/or secondary tissue damage associated with activation, proliferation and migration of one or more cells by administering a conjugate, such as any conjugate provided herein. In the method, the conjugate binds to one or more cell surface receptors expressed on one or more cells resulting in internalization of the targeted agent in cells bearing the receptor thereby inhibiting the activation, proliferation or migration of one or more cells. In one example, treatment is of a mammal. In another example, treatment is of a human.

In the methods, the one or more cells can be an immune effector cell, or other cell associated with the immune or inflammatory condition. In one example, the immune effector cell is a leukocyte. In another example, the other cell associated with the immune or inflammatory condition is a tissue residential cells (TRC). The cells include, but are not limited to, monocytes, macrophages, dendritic cells, T cells, B cells, eosinophils, basophils, mast cells, natural killer (NK) cells, and neutrophils. Included among macrophages are tissue macrophages such as alveolar macrophages, microglia, or kupffer cells. Included among dendritic cells are immature dendritic cells, mature dendritic cells, or langerhans cells. Included among T cells are CD4+ (such as Th1, Th2 or Th17 cells) and CD8+ T cells. Included among TRC are mesangial cells, glial cells, epithelial cells, tumor cells, fibroblasts, and synoviocytes. In some cases, the one or more cells is activated, such that, for example, cell surface receptors expressed on the cells are upregulated.

In one aspect of the method of inhibiting a disease or disorder herein, the conjugate inhibits the activation, proliferation or migration of one or more cells involved in a disease or disorder such as, but not limited to, CNS injury, CNS inflammatory diseases, neurodegenerative disorders, heart disease, inflammatory eye diseases, inflammatory skin diseases, inflammatory bowel diseases, inflammatory joint diseases, inflammatory kidney or renal diseases, inflammatory lung diseases, inflammatory nasal diseases, inflammatory systemic diseases, inflammation in obesity and associated diseases, inflammatory thyroid diseases, inflammatory responses associated with bacterial or viral infections, cancers, and angiogenesis-mediated disease.

In one example, the CNS inflammatory diseases and/or neurodegenerative disorders include, but are not limited to, closed head injury, leukoencephalopathy, choriomeningitis, meningitis, adrenoleukodystrophy, AIDS dementia complex, Alzheimer's Disease, Down's Syndrome, chronic fatigue syndrome, encephalitis, encephalomyelitis, spongiform encephalopathies, multiple sclerosis, Parkinson's disease and spinal cord injury/trauma (SCI). In another example, the heart disease is atherosclerosis. Inflammatory eye diseases, include but are not limited to proliferative diabetes retinopathy, proliferative vitreoretinopathy, retinitis, scleritis, scleroiritis, choroiditis and uveitis. Inflammatory skin diseases include, but are not limited to, psoriasis, eczema and dermatitis. The inflammatory bowel disease can include, but is not limited to, chronic colitis, Crohn's disease and ulcerative colitis. The inflammatory joint disease includes, but is not limited to, juvenile rheumatoid arthritis, osteoarthritis, rheumatoid arthritis and spondylarthropathies such as, ankylosing spondylitis, Reiter's syndrome, reactive arthritis, psoriatic arthritis, spondylitis, undifferentiated spondylarthopathies and Behcet's syndrome. The inflammatory kidney or renal disease includes, but is not limited to, glomerulonephritis, IgA nephropathy and lupus nephritis. The inflammatory lung disease includes, but is not limited to, acute respiratory distress syndrome, eosinophilic lung disease, chronic eosinophilic pneumonia, acute eosinophilic pneumonia, bronchoconstriction, bronchopulmonary dysplasia, bronchoalveolar eosinophilia, allergic bronchopulmonary, aspergillosis, pneumonia and fibrotic lung disease. Inflammatory nasal diseases include, but are not limited to, polyposis, sinusitis and rhinitis. The inflammatory thyroid disease includes, but is not limited to, thyroiditis. The cancers include, but are not limited to, gliomas, atheromas carcinomas, adenocarcinomas, granulomas, glioblastomas, granulomatosis, lymphomas, leukemias, lung cancers, melanomas, myelomas, sarcomas, sarcoidosis, microgliomas, meningiomas, astrocytomas, oligodendrogliomas, gastric or stomach cancer including gastrointestinal cancer, pancreatic cancer, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

In one aspect, the disease or disorder selected is a kidney disease, spinal cord injury, or a delayed type hypersensitivity disease or disorder.

In the method of treatment or inhibition of a disease or disorder herein, the targeting agent of the conjugate includes, but is not limited to, MCP-1, Eotaxin-1, SDF-1β, GRO-α, MIP-1β, IL-8, IP-10, MCP-3, MIP-3α, MDC, MIP-1α, and BCA-1, and allelic or species variants thereof, and the targeted agent is a modified Shiga Toxin. In one example, the targeting agent is MCP-1. Exemplary of conjugates include, but are not limited to, LPM1c, LPM1d, LPM2, LPM3, LPM4, LPM5, LPM6, LPM7, LPM8, LPM9, LPM10, and LPM11. Such conjugates have a sequence of amino acids set forth in any of SEQ ID NOS: 42, 44, 46, 48, 50, 52, 54, 56, 58, 60, 62, 64 or 67, respectively.

For example, the disease can be multiple sclerosis (MS). For such embodiments, the targeted cells are those that express receptors that are upregulated in MS. For example, targeted cells include those that express receptors selected from among, for example, one or more, such as one or at least two, of CCL1-8, CXCL8-13, CCR1-3,5, 6 and CXCR1-3, 4. The conjugate for treatment of MS contains a targeting agent, such as a chemokine or fragment thereof sufficient for binding and internalization of linked (directly or indirectly) agents, that binds to and is internalized by such receptors. Hence, for example, the conjugates can contain a chemokine or fragment thereof sufficient for binding and internalization by a receptor therefor; and the chemokine, for example, is selected from among, for example, 1-309, MCP-1, MIP-1α, MIP-1β, RANTES, MCP-3, MCP-2, IL-8, MIG, IP-10, I-TAC, SDF-1α, SDF-1β, BCA-1, an Eotaxin, MCP-4, MCP-5, C10, LEC and MIP-1b2. Exemplary of such conjugates is LPM1d.

In one example of the methods herein, the targeting agent of the conjugate contains a PF-4 or allelic or species variants thereof, and the disease or condition is an angiogenesis-mediated disease. In another example, the targeting agent of the conjugate is a VEGF or allelic or species variants thereof, and the disease or condition is an angiogenesis-mediated disease.

### DETAILED DESCRIPTION

### Outline

A. Definitions

B. Ribosome Inactivating Proteins (RIPs), Selection, Expression and Production Thereof

C. Ribosome Inactivating Proteins (RIPs) and Methods of Action
1. Exemplary RIPs
   Shiga Toxin
2. RIP Toxin Inhibitors
   4-APP and other adenine analogs

D. Methods of Selecting Modified Toxins or Conjugates Thereof
1. Candidate RIP Proteins or Conjugates Thereof
2. Introduction of RIPs or conjugates thereof into host cells
   a. Transfection
   b. Transformation
   c. Electroporation
3. Expression, Selection and Identification
4. Activity Assessment
   a. Protein Synthesis assays
   b. Depurination Assays
   c. Cell growth/survival/viability assays

E. Exemplary Modified Toxins
Modified SA1 Toxins

F. Targeting Agents and Conjugates Thereof
1. Targeting Agents
   a. Chemokines
      i. Ligands
      ii. Chemokine Receptors
      iii. Chemokine/Chemokine Receptor Cellular Profile
      iv. Exemplary Chemokine Targeting Agents
   b. Non-Chemokine Cytokines
   c. Antibody Ligand Moieties
   d. Other targeting agents and receptor targets
      Growth Factors
2. Linker Moieties
   a. Exemplary Linkers
      i. Heterobifunctional Cross-linking Reagents
      ii. Acid Cleavable, Photocleavable and Heat Sensitive Linkers
      iii. Other Linkers for Chemical Conjugation
      iv. Peptide Linkers
3. Exemplary Leukocyte Population Modulator (LPM) Conjugates

G. Preparation of Modified Rip Toxins and Conjugates Thereof
1. Methods of Generating and Cloning Toxin Polypeptides, or Conjugates Containing Toxin Polypeptides
2. Production of Conjugates Containing Fusion Proteins and Expression Systems
   a. Plasmids and host cells for expression
      i. Bacterial cell expression systems
      ii. Insect cell expression systems
      iii. Yeast cell expression systems
      iv. Plant cell expression systems
      v. Mammalian cell expression systems
   b. Purification
3. Production of chemical conjugates

H. Methods to Increase Production of RIP Polypeptides, or Conjugates thereof.
Additional Methods to Increase Protein Production

I. In vitro and In vivo Assays to measure activity of toxins or conjugates thereof
1. In vitro activity assays
   a. Cell-Based Toxicity Assays
   b. Receptor Binding Assays and Internalization
   c. Chemotaxis Assays
2. In vivo Animal Models for Testing of Conjugates
   a. Spinal cord injury (SCI)
   b. Traumatic brain injury and stroke
   c. Alzheimer's Disease
   d. Multiple Sclerosis
   e. Arthritis and autoimmune disease
   f. Inflammatory lung diseases
   g. Inflammation after gene therapy
   h. Angiogenesis
   i. Tumor growth
   j. Human Immunodeficiency Virus (HIV)
   k. kidney disease
   l. hypersensitivity

J. Formulation and Administration of Compositions Containing Toxins and Conjugates Thereof

K. Methods of Treatment of Diseases and Disorders Using Toxins or Conjugates Thereof
1. The Immune Host Defense System and Inflammation
   a. Homeostatic inflammation
   b. Pathological inflammation
2. Candidate Therapeutics and Limitations Thereof
3. Ligand-toxin conjugates (i.e. LPMs)
   Selection of Ligand-Toxin Conjugate for Treatment of Selected Diseases or Disorders
   Selection and Design of Leukocyte Population Modulators
4. Exemplary Diseases
   a. Cancer
   b. Kidney Disease
   c. Spinal Cord Injury (SCI)
   d. Hypersensitivity
   e. HIV infection and AIDS and infections with other pathogens
   f. Inflammatory Joint Disease and Autoimmune Disease
   g. Pulmonary Disease
   h. Other Disease mediated by Secondary Tissue Damage
5. Combination Therapies

L. Examples

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, toxin (also referred to as a cytotoxin) refers to a molecule such as a polypeptide or drug, that when internalized into a cell inhibits cell function, such as by inhibiting cell growth and/or proliferation. The toxin can inhibit proliferation or is toxic to cells. Any molecule that when internalized by a cell interferes with or detrimentally alters cellular metabolism or in any manner inhibits cell growth or proliferation are included within the ambit of this term, including, but are not limited to, molecules whose toxic effects are mediated when transported into the cell and also those whose toxic effects are mediated at the cell surface. A variety of cytotoxins are known and include those that inhibit protein synthesis and those that inhibit expression of certain genes essential for cellular growth or survival. Toxins include those that result in cell death and those that inhibit cell growth, proliferation and/or differentiation or otherwise detrimentally alter cellular metabolism. For example, toxins, include, but are not limited to, ribosome-inactivating proteins (RIPs). The RIPs, upon internalization into a cell, alter metabolism or gene expression in the cell, regulate or alter protein synthesis, inhibits proliferation, kill the cell or otherwise detrimentally affect the cell. For purposes herein, a toxin, for example, a RIP protein, such as a modified RIP protein provided herein, is a targeted agent. The toxins inhibit growth and proliferation or interfere with or detrimentally alter cellular metabolism or in any manner of host cells in which they are expressed when the cells are cultured under standard or normal conditions for such cells.

As used herein, growth under standard conditions with reference to host cells, refers to conditions under which such cells are normally grown to express encoded proteins or recombinant proteins.

As used herein, ribosome inactivating protein (RIP) refers to a class of proteins expressed in plants and bacteria that are potent inhibitors of eukaryotic and prokaryotic protein synthesis. RIPs also degrade cellular DNA upon import into the nucleus. RIPs are N-glycosidases or polynucleotide:adenosine glycosidases and are able to inactivate ribosomal and nonribosomal nucleic acid substrates.

As used herein, reference to RIP polypeptides refers to any polypeptide that exhibits N-glycosidase activity and inactivates ribosomes. These include polypeptides isolated from natural sources as well as those made synthetically, such as by recombinant methods, by chemical synthesis or any method. They also include variants, wildtype, species and allelic variants. Exemplary RIPs include, but are not limited to, any Type I or Type II RIPs including, but not limited to, Shiga toxin including Shiga toxin 1 (Stx1), Stx2, Saporin 6, Barley RIP I, Barley RIP II, Gelonin, Ricin A, Momordin I, Momordin II, Bryodin I, Bryodin II, Pap-S, Luffin, Trichosanthin, Clavin, Abrin-a, Maize RIP 3, Maize RIP 9, Maize RIP X, Tritin, MAP, Dianthin 30, Nigrin b, Nigrin I, Ebulin, cytotoxically active fragments of these toxins, and other RIPs known to those of skill in this art. RIP polypeptides also encompass variants and other modified forms, such as muteins, of RIP polypeptides. Typically variants and modified forms possess N-glycosidase activity. Variants include, for example, allelic and species variants and also those with insertions or deletions of amino acid residues. Exemplary sequences of RIP proteins are any that include amino acid residues having an amino acid sequence set forth in any of SEQ ID NOS: 1, 5, 89-111 as well as allelic and/or species variants thereof and homologs and modified versions thereof that have at least 40, 50, 60, 65, 70, 75, 80, 85, 90, 95, 96, 97, 98, 99 or more sequence identity, particularly any that retain N-glycosidase activity. Exemplary RIP variants include any known in the art or those provided herein, such as a RIP protein having any one or more amino acid variations as set forth in SEQ ID NOS: 3, 6-21, 162-169.

As used herein, a “functional activity” or “activity” of a RIP polypeptide refers to any activity exhibited by a RIP polypeptide that can be assessed. Such activities can be tested in vitro and/or in vivo and include, but are not limited to N-glyocosidase activity and/or polynucleotide:adenosine glycosidase activity including RNAase and DNAase activity. Other activities include, but are not limited to, superoxide dismuatase activity, phospholipase activity, chitinase activity and anti-viral activity. Assays to determine activity of RIP polypeptides, modified forms thereof or conjugates thereof, are known to those of skill in the art. For example, activity can be assessed by assaying for protein synthesis, depurination and/or cell growth/viability. In addition, the polynucleotide:adenosine glycosidase activity can be assessed, for example, by purifying the DNA from cells treated with a RIP polypeptide and visualizing by staining with ethidium bromide. Exemplary assays to assess the activity of a RIP polypeptide, such as a modified SA1 polypeptide, or conjugates thereof are set forth herein or described in Examples 2 and 5.

As used herein, an active fragment (used interchangeably with an active portion) of a toxin refers to a fragment that has an activity, such as a toxic activity, or a catalytic activity. Hence reference is made to catalytically active fragments of toxins, such RIPs, and fragments that retain toxin activity. Where a modified toxin, such as a modified Shiga toxin, is provided, the active fragment includes a modification.

As used herein, variant toxin polypeptides, such as variant RIPs, refer collectively to RIPs prior to modification to reduce toxicity as described herein. Variant toxin is any form of that polypeptide that differs from a wildtype form, and includes allelic and/or species variants, polypeptides encoded by splice variants, and/or modified forms, particularly variants with changes in the primary structure. Variants include those that contain deletion, replacement, or addition of amino acids compared to a wildtype form of the protein. For example, variants of SA1 include those that contain amino acid mutations or are truncated compared to the wildtype SA1 corresponding to amino acids 1-251 of the mature A domain set forth in SEQ ID NO:5, as well as allelic or species variations thereof. Exemplary of truncations are variants 1 and variants 2 set forth in SEQ ID NOS: 22 and 24, respectively.

As used herein, species variants refer to variants in polypeptides among different species, including different bacterial species, such as Escherichia and Shigella.

As used herein, allelic variants refer to variations in proteins among members of the same species.

As used herein, an unmodified RIP polypeptide refers to a starting protein that is selected for modification. The starting target polypeptide can be the naturally-occurring, wild-type form of a polypeptide. In addition, the starting target polypeptide can have been previously altered or mutated, such that it differs from the native wild type isoform but is nonetheless referred to herein as a starting unmodified target protein relative to the subsequently modified proteins produced herein. Thus, proteins known that have been modified to have a desired increase or decrease in a particular activity or property compared to an unmodified reference protein can be used as the starting unmodified target protein. For purposes herein, an unmodified RIP polypeptide includes the RIP polypeptide alone, or an active fragment thereof, or conjugates containing a REP polypeptide or active fragment thereof.

As used herein, a “modified” or “mutant” RIP polypeptide refers to a polypeptide that has one or more modifications in primary sequence compared to a reference starting protein or unmodified polypeptide, such as a wildtype polypeptide, or other starting RIP polypeptide including allelic variants, of a particular species and other variants. The modification or mutation alters toxicity (i.e., the ability to alter metabolism or gene expression in the cell, regulate or alter protein synthesis, inhibit proliferation, kill the cell or otherwise detrimentally affect the cell). Hence a modified or mutant RIP contains mutations, including insertions and deletions of amino acid residues in any RIP, whereby toxicity is reduced compared to the starting RIP. The one or more mutations include one or more amino acid replacements (substitutions), insertions, deletions and any combination thereof. A modified RIP polypeptide can have 1, 2, 3, 4, 5, 6, 7, 8, 9, 10, 11, 12, 13, 14, 15, 16, 17, 18, 19, 20 or more modified positions. Generally, these mutations change the toxicity and/or one or more other activities of the RIP polypeptide. Such modification include those identified in the selection methods herein. In addition to containing modifications that alter the toxicity of the polypeptide, a modified RIP polypeptide also can contain other modifications. A modified RIP polypeptide typically has 60%, 70%, 80%, 85%, 90%, 91%, 92%, 93%, 94%, 95%, 96%, 91%, 98%, 99% or more sequence identity to a corresponding sequence of amino acids of a wildtype or starting unmodified RIP polypeptide.

As used herein, Shiga-Toxin refers to a RIP polypeptide originally isolated from bacteria, particularly members of the genus Shigella and other related genuses, such as Shigella dysenteriae. Shiga Toxin is a multisubunit protein made up of an A subunit, which becomes cleaved into A1 and A2 to form the active Shiga Toxin A1 (SA1) moiety, and five B subunits. The B subunits are linked to the A2 moiety and are required for entry of Shiga Toxin into cells (Sandvig and van Deurs, EMBO J., 19: 5943-50, 2000). In the conjugates herein, the B subunits are replaced with a targeting agent for entry into a cell. Hence the conjugates include the toxin subunit, particularly subunit A, and most particularly, the catalytically active fragment (SA1) or an active fragment thereof. An exemplary precursor sequence of an A subunit of Shiga Toxin is set forth in SEQ ID NO: 1, and the mature sequence is set forth in SEQ ID NO:5. The catalytically active A1 fragment (SA1) corresponds to amino acids 1-251 of the sequence set forth in SEQ ID NO:5, and the A2 fragment corresponds to amino acids 252-293 of the sequence set forth in SEQ ID NO:5.

Shiga toxins also exhibit allelic and species variations. Examples of shiga toxins include those produced by Shigella species and allelic and species variants there, such as, but not limited to, those produced in Shigella dysenteriae, E. coli, Citrobacter freundii, Aeromononas hydrophila, Aeromononas caviae, and Enterobacter cloacae. Exemplary sequences of the precursor or mature form of the A chains of various Shiga Toxins are set forth in any of SEQ ID NOS: 1, 3, 5 and 7-21. Other variants in the Shiga Toxin A chain are set forth in SEQ ID NO:6.

As used herein, “enzymatic subunit” or “catalytically active subunit” or “active subunit” of a RIP polypeptide refers to the portion of the polypeptide that mediates a toxic activity. The toxic activity can be any property or activity of the polypeptide, such as due to inhibitory activity against rRNA by virtue of an N-glycosidase activity, or depurination of DNA, mRNA, or viral DNA or viral RNA. For example, for Shiga Toxin, the active portion is the A1 subunit (SA1), which is activated by cleavage of the A subunit into A1 and A2 fragments. Hence, an active portion of the A-chain of Shiga Toxin is the A1 subunit also referred to as SA1.

Active portions of Shiga Toxins, as well as of any RIP, are known or can be empirically identified using, in in vitro or in vivo activity assays that assess activity (see, e.g., Stirpe et al., Bio/Technology 10:405-12, 1992; and Sandvig and Van Deurs, Physiol. Rev. 76:949-66, 1996; Stirpe and Battelli, Cell Mol Life Sci., 63: 1850-66, 2006). The A subunits of exemplary RIPs are set forth in Table 3 herein, and or are known or could be identified by one of skill in the art.

As used herein, an “active portion thereof” or “active fragment thereof” of a REP toxin refers to a polypeptide that contains at least the minimal amino acid residues to manifest a toxic activity. Typically an active portion contains contiguous amino acids from a RIP polypeptide, such as the minimal portion of the A subunit or A1 subunit, required to provide a toxic activity. Active fragments and the minimal amino acid residues can be empirically determined by producing and testing truncations of one or both of the N- or C-termini of a RIP polypeptide A subunit or A1 subunit to determine those that display an activity. Activity can be assessed by various assays described herein or known in the art including, but not limited to, protein synthesis assays, depurination assays, or cell growth/viability assays. Activity can be any percentage of activity (more or less) of the full-length polypeptide, including but not limited to, 1% of the activity, 2%, 3%, 4%, 5%, 10%, 20%, 30%, 40%, 50%, 60%, 70%, 80%, 90%, 95%, 96%, 97%, 98%, 99%, 100%, 200%, 300%, 400%, 500%, or more activity compared to the full polypeptide.

Typically, an active fragment of a RIP toxin is a truncated fragment in which about 1, 2, 3, 4, 5, 6, 7, 8, 9, 10, 11, 12, 13, 14, or 15 amino acids at the N- or C-terminus of the A-chain of the polypeptide are missing. Exemplary active fragments of the catalytically active SA1 subunit, or an active fragment thereof of a Shiga Toxin are set forth in SEQ ID NO:22 or SEQ ID NO:24.

As used herein, toxicity refers to the ability of a molecule, including a peptide, protein, chemical, or other molecule to alter metabolism or gene expression in the cell, regulate or alter protein synthesis, inhibit proliferation, kill the cell or otherwise detrimentally affect the cell. For purposes herein, with respect to RIPs, toxicity refers to the ability of a RIP, or subunit thereof or fragment thereof, to, upon internalization into a cell to alter metabolism or gene expression in the cell, regulate or alter protein synthesis, inhibit proliferation, kill the cell or otherwise detrimentally affect the cell. For example, RIP polypeptides, or conjugates thereof, exhibit cellular toxicity via a variety of activities including, but not limited to, their N-glycosidase activity and/or polynucleotide:adenosine glycosidase activity.

As used herein, N-glycosidase activity refers to polypeptide enzymes that cleave nucleotide N-glycosidic bonds. RIP polypeptides exhibit glycosidase activity by removing a specific adenine residue from ribosomal rRNA. Such activity results in the inhibition of protein synthesis and subsequent cell death by preventing the binding of elongation factors to the ribosome.

As used herein, corresponding residues refers to residues that occur at aligned loci. Related or variant polypeptides are aligned by any method known to those of skill in the art. Such methods typically maximize matches, and include methods, such as using manual alignments and by using the numerous alignment programs available (for example, BLASTP) and others known to those of skill in the art. By aligning the sequences of polypeptides, one skilled in the art can identify corresponding residues, using conserved and identical amino acid residues as guides. For example, one of skill in the art recognizes that the referenced positions of a mature Shiga toxin A-chain set forth in SEQ ID NO: 5 differs by twenty two amino acid residues when compared to a precursor Shiga toxin A-chain set forth in SEQ ID NO: 1, due to the presence of a signal sequence. Thus, the amino acid at position twenty three of SEQ ID NO: 1 “corresponds to” the first amino acid residue of SEQ ID NO: 5. Further, one skilled in the art also can employ conserved amino acid residues as guides to find corresponding amino acid residues between and among human and non-human sequences. Corresponding positions also can be based on structural alignments, for example by using computer simulated alignments of protein structure. In other instances, corresponding regions can be identified. One skilled in the art also can employ conserved amino acid residues as guides to find corresponding amino acid residues between and among human and non-human sequences.

As used herein, “primary sequence” refers to the sequence of amino acid residues in a polypeptide.

As used herein, the terms “homology” and “identity” are used interchangeably, but homology for proteins can include conservative amino acid changes. In general to identify corresponding positions the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073).

As use herein, “sequence identity” refers to the number of identical amino acids (or nucleotide bases) in a comparison between a test and a reference polypeptide or polynucleotide. Homologous polypeptides refer to a pre-determined number of identical or homologous amino acid residues. Homology includes conservative amino acid substitutions as well as identical residues. Sequence identity can be determined by standard alignment algorithm programs used with default gap penalties established by each supplier. Homologous nucleic acid molecules refer to a pre-determined number of identical or homologous nucleotides. Homology includes substitutions that do not change the encoded amino acid (i.e., “silent substitutions”) as well as identical residues. Substantially homologous nucleic acid molecules hybridize typically at moderate stringency or at high stringency all along the length of the nucleic acid or along at least about 70%, 80% or 90% of the full-length nucleic acid molecule of interest. Also contemplated are nucleic acid molecules that contain degenerate codons in place of codons in the hybridizing nucleic acid molecule or in the molecule with a specified sequence identity. For determination of homology of proteins, conservative amino acids can be aligned as well as identical amino acids; in this case, percentage of identity and percentage homology varies. Whether any two nucleic acid molecules have nucleotide sequences (or any two polypeptides have amino acid sequences) that are at least 80%, 85%, 90%, 95%, 96%, 97%, 98% or 99% “identical” can be determined using known computer algorithms such as the “FAST A” program, using for example, the default parameters as in Pearson et al. Proc. Natl. Acad. Sci. USA 85: 2444 (1988) (other programs include the GCG program package (Devereux, J., et al., Nucleic Acids Research 12(I): 387 (1984)), BLASTP, BLASTN, FASTA (Atschul, S. F., et al., J. Molec. Biol. 215:403 (1990); Guide to Huge Computers, Martin J. Bishop, ed., Academic Press, San Diego (1994), and Carillo et al. SIAM J Applied Math 48: 1073 (1988)). For example, the BLAST function of the National Center for Biotechnology Information database can be used to determine identity. Other commercially or publicly available programs include DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. J. Mol. Biol. 48: 443 (1970), as revised by Smith and Waterman (Adv. Appl. Math. 2: 482 (1981)). Briefly, a GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and 0 for non identities) and the weighted comparison matrix of Gribskov et al. Nucl. Acids Res. 14: 6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps.

As used herein, the term “identity” represents a comparison between a test and a reference polypeptide or polynucleotide. In one non-limiting example, “at least 90% identical to” refers to percent identities from 90 to 100% relative to the reference polypeptides. Identity at a level of 90% or more is indicative of the fact that, assuming for exemplification purposes a test and reference polynucleotide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often without relying on software.

As used herein, it also is understood that the terms “substantially identical” or “similar” varies with the context as understood by those skilled in the relevant art, but that those of skill can assess such.

As used herein, a “selection method” refers to any method where a protein is identified based on a particular attribute, property or activity. For purposes herein, RIP polypeptides, or active fragments thereof, identified in the selection method herein include those that display a reduced toxicity compared to an unmodified or starting protein.

As used herein, production by recombinant methods refers to using recombinant DNA methods to express a recombinant polypeptide. Such methods are well-known to one of skill in the art and typically include methods of molecular biology for expressing proteins encoded by cloned DNA.

As used herein, “increased yield” refers to the amount of a REP produced, such as mg/l or absolute amount, with reference to the amount of a REP produced in the presence of a RIP inhibitor compared to in the absence of the RIP inhibitor.

As used herein, “isolated” with reference to cells refers to the separation of a cell, colony of cells, or population of cells away from other cell colonies or populations of cells. Isolation can be effected by any procedure which separates cells, such as plating conditions, purification techniques such as the use of magnetic beads, particular cellular characteristics such as granularity, or other similar techniques. For example, isolation can be effected by plating out or spreading a sample of a cell culture, such as a bacterial cell culture, on a nutrient agar surface under conditions where each viable cell grows and forms a colony of cells. Plating conditions can be optimized, such as by diluting of the cell culture, so that a single colony of cells is detected as a discrete colony. Cells or colonies of cells can be individually picked or selected as a single cell.

As used herein, “isolated” with reference to a nucleic acid molecule or polypeptide or other biomolecule means that the nucleic acid or polypeptide has separated from the genetic environment from which the polypeptide or nucleic acid or cell were obtained. It also can mean altered from the natural state. For example, a polynucleotide or a polypeptide naturally present in a living animal is not “isolated,” but the same polynucleotide or polypeptide separated from the coexisting materials of its natural state is “isolated,” as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been partially or substantially purified from a recombinant host cell or from a native source. For example, a recombinantly produced version of a compound can be substantially purified by the one-step method described in Smith et al., Gene, 67:31-40 (1988). The terms isolated and purified can be used interchangeably.

Thus, by “isolated” it is meant that the nucleic acid is free of coding sequences of those genes that, in the naturally-occurring genome of the organism (if any), immediately flank the gene encoding the nucleic acid of interest. Isolated DNA can be single-stranded or double-stranded, and can be genomic DNA, cDNA, recombinant hybrid DNA or synthetic DNA. It can be identical to a starting DNA sequence or can differ from such sequence by the deletion, addition, or substitution of one or more nucleotides.

As used herein, “purified” preparations made from biological cells or hosts mean at least the purity of a cell extract containing the indicated DNA or protein including a crude extract of the DNA or protein of interest. For example, in the case of a protein, a purified preparation can be obtained following an individual technique or a series of preparative or biochemical techniques, and the DNA or protein of interest can be present at various degrees of purity in these preparations. The procedures can include, but are not limited to, ammonium sulfate fractionation, gel filtration, ion exchange chromatography, affinity chromatography, density gradient centrifugation, and electrophoresis.

As used herein, a preparation of DNA or protein that is “substantially pure” or “isolated” refers to a preparation substantially free from naturally-occurring materials with which such DNA or protein is normally associated in nature and generally contains 5% or less of the other contaminants.

As used herein, a cell extract that contains the DNA or protein of interest refers to a homogenate preparation or cell-free preparation obtained from cells that express the protein or contain the DNA of interest. The term “cell extract” is intended to include culture medium, especially spent culture medium from which the cells have been removed.

As used herein, a “selective agent” or “selection agent” refers to any factor to which cells or populations of cells are sensitive or susceptible, and which, by virtue of the sensitivity can be used to identify cells that exhibit resistance to the agent or to the effects of the agent on the cells. Typically, selection agents are used in combination with expression systems to select for expressed polypeptides that confer resistance to the host cell to the specific selective agent. Exemplary of selective agents are antibiotics.

As used herein, a “selection modulating agent” or “selection modulator” or “agent that modulates selection” refers to any factor or agent used in a selection method that improves or increases the ability to select a particular attribute, property or activity, such as an attribute, property or activity of a recombinant polypeptide. For purposes herein, an agent that modulates selection can be used in the methods of selection to improve the selection of RIP polypeptides, or active fragments thereof, which exhibit altered toxicity. Exemplary of selection modulators are RIP inhibitors. For example, a RIP inhibitor, such as an adenine analog, decreases or eases the toxicity of a RIP polypeptide to a host cell, thereby allowing for expression of the RIP in the host cell. The selection modulator chosen, its concentration and incubation time are factors that can influence the ability of a selection modulator to enhance the ability to select for a particular attribute, property or activity. Selection modulators thus differ from selection agents

As used herein, an “induction agent” refers to any factor that is used to initiate recombinant protein expression in a host cell. Factors that can be used as inducers include, but are not limited to, changes in temperature or the administration of a small molecules, peptides or polypeptides. The choice of induction agent depends on the host cell used for recombinant protein expression and on the specific promoter used to express the protein. One of skill in the art is familiar with various induction agents. For example, in the pET expression system, the T7 RNA polymerase required for gene expression is under the control of the IPTG-inducible T7 promoter. Protein expression does not occur in host cells, typically E. coli BL21 (DE3) cells, transformed with a pET vector containing a cloned gene, until induction by IPTG.

As used herein, a RIP inhibitor is any chemical, such as a peptide, polypeptide, oligonucleotide or other molecule or condition, that inhibits the activity of a RIP polypeptide. Typically, RIP inhibitors include any that inhibit the N-glycosidase activity of a RIP polypeptide. Hence, RIP inhibitors are any agent, polypeptide, or other molecule that reduces the activity of a RIP polypeptide. Such agents are known and include any that reduce the activity of a RIP polypeptide. Exemplary of a RIP inhibitor is 4-aminopyrazolo[3,4-d]pyrimidine (4-APP).

As used herein, “effective to inhibit the toxicity of a RIP polypeptide” when referring to a RIP inhibitor means that in the presence of the inhibitor, a RIP polypeptide retains no to little activity or its activity is reduced when incubated in the presence of the RIP inhibitor. For example, a RIP polypeptide whose toxicity is inhibited exhibits a 50%, 60%, 70%, 80%, 90%, 91%, 92%, 93%, 94%, 95%, 96%, 97%, 98%, 99%, or 100% reduction in toxicity compared to the toxic activity of the RIP polypeptide in the absence of the RIP inhibitor.

As used herein, “retains toxic activity” refers to a RIP polypeptide or active portion thereof that exhibits an activity of a RIP polypeptide, which activity is typically reduced compared to a wild-type, starting or reference form of a RIP polypeptide. For purposes herein, an activity is retained if it is sufficient enough to exhibit a toxic activity against a ribosome, DNA, mRNA, tRNA or target host cell. For example, a RIP polypeptide or active portion thereof retains an activity if it displays at least 1%, 2%, 3%, 4%, 5%, 6%, 7%, 8%, 9%, 10%, 20%, 30%, 40%, 50%, 60%, 70%, 80%, 90%, 95%, or more activity compared to a wild-type, starting or reference RIP polypeptide. A RIP or other toxin can exhibit a substantial reduction in activity, even to less than 1% of its original activity, as long as a conjugate containing such RIP is effective for treatment.

As used herein, a conjugate refers to the molecules provided herein that include one or more targeting moieties linked directly or indirectly to one or more targeted agents that are modified RIP toxins. These conjugates also are referred to herein as ligand-toxin conjugates and include, for example, leukocyte population modulators (LPMs). Such conjugates include fusion proteins, those produced by chemical conjugates and those produced by any other method whereby at least one modified toxin is linked, directly or indirectly to a targeting agent, whereby upon binding to a cell surface receptor the toxin is internalized into the targeted cell.

As used herein, a leukocyte population modulator (LPM) is a ligand-toxin conjugate where the targeting agent is a polypeptide portion that is sufficient to target the conjugate to one or more chemokine receptors expressed on a cell thereby effecting internalization of a linked or otherwise associated targeted agent. Generally, the polypeptide portion of an LPM is a chemokine ligand, fragment or allelic, species or splice variant thereof, that targets the conjugate to one or more chemokine receptors. Typically, via cell surface expressed chemokine receptors, such a conjugate is targeted to one or more than one leukocyte.

As used herein, a fusion protein refers to a polypeptide that contains at least two polypeptide components, such as a targeting moiety (i.e. a chemokine) and a targeted agent, the toxin, and optionally a peptide or polypeptide linker. Such proteins can be produced by expression of a nucleic acid encoding the conjugate in host cells.

As used herein, a targeted agent is any agent that is intended for internalization by linkage to a targeting moiety, as defined herein, and that upon internalization in some manner alters or affects cellular metabolism, growth, activity, viability or other property or characteristic of the cell. The targeted agents herein are the modified toxins. Exemplary of targeted agents provided herein are SA1 or active fragments thereof, including modified SA1 polypeptides.

As used herein, to target a targeted agent means to direct it to a cell that expresses a selected receptor by linking the agent to a targeting moiety. Upon binding to the receptor the targeted agent or targeted agent linked to the receptor binding moiety is internalized by the cell.

As used herein, “immune cell” or “immune effector cell” refers to any cell that helps defend the body against infectious disease and foreign materials as part of the immune system. Such cells include those found in the blood, in the lymphatic system, and in other body tissues. These include, but are not limited to, leukocytes and other tissue resident cells such as kupffer cells, microglia, alveolar macrophage or other tissue associated immune cell.

As used herein, leukocyte refers to a white blood cell that plays a role in the body's host immune defense system. Leukocytes include, but are not limited to, monocytes, macrophages, dendritic cells, mast cells, natural killer cells, granulocytes (basophils, eosinophils, neutrophils), and lymphocytes (B and T lymphocytes).

As used herein, tissue residential cell (TRC) refer to specialized cells that reside in or is specific to particular tissues or organs. Many tissue residential cells play a role in the body's immune defenses, particularly with respect to the specific tissue. Included among such TRC are Kupffer cells of the liver, microglia of the brain and alveolar macrophages of the lung.

As used herein, activated cells with reference to immune cells or leukocytes refers to cells that, upon stimulation, exhibit an altered gene expression profile compared to cells that were not stimulated. Typically, such cells secrete or produce or upregulate expression of soluble or cell surface-bound peptide or polypeptide mediators, such as inflammatory or other immune mediators, for example, cytokines, chemokines or other chemical messenger proteins or receptors therefor, which expression or production is greater than prior to stimulation.

As used herein, a targeting agent refers to any cell binding ligand polypeptide, or portion thereof, that binds to a targeted cell by binding to a cell surface receptor followed by internalization thereof. A targeting agent is any agent that facilitates internalization of the targeted moiety. Hence, it is any agent that binds to an endocytic cell surface receptor. Targeting moieties can include any polypeptide, or portion thereof, that binds to any cellular receptor or cellular ligand so long as the polypeptide is internalized by the cell following binding to the cell surface molecule. For example, targeting moieties include, but are not limited to, antibodies, growth factors, cytokines, chemokines, and others. Exemplary of targeting agents are those agents that target to chemokine receptors.

As used herein, chemokine receptors refer to receptors that specifically interact with a naturally-occurring member of the chemokine family of proteins and transport it into a cell bearing such receptors. These include, but are not limited to, the receptors (CXCR1-7, including CXCR3A and CXCR3B) to which CXC chemokines bind and the receptors (CCR1-10, including CCR2A and CCR2B) to which CC chemokines bind, and any other receptors to which any chemokine specifically binds and facilitates internalization of a linked targeted agent.

As used herein, a chemokine receptor targeting agent refers to any molecule or ligand that specifically binds to a chemokine receptor on a cell and effects internalization of a linked or otherwise associated targeted agent. Chemokine receptor binding moieties, include, but are not limited to, any polypeptide that is capable of binding to a cell-surface protein to which a chemokine would be targeted, and is capable of facilitating the internalization of a ligand-containing fusion protein into the cell. Such polypeptides include chemokines, antibodies, or fragments thereof so long as the polypeptide binds to one or more chemokine receptors and effects internalization of any linked targeted agent. Identification of fragments or portions of a polypeptide, such as a chemokine or antibody, that is effective in binding to one or more chemokine receptors and internalizing a linked targeted agents can be done empirically, by testing, for example, a fragment linked to a cytotoxic agent, and looking for cell death using any of the assays therefor described herein or known to those of skill in the art. Hence, a chemokine receptor targeting agent includes all of the peptides characterized and designated as chemokines, which include, but are not limited to, classes described herein, and truncated versions and portions thereof that are sufficient to direct a linked targeted agent to a cell surface receptor or protein to which the full-length chemokine specifically binds and to facilitate or enable internalization by the cell on which the receptor or protein is present.

As used herein, the term “cytokine” refers to polypeptides that include interleukins, chemokines, lymphokines, monokines, colony stimulating factors, growth factors, adipokines and receptor associated proteins, and functional fragments thereof. For purposes herein, non-chemokine cytokines refer to all cytokines, most typically the classic cytokines and does not include the chemokines, which have chemoattractant and other activities not generally exhibited by other (classic) cytokines. Chemokines, as recognized by those skill in the art and discussed herein below, however, are a distinct class of polypeptides.

As used herein, chemokines refers to a family of small proteins secreted from cells that promote the movement or chemotaxis of nearby cells. Some chemokines are considered pro-inflammatory and can be induced during an immune response while others are considered homeostatic. Typically, chemokines exert their chemoattractant function and other functions by binding to one or more chemokine receptors. Chemokines include proteins isolated from natural sources as well as those made synthetically, by recombinant means or by chemical synthesis. Exemplary chemokines (set forth in SEQ ID NOs: 112-161) include, but are not limited to, MCP-1, Eotaxin, SDF-1β, GRO-α, MIP-1β, IL-8, IP-10, MCP-3, MIP-3α, MDC, MIP-1α, BCA-1, GCP-2, ENA-78, PBP, MIG, PF-4, PF-4-var1, SDF-2, MCP-2, MCP-4, MIP-4, MIP-3β, MIP-2α, MIP-2β, MIP-5, HCC-1, RANTES, Eotaxin-2, TARC, I-309, Lymphotactin, Lungkine, C10, MIP-1γ, MCP-5, LEC, Exodus-2, MIP-3, TECK, Eotaxin-3, CTACK, MEC, SCM-1β, I-TAC, BRAK, SR-PSOX, Fractalkine, LD78-β, MIP-1b2, and others known to those of skill in the art. References to chemokines typically includes monomeric forms of such chemokines. Chemokines also include dimeric or other multimeric forms.

Chemokine encompasses variants or muteins of chemokines that possess the ability to target a linked targeted agent to chemokine-receptor bearing cells. Muteins of chemokines also are contemplated as targeting agents for use in the conjugates. Such muteins can have conservative amino acid changes, such as those set forth below in the following Table 1. Nucleic acids encoding such muteins will, unless modified by replacement of degenerate codons, hybridize under conditions of at least low stringency to DNA, generally high stringency, to DNA encoding a wild-type protein. Muteins and modifications of the proteins also include, but are not limited to, minor allelic or species variations and insertions or deletions of residues. Examples of chemokine variants are set forth in SEQ ID NOs: 170-191. Suitable conservative and non-conservative substitutions of amino acids are known to those of skill in this art and can be made generally without altering the activity of the resulting molecule. Those of skill in this art recognize that, in general, single amino acid substitutions in non-essential regions of a polypeptide do not substantially alter activity (see, e.g., Watson et al. Molecular Biology of the Gene, 4th Edition, 1987, The Benjamin/Cummings Pub. co., p. 224). Such substitutions can be made in accordance with those set forth as follows:

TABLE 1

| Original residue | Conservative substitution |
|---|---|
| Ala (A) | Gly; Ser |
| Arg (R) | Lys |
| Asn (N) | Gln; His |
| Cys (C) | Ser; neutral amino acid |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative or non-conservative substitutions. Any such modification of the polypeptide can be effected by any means known to those of skill in this art.

As used herein, a portion of a chemokine refers to a fragment or piece of chemokine that is sufficient, either alone or as a dimer with another fragment or a chemokine monomer, to bind to a chemokine receptor for internalization of a linked targeted agent. Various in vitro assays for identification of chemokines and chemokine activity, particularly chemotactic activities, are known to those of skill in the art (see, e.g., Walz et al. (1987) Biochem. Biophys. Res. Commun. 149:755 to identify chemotaxis of neutrophils; Larsen et al. (1989) Science 243:1464 and Carr et al. (1994) Proc. Natl. Acad. Sci. U.S.A. 91:3652 to assay chemotaxis of lymphocytes; see, also International PCT application No. WO 99/33990, which describes numerous assays and exemplifies means to identify chemokines). Such assays can be used to identify chemokines, modified chemokines and active fragments thereof. Binding assays, as described herein and known to those of skill in the art can be used to identify moieties that will specifically recognize chemokine receptors, and cytotoxic assays can be used to identify those that also internalize linked or associated targeted agents.

As used herein, nucleic acid encoding a chemokine peptide or polypeptide refers to any of the nucleic acid fragments set forth herein as coding such peptides, to any such nucleic acid fragments known to those of skill in the art, any nucleic acid fragment that encodes a chemokine that binds to a chemokine receptor and is internalized thereby and can be isolated from a human cell library using any of the preceding nucleic acid fragments as a probe or any nucleic acid fragment that encodes any of the known chemokine peptides, including those set forth in SEQ ID NOs:112-161, 170-191 and any DNA fragment that can be produced from any of the preceding nucleic acid fragments by substitution of degenerate codons. It is understood that once the complete amino acid sequence of a peptide, such as a chemokine peptide, and one nucleic fragment encoding such peptide are available to those of skill in the art, it is routine to substitute degenerate codons and produce any of the possible nucleic fragments that encode such peptide. It also is generally possible to synthesize nucleic acids encoding such peptides based on the amino acid sequence.

As used herein, a linker is a peptide or other molecule that links a targeting agent (i.e. chemokine polypeptide) to the targeted agent. The linker can be bound via the N- or C-terminus or an internal reside near, typically within about 20 amino acids, of either terminus of a targeted agent, if the agent is a polypeptide or peptide. Typically, where the targeted agent is a chemokine, linkage herein is at the C-terminus. The linkers used herein can serve merely to link the components of the conjugate, to increase intracellular availability, serum stability, specificity and solubility of the conjugate or provide increased flexibility or relieve steric hindrance in the conjugate. For example, specificity or intracellular availability of the targeted agent can be conferred by including a linker that is a substrate for certain proteases, such as a protease that is present at higher levels in tumor cells than normal cells.

As used herein, peptide and/or polypeptide means a polymer in which the monomers are amino acid residues which are joined together through amide bonds, alternatively referred to as a polypeptide. When the amino acids are alpha-amino acids, either the L-optical isomer or the D-optical isomer can be used, the L-isomers being preferred. Additionally, unnatural amino acids such as beta-alanine, phenylglycine, and homoarginine are meant to be included. Commonly encountered amino acids that are not gene-encoded also can be used in ligand-toxin chimeras provided herein, although preferred amino acids are those that are encodable.

As used herein, the “amino acids,” which occur in the various amino acid sequences appearing herein, are identified according to their well-known, three-letter or one-letter abbreviations (see Table 1). The nucleotides, which occur in the various DNA fragments, are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids, and amino acid analogs (e.g., amino acids wherein the α-carbon has a side chain).

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§ 1.821-1.822, abbreviations for amino acid residues are shown in Table 2.

TABLE 2
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

All sequences of amino acid residues represented herein by a formula have a left to right orientation in the conventional direction of amino-terminus to carboxyl-terminus. In addition, the phrase “amino acid residue” is defined to broadly include the amino acids listed in the Table of Correspondence (Table 2) and modified, non-natural and unusual amino acids, such as those referred to in 37 C.F.R. §§ 1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

As used herein, “naturally occurring amino acids” refer to the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally occurring amino acids and include, but are not limited to, the D-isostereomers of amino acids. Exemplary non-natural amino acids are known to those of skill in the art.

As used herein, vector or plasmid refers to discrete elements that are used to introduce heterologous DNA into cells for either expression of the heterologous DNA or for replication of the cloned heterologous DNA. Selection and use of such vectors and plasmids are well within the level of skill of the art.

As used herein, expression refers to the process by which nucleic acid is transcribed into mRNA and translated into peptides, polypeptides, or proteins. If the nucleic acid is derived from genomic DNA, expression can, if an appropriate eukaryotic host cell or organism is selected, include splicing of the mRNA.

As used herein, expression vector includes vectors capable of expressing DNA fragments that are in operative linkage with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or can integrate into the host cell genome.

As used, the term “nucleotide sequence coding for expression of” a polypeptide refers to a sequence that, upon transcription and subsequent translation of the resultant mRNA, produces the polypeptide.

As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, and maintenance of the correct reading frame of a protein-encoding gene to permit proper translation of the mRNA, and stop codons. In addition, DNA sequences encoding a fluorescent indicator polypeptide, such as a green or blue fluorescent protein, can be included in order to select positive clones (i.e., those host cells expressing the desired polypeptide).

As used herein, “host cells” are cells in which a vector can be propagated and its nucleic acid expressed. The term also includes any progeny of the subject host cell. It is understood that all progeny may not be identical to the parental cell since there can be mutations that occur during replication. Such progeny are included when the term “host cell” is used.

As used herein, secretion signal refers to a peptide region within the precursor protein that directs secretion of the precursor protein from the cytoplasm of the host into the periplasmic space or into the extracellular growth medium. Such signals can be either at the amino terminus or carboxy terminus of the precursor protein. The preferred secretion signal is linked to the amino terminus and can be heterologous to the protein to which it is linked. Typically signal sequences are cleaved during transit through the cellular secretion pathway. Cleavage is not essential or need to be precisely placed as long as the secreted protein retains its desired activity.

As used herein, transfection refers to the taking up of DNA or RNA by a host cell. Transformation refers to this process performed in a manner such that the DNA is replicable, either as an extrachromosomal element or as part of the chromosomal DNA of the host. Methods and means for effecting transfection and transformation are well known to those of skill in this art (see, e.g., Wigler et al. (1979) Proc. Natl. Acad. Sci. USA 76:1373-1376; Cohen et al. (1972) Proc. Natl. Acad. Sci. USA 69:2110).

As used herein, the term “functional fragment” refers to a polypeptide which possesses an activity that can be identified through a defined functional assay and that is associated with a particular biologic, morphologic, or phenotypic alteration in a cell or cell mechanism or cell activity.

As used herein, activity refers to any activity of a polypeptide exhibited in vitro and/or in vivo.

As used herein, biological activity refers to the in vivo activities of a compound or physiological responses that result upon in vivo administration of a compound, such as the conjugates provide herein, composition or other mixture. Biological activity, thus, encompasses therapeutic effects and pharmaceutical activity of such compounds, compositions and mixtures. Such biological activity can, however, be defined with reference to particular in vitro activities, as measured in a defined assay. Thus, for example, reference herein to the biological activity of a chemokine monomer, dimer or fragment thereof, or other combination of chemokine monomers and fragments, refers to the ability of the chemokine to bind to cells bearing chemokine receptors and internalize a linked agent. Such activity is typically assessed in vitro by linking the chemokine (dimer, monomer or fragment) to a cytotoxic agent, such as a modified shiga-A1 subunit, contacting cells bearing chemokine receptors, such as leukocytes, with the conjugate and assessing cell proliferation or growth. Such in vitro activity should be extrapolative to in vivo activity. Numerous animal models are referenced and described herein.

As used herein, the term biologically active, or reference to the biological activity of a conjugate made up of a targeting agent, such as a conjugate containing a chemokine and a targeted agent, such as a modified shiga-A1 subunit, refers in that instance to the ability of such polypeptide to enzymatically inhibit protein synthesis by inactivation of ribosomes either in vivo or in vitro or to inhibit the growth of or kill cells upon internalization of the toxin-containing polypeptide by the cells. Such biological or cytotoxic activity can be assayed by any method known to those of skill in the art including, but not limited to, the in vitro assays that measure protein synthesis and in vivo assays that assess cytotoxicity by measuring the effect of a test compound on cell proliferation or on protein synthesis. Particularly preferred, however, are assays that assess cytotoxicity in targeted cells.

As used herein, specifically binds to a targeted receptor means to bind with sufficient affinity for the receptor to effect internalization. Typically binding is with an affinity (Ka) of 10⁷ l/mol, 10⁸ l/mol greater.

As used herein, to bind to a receptor refers to the ability of a ligand to specifically recognize and specifically bind or detectably bind, as assayed by standard in vitro assays, to such receptors. For example, binding measures the capacity of the chemokine conjugate, chemokine monomer, or other chemokine receptor targeting agent to recognize a chemokine receptor on cells known to express such chemokine receptors. Such cells include cell lines or various primary leukocyte cell subtypes such as, but not limited to, microglia, monocytes, macrophages, neutrophils, eosinophils, basophils, natural killer cells, B cells, mast cells, dendritic cells and T-cells, or other tissue residential cells, or activated forms of such cells using well described ligand-receptor binding assays, chemotaxis assays, histopathologic analyses, flow cytometry and confocal microscopic analyses, and other assays known to those of skill in the art and/or exemplified herein.

As used herein, a culture means a propagation of cells in a medium conducive to their growth, and all sub-cultures thereof. The term subculture refers to a culture of cells grown from cells of another culture (source culture), or any subculture of the source culture, regardless of the number of subculturings that have been performed between the subculture of interest and the source culture. The term “to culture” refers to the process by which such culture propagates.

As used herein, a composition refers to any mixture of two or more products or compounds (e.g., agents, modulators, regulators, etc.). It can be a solution, a suspension, liquid, powder, a paste, aqueous, non-aqueous formulations or any combination thereof.

As used herein, a combination refers to any association between two or more items.

As used herein an effective amount of a compound for treating a particular disease is an amount that is sufficient to ameliorate, or in some manner reduce the symptoms associated with the disease. Such amount can be administered as a single dosage or can be administered according to a regimen, whereby it is effective. The amount can cure the disease but, typically, is administered in order to ameliorate the symptoms of the disease. Repeated administration can be required to achieve the desired amelioration of symptoms.

As used herein, pharmaceutically acceptable salts, esters or other derivatives of the conjugates include any salts, esters or derivatives that can be readily prepared by those of skill in this art using known methods for such derivatization and that produce compounds that can be administered to animals or humans without substantial toxic effects and that either are pharmaceutically active or are prodrugs.

As used herein, treatment means any manner in which the symptoms of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

As used herein, the term “subject” refers to an animals, including a mammal, such as a human being.

As used herein, a patient refers to a human subject.

As used herein, the term “antibody” as used herein includes intact molecules as well as functional fragments thereof, such as Fab, F(ab′)2, and Fv that are capable of binding the epitopic determinant. These functional antibody fragments retain some ability to selectively bind with their respective antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) F(ab′)2, the fragment of the antibody that can be obtained by treating whole antibody with the enzyme pepsin without subsequent reduction; F(ab′)2 is a dimer of two Fab′ fragments held together by two disulfide bonds;
(4) Fv, defined as a genetically engineered fragment containing the variable region of the light chain and the variable region of the heavy chain expressed as two chains; and
(5) Single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art (see, for example, Harlow and Lane, Antibodies: A Laboratory Manual, Cold Spring Harbor Laboratory, New York, 1988, incorporated herein by reference).

As used herein, the term “epitope” means any antigenic determinant on an antigen to which the paratope of an antibody binds. Epitopic determinants contain chemically active surface groupings of molecules such as amino acids or carbohydrate side chains and usually have specific three dimensional structural characteristics, as well as specific charge characteristics.

As used here, the singular forms “a,” “an” and “the” include plural referents unless the context clearly dictates otherwise. Thus, for example, reference to compound, “comprising an extracellular domain” includes compounds with one or a plurality of extracellular domains.

As used herein, ranges and amounts can be expressed as “about” a particular value or range. About also includes the exact amount. Hence “about 5 bases” means “about 5 bases” and also “5 bases”.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds are, unless indicated otherwise, in accord with their common usage, recognized abbreviations, or the IUPAC-IUB Commission on Biochemical Nomenclature (1972) Biochem., 11: 942-944.

### B. RIBOSOME INACTIVATING PROTEINS (RIPS), SELECTION, EXPRESSION AND PRODUCTION THEREOF

Provided are methods for selecting, identifying, purifying and/or isolating for ribosome inactivating protein (RIP) toxins with reduced toxicity, the resulting modified RIPs, and methods of expressing RIPs and modified RIPs and conjugates thereof. Toxicity is reduced sufficiently to increase expression of the protein, but sufficient toxicity remains for the RIP to exhibit the therapeutic effect (inhibition or killing of cells). Since RIPs are so toxic, a reduction in activity of 10-, 100-, 1000-fold, or more does not substantially impact on the use of the toxin as a toxin in the conjugates for inhibiting or killing cells or affecting cellular metabolism.

The methods provided herein employ RIP inhibitors, such as 4-aminopyrazolo[3,4-d]-pyrimidine (4-APP), to modulate the selection of RIP toxins and to increase the high-yield production thereof. Also provided are ligand-toxin conjugates containing all or part of a modified RIP sufficient to exert toxic activity, for example, any provided herein. The selected modified RIPs, and conjugates containing modified RIPs, exhibit less toxicity to host cells resulting in an increased yield of protein product following expression thereof. The increased yield is associated with less inherent toxicity and/or inhibition of activity with 4-APP as noted and described in detail herein below.

RIPs are toxins that promote cellular toxicity and death by depurinating eukaryotic and prokaryotic ribosomal RNA (rRNA) resulting in protein synthesis inhibition. Generally, RIPs, including ricin and Shiga Toxin, have toxic activity against eukaryotic ribosomes. Some RIPs, however, can attack eukaryotic and prokaryotic ribosomes. These include, for example, Shiga toxin, which exhibits toxicity toward E. coli cells (Skinner and Jackson, Microb. Pathol., 24: 117-22, 1998; Suh et al. (1998) Biochemistry 37: 9394-8). Thus, the expression of RIPs, and therefore high yield protein production thereof, is often hampered by the cytotoxic effects of RIPs on either one or both of prokaryotic or eukaryotic host cells used for recombinant protein expression thereof.

Given the general toxicity of RIPs to eukaryotic cells, RIPs, or conjugates containing RIPs, are typically produced in E. coli. For example, several RIP containing fusion proteins have been previously expressed in E. coli. These include for example fusions with saporin, pokeweed antiviral protein, or shiga toxin as the toxin moiety. Generally, relatively low levels of expression are obtained. In some cases, this is due to a leaky promoter system that releases a sufficient amount of toxin to interfere with cell viability. Strategies to optimize expression have been employed.

Generally, induction systems are used to suppress expression until the host cells have grown sufficiently. This allows for a tightly controlled means to allow for sufficient growth of transformed cells to occur before induction of the toxin begins to kill the host culture thereby limiting the overall production of the RIP toxin. For example, a standard method for the production of toxins, or conjugates thereof, is via expression under the control of a T7 late promoter in transformed E. coli BL21(DE3) cells after induction by isopropyl β-D-thiogalactoside (IPTG). Under this system, expression of RIPs, or conjugates thereof, has been further optimized using BL21(DE3)pLysS bacterial cells, which strongly represses expression from the pET vector in the absence of induction (Joshi et al. (2005), Prot. Exp. Purif., 39:189-198). In both systems, the resulting protein remains intracellular in association with inclusion bodies and requires de- and renaturation procedures upon purification (Barth et al. (2000) App Environ. Microbiol, 66:1572-1579). Other induction systems also have been used. For example, the gene for the Mirabilis Antiviral Protein (MAP) has been expressed under the control of a temperature-regulated promoter whereby expression of the MAP gene is induced by elevating the culture temperature from 30 to 42° C. at the log-phase of \88-10992. Often, even using such inducible systems, the RIPs can be toxic to the host cells. In some cases, no transformants can be obtained or transformants grow very poorly, which indicates that the inducible system is leaky and/or that the toxin moiety of the products can be responsible for killing the host cells.

Other strategies also have been employed to increase the expression and/or the yield of active protein. In one example, the expression vector can be designed to achieve secretion of the protein product promptly from the cytosol of the host to reduce the toxic effects on the host cell ribosomes. For example, for E. coli to secrete a protein a signal sequence is required. OmpA is a major outer membrane protein in E. coli that is produced in large quantities and secreted by E. coli (Habuka et al. (1990) J. Biol. Chem., 265:10988-10992). Hence, secretion and production of the MAP protein has been achieved by operatively linking the signal sequence of E. coli OmpA to the sequence encoding MAP. In other cases, RIPs or conjugates containing RIPs, have been expressed using other bacterial expression systems, such as for example, ones that direct the periplasmic expression of the toxin. In contrast to the bacterial cytoplasm, the bacterial periplasm is a nonreducing environment which permits disulfide bond formation required for the native conformation of some proteins. Although this strategy can be beneficial for those proteins that require disulfide bond formation, protein insolubility in the periplasmic environment can affect the protein yield and thereby require the use of compatible solutes during expression and purification (Barth et al. (2000) App Environ. Microbiol, 66:1572-1579). RIPs, or conjugates containing RIPs, also have been expressed in yeast Pichia pastoris, although this requires de novo design and construction of synthetic genes to optimize heterologous expression in yeast (Gurkan et al. (2005) Microbial Cell Factories, 4:33).

Although combinations of each of the above strategies are sometimes or somewhat effective, depending on the RIP or host cell used, in many cases host cells continue to be susceptible to the toxic effects of RIPs. In such cases, other strategies have been employed in attempts to express and produce toxins from host cells, although each has its limitations. For example, Fabrini et al. (FASEB J. 14:391-398 (2000)) have proposed the use of anti-RIP antibodies as neutralizing agents in eukaryotic cells to protect host ribosomes from inactivation while still allowing the majority of the synthesized polypeptide to be secreted in a biologically active form. Although neutralizing anti-saporin (SAP) antibodies have been used in the generation of a SAP conjugate, such a strategy requires the constitutive and stable expression of anti-RIP antibody fragments in host cells.

Thus, provided herein are methods to produce RIPs, or ligand-toxin conjugates containing RIPs, to overcome these limitations by taking advantage of the N-glycosidase mechanism by which RIPs mediate their toxic effects on prokaryotic and eukaryotic host cells. Adenine and several analogs thereof are capable of inhibiting RIP activity as measured by in vitro ribosome inactivation, including for example, inhibition by 4-aminopyrazolo[3,4-d]-pyrimidine (4-APP) (Brigotti et al. (2000) Nucleic Acids Res., 28: 2383-8; Brigotti et al. (2000) Life Sci., 68: 331-6). It is recognized herein that the use of adenine analogs (e.g., 4-APP) can be used in the selection of cellular expression clones, for example, bacterial clones, and in the large scale expression of toxins and ligand-toxin conjugate molecules including, for example, leukocyte population modulators (LPMs).

The methods provided herein are designed to 1) select for modified RIP toxins that exhibit reduced toxicity for host cells, while still maintaining sufficient toxic activity, which selection can be modulated in the presence of adenine analogs and 2) express the selected modified RIP toxins, or conjugates containing the modified RIP toxins, in host cells in the presence of one or more adenine analogs. Such methods allow for the identification of selected modified RIP toxins, which can be tested to identify those that retain sufficient toxic activity against target host cell ribosomes. Further, methods are provided herein which allow for the large scale expression and generation of RIP toxins, and conjugates containing the RIP toxins, in the presence of one or more RIP inhibitor, such as 4-APP.

Hence, the methods allow for the identification of modified RIP toxins that can be used in the design of ligand-toxin conjugates containing modified RIP toxins that exhibit reduced cytotoxicity to the host expressing bacterial strain and thereby provide a viable expression strategy for the production of greater quantities of product for use in preclinical and clinical studies. The suitability of the modified ligand-toxin conjugates to treat diseases and disorders such as inflammatory disease states associated with proliferation, migration and/or physiological activity of cells that promote inflammatory responses including secondary tissue damage can be assessed using in vitro and in vivo assays that assess an activity or biological activity.

### C. RIBOSOME INACTIVATING PROTEINS (RIPS) AND METHODS OF ACTION

Ribosome inactivating proteins (RIPs) are a class of proteins expressed in plants, fungi and bacteria that are potent inhibitors of eukaryotic and prokaryotic protein synthesis via a conserved mechanism. RIPs are N-glycosidases or polynucleotide:adenosine glycosidases and are able to inactivate ribosomal and nonribosomal nucleic acid substrates. RIPs are classified into two groups. Type I RIPs (also called holo-RIPs; i.e. trichosanthin and luffin) have a single polypeptide chain of ˜30 kDa having ribosome inactivating activity. Type II RIPs (also called chimero-RIPs; i.e. ricin, abrin, as well as bacterial toxins such as Shiga toxin) contain two polypeptide chains or species, denoted A (usually a single subunit) and B (single or multiple subunits), linked by a disulfide bond. The B chain of type II RIPs is required for cell entry, but can be substituted by a polypeptide that effects cellular entry. There also are examples of other RIPs that do not fall into either the type-I or type-II family. These are called two-chain type-I RIPs that contain only an A-chain but require proteolytic processing, and the type-III RIP proteins that are proteins structurally and functionally related to the barley RIP JIP60 (Peumans et al. (2001) The FASEB Journal, 15: 1493).

The B-chains of the type II RIPs bind to galactose-containing receptors on the cell surface and allow the A-chains to enter the cytoplasm where they inactivate ribosomes. Typically, type II RIPs are synthesized as a prepropolypeptide that contains A and B chains. Following targeting of the prepropolypeptide to the endoplasmic reticulum (ER), the signal sequence is cleaved off to yield a propolypeptide. In the ER, the protein undergoes disulfide bond formation between the two chains, and N-glycosylation occurs. The propolypeptide is transported through the Golgi apparatus into protein bodies where it is proteolytically cleaved by an endopeptidase within the protein bodies. The endopeptidase splits the propolypeptide into an A-chain and a B-chain or chains that remain linked by a single disulfide bond. Processing of the RIPs in this manner ensures that the toxins avoid poisoning its own host cell ribosomes, such as by leakage into the cytosol, during synthesis and transport.

Toxic activity of RIPs requires internalization of the catalytic subunit into the cytosol of a host cell. Cell entry of type II RIPs is facilitated by the B-subunit(s) whereas type I RIPs, which are not specifically recognized by hematogenous, tissue residing and intrinsic tissue cells, are less efficient in their toxic activity than type II RIPs. A variety of cell entry mechanisms exist for toxin internalization including, but not limited to, clathrin-dependent and clathrin-independent endocytosis, caveolae-independent endocytosis, and macropinocytosis. In addition, upon entry into the cells, toxins are transported to the cytosol via diverse mechanisms (Sandvig et al. (2005) Gene Therapy, 12: 865-872). Once inside the cytosol, RIPs catalyze the depurination of ribosomes thereby disrupting protein synthesis.

Type I RIPs and the A-chain of type II RIPs are responsible for the enzymatic activity of these toxins by inhibiting protein synthesis by removing a specific adenine from 28 S rRNA of eukaryotic and prokaryotic ribosomes. Generally, type II RIPs are considered active only against eukaryotic ribosomes, while type I RIPs are active against eukaryotic and prokaryotic ribosomes. Some type II RIPS, such as for example Shiga Toxin (STX), also inhibit prokaryotic ribosomes (Skinner et al. (1998), Microbial Pathogenesis, 24: 117-122).

The toxic activity of RIPs, either single-chain (type I) or two-chain (type II, mediated via the A-chain) is mediated by the N-glycosidase activity of the proteins. This enzymatic activity results in the removal of one adenine from adenosine in a precise position (A₄₃₂₄ in the case of rat liver 28S rRNA, A₂₆₆₀ of E. coli rRNA) in a universally conserved GAGA tetraloop of the major rRNA, also called the alpha-sarcin/ricin loop (see e.g., Endo et al. (1987) J. Biol. Chem., 262:8128; Barbieri et al. (1993) Biochim. Biophys. Acta., 1154:237; Sandvig et al. (2001) Toxicon, 39: 1629-1635; Ippoliti et al. (2004) The Italian Journal of Biochemistry, 53: 92; Stirpe and Battelli, Cell Mol Life Sci., 63: 1850-66, 2006). The removal of the adenine base results in the inability of the ribosome to bind elongation factor 2 and thus termination of RNA translation. The GAGA sequence is present in prokaryotic and eukaryotic ribosomes.

The enzymatic activity of RIP toxins is mediated by the interaction of the catalytic chain with ribosomal proteins. The interaction with the adenine occurs in an active site cleft of the toxin proteins. Differences in substrate binding between toxins can be due to amino acid differences in the active site cleft. For example, although X-ray crystallography data shows that the active site cleft between the A-subunits of Stx and ricin are similar, there are at least seven invariant residues in the active site of these proteins (Brigotti et al. (2000) Nucleic Acids Research, 28:2383-2388). Further, differences in substrate specificity between eukaryotic and prokaryotic cells among toxins are believed to be due to differing abilities of RIPs to interact with different ribosomal proteins. For example, the rat liver proteins L9 and L10e are the binding targets of the ricin A-chain, while the ribosomal protein L3 is the binding factor of pokeweed antiviral protein (PAP). L3 is a highly conserved ribosomal protein, which explains the broad specificity of PAP towards ribosomes of different species (Peuman et al. (2001) The FASEB Journal, 15: 1493-1496). The removal of adenine results in a conformational change of the rRNA and prevents the binding of elongation factor 2. Thus, depurinated ribosomes are unable to elongate the nascent peptide chain.

In addition to inactivating ribosomes and inhibiting protein synthesis, RIPs also have other functions due to their interaction with other substrates besides rRNA. RIPs can depurinate DNA, mRNA, and viral polynucleotides (Ippoliti et al. (2004) The Italian Journal of Biochemistry, 53: 92; Parikh et al. (2004) Mini-Reviews in Medicinal Chemistry, 4:523-543). Hence, in addition to N-glycosidase activity, RIPs have been demonstrated to have polynucleotide:adenosine glycosidase activity due to their ability to deadenylate adenine-containing polynucleotides, single-stranded DNA, double-stranded DNA, and mRNA. For example, RIPs have been reported to degrade supercoiled DNA (see e.g., Li et al. (1991) Nucleic Acid Res., 22:6309; Ling et al. (1994) FEBS Lett., 345:143; Roncuzzi et al. (1996) FEBS Lett., 392:16) and fragment genomic DNA (Bagga et al. (2003) J Biol. Chem., 278:4813-4820). Moreover, some RIPs release more than one adenine residue from ribosomes (Barbieri et al. (1992) Biochem. J., 286:1), act on RNA species other than ribosomal RNA, including viral RNAs, or also act on poly(A) and on DNA (Barbieri et al. (1994) Nature, 372:624; Stirpe et al. (1996) FEBS Lett., 382:309; Picard et al. (2005) J Biol. Chem., 280:20069-20075). Additionally several RIPs have been shown to inhibit the 3′-end processing and strand-transfer activities of HIV-1 integrase which in turn inhibits the insertion of the viral genome into the host cell genome (Au et al., FEBS Lett, 471: 169-72, 2000). Hence viral propagation is inhibited. Consequently, some RIPs exhibit anti-viral activity in addition to or instead of protein synthesis inhibition via inactivation of ribosomes (Parikh et al. (2004) Mini-Reviews in Medicinal Chemistry, 4:523-543; Erice et al. (1993) Antimicrobial Agents and Chemotherapy, 37: 835-838). Thus many, if not all, RIPs have one or more of N-glycosidase activity, RNase activity, DNase activity, and other activities such as, but not limited to, superoxide dismutase, phospholipase activity, chitinase activity and anti-viral activity (Park et al. (2004) Planta, 219:1093-1096; Bagga et al. (2003) J Biol. Chem., 278:4813-4820; Parikh et al. (2004) Mini-Reviews in Medicinal Chemistry, 4:523-543; Au et al., FEBS Lett, 471: 169-72, 2000).

1. Exemplary RIPs

Exemplary toxins used in the methods provided herein for selection of modified toxins with reduced toxicity such as for improved production of toxins, or conjugates thereof, or in the generation of ligand-toxin conjugates, can be any toxin that exhibits cellular toxicity due to N-glycosidase enzymatic activity via depurination of rRNA. Such toxins are known to those of skill in the art and typically include the RIP family of toxins. For example, over 400 RIPs have been proposed, of which more than 50 type I RIPs and 15 Type-II RIPs have been sequenced and/or cloned (Peumans et al. (2001) The FASEB Journal, 15: 1493). Exemplary type I RIPs include, but are not limited to, dianthin 30, dianthin 32, lychnin, saporin-1, saporin-2, saporin-3, saporin-4, saporin-5, saporin-6, saporin-7, saporin-8, saporin-9, PAP, PAP II, PAP-R, PAP-S, PAP-C, mapalmin, dodecandrin, bryodin-L, bryodin, colicin-1, colicin-2, luffin-A, luffin-B, luffin-S, 19K-PSI, 15K-PSI, 9K-PSI, alpha-kirilowin, beta-kirilowin, gelonin, momordin, momordin-II, momordin-Ic, MAP-30, alpha-momorcharin, beta-momorcharin, trichosanthin, TAP-29, trichokirin, barley RIP, tritin, flax RIP, corn RIP, asparin-1, and asparin 2. Exemplary type II RIPs include, but are not limited to, volkensin, ricin, Shiga toxin, nigrin-CIP-29, abrin, vircumin, modeccin, ebulitin-α, ebulitin-β, ebulitin-γ, and porrectin. Generally, the A-chain, or an active fragment thereof, is sufficient for the enzymatic activity of type II RIPs.

The discussion of various RIP toxin polypeptides is not meant to limit the scope of the embodiments provided. It is understood that any RIP polypeptide known to one of skill in the art, or subsequently identified hereto, is contemplated in the methods provided herein. Those of skill in the art are familiar with the identification and functional characterization of RIP toxins. A list of exemplary RIP toxin polypeptides and their corresponding SEQ ID NOs is set forth in Table 3.

TABLE 3
Exemplary RIP Toxins

| | | | | Enzymatic | SEQ |
|---|---|---|---|---|---|
| | | UniProt | Signal | subunit (i.e. | ID |
| RIP Toxin | Synonyms | NO: | Sequence | A chain) | NO: |
| Shiga toxin A- | StxA; StxI; Stx1; Shiga-like | P10149 | 1-22 | 23-315 | 1 |
| chain (Stx) | toxin I subunit A; SLT-A; | | | | |
| | SLT-I; SLT-1; Verotoxin 1 | | | | |
| | subunit A; VT1 | | | | |
| Shiga-like toxin II | StxA2; Stx2A; Verotoxin 2 | P09385 | 1-22 | 23-319 | 3 |
| subunit A (Stx2) | subunit A; VT2; SLT-IIA; | | | | |
| | SLT2 | | | | |
| Saporin 6 | SAP-6; SO-6 | P20656 | 1-24 | 25-277 | 89 |
| Barley RIP I | Protein synthesis inhibitor I; | P22244 | | 1-280 | 90 |
| | RIP30 | | | | |
| Barley RIP II | Protein synthesis inhibitor II; | P04399 | | 1-280 | 91 |
| | RIP30A | | | | |
| Gelonin | GEL | P33186 | 1-26 | 47-297 | 92 |
| Ricin A | | P02879 | 1-35 | 36-302 | 93 |
| Momordin I | α-momorcharin; α-MMC | P16094 | 1-23 | 24-269 | 94 |
| Momordin II | | P29339 | 1-23 | 24-286 | 95 |
| Bryodin I | BD1 | P33185 | 1-23 | 24-270 | 96 |
| Bryodin II | BD2 | P98184 | 1-21 | 22-282 | 97 |
| Pap-S | Pokeweed antiviral protein S | P23339 | | 1-261 | 98 |
| Luffin | Luffin-α | Q00465 | 1-19 | 20-277 | 99 |
| Trichosanthin | α-trichosanthin; α-TCS | P09989 | 1-23 | 24-270 | 100 |
| Clavin | | P49074 | 1-27 | 28-177 | 101 |
| Abrin-a | | P11140 | | 1-251 | 102 |
| Maize RIP 3 | CRIP3 | P25891 | | 1-300 | 103 |
| Maize RIP 9 | CRIP9 | P25892 | | 1-304 | 104 |
| Maize RIP X | | P28522 | 1-16 | 17-161 | 105 |
| Tritin | Trig7; Wheat RIP | Q07810 | | 1-275 | 106 |
| MAP | | P21326 | 1-28 | 29-278 | 107 |
| Dianthin 30 | DAP-30 | P24476 | 1-23 | 24-293 | 108 |
| Nigrin b | Agglutinin V; SNAV | P33183 | 1-25 | 26-297 | 109 |
| Nigrin I | | Q8GT32 | 1-25 | 26-274 | 110 |
| Ebulin | Ebu1 | Q9AVR2 | 1-25 | 26-298 | 111 |

Shiga Toxin

Shiga toxins (STX) are a family of RIP proteins that are produced by bacteria. Shiga toxins are classified into three different groups. Shiga toxin (Stx) is produced by Shigella dysenteriae and is a type-II RIP protein containing a 32-kDa enzymatic A subunit (StxA), noncovalently associated with a ring of five 7.7 kDa B subunits (StxB). Stx is identical in amino acid sequence to Shiga-like toxin 1 (Stx1, also called Verotoxin, SLT1 or VT1), produced by E. coli. The A-chain precursors of Stx and Stx1 are 315 amino acids in length (set forth in SEQ ID NO:1) and contain a signal sequence of 22 amino acids in length corresponding to amino acids 1-22 of SEQ ID NO:1. The mature Stx/Stx1 A chain is 293 amino acids in length corresponding to amino acids 23-315 of SEQ ID NO:1 and is set forth in SEQ ID NO:5. The third Stx is Shiga-like toxin 2 (Stx2, also called Verotoxin 2, SLT2 or VT2), which exhibits sequence differences compared to Stx and Stx1. The A-chain precursor of Stx2 is 319 amino acids in length (set forth in SEQ ID NO:3) and contains a signal sequence 22 amino acids in length corresponding to amino acids 1-22 of SEQ ID NO:3. The mature Stx2 A chain is 297 amino acids in length corresponding to amino acids 23-319 of SEQ ID NO:3. The B subunits of Stx/Stx1 and Stx2 are 89 amino acids in length (set forth in SEQ ID NOs:2 and 4, respectively). Shiga-like toxins also have been reported to be produced in Citrobacter freundii, Aeromononas hydrophila, Aeromononas caviae, and Enterobacter cloacae (Sandvig et al. (2001) Toxicon, 39: 1629-1635).

The A chain of Stx (StxA) has an enzymatically active A fragment that contains an internal disulfide bond formed between C242 and C261 of the sequence set forth in SEQ ID NO:5 (corresponding to C264 and C283, respectively, of the sequence set forth in SEQ ID NO:1). The sequence ²⁴⁸Arg-Val-Ala-Arg²⁵¹ in SEQ ID NO:5, which is located in a loop between the two cysteines, is recognized by trypsin or by the cellular protease furin. Furin is found in the trans golgi network (TGN) and in endosomes and likely cleaves StxA during its posttranslational processing. Trypsin or furin cleaves StxA at the COOH-terminal side of Arg²⁵¹ in the sequence set forth in SEQ ID NO:5, separating the A-chain into A1 and A2 fragments (Sandvig et al. (2001) Toxicon, 39: 1629-1635; Garred et al. (1995) J Biol. Chem., 270: 10817-10821). Hence, the cleaved A1 fragment of Stx (SA1) corresponds to amino acids 1 to 251 and the A2 fragment of Stx (SA2) corresponds to amino acids 252-293 of the sequence of amino acids set forth in SEQ ID NO:5.

Furin cleavage activates the A1 fragment (SA1). The A1 domain remains associated with the A2/B subunits due to the disulfide bond between C242 and C261 until transport through the ER where the disulfide bond is reduced and the A1 fragment is allowed to retrotranslocate to the cytosol (LaPointe et al. (2005) J Biol. Chem., 280:23310-8). The A1 fragment of Stx is 6- to 400-fold more active than the intact Stx protein (Suh et al. (1998) Biochemistry, 37:9394-9398). As such, SA1 contains the RIP enzymatic activity and is responsible for inhibiting protein synthesis by depurination of the 28S RNA of the 60S ribosomal subunit. The first 239 amino acids of the A1 chain represent the minimal catalytically active region of the StxA1 REP domain (LaPointe et al. (2005) J Biol. Chem., 280:23310-8). SA1 truncations retaining catalytic activity include, for example, the variant 1 SA1 sequence set forth in SEQ ID NO:22 and encoded by a sequence of nucleotides set forth in SEQ ID NO:23 and a variant 2 sequence set forth in SEQ ID NO:24 and encoded by a sequence of nucleotides set forth in SEQ ID NO:25.

Like other RIPs, the active SA1 subunit of Stx attacks eukaryotic ribosomes; however, it also has activity against bacterial ribosomes. For example, various groups have reported that the growth of E. coli cells is reduced in the presence of SA1 (see e.g., Skinner et al. (1998) Microbial Pathogenesis, 24:117-122; Suh et al. (1998) Biochemistry, 37:9394-9398). The toxic activity of SA1 on prokaryotic cells requires expression of the toxin in the cytoplasm, such as due to the absence of its native signal sequence; no Stx-mediated toxicity is observed in cells following the export of SA1 into the periplasm by its signal sequence. The toxic activity of SA1 on prokaryotic cells is comparable to its toxic activity on eukaryotic cells. Other RIPs also target prokaryotic cells, including for example, the plant RIPs PAP and MAP, although in most cases the toxic activity of such plant RIPS is about 100 times more efficient against eukaryotic ribosomes (Suh et al. (1998) Biochemistry, 37:9394-9398). In contrast, other RIPs, such as RTA (the enzymatic subunit of ricin) displays no toxicity towards prokaryotic cells.

2. RIP Toxin Inhibitors

Inhibitors are known or can be identified that inactivate toxic RIPs. Studies of such inhibitors have provided insight about the structure of the active site of the toxins. In addition, there is an interest in identifying and developing REP inhibitors for various reasons, including but not limited to, diagnostic purposes, antidotes in poisoning or as prophylactic and therapeutic agents in infections triggered by RIP-expressing bacteria (Brigotti et al. (2000) Life Sciences, 68: 331-336, U.S. Pat. No. 6,562,969). Some RIP inhibitors target the conserved N-glycosidase activity of RIP toxins. Included among such RIP toxin inhibitors are RIP-specific oligonucleotide inhibitors, such as RNA aptamers (see e.g., Hesselberth et al. (2000) J. Biol. Chem., 275:4937-4942; Hirao et al. (2000) J. Biol. Chem., 275: 4943-4948), RIP-specific antibodies, and/or adenine isomers including, for example, adenine, 4-aminopyrazolo[3,4-d]pyrimidine (4-APP), and other similar isomers (Pallanca et al. (1998) Biochimica et Biophysica Acta, 1384: 277-284; Brigotti et al. (2000) Nucleic Acids Research, 28: 2383-2388; Brigotti et al. (2000) Life Sci., 68: 331-6; U.S. Pat. No. 6,562,969).

4-APP and Other Adenine Analogs

Adenine is a base in the natural substrate for RIP toxins (i.e. the first adenine base in the loop sequence of GAGA). Hence, adenine and analogs thereof inhibit RIP toxic activity (Pallanca et al. (1998) Biochimica et Biophysica Acta, 1384: 277-284; Brigotti et al. (2000) Nucleic Acids Research, 28: 2383-2388; Brigotti et al. (2000) Life Sci., 68: 331-6) by acting as an inhibitor of the RNA N-glycosidase activity. Typically, an adenine analog includes any fused bicyclic compound where one of the rings is 6-aminopyrimidine, and the other ring is a 5-membered heterocyclic ring that contains at least two adjacent carbon atoms, including but not limited to, pyrrole, pyrazole, imidazole, triazole, oxazole, isoxazole, thiazole, isothiazole, furan and thiophene. Typically, fusion of the rings occurs between the carbon atoms at the 4 and 5 positions of 6-aminopyrimidine, and any two adjacent carbon atoms of the 5-membered ring, in either mode of attachment. This includes, for example, adenine itself whose 5-membered ring is in the imidazole configuration. The structure of adenine is as follows:

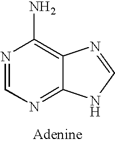

Further, such analogs also include any with rearrangements of the nitrogen atoms of the 5-membered ring from the imidazole to the pyrazole configuration including, for example, 4-APP and formycin base, which only differ in the mode of attachment of the 6-aminopyrimidine to the 5-membered pyrazole ring (see e.g., Brigotti et al. (2000) Life Sci., 68: 331-6). In additions, inhibitors provided herein include the ribonucleoside and deoxyribonucleoside analogs of formycin A base, such as the ribonucleotide 5′mono-, 5′ di-, 5′ tri and 3′ monophosphate analogs of formycin A bases, as well as the deoxyribonucleotides 5′ mono-, 5′ di-, 5′ tri and 3′ monophosphate analogs of formycin A, or any other similar or known compound such as any subsequently identified hereto (see e.g., U.S. Pat. No. 6,562,969). The structure of 4-APP and formycin A base are as follows:

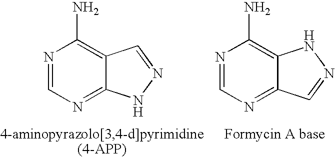

Despite the conserved N-glyosidase activity of RIP toxins, adenine and analogs of adenine, such as 4-APP, exhibit differential abilities to protect ribosomes from inactivation by RIPs (Pallanca et al. (1998) Biochimica et Biophysica Acta, 1384: 277-284; Brigotti et al. (2000) Nucleic Acids Research, 28: 2383-2388; Brigotti et al. (2000) Life Sci., 68: 331-6). For example, 4-APP is a strong inhibitor of Stx, momordin, and other plant RIPs, but exhibits little inhibition of ricin. Further, 4-APP exhibits greater inhibitory activity on Stx than does adenine, however, 4-APP and adenine display comparable inhibitory activity to the RIP toxin momordin. Also, adenine protects ribosomes from inactivation by ricin, whereas 4-APP displays little inhibitory action on the toxic activity of ricin. Hence, RIP toxins differ in their abilities to be inhibited by various adenine isomers indicating that RIP toxins do not share a common active site binding cleft. The inhibitory activity of adenine isomers on RIP activity are known (Pallanca et al. (1998) Biochimica et Biophysica Acta, 1384: 277-284; Brigotti et al. (2000) Nucleic Acids Research, 28: 2383-2388; Brigotti et al. (2000) Life Sci., 68: 331-6), or can be determined by one of skill in the art such as by determining the RNA N-glycosidase activity (i.e. REP activity) of the toxin in the presence of the inhibitor.

As is described in detail below, RIP inhibitors, such as adenine and analogs thereof including, for example 4-APP, can be used in methods to select for modified forms of RIPs and also can be used in methods of improving the production of a RIP toxin, or conjugate thereof, such as any modified RIP toxin provided herein or identified by the selection methods provided herein.

### D. METHODS OF SELECTING MODIFIED TOXINS OR CONJUGATES THEREOF

Provided herein are methods of selecting modified RIP toxins that exhibit reduced cytotoxicity to the host expressing cells. In the methods herein, it has been found that because of the toxicity of the RIPs to particular host cells, the RIP is often expressed at low levels in a culture of cells, even under conditions in which it is toxic ultimately to all, or substantially all, of the cells. Typically, RIPs are expressed because a requisite amount is required to exhibit toxicity to the cells, some cells could become resistant to the toxic affects of RIPs, and, as shown herein, the RIPs mutate. As a result, in a culture of cells containing nucleic acids encoding a RIP, RIP is expressed, but at relatively low levels.

Accordingly, the methods are designed to select and identify those REP toxins produced by host cells under conditions where the starting RIP protein is not produced or is produced at low levels. To perform the methods provided herein, a nucleic acid encoding an unmodified or starting form of a RIP toxin is introduced into a host cell, the host cell is allowed to grow, cells that grow are isolated and those RIP toxins that are expressed in the cells are identified and tested for activity, such as for example, N-glycosidase activity and/or other RIP activities including, but not limited to, RNase activity, DNase activity, superoxide dismutase and phospholipase activities. In some examples, selection is additionally performed in the presence of a selection modulator, such as a RIP inhibitor.

Generally, such identified REP toxins are modified compared to the starting RIP protein and, by virtue of the modification, the REP toxin has an altered activity, such as an altered toxic activity or other activity, compared to the starting RIP toxin. Generally, the toxicity of the modified RIP polypeptide is reduced. In some examples, the modified RIP polypeptide identified in the selection methods herein exhibits no toxic activity. Typically, however, a modified RIP toxin, or conjugate thereof, retains 0.5%, 1%, 1.5%, 2%, 5%, 10%, 20%, 30%, 40%, 50%, 60%, 70%, 80%, 90%, 95%, or 100% of the toxic activity compared to a wild-type form of the toxin, or conjugate thereof. Due to the retained cytotoxic activity, conjugates containing such modified toxins can be designed to target specific cells, thereby resulting in killing of the targeted cell or cells upon internalization of the conjugate. For example, as is described in detail below, conjugates containing a modified REP toxin can be used in methods of treating various diseases or disorders by targeting one or more cells or populations of cells involved in the disease process. Such modified toxins also can be used in methods to express and produce RIP toxins or conjugates thereof, thereby enabling high yield protein production.

1. Candidate RIP Proteins or Conjugates Thereof

Generally, because many RIP proteins exhibit toxic activity to prokaryotic and/or eukaryotic cells they inhibit protein synthesis by cellular ribosomes, they cannot be expressed at high levels in some or all host cell systems. Provided herein are methods to reduce the toxicity of RIPs so that they can be expressed at higher levels, but still exhibit sufficient toxicity to be used therapeutically in conjugates that employ RIPs. Included are the conjugates in U.S. Pat. Nos. 7,166,702, 7,157,418 and 7,192,736 as well conjugates of cytokines, such as growth factors, including FGF, VEGF, EGF and others.

In the methods provided herein, a REP toxin or conjugate thereof is produced that exhibits a reduced toxicity in a host cell. Such modified RIP toxins or conjugates thereof are thereby less toxic to cells. Selecting for RIP proteins, or conjugates thereof, that are modified to exhibit reduced toxicity allows for the expression of such toxin by host cells and improved yields. Accordingly, such a method allows for the generation of RIP toxins, or conjugates thereof that can be produced effectively and efficiently and thereby used in methods to treat diseases or disorders for which they are designed.

RIP proteins to be modified by the selection method provided herein can be any RIP protein, or any polypeptide containing a RIP protein or active portion thereof, which under standard or normal growth conditions, is not expressed or is expressed at low levels in a host cell due to toxic activity against the host cell ribosomes. The proteins are modified and then, under the same conditions, are expressed at higher levels. Candidate RIP protein for selection includes wildtype or variant forms of a wildtype RIP protein, or active portions thereof exhibiting toxic activity, including allelic or species variants and isoforms of a RIP protein that have not been selected by the methods herein.

Included among such RIP proteins are any set forth in Table 3 above, such as any having a sequence of amino acids set forth in any of SEQ ID NOS: 5, 89-111, particularly the active portion of the A-chain of such RIP proteins, such as the A1 chain of Shiga toxin (i.e. SA1), or any active fragment thereof. For example, a starting protein used in the methods provided herein can be any that are truncated in their A-chain or A1 chain, but that still exhibit catalytic activity. Also included as a starting protein in the methods provided herein is any polypeptide containing a variant form of a RIP protein, such as an allelic or species variant thereof. Exemplary variants of RIP proteins are set forth in any of SEQ ID NOS: 6, 9-21, or 162-169. Conjugates containing such proteins linked to a targeting agent also are provided.

Also included as a starting protein in the methods herein are conjugates containing any such RIP toxin noted above, or an active portion of such a RIP toxin, linked directly or indirectly to another polypeptide moiety. For example, such conjugates include ligand-toxin conjugates, including those where the RIP toxin is linked directly or indirectly to a chemokine, cytokine, antibody, growth factor, or other such ligand protein that is capable of binding to a cell surface receptor. Typically, such conjugates are encoded by a nucleic acid molecule encoding a fusion protein.

Exemplary RIP proteins used as starting proteins in the methods provided herein include SA1, for example having a sequence of amino acids corresponding to amino acids 1-251 of SEQ ID NO:5, or truncations thereof such as an SA1 having an amino acid sequence set forth in SEQ ID NO: 22 (i.e. variant 1 SA1) or SEQ ID NO:24 (i.e. variant 2 SA1), respectively, or any allelic or species variants thereof. Exemplary of such conjugates are any containing any of the SA1 moiety noted above, where the SA1 moiety is linked directly or indirectly to a ligand or other cell receptor binding molecule. For example, such conjugates include chemokine conjugates (i.e. leukocyte population modulators) such as set forth and described in U.S. Pat. Nos. 7,166,702, 7,157,418 and 7,192,736. These include, for example, one having an MCP-1 chemokine linked to SA1. An exemplary sequence of an MCP-1-SA1 conjugate linked to a variant 1 SA1 RIP protein (i.e. LPM1a) is set forth in SEQ ID NO:38 and is encoded by a sequence of nucleotides set forth in SEQ ID NO:37. An additional exemplary sequence of an MCP-1-SA1 conjugate linked to a variant 2 SA1 RIP protein (i.e. LPM1b) is set forth in SEQ ID NO: 40 and is encoded by a sequence of nucleotides set forth in SEQ ID NO:39.

2. Introduction of RIPs or Conjugates Thereof into Host Cells

Nucleic acids encoding a desired starting RIP protein, or a conjugate thereof, are introduced into any desired host cell. Typically, a host cell chosen in the selection method is one which is susceptible to the toxic effects of the starting RIP protein, or conjugate thereof, such that protein synthesis of the host cell is abolished or significantly impaired upon expression of the RIP in the host cell. Included among host cells for use in the selection methods herein include any prokaryotic cell including, but not limited to, any bacterial cell such as E. coli. Also included among host cells are any eukaryotic cells including, but not limited to, yeast such as Pichia pastoris, Xenopus oocytes, and mammalian cells, such as for example, Vero, Hep2, Chang, A549, COS-1, and HeLa cells. In deciding an appropriate host cell to use in the selection methods herein, the influence of a RIP protein, or conjugate thereof, on recombinant protein expression in the host cell can be determined by various methods described herein below or known to those of skill in the art. Assays to assess effects on protein synthesis include, for example, depurination assays (i.e. release of adenine), cell-free protein synthesis assays, such as a rabbit reticulocyte lysate or a wheat germ lysate protein synthesis assay, or cell growth/viability assays. For example, by using such assays, it is known that SA1 displays significant toxic activity to eukaryotic and prokaryotic ribosomes (Suh et al. (1998) Biochemistry, 37:9394). Hence, in one example, selection of a modified form of SA1, or active form thereof, is performed in eukaryotic cells. In another example, selection of a modified form of SA1, or active portion thereof, is performed in bacterial cells, such as in E. coli. Various E. coli host strains are available and include but are not limited to BL21(DE3) or BL21(DE3)pLysS cells.

A nucleic acid molecule encoded a starting RIP protein, or conjugate thereof, for use in the methods herein, can be produced or isolated by any method known in the art including isolation from natural sources, generation by standard recombinant DNA techniques such as via standard cloning procedures from cells, tissues and organisms, and by other recombinant methods and by methods including in silico steps, synthetic methods and any methods known to those of skill in the art. Such nucleic acid molecules can include additional sequences such as restriction enzyme sequences, linkers, tags, or other such sequences. Exemplary of nucleic acid molecules include any encoding a RIP protein, active forms thereof, or variant thereof such as any encoding a polypeptide set forth in any of SEQ ID NOS: 1, 3, 5, 7-22, 24, 89-111, or 162-169, or any encoding a conjugate containing any such RIP protein. Exemplary nucleic acid sequences include, for example, sequences of a variant 1 or variant 2 form of SA1 such as is set for in SEQ ID NO: 23 or SEQ ID NO: 25, respectively. Other exemplary nucleic acid sequences include sequences encoding a conjugate such as, for example, a conjugate of a chemokine such as MCP-1 linked to an SA1 variant. For example, nucleic acid sequences encoding an LPM1a or LPM1b conjugate can be used in the methods provided herein and include the sequences set forth in SEQ ID NO:37 and 39, respectively.

Typically, a nucleic acid molecule used to introduce host cells with sequences for selection and expression of a modified RIP protein or conjugate thereof are in the form of an expression vector including those having expression control sequences operatively linked to a nucleic acid sequence coding for expression of the polypeptide. Such expression vectors are described in detail herein below. The appropriate vector can be chosen depending on the host cell and/or any desired transcription/translation elements including, for example, constitutive and inducible promoters, transcription enhancer elements, transcription terminators, etc. In one example, inducible expression systems are used in the methods herein which allow for optimal growth of the host cell before expression of the toxin. Exemplary of an inducible system in E. coli is pET vectors, such as the PET9c plasmid, which are under the control of a T7 late promoter and require induction by IPTG. In other examples, the host cells used for expression of the encoding nucleic acids introduced thereto can be chosen which themselves also carry further components that optimize toxin expression. For example, where the host cell is E. coli, a cell line BL21(DE3)pLysS can be used which strongly repress expression from the T7 promoter (such as in a pET vector) in the absence of induction, compared to the parental host cells BL21(DE3) which can be leaky.

Nucleic acid molecules encoding RIP proteins, active forms thereof, or conjugates thereof can be introduced into a host cell by any method known to those of skill in the art. Such methods are chosen depending on the chosen host cell and include, but are not limited to, transfection, transformation, electroporation, and any other suitable method. In some cases, DNA also can be introduced into cells by transduction using viral vectors. Typically, when introducing DNA into bacterial cells, transformation or electroporation methods are used.

a. Transfection

Transfection can be used to introduce a nucleic acid into eukaryotic or prokaryotic cells. Transfection can be achieved by various methodologies, but typically involves the opening of transient “holes” into the cell to allow entry of the DNA, which then becomes transiently expressed in the host cell. Examples of methodologies to introduce DNA by transfection include, but are not limited to, calcium phosphate methods, lipofection, and gene gun approaches. For example, in the lipofection approach, DNA is included in liposomes or by using lipid-cation reagents which are then able to fuse with the cell membrane releasing the DNA into the cell. Examples of cationic lipids include, but are not limited to, Lipofectin (Life Technologies, Inc., Burlington, Ont.)(1:1 (w/w) formulation of the cationic lipid N-[1-(2,3-dioleyloxy)propyl]-N,N,N-trimethylammonium chloride (DOTMA) and dioleoylphosphatidylethanolamine (DOPE)); LipofectAMINE (Life Technologies, Burlington, Ont., see U.S. Pat. No. 5,334,761) (3:1 (w/w) formulation of polycationic lipid 2,3-dioleyloxy-N-[2(spermine-carboxamido)ethyl]-N,N-dimethyl-1-propanaminiumtrifluoroacetate (DOSPA) and dioleoylphosphatidylethanolamine (DOPE), LipofectAMINE PLUS (Life Technologies, Burlington, Ont. see U.S. Pat. Nos. 5,334,761 and 5,736,392; see, also U.S. Pat. No. 6,051,429) (LipofectAmine and Plus reagent), LipofectAMINE 2000 (Life Technologies, Burlington, Ont.; see also International PCT application No. WO 00/27795) (Cationic lipid), Effectene (Qiagen, Inc., Mississauga, Ontario) (Non liposomal lipid formulation), Metafectene (Biontex, Munich, Germany) (Polycationic lipid), Eu-fectins (Promega Biosciences, Inc., San Luis Obispo, Calif.) (ethanolic cationic lipids numbers 1 through 12: C₅₂H₁₀₆N₆O₄.4CF₃CO₂H, C₈₈H₁₇₈N₈O₄S₂.4CF₃CO₂H, C₄₀H₈₄NO₃P.CF₃CO₂H, C₅₀H₁₀₃N₇O₃.4CF₃CO₂H, C₅₅H₁₁₆N₈O₂.6CF₃CO₂H, C₄₉H₁₀₂N₆O₃.4CF₃CO₂H, C₄₄H₈₉N₅O₃.2CF₃CO₂H, C₁₀₀H₂₀₆N₁₂O₄S₂.8CF₃CO₂H, C₁₆₂H₃₃₀N₂₂O₉.13CF₃CO₂H, C₄₃H₈₈N₄O₂.2CF₃CO₂H, C₄₃H₈₈N₄O₃.2CF₃CO₂H, C₄₁H₇₈NO₈P); Cytofectene (Bio-Rad, Hercules, Calif.) (mixture of a cationic lipid and a neutral lipid), GenePORTER (Gene Therapy Systems Inc., San Diego, Calif.) (formulation of a neutral lipid (Dope) and a cationic lipid) and FuGENE 6 (Roche Molecular Biochemicals, Indianapolis, Ind.) (Multi-component lipid based non-liposomal reagent).

b. Transformation

Transformation is distinguished from transfection in that the introduced DNA is incorporated into the cell's genome for expression of the genetic material. Typically, expression vectors that are used for stable transformation have a selectable marker, such as for example, antibiotic resistance, which allows selection and maintenance of the transformed cells. Transformation requires the transfer of DNA into the cell which is achieved in cells that are naturally competent or are rendered competent to take up DNA across the cell's membranes or membranes. Calcium chloride is one method used to render cells, such as E. coli cells, more competent. Following heat-shock of bacterial cells, they are induced to take in the DNA. Transformation is not limited to bacteria, but also can be performed in yeast, plants, and mammalian cells including embryonic stem cells. Methods of transformation are well known (see e.g., Mello et al. (1995) Methods Cell Biol., 48:451-82).

c. Electroporation

Electroporation temporarily opens up pores in a cell's outer membrane by use of pulsed rotating electric fields. Methods and apparatus used for electroporation in vitro and in vivo are well known (see, e.g., U.S. Pat. Nos. 6,027,488, 5,993,434, 5,944,710, 5,507,724, 5,501,662, 5,389,069, 5,318,515). Standard protocols can be employed.

3. Expression, Selection and Identification

Introduction of host cells with a DNA molecule results in amplification of the gene product and thereby enables multiple copies of the gene to be expressed. Since the starting RIP toxins, or conjugates thereof, used in the selection methods herein are normally toxic to the chosen host cell, amplification and expression of the starting proteins does not typically occur, for example, due to cell death. Hence, the methods provided herein use the normal toxicity of the starting proteins as a selection method to select for those modified forms of the protein that exhibit less toxicity to the host cell and are thereby expressed. Typically, such expressed proteins are modified in their primary sequence by one or more amino acid mutations that render the protein less toxic. In some cases, the expressed proteins are modified via truncation of the amino acid sequence compared to the starting protein, which renders the protein less toxic. In most host cell expression systems, the gene encoding the modified RIP toxin can be obtained in large quantities by growing transformants, isolating the recombinant DNA molecules from the transformants and, when necessary, retrieving the inserted gene from the isolated recombinant DNA.

In some examples, an additional agent or agents is added to the selection method in order to modulate selection to optimize for recovery of a modified RIP toxin or conjugate thereof. Such selection modulators typically are any that reduce the toxic activity of the RIP toxin or conjugate thereof. For example, any RIP inhibitor can be used to modulate selection. Any RIP toxin inhibitor known to one of skill in the art, or subsequently identified hereto, which can inactivate a RIP toxin, can be used in the methods provided herein. Typically, such RIP toxin inhibitors are any that inhibit toxic activity by targeting, for example, the conserved N-glycosidase activity of REP toxins. Other RIP toxin inhibitors can be chosen that target any one or more other RIP activities including, but not limited to, RNase activity, DNase activity, and superoxide dismutase and phospholipase activities. For purposes herein, any RIP inhibitor, such as adenine or any analog thereof, can be used in the methods herein so long as the inhibitor exhibits an inhibitory activity against the starting form of the RIP toxin, for example, the wildtype form of the RIP toxin or active fragment thereof. For example, 4-APP can be used in the methods herein to select for a modified RIP including, but not limited to, a modified SA1, saporin, momordin, or bryodin (Brigotti et al. (2000) Life Sciences, 68:331-336). Typically, 4-APP is used in the methods herein to select for a modified SA1. It also is contemplated that other inhibitors can be used to select for a modified SA1.

The amount of RIP inhibitor used in the selection methods can be empirically determined based on its known effects on the toxic activity of a RIP protein or conjugate thereof. It is important that the REP inhibitor used in the methods herein is itself not toxic to the specific host cell, which toxicity is known or can be determined by one of skill the art depending on the host cell chosen. Further, to ensure that a RIP inhibitor effectively modulates selection of a modified RIP toxin or conjugate thereof, a concentration of RIP inhibitor is chosen such that it inhibits the toxic activity of the starting protein. Typically, a concentration of REP inhibitor is chosen such that the starting RIP protein retains some activity in the presence of the RIP inhibitor, thereby allowing for some degree of selective pressure or modulation of the RIP inhibitor in the selection method. Generally, a concentration of the RIP inhibitor is chosen that inhibits at least or about 1%, 2%, 3%, 4%, 5%, 6%, 7%, 8%, 9%, 10%, 20%, 30%, 40%, 50%, 60%, 70%, 80%, or 90% of the toxic activity of a RIP toxin, or conjugate thereof, but less than 100% of the toxic activity. Various assays known to one of skill in the art can be used to test the affects of various concentrations of RIP inhibitors on the activities of host cells or RIP proteins.

Typically, in the selection methods herein, a RIP inhibitor, such as for example 4-APP, is added to modulate selection at about or at 0.1 mM, 0.2 mM, 0.3 mM, 0.4 mM, 0.5 mM, 0.6 mM, 0.7 mM, 0.8 mM, 0.9 mM, 1.0 mM, 1.5 mM, 2.0 mM, 3.0 mM, 4.0 mM, 5.0 mM, 6.0 mM, 7.0 mM, 8.0 mM, 9.0 mM, 10.0 mM, or more so long as the inhibitor is itself not toxic to the host cell chosen. It is understood that the concentration of the RIP inhibitor chosen can vary depending on the host cell chosen or the conditions used for recombinant expression. For example, exemplary of a chosen concentration of 4-APP for use in the selection methods herein in an E. coli cell expression system is at or about 0.2 to 0.8 mM, generally 0.5 mM of inhibitor.

The RIP inhibitor can be added before, during, or after treatment of the host cells with the starting protein RIP toxin or conjugate thereof. In some examples, the RIP inhibitor is added to a liquid culture or medium such as for example to cell culture medium. In other examples, the RIP inhibitor is added to a medium capable of solidifying such as a solid agar. For example, a REP inhibitor, such as for example, 4-APP, can be added to luria broth (LB) agar for the generation of agar plates containing the RIP inhibitor. The RIP inhibitor can be used as a selective modulator alone or can be used in the presence of other selective modulators or selective agents such as, but not limited to, other RIP inhibitors or antibiotics conferring antibiotic resistance.

The selected modified toxins expressed from the host cell transformants can be amplified to facilitate identification of the selected modified RIP toxin or conjugate thereof. Such methods include general recombinant DNA techniques and are routine to those of skill in the art. The vector from the host cell transformants containing the modified toxin DNA can be isolated to enable purification of the selected protein. For example, following transformation of E. coli host cells with a REP starting protein as set forth above, the cell transformants grow as individual clones which can be isolated such as by individually picking a colony and growing it up for plasmid purification using any method known to one of skill in the art, and if necessary can be prepared in large quantities, such as for example, using the Midi Plasmid Purification Kit (Qiagen). The purified plasmid can be used for DNA sequencing to identify the sequence of the modified toxin, or can be used to transfect into any cell for further expression and production thereof, such as but not limited to, a prokaryotic or eukaryotic expression system. In some examples, a one or two-step PCR can be performed to amplify the selected sequence, which can be subcloned into an expression vector of choice. The PCR primers can be designed to facilitate subcloning, such as by including the addition of restriction enzyme sites.

Following further expression and production of selected toxins in any desired cell expression system, conditioned medium containing the RIP toxin polypeptide or conjugate thereof can be tested in activity assays or can be used for further purification. Typically, any further expression and production of the selected modified RIP toxin or conjugate thereof is performed in the presence of a RIP inhibitor. Such a method is described in detail below under Section G for the improved production of RIP toxins or conjugates thereof.

4. Activity Assessment

Modified RIP toxins, or conjugates thereof, selected in the methods provided herein can be tested to determine if, following selection, they retain toxic activity against host cell ribosomes. Typically, such modified toxins are selected for because they exhibit a reduced toxic activity compared to a starting REP protein or conjugate thereof. Generally, however, selected modified toxins retain some activity of the starting toxin protein. Modified RIP toxins, or conjugates thereof, provided herein retain 0.5%, 1%, 1.5%, 2%, 5%, 10%, 20%, 30%, 40%, 50%, 60%, 70%, 80%, 90%, 95%, or more of the toxic activity compared to a reference or starting form of the toxin, or conjugate thereof. Exemplary of assays can be any assay that tests for an activity of a RIP polypeptide including, for example, assays that assess N-glycosidase activity, DNAase activity, RNAase activity, or other activities. Any method known to one of skill in the art can be used to assess toxic activity of a RIP protein or conjugate thereof, and typically include any that assay for effects of the N-glycosidase activity of the RIP protein. Exemplary assays to assess the toxic activity of any RIP toxin or conjugate thereof, including modified forms of such toxins, are described below.

a. Protein Synthesis Assays

The activity of RIP toxins or conjugates thereof, such as any modified RIP, including, for example, any modified toxin identified in the selection method provided herein, can be measured to determine effects of the toxin on translation using a protein synthesis assay. Such assays are routine and are known to one of skill in the art. Exemplary of such an assay is a rabbit reticulocyte lysate assay. Typically the rabbit reticulocyte lysate contains or is supplemented with components needed for efficient transcription and translation such as magnesium and potassium ions and NTPs. A template RNA also is added which is the source of the synthesized protein. Such an assay allows for the coupled in vitro transcription and translation of proteins by rabbit ribosomes which can be detected. In one method, detection can be achieved via incorporation of radioactivity such as [³H]Leu, [³⁵S]Met or [³⁵S]Met-Cys which is incorporated into the synthesized protein and can be measured following precipitation with trichloroacetic acid (TCA; Baas et al. (1992) The Plant Cell, 4: 225-234; Zhao et al. (2005) Journal of Microbiology, 54: 1023-1030). In another method, luciferase DNA can be used as the template, which is then, detected using a luminometer. Exemplary of rabbit reticulocyte lysate systems are those sold by Promega including, for example, the TNT® Coupled Reticulocyte Lysate Systems. Such an assay is described in Example 2. The assay can be adapted to be used with other translation systems including wheat or maize reticulocyte lysates, or can be adapted in translation reactions containing intact cell lysates or lysates reconstructed from various supernatant fractions and purified ribosomes or polyribosomes (Baas et al. (1992) The Plant Cell, 4: 225-234). In some methods the assay can be adapted to assess effects on protein synthesis in whole cells, where detection of protein synthesis can be facilitated by adenoviral expressed luciferase (Zhao et al. (2005) Journal of Microbiology, 54: 1023-1030). In addition, kinetic analysis and dose response curves can be performed to determine the relative activity of the toxin as determined by the concentration of the toxin necessary to give one-half the maximum response (RIC50).

b. Depurination Assays

The activity of RIP toxins or conjugates thereof, such as any modified RIP, including, for example, any modified toxin identified in the selection method provided herein, can be determined in a depurination assay. RIP-mediated depurination of the large ribosomal subunit of RNA increases susceptibility of the sugar-phosphate backbone to hydrolysis at the depurination site (Tumer et al. (1997) Proc. Natl. Acad. Sci., 94: 3866-3871). Following treatment with aniline, hydrolysis can be observed typically by release of a small fragment. Thus, ribosomes can be treated in the presence or absence of increasing concentration of toxin, the RNA extracted and treated with aniline, and analyzed by gel electrophoresis. Fragments can be visualized by staining with ethidium bromide (Tumer et al. (1997) Proc. Natl. Acad. Sci., 94: 3866-3871; Hartley et al. (1991) FEBS, 290:1:65-68). The percent dupurination can be determined by scanning negatives of photographs of the RNA gels (see e.g., Taylor et al. (1994) The Plant Journal, 5: 827-835).

c. Cell Growth/Survival/Viability Assays

The activity of RIP toxins or conjugates thereof, such as any modified RIP, including, for example, any modified toxin identified in the selection method provided herein, can be determined by directly assessing effects on cell growth. In such an assay, any prokaryotic or eukaryotic cell can be introduced with DNA encoding a toxin such as in the form of a suitable expression vector. Alternatively, any prokaryotic or eukaryotic cell can be administered directly with a RIP polypeptide, or a conjugate thereof such as, for example, a ligand-toxin conjugate. Any cell can be tested, including but not limited to, any primary cell such as directly obtained from a subject, i.e. from the blood, serum, or other tissue source. Included among such cells are any leukocyte subtypes or activated leukocytes thereof. Cell lines also can be used in assays to assess the toxicity of RIP polypeptides, or conjugates thereof. Exemplary of a cell line is THP-1, U251 or HT-29 cells.

Cell growth can be monitored by assaying for cell proliferation, cell viability or cell survival. Growth can be monitored over time and in the presence or absence of increasing concentrations of the toxin. For example, cell growth can be monitored by counting the cells in a Coulter Counter, measuring the optical density of the cells over time (Suh et al. (1998) Biochemistry, 37: 9394-9398), using a DNA dye such as MTT which is reduced by live cells to form insoluble purple formazan crystals that can be measured (Arora et al. (1999) Cancer Research, 59: 183-188; McDonald et al. (2001) IDrugs, 4:427-442), or by using a dye such as trypan blue which is excluded from viable cells but not dead cells (McDonald et al. (2001) IDrugs, 4:427-442). In another example, cell viability can be assessed by measuring the amount of ATP released into the cell culture medium. Exemplary of such an assay is the CellTiter-Glo™ Luminescent Cell Viability Assay Kit (Promega, Madison Wis.) such as is described, for example, in Example 5. Upon lysis of the cells with the ATP reaction mixture (supplied by the manufacturer as CellTiter-Glo® Reagent), ATP drives the oxygenation of luciferin resulting in a luminescent signal which is proportional to ATP concentrations in the wells. This is directly proportional to the number of viable cells in the culture.

### E. EXEMPLARY MODIFIED TOXINS

Provided herein are modified RIP toxin polypeptides, or conjugates thereof, that exhibit reduced toxic activity compared to a wildtype RIP polypeptide. For example, a modified RIP toxin polypeptide, or conjugate thereof, exhibits 0.5%, 1%, 1.5%, 2%, 5%, 10%, 20%, 30%, 40%, 50%, 60%, 70%, 80%, 90%, 95%, or more of the toxic activity compared to a reference or starting form of the toxin, or conjugate thereof. By virtue of the reduced toxicity, such modified RIP toxin polypeptides are expressed by host cells, and can be purified, isolated and/or identified therefrom. The modified toxins, or conjugates thereof, exhibit reduced toxicity to eukaryotic or prokaryotic cells. Generally, the modified RIP toxins or conjugates thereof exhibit reduced toxicity to bacterial cells, such as E. coli, which thereby permit a source of toxin that can be used in production methods in E. coli.

Typically, such modified RIP toxin polypeptides, or conjugates thereof, retain one or more activities of the starting or wildtype form of the protein (i.e. unmodified polypeptide). For example, the modified RIP toxin polypeptides, or conjugates thereof, retain at least or about 0.5%, 1%, 1.5%, 2%, 5%, 10%, 20%, 30%, 40%, 50%, 60%, 70%, 80%, 90%, 95%, or more activity of one or more than one RIP activity compared to the unmodified or wildtype RIP polypeptide, or conjugate thereof. Activities of a RIP polypeptide include, but are not limited to, any one or more of N-glycosidase activity, polynucleotide:adenosine glycosidase activity including RNAase activity and DNAase activity, superoxide dismutase activity, phospholipase activity, chitinase activity and anti-viral activity. Activity can be assessed in vitro or in vivo and can be compared to the activity of the starting RIP polypeptide.

In some examples, such modified RIP polypeptides are identified in the selection methods herein such as by virtue of their expression by a host cell compared to a starting RIP polypeptide, or a polypeptide conjugate containing a RIP polypeptide, that is not expressed by the host cell or is expressed at low levels. As is described above, such modified RIP toxin polypeptides are identified following introduction of a nucleic acid encoding a starting or wildtype RIP polypeptide, for example, any nucleic acid encoding a RIP polypeptide set forth in Table 3, or an active fragment thereof, followed by selection and identification of expressed RIP polypeptides. In some examples, the modified RIP toxin polypeptides provided herein are identified following expression of a toxin from a host cell introduced with nucleic acid encoding a starting RIP toxin or active portion thereof. In other examples, the modified RIP toxin polypeptides provided herein are identified following expression of a RIP toxin conjugate polypeptide (i.e. ligand-toxin conjugate) from a host cell that is introduced with a starting conjugate encoding a polypeptide containing a RIP toxin, or active portion thereof. Typically, such a conjugate is a fusion protein, thereby enabling introduction of a nucleic acid molecule encoding the fusion protein into the cell.

Using methods described herein, the particular modification in the expressed RIP polypeptide can be identified, such as for example, by sequencing of the polypeptide. Typically, modifications identified in the methods herein include any that alter the primary sequence of the unmodified polypeptide and include, but are not limited, any one or more amino acid replacements, amino acid deletions and/or amino acid truncations. For example, modifications include any one or more amino acid mutations in the primary sequence, or truncation of the primary sequence, or any combination thereof. Hence, modifications of amino acid residues in a RIP polypeptide, or active fragment thereof, can be identified that confer reduced cell toxicity by virtue of a change to the primary sequence of the polypeptide.

The modification(s) identified in the selection method herein in an expressed RIP polypeptide can be made in a corresponding position(s) of any target protein, for example, any related polypeptide such as, but not limited to, any allelic, species, truncated or other variant form of the expressed RIP polypeptide. Such modifications can be made by standard recombinant DNA techniques such as are routine to one of skill in the art. Any method known in the art to effect mutation of any one or more amino acids in a target protein can be employed. Methods include standard site-directed mutagenesis (using e.g., a kit, such as QuikChange available from Stratagene) of encoding nucleic acid molecules, or by solid phase polypeptide synthesis methods.

In addition, any modified RIP polypeptide identified in the methods herein, or generated based on a modification identified in the methods herein, can be used to generate a fusion protein or conjugate. For example, a ligand-toxin conjugate can be generated having the modified toxin moiety as the targeted agent (see Section F below) linked directly or indirectly to any moiety that targets to a cell surface receptor for internalization thereof. Such conjugates can be generated by routine recombinant DNA techniques. For example, conjugates can be generated using restriction enzymes and cloning methodologies for routine subcloning of the desired conjugate components. Any modified polypeptide generated, including any conjugate, having a modification identified in the selection methods herein retains toxic activity. Such modified polypeptides generally retain or exhibit any one or more RIP activities. The toxic activity and other activities of the conjugates can, be tested.

Other modifications that are or are not in the primary sequence of the polypeptide also can be included in a modified RIP polypeptide, or conjugate thereof, such as, but not limited to, the addition of a carbohydrate moiety, the addition of a polyethylene glycol (PEG) moiety, the addition of an Fc domain, etc. For example, such additional modifications can be made to increase the stability or half life of the protein.

Modified SA1 Toxins

Exemplary of RIP toxins provided herein are modified forms of SA1, including modifications in any active form thereof, for example any truncated form thereof so long as the truncated polypeptide exhibits a RIP activity, and allelic or species variants thereof. For example, a modified SA1 polypeptide can include any one or more modifications in a truncated variant of SA1 such as is set forth in SEQ ID NO:22 or SEQ ID NO:24, or any allelic or species variant thereof. Modified SA1 toxins can be truncated, or can express amino acid mutations compared to the starting SA1 toxin used in the selection methods. Typically, such modified toxins retain one or more activities compared to the starting SA1 polypeptide. Accordingly, such modified SA1 polypeptides can be used in methods to improve production of an SAI polypeptide and/or can be used in fusion proteins to generate conjugate proteins containing the modified SA1 polypeptide.

The modified SA1 polypeptides can be identified in the selection methods herein. In one example, the modified SA1 polypeptide can be identified following introduction of nucleic acid encoding an SA1 polypeptide, or active fragment thereof. For example, selection of a modified SA1 polypeptide can be achieved following introduction of nucleic acid encoding a variant 1 SA1 polypeptide such as is set forth in SEQ ID NO:22. In another example, selection of a modified SA1 polypeptide can be achieved following introduction of nucleic acid encoding a variant 2 SA1 polypeptide. The variant 2 SA1 is a form of SA1 made to lack the five C-terminal amino acids (CHHHA) compared to the variant 1 SA1 set forth in SEQ ID NO:22 in order to avoid cysteine-induced dimerization. The amino acid sequence of variant 2 SA1 is set forth in SEQ ID NO:24 and encoded by a sequence of nucleic acids set forth in SEQ ID NO:25.

In some cases, selection for a modified SA1 polypeptide can be achieved following introduction of nucleic acid encoding a conjugate containing an SA1 polypeptide portion. The conjugates can include any ligand-toxin conjugate or other conjugate so long as the conjugate contains an SA1 polypeptide, or active fragment thereof. For example, chemokine conjugates described in U.S. Pat. Nos. 7,166,702, 7,157,418 and 7,192,736 can be used as a starting protein to identify modified forms of a variant 1 SA1 polypeptide (set forth in SEQ ID NO:22 and encoded by a sequence of nucleic acids set forth in SEQ ID NO:23). In one example, an LPM1a polypeptide is used as a starting protein, which is a conjugate of the chemokine MCP-1 linked indirectly to the variant 1 SA1 polypeptide. The LPM1a conjugate is set forth in SEQ ID NO: 38 and encoded by a sequence of nucleic acids set forth in SEQ ID NO:37. In another example, a conjugate containing the chemokine MCP-1 linked with a variant 2 form of SA1 can be used as the starting unmodified protein, also termed LPM1b herein. The LPM1b conjugate is set forth in SEQ ID NO: 40 and encoded by a sequence of nucleic acids set forth in SEQ ID NO:39.

Provided herein is a modified SA1 toxin that contains an amino acid mutation at position 38, corresponding to position 38 of a variant SA1 polypeptides set forth in SEQ ID NO: 22. For example, amino acid modifications can correspond to position L38. An exemplary amino acid mutation in SA1 identified in the methods provided herein correspond to modification L38R in a variant SA1 polypeptide such as set forth in SEQ ID NOS: 22. In some examples, the corresponding L38R mutation is identified or made in other SA1 variant forms, including allelic or species variants. For example, a corresponding L38R mutation can be made in a variant 2 sequence of SA1 set forth in SEQ ID NO:24. An exemplary SA1 toxin having an amino acid mutation of L38R is set forth in SEQ ID NO: 26 and encoded by a sequence of amino acids set forth in SEQ ID NO:27. This modified SA1 also is termed mutant variant 1 (also called variant 3) herein. The mutant variant 1 SA1 polypeptide can be used to generate further toxin conjugates, which can be used, for example, in methods to treat disease or disorders for which the conjugate is designed. Additionally, the mutant variant 1 SA1 polypeptide can be used in methods to improve production of SA1 or conjugates thereof.

In another example, provided herein is a modified SA1 toxin that contains an amino acid mutation at position 219, corresponding to position 219 of a variant SA1 polypeptides set forth in SEQ ID NO: 22. For example, amino acid modifications can correspond to position V219. An exemplary amino acid mutation in SA1 identified in the methods provided herein correspond to modification of V219A in a variant SA1 polypeptide set forth in SEQ ID NOS:22. In some examples, the corresponding V219A mutation is identified or made in other SA1 variant forms, including allelic or species variants. For example, a corresponding V219A mutation can be made in a variant 2 sequence of SA1 set forth in SEQ ID NO:24. An exemplary modified SA1 polypeptide having an amino acid mutation of V219A is set forth in SEQ ID NO: 28 and encoded by a sequence of amino acids set forth in SEQ ID NO:29. This modified SA1 also is termed mutant variant 2 (also called variant 4) herein. The mutant variant 2 SA1 polypeptide can be used to generate further toxin conjugates, which can be used, for example, in methods to treat disease or disorders for which the conjugate is designed. Additionally, the mutant variant 2 SA1 polypeptide can be used in methods to improve production of SA1 or conjugates thereof.

In addition, any of the mutations identified in a modified RIP polypeptide in the selection methods herein can be combined. For example, a modified SA1 polypeptide, or conjugate thereof, can be generated having a modification of L38 and V219, corresponding to positions in a variant SA1 set forth in any of SEQ ID NO: 22. Such a modification can be made corresponding position in any SA1 polypeptide, such as an SA1 polypeptide set forth in SEQ ID NOS:22 or 24, respectively, any allelic or species variants thereof, or any other SA1 variants known to one of skill in the art. Further, any of the mutations identified in a modified RIP polypeptides in the selection method herein can be combined with any other mutations in the RIP polypeptide known to skill of the art or subsequently identified hereto. Typically, any such combination mutant in a RIP polypeptide, or a conjugate thereof, exhibits reduced toxicity to a host cell compared to a wild-type or starting form of a RIP polypeptide, yet retains 0.5%, 1%, 1.5%, 2%, 5%, 10%, 20%, 30%, 40%, 50%, 60%, 70%, 80%, 90%, 95%, or more of the toxic activity compared to a reference or starting form of the toxin, or conjugate thereof.

### F. TARGETING AGENTS AND CONJUGATES THEREOF

Provided herein are conjugates containing RIP polypeptide toxins, or active fragments thereof, linked directly or indirectly to one or more moieties or agents, such as any chemical, polypeptide, or peptide moiety, or portions thereof. Typically, the toxins for use in the conjugates provided herein are those containing any RIP toxin variant, including any modified RIP toxin variant. Such modified toxins include any modified RIP toxin provided herein or identified using the methods of selection provided herein, such as for example, a modified SA1 toxin, or allelic variants or fragments thereof. Included among such a modified SA1 is a mutant variant 1 SA1 (i.e. variant 3) or a mutant variant 2 SA1 (i.e. variant 4) identified in the selection methods herein.

Typically, a modified RIP toxin is linked directly or indirectly to a targeting agent, including any agent that targets the conjugate to one or more cell types by selectively binding to a cell surface receptor (i.e. referred to herein as ligand-toxin conjugates). Hence, any polypeptide or molecule that binds to a cell surface receptor and is internalized by a cell is intended for use herein. Such targeting agents include, but are not limited to, growth factors, cytokines, chemokines, antibodies, and hormones, or allelic variants, muteins, or fragments thereof so long as the targeting agent is internalized by a cell surface receptor. Further, the conjugates provided herein can optionally include additional components, such as for example, but not limited to, additional sequences or moieties to facilitate cloning, expression, post-translational modification, purification, detection, and administration. These include, for example, restriction enzyme sequences, translational start or stop codon sequences, His tags, or other such components. For example, a starting methionine codon or Kozak sequence can be added to the coding nucleic acid sequence to permit or enhance translation of a mature polypeptide or a fragment polypeptide. Furthermore, as described above in Section D, any nucleic acid molecule that encodes a ligand-toxin conjugate containing a targeting agent linked to a target agent, such as an SA1 subunit, or active portion thereof, can be used to screen for variants of the targeted agent, such as a modified SA1 as described above and in the Examples herein.

1. Targeting Agents

Generally, the modified RIP toxin conjugates provided herein contain a targeting agent that targets the conjugate to a receptor or receptors on a cell or a population of cells involved in the pathology of various disease processes. Depending on the disease or disorder, such cells are activated cells that are a function of the disease as well as the disease process, or are bystander cells that support the disease process. Consequently, targeting these receptors and the cells that express these receptors permits the therapy to be tailored to the particular disease and also to the progress of the disease. Hence, conjugates provided herein can be used as therapeutics in the treatment of various diseases.

For example, conjugates provided herein include ligand-toxins which contain a targeting moiety that binds to receptors on specific cells types involved in the immune response, including various leukocyte subtypes involved in inflammatory diseases. For example, as a component of the conjugates provided herein, a targeting moiety can include a ligand that targets one or more cell surface receptors expressed on cells of the immune system, such as any cell of the leukocyte lineage or other tissue residential cells, including on activated cells involved in diseases processes. Examples of cell types that can be targeted herein by the conjugates include, but are not limited to leukocytes including, but not limited to, monocytes, macrophages including tissue macrophages such as microglia of the brain, kupfer cells of the liver or alveolar macrophages of the lung, B cells, T cells including Th1 and Th2 cells, basophils, eosinophils, dendritic cells including immature and mature dendritic cells and langerhans cells, mast cells, natural killer cells, and neutrophils. Other examples of cell types that can be targeted herein include, platelets, astrocytes, endothelial cells, neurons, epithelial cells and adipose cells.

a. Chemokines

Provided herein are conjugates whereby the targeting agent used in the ligand-toxin conjugate is selected from the family of chemokines. To appreciate the use of a chemokine as a targeting agent in the conjugates herein, an understanding of the function and interaction of chemokine ligands and their receptors is helpful. The following discussion provides such background.

Chemokines are a family of forty or more small proteins that typically are secreted by cells and stimulate the activation and/or migration (“chemotaxis”) of nearby responsive cells, typically leukocytes, which express cognate chemokine receptors. Together, chemokines target the entire spectrum of leukocyte subtypes; individually each targets a part of the spectrum. Although some chemokines are constitutive and involved in homeostatic immune responses, many chemokines are termed inflammatory chemokines and are induced from a wide variety of cells in response to bacterial infection, viruses and other stimulatory agents.

Chemokines have a variety of biological activities. They were initially isolated by their ability to stimulate leukocyte migration and activation. Chemokines, in association with adhesion molecules, recruit subsets of leukocytes to specific sites of inflammation and tissue injury. For example, chemokines function mainly as chemoattractants via stimulation of chemokine receptors expressed on a variety of leukocytes including those in innate immunity thereby recruiting monocytes, neutrophils and other effector cells from the blood to sites of infection or damage, and also those in adaptive immunity including recruitment of lymphocytes to sites of immune reactions. Generally, chemokines and chemokine receptor expression are upregulated in disease, with chemokines acting in an autocrine or paracrine manner (Glabinski et al. (1995) Int. J. Dev. Neurosci., 13:153-65 Furie and Randolph (1995) Am. J. Pathol., 146:1287-301; Benveniste E. N. (1997) J. Mol. Med., 75:165-73; Schall et al. (1994) Current Biol., 6:865-73; Taub et al. (1994) Ther. Immunol., 1:229-46; Baggliolini et al. (1994) Adv. Immunol., 55:97-179; and Haelens et al. (1996) Immunobiol., 195: 499-521; Taub, Cytokine Growth Factor Res., 7: 355-76, 1996; Dong et al., Eur. J. Dermatol., 13: 224-30, 2003; Pastore et al., Eur. J. Dermatol., 14: 203-8, 2004: Charo and Ransohoff, N Eng J Med., 354: 6100-21, 2006).

Chemokines also induce activation of cells, including but not limited to, microglia and macrophages. Thus, chemokines are thought to induce the production and release of reactive oxygen species, degradative enzymes, and inflammatory and toxic cytokines from various leukocyte populations. In addition, chemokines have been shown to regulate negative hematopoietic progenitor proliferation, and several CXC chemokines can regulate angiogenesis. Chemokines also play a role in many diseases that involve inflammatory tissue destructions, such as adult respiratory distress syndrome, myocardial infarction, rheumatoid arthritis, and atherosclerosis.

Chemokines were originally named, for example, according to their functions or origins. The most common name for a given ligand is used in the text herein. Recent systematic nomenclature has been adopted which uses the chemokine group name followed by a number. For example the ligands monocyte chemoattractant protein (MCP)-1 and interleukin (IL)-8 are systematically referred to as CCL2 and CXCL8, respectively. Their receptors are referred to as CCR2 and CXCR1/2, respectively (Table x and 7; Bacon, et al., J. Interferon Cytokine Res., 22: 1067-8, 2002; Murphy, Pharmacol. Rev, 54: 227-9, 2002; Murphy et al., Pharmacol. Rev., 52: 145-76, 2000). Chemokines and chemokine receptors are referred to herein interchangeably with reference to the common and systematic nomenclature. Given one name, one of skill in the art knows or could determine the corresponding names.

i. Ligands

Chemokines, as noted above, are a superfamily of small (approximately about 6 to about 14 kDa), inducible and secreted, chemoattractant cytokines that act primarily on leukocyte subtypes. Chemokine ligands have between 15 and 50% identity in their primary structures but it is their shared highly conserved three-dimensional structure that is responsible for receptor binding and activation. The superfamily is divided into four sub-families based upon the position (or existence) of four conserved cysteine residues in the primary sequences. Three of the groups contain four cysteines, the other group does not. The groups are defined by the arrangement of the first two cysteines. If the first two cysteines are separated by a single amino acid they are members of the CXC family (also called alpha); if the cysteines are adjacent, they are classified in the CC family (also called beta); if the cysteines are separated by three amino acids CX₃C, they are members of the third group (also called delta). The fourth group of chemokines, C or gamma, contain two cysteines, corresponding to the first and third cysteines in the other groups.

Structural analysis demonstrates that most chemokines function as monomers and that the two regions necessary for receptor binding reside within the first 35 amino acids of the flexible N-terminus of the mature polypeptide (Clark-Lewis et al. (1995) J Leukocyte Biol., 57:703-11; Beall et al. (1996) Biochem. J. 313:633-40; and Steitz et al. (1998) FEBS Lett 430: 158-64). Dimers of chemokines can form, which formation varies between chemokines. The formation of dimers typically occurs at high concentration in solution (Baggiolini et al. (2001) J Int. Med., 250:91-104). Dimers, however, dissociate upon dilution and the monomers constitute the biologically active molecule.

In general, the alpha chemokine members preferentially are active on neutrophils and T-lymphocytes, and the beta chemokines are active on monocytes, macrophages, eosinophils and T-lymphocytes. Additionally, several members of the alpha and beta chemokines sub-families are active on dendritic cells, which are migratory cells that exhibit potent antigen-presenting properties following their activation and maturation from immature phagocytic cells, and are thought to participate in the pathophysiology of many inflammatory diseases (e.g., Xu et al., J. Leukoc. Biol., 60: 365-71, 1996; and Sozzani et al., J. Immunol., 159: 1993-2000, 1997; Hashimoto et al., J Dermatol Sci., 44: 93-9, 2006; van Rijt et al., J Exp Med, 201: 981-91, 2005). A fourth human CX3C-type chemokine referred to as fractalkine has recently been reported (Bazan et al., Nature, 385:640-4, 1997; Imai et al., Cell, 91:521-30, 1997; Mackay, Curr. Biol. 7: R384-6, 1997). Unlike other chemokines, fractalkine exists in membrane and soluble forms. The soluble form is a potent chemoattractant for monocytes and T-cells. The cell surface receptor for this chemokine is termed CX3CR1. It should be noted that there can be subtle differences between the chemical nature and physiological effects of chemokines derived from different species (Baggliolini et al., Adv. Immunol., 55: 97-179, 1994; and Haelens et al., Immunobiol., 195: 499-521, 1996).

Table 4 sets forth exemplary chemokines, including synonyms therefor, and exemplary SEQ ID NOS. Further, Table 4 sets forth the signal sequence and amino acid positions coding for the mature chemokine with reference to positions in the respective SEQ ID NO. It is noted that, the description of amino acid positions are for illustrative purposes and are not meant to limit the scope of the embodiments provided. It is understood that polypeptides and the description thereof are theoretically derived based on homology analysis and alignments with similar polypeptides. Thus, the exact locus can vary, and is not necessarily the same for each polypeptide. Allelic variant or species variants of chemokines also are known. Examples of allelic variations in exemplary chemokines are set forth in any of SEQ ID NOS:170-191.

TABLE 4
Exemplary Chemokine Ligands

| | | UniProt | Signal | Mature | |
|---|---|---|---|---|---|
| Chemokine | Synonyms | NO: | Sequence | Chemokine | SEQ ID NO: |
| MCP-1 (Monocyte | CCL2; Small | P13500 | 1-23 | 24-99 | 112 |
| chemoattractant | inducible cytokine | | | | |
| protein-1) | A2; MCAF; | | | | |
| | Monocyte secretory | | | | |
| | protein JE; HC11; | | | | |
| | SCYA2 | | | | |
| Eotaxin (Eosinophil | CCL11; Small | P51671 | 1-23 | 24-97 | 113 |
| chemotactic | inducible cytokine | | | | |
| protein) | A11; SCYA11 | | | | |
| SDF-1β (Stromal | CXCL12; Pre-B cell | P48061 | 1-21 | 22-93 | 114 |
| cell-derived factor | growth-stimulating | | | | |
| 1) | factor (PBSH); hIRH | | | | |
| GRO-α (Growth- | CXCL1; Melanoma | P09341 | 1-34 | 35-107 | 115 |
| regulated protein | growth stimulatory | | | | |
| alpha) | activity (MGSA); | | | | |
| | Neutrophil-activating | | | | |
| | protein 3 (NAP-3); | | | | |
| | SCYB1 | | | | |
| MIP-1β | CCL4; Small | P13236 | 1-23 | 24-92 | 116 |
| (Macrophage | inducible cytokine | | | | |
| inflammatory | A4; T-cell activation | | | | |
| protein 1-beta) | protein 2; ACT-2; | | | | |
| | PAT 744; H400; SIS- | | | | |
| | γ; Lymphocyte | | | | |
| | activation gene 1 | | | | |
| | protein (LAG-1); | | | | |
| | HC21; G-26 T- | | | | |
| | lymphocyte-secreted | | | | |
| | protein; SCYA4 | | | | |
| IL-8 (Interleukin-8) | CXCL8; Monocyte- | P10145 | 1-20 | 21-99 | 117 |
| | derived neutrophil | | | | |
| | chemotactic factor | | | | |
| | (MDNCF); T-cell | | | | |
| | chemotactic factor; | | | | |
| | Neutrophil-activating | | | | |
| | protein 1 (NAP-1); | | | | |
| | Protein 3-10C; | | | | |
| | Granulocyte | | | | |
| | chemotactic protein 1 | | | | |
| | (GCP-1); Monocyte- | | | | |
| | derived neutrophil- | | | | |
| | activating peptide | | | | |
| | (MONAP); | | | | |
| | Emoctakin | | | | |
| IP-10 (Interferon- | CXCL10; Small | P02778 | 1-21 | 22-98 | 118 |
| inducible protein- | inducible cytokine | | | | |
| 10) | B10; 10 kDa | | | | |
| | interferon-gamma- | | | | |
| | induced protein; γ- | | | | |
| | IP10; SCYB10 | | | | |
| MCP-3 (Monocyte | CCL7; Small | P80098 | 1-23 | 24-99 | 119 |
| chemotactic protein | inducible cytokine | | | | |
| 3) | A7; NC28; SCYA7 | | | | |
| MIP-3α | CCL20; Small | P78556 | 1-26 | 27-96 | 120 |
| (Macrophage | inducible cytokine | | | | |
| inflammatory | A20; Liver and | | | | |
| protein 3 alpha) | activation-regulated | | | | |
| | chemokine (LARC); | | | | |
| | Beta chemokine | | | | |
| | exodus-1; SCYA20 | | | | |
| MDC | CCL22; Small | O00626 | 1-24 | 25-93 | 121 |
| (Macrophage- | inducible cytokine | | | | |
| derived chemokine) | A22; Stimulated T- | | | | |
| | cell chemotactic | | | | |
| | protein 1 (STCP-1); | | | | |
| | SCYA22 | | | | |
| MIP-1α | CCL3; Small | P10147 | 1-23 | 24-92 | 122 |
| (Macrophage | inducible cytokine | | | | |
| inflammatory | A3; Tonsillar | | | | |
| protein 1-alpha) | lymphocyte LD78 | | | | |
| | alpha protein; G0/G1 | | | | |
| | switch regulatory | | | | |
| | protein 19-1 (G0S19- | | | | |
| | 1 protein); SIS-β; | | | | |
| | PAT 464.1; SCYA3 | | | | |
| BCA-1 (B cell- | CXCL13; Small | O43927 | 1-22 | 23-109 | 123 |
| attracting | inducible cytokine | | | | |
| chemokine 1) | B13; B lymphocyte | | | | |
| | chemoattractant; CXC | | | | |
| | chemokine BLC; | | | | |
| | ANGIE; SCYB13 | | | | |
| GCP-2 | CXCL6; Small | P80162 | 1-37 | 38-114 | 124 |
| (Granulocyte | inducible cytokine | | | | |
| chemotactic protein | B6; Chemokine alpha | | | | |
| 2) | 3 (CKA-3); SCYB6 | | | | |
| ENA-78 | CXCL5; Small | P42830 | 1-36 | 37-114 | 125 |
| (Epithelial-derived | inducible cytokine | | | | |
| neutrophil- | B5; SCYB5 | | | | |
| activating protein | | | | | |
| 78) | | | | | |
| PBP (Platelet basic | CXCL7; Small | P02775 | 1-34 | 35-128 | 126 |
| protein) | inducible cytokine | | | | |
| | B7; Leukocyte- | | | | |
| | derived growth factor | | | | |
| | (LDGF); | | | | |
| | Macrophage-derived | | | | |
| | growth factor | | | | |
| | (MDGF); Connective | | | | |
| | tissue-activating | | | | |
| | peptide III (CTAP- | | | | |
| | III); Low-affinity | | | | |
| | platelet factor IV | | | | |
| | (LA-PF4); TC-2; β- | | | | |
| | thromboglobulin; | | | | |
| | Neutrophil-activating | | | | |
| | peptide 2 (NAP-2); | | | | |
| | SCYB7 | | | | |
| MIG (Gamma | CXCL9; Small | Q07325 | 1-22 | 23-125 | 127 |
| interferon-induced | inducible cytokine | | | | |
| monokine) | B9; SCYB9 | | | | |
| PF-4 (Platelet | CXCL4; Oncostatin | P02776 | 1-31 | 32-101 | 128 |
| factor 4) | A; Ironplact; SCYB4 | | | | |
| PF-4var1 (Platelet | CXCL4L1; PF4alt; | P10720 | 1-30 | 31-104 | 129 |
| factor 4 variant) | PF4V1; SCYB4V1 | | | | |
| SDF-2 (Stromal | | Q99470 | 1-18 | 19-211 | 130 |
| cell-derived factor | | | | | |
| 2) | | | | | |
| MCP-2 (Monocyte | CCL8; Small | P80075 | 1-23 | 24-99 | 131 |
| chemotactic protein | inducible cytokine | | | | |
| 2) | A8; HC14; SCYA10; | | | | |
| | SCYA8 | | | | |
| MCP-4 (Monocyte | CCL13; Small | Q99616 | 1-16 | 17-98 | 132 |
| chemotactic protein | inducible cytokine | | | | |
| 4) | A13; CK-beta-10; | | | | |
| | NCC-1; SCYA13 | | | | |
| MIP-4 | CCL18; Small | P55774 | 1-20 | 21-89 | 133 |
| (Macrophage | inducible cytokine | | | | |
| inflammatory | A18; Pulmonary and | | | | |
| protein 4) | activation-regulated | | | | |
| | chemokine (PARC); | | | | |
| | Alternative | | | | |
| | macrophage | | | | |
| | activation-associated | | | | |
| | CC chemokine 1 | | | | |
| | (AMAC-1); Dendritic | | | | |
| | cell chemokine 1 | | | | |
| | (DC-CK1) | | | | |
| MIP-3β | CCL19; Small | Q99731 | 1-21 | 22-98 | 134 |
| (Macrophage | inducible cytokine | | | | |
| inflammatory | A19; EBI1-ligand | | | | |
| protein 3-beta) | chemokine (ELC); | | | | |
| | Beta chemokine | | | | |
| | exodus-3; CK β-11; | | | | |
| | SCYA19 | | | | |
| MIP-2α | CXCL2; Growth- | P19875 | 1-34 | 35-107 | 135 |
| (Macrophage | regulated protein beta | | | | |
| inflammatory | (GRO-β); GRO2; | | | | |
| protein 2-alpha) | GROB; SCYB2 | | | | |
| MIP-2β | CXCL3; Growth- | P19876 | 1-34 | 35-107 | 136 |
| (Macrophage | regulated protein | | | | |
| inflammatory | gamma (GRO-γ); | | | | |
| protein 2-beta) | GRO3; GROG; | | | | |
| | SCYB3 | | | | |
| MIP-5 | CCL15; Small | Q16663 | 1-21 | 22-113 | 137 |
| (Macrophage | inducible cytokine | | | | |
| inflammatory | A15; Chemokine CC- | | | | |
| protein 5) | 2; HCC-2; NCC-3; | | | | |
| | MIP-1δ; Leukotactin; | | | | |
| | LKN-1; Mrp-2b; | | | | |
| | SCYA15 | | | | |
| HCC-1 | CCL14; Small | Q16627 | 1-19 | 20-93 | 138 |
| (Hemofiltrate CC | inducible cytokine | | | | |
| chemokine 1) | A14; Chemokine CC- | | | | |
| | 1/CC-3; HCC-1/HCC- | | | | |
| | 3; NCC-2; SCYA14 | | | | |
| RANTES | CCL5; Small | P13501 | 1-23 | 24-91 | 139 |
| (Regulated upon | inducible cytokine | | | | |
| activation, normal | A5; SIS-δ; T cell- | | | | |
| T-cell expressed | specific protein P228 | | | | |
| and secreted) | (TCP228); SCYA5 | | | | |
| Eotaxin-2 | CCL24; Small | O00175 | 1-26 | 27-119 | 140 |
| (Eosinophil | inducible cytokine | | | | |
| chemotactic protein | A24; Myeloid | | | | |
| 2) | progenitor inhibitory | | | | |
| | factor 2 (MPIF-2); | | | | |
| | CK-β-6; SCYA24) | | | | |
| TARC (Thymus | CCL17; Small | Q92583 | 1-23 | 24-94 | 141 |
| and activation- | inducible cytokine | | | | |
| regulated | A17; SCYA17 | | | | |
| chemokine) | | | | | |
| T lymphocyte- | CCL1; Small | P22362 | 1-23 | 24-96 | 142 |
| secreted protein I- | inducible cytokine | | | | |
| 309 | A1; SCYA1 | | | | |
| Lymphotactin | XCL1; Small | P47992 | 1-21 | 22-114 | 143 |
| | inducible cytokine | | | | |
| | C1; Cytokine SCM-1; | | | | |
| | ATAC; Lymphotaxin; | | | | |
| | SCM-1-alpha; XC | | | | |
| | chemokine ligand 1; | | | | |
| | SCYC1 | | | | |
| Lungkine | CXCL15; Small | Q9WVL17 | 1-25 | 26-167 | 144 |
| | inducible cytokine | | | | |
| | B15; SCYB15 | | | | |
| C10 | CCL6; Small | P27784 | 1-21 | 22-116 | 145 |
| | inducible cytokine | | | | |
| | A6; SCYA6 | | | | |
| MIP-1γ | CCL9; CCL10; Small | P51670 | 1-21 | 22-122 | 146 |
| (Macrophage | inducible cytokine | | | | |
| inflammatory | A9; Macrophage | | | | |
| protein 1-gamma) | inflammatory protein- | | | | |
| | related protein 2 | | | | |
| | (MRP-2); CCF18; | | | | |
| | SCYA9; SCYA10 | | | | |
| MCP-5 (Monocyte | CCL12; Small | Q62401 | 1-22 | 23-104 | 147 |
| chemotactic protein | inducible cytokine | | | | |
| 5) | A12; MCP-1-related | | | | |
| | chemokine; SCYA12 | | | | |
| LEC (Liver- | CCL16; Small | O15467 | 1-23 | 24-120 | 148 |
| expressed | inducible cytokine | | | | |
| chemokine) | A16; IL-10-inducible | | | | |
| | chemokine; | | | | |
| | Monotactin-1 (MTN- | | | | |
| | 1); HCC-4; NCC-4; | | | | |
| | Lymphocyte and | | | | |
| | monocyte | | | | |
| | chemoattractant | | | | |
| | (LMC); LCC-1; | | | | |
| | ILINCK; SCYA16 | | | | |
| Exodus-2 | CCL21; Small | O00585 | 1-23 | 24-134 | 149 |
| | inducible chemokine | | | | |
| | A21; 6Ckine; | | | | |
| | Secondary lymphoid- | | | | |
| | tissue chemokine | | | | |
| | (SLC); SCYA21 | | | | |
| MIP-3 | CCL23; Small | P55773 | 1-21 | 22-120 | 150 |
| (Macrophage | inducible cytokine | | | | |
| inflammatory | A23; Myeloid | | | | |
| protein 3) | progenitor inhibitory | | | | |
| | factor 1 (MPIF1); | | | | |
| | CK-beta-8; CKB-8; | | | | |
| | SCYA23 | | | | |
| TECK (Thymus | CCL25; Small | O15444 | 1-23 | 24-150 | 151 |
| expressed | inducible cytokine | | | | |
| chemokine) | A25; SCYA25 | | | | |
| Eotaxin-3 | CCL26; Small | Q9Y258 | 1-23 | 24-94 | 152 |
| | inducible cytokine | | | | |
| | A26; Macrophage | | | | |
| | inflammatory protein | | | | |
| | 4-alpha (MIP-4- | | | | |
| | alpha); Thymic | | | | |
| | stroma chemokine-1 | | | | |
| | (TSC-1); IMAC; | | | | |
| | SCYA26 | | | | |
| CTACK | CCL27; Small | Q9Y4X3 | 1-24 | 25-112 | 153 |
| (Cutaneous T-cell- | inducible cytokine | | | | |
| attracting | A27; ILC; IL-11 R- | | | | |
| chemokine) | alpha-locus | | | | |
| | chemokine; Skinkine; | | | | |
| | ESkine; SCYA27 | | | | |
| MEC (Mucosae- | CCL28; Small | Q9NRJ3 | 1-19 | 20-127 | 154 |
| associated epithelial | inducible cytokine | | | | |
| chemokine) | A28; CCK1; SCYA28 | | | | |
| SCM-1β (Single C | XCL2; XC chemokine | Q9UBD3 | 1-21 | 22-114 | 155 |
| motif-1 beta) | ligand 2; SCM-1b; | | | | |
| | SCYC2 | | | | |
| I-TAC (Interferon- | CXCL11; Small | O14625 | 1-21 | 22-94 | 156 |
| inducible T-cell | inducible cytokine | | | | |
| alpha | B11; Interferon- | | | | |
| chemoattractant) | gamma-inducible | | | | |
| | protein 9 (IP-9); | | | | |
| | H174; β-R1; | | | | |
| | SCYB9B; SCYB11 | | | | |
| BRAK (Breast and | CXCL14; Small | O95715 | 1-22 | 23-99 | 157 |
| kidney-expressed | inducible cytokine | | | | |
| chemokine) | B14; Bolekine; | | | | |
| | NJAC; SCYB14 | | | | |
| SR-PSOX | CXCL16; Small | Q9H2A7 | 1-29 | 30-254 | 158 |
| (Scavenger receptor | inducible cytokine | | | | |
| for | B16; SCYB16 | | | | |
| phosphatidylserine | | | | | |
| and oxidized low | | | | | |
| density lipoprotein) | | | | | |
| Fractalkine | CX3CL1; Small | O35188 | 1-24 | 25-395 | 159 |
| | inducible cytokine | | | | |
| | D1; Neurotactin; | | | | |
| | CX3C membrane- | | | | |
| | anchored chemokine; | | | | |
| | FKN; SCYD1 | | | | |
| LD78-β | CCL3L1; Small | P16619 | 1-23 | 24-93 | 160 |
| | inducible cytokine | | | | |
| | A3-like 1; Tonsillar | | | | |
| | lymphocyte LD78 | | | | |
| | beta protein; G0/G1 | | | | |
| | switch regulatory | | | | |
| | protein 19-2 (G0S19- | | | | |
| | 2 protein); PAT | | | | |
| | 464.2; SCYA3L1 | | | | |
| MIP-1b2 | CCL4L1; CC | Q8NHW4 | 1-23 | 24-92 | 161 |
| (Macrophage | chemokine ligand 4L1 | | | | |
| inflammatory | | | | | |
| protein-1b2) | | | | | |

ii. Chemokine Receptors

Chemokines mediate their activities via G-protein-coupled, seven transmembrane, rhodopsin-like cell surface receptors. Typically, the CXC chemokines bind to one or more of seven CXC-receptors (CXCR1, 2, 3A, 3B, 4, 5, 6), while the CC chemokines bind to one or more of eleven CC-receptors (CCR1, 2A, 2B, 3-10). Other chemokine receptors include XCR1, CX3CR, D6, CKX-CKR and Duffy (also known as Duffy antigen receptor for chemokines, or DARC). DARC, D6 and CKX-CKR are scavenger chemokine receptors which can bind chemokines ligands from all four groups (Hansell et al., Biochem Soc Trans., 34: 1009-13, 2006; Locati et al., Cytokine Growth Factor Rev., 16: 679-86, 2005). Exemplary chemokine receptors include, but are not limited to, Duffy antigen receptor for chemokines (DARC), CXCR-1, CXCR-2, CXCR-3A, CXCR3B, CXCR-4, CXCR-5, CXCR-6, CXCR-7, CCR-1, CCR-2A, CCR-2B, CCR-3, CCR-4, CCR-5, CCR-6, CCR-7, CCR-8, CCR-9, CCR10, CX3CR-1, XCR1, D6 and other chemokine receptors.

The receptor binding of chemokines to their target cells is a complex and an ever-evolving area of investigation. Generally, the receptors bind to the various ligands in an overlapping and complex manner. Inflammatory cells typically express several chemokine receptors, and more than one chemokine can bind to one receptor. For example, the beta chemokine receptor CCR3 binds to not only MCP-3, MCP-4 and RANTES, but also to three other CC chemokines, Eotaxin, Eotaxin-2 and Eotaxin-3 (He et al., Nature, 385: 645-49, 1997; Jose et al., J. Exp. Med., 179: 881-7, 1994; Jose et al., Biochem. Biophys. Res. Commun., 205: 788-94, 1994; Ponath et al., J. Clin. Invest., 97: 604-12, 1996; Daugherty et al., J. Exp. Med. 183: 2349-54, 1996; and Forssman et al., J. Exp. Med., 185: 2171-6, 1997). Eotaxin, Eotaxin-2 and -3 are CCR3-specific (Ponath et al., J. Clin. Invest., 97: 604-12, 1996; Daugherty et al., J. Exp. Med. 183: 2349-54, 1996; and Forssman et al., J. Exp. Med., 185: 2171-6, 1997; Kitaura et al., J Biol. Chem., 274: 27975-80, 1999). A second example is the alpha-chemokine CXCR4 (fusin) HIV co-receptor. Several isoforms of the chemokine stromal cell-derived factor (SDF) including SDF-1α, SDF-1β, and SDF-2 have been identified that specifically bind to this receptor, which is present on various subsets of inflammatory cells and are highly potent MNP cell attractants (Ueda et al., J. Biol. Chem., 272: 24966-70, 1997; Yi et al., J. Virol., 72: 772-7, 1998; Shirozu et al., Genomics, 28: 495-500. 1995; Shirozu et al., Genomics, 37: 273-80, 1996; Bleul et al., J. Exp. Med., 184: 1101-9, 1996; Tanabe et al., J. Immunol. 159: 905-11, 1997; and Hamada et al., Gene, 176: 211-4, 1996; Yu et al., Gene 374: 174-9, 2006).

In some examples, binding of chemokines to specific receptors is affected by the presence or absence of particular amino acid motifs, such as, for example a tripeptide ELR motif (Glu-Leu-Arg). CXC-receptor binding is affected by such a motif. ELR positive chemokines generally bind to the CXCR2 receptor, are angiogenic and preferentially target neutrophils. In contrast, ELR negative chemokines bind to CXCR3 and 5, are anti-angiogenic and preferentially target T-lymphocytes, NK cells, immature dendritic cells (IDC) and activated endothelial cells. The ELR negative chemokine SDF-1β (CXCR4) as well as some CC chemokines including MCP-1 (CCR2) also are angiogenic (Strieter et al. (2005) Cytokine Growth Factor Res., 16:593-609; Salcedo et al. (2000) Blood 96: 34-40). Chemokines also bind to cell surface heparin and glycosaminoglycans in a way that is thought to facilitate the maintenance of a gradient needed for leukocyte activation and transportation (extravasation) from the circulation into the inflamed tissue (Schall et al., Current Biol., 6: 865-73, 1994; and Tanaka et al., Immunology Today, 14: 111-15, 1993; Neel et al., Cytokine Growth Factor Rev., 16: 637-58, 2005; Johnson et al., Biochem. Soc. Trans., 32: 366-77, 2004).

Table 5 sets forth chemokine/chemokine agonistic specificities for exemplary chemokines and their receptors. It must be noted that certain chemokines have been shown to bind different chemokine receptors in an antagonistic fashion. The data in Table 5 pertains to humans. There can be species differences between chemokine receptor specificities, and the chemokines can have different affinities for different receptors. Hence, species-specific, as well as receptor-specific, conjugates can be prepared. There also can be allelic differences in receptors among members of a species, and, if necessary, allele-specific conjugates can be prepared. In addition, different species express homologs of human chemokines. For example, TCA-3 is the murine homolog of human I-309 (I. Goya et al. (1998) J. Immunol., 160:1975-81).

TABLE 5
Exemplary Chemokine Ligand-Receptor Specificities

| Chemokine Ligand | Chemokine Receptor(s) |
|---|---|
| MCP-1 (Monocyte chemoattractant protein-1) | CCR2; D6 |
| Eotaxin (Eosinophil chemotactic protein) | CCR3; CCR5 |
| SDF-1β (Stromal cell-derived factor 1) | CCR4, CXCR7 |
| GRO-α (Growth-regulated protein alpha) | CXCR2; CXCR1; Duffy |
| MIP-1β (Macrophage inflammatory protein 1- | CCR5; D6 |
| beta) | |
| IL-8 (Interleukin-8) | CXCR1; CXCR2; Duffy |
| IP-10 (Interferon-inducible protein-10) | CXCR3A; CXCR3B |
| MCP-3 (Monocyte chemotactic protein 3) | CCR1; CCR2; CCR3 |
| MIP-3α (Macrophage inflammatory protein 3 | CCR6 |
| alpha) | |
| MDC (Macrophage-derived chemokine) | CCR4 |
| MIP-1α (Macrophage inflammatory protein 1- | CCR1; CCR5 |
| alpha) | |
| BCA-1 (B cell-attracting chemokine 1) | CXCR5 |
| GCP-2 (Granulocyte chemotactic protein 2) | CXCR1; CXCR2 |
| ENA-78 (Epithelial-derived neutrophil- | CXCR2 |
| activating protein 78) | |
| MIG (Gamma interferon-induced monokine) | CXCR3A; CXCR3B |
| SDF-2 (Stromal cell-derived factor 2) | Unknown |
| MCP-2 (Monocyte chemotactic protein 2) | CCR1; CCR2; CCR3; |
| | CCR5; D6 |
| MCP-4 (Monocyte chemotactic protein 4) | CCR1; CCR2; CCR3; D6 |
| MIP-4 (Macrophage inflammatory protein 4) | Unknown |
| MIP-3β (Macrophage inflammatory protein 3- | CCR7; CCR11 |
| beta) | |
| MIP-2α (Macrophage inflammatory protein 2- | CXCR2 |
| alpha) | |
| MIP-2β (Macrophage inflammatory protein 2- | CXCR2 |
| beta) | |
| MIP-5 (Macrophage inflammatory protein 5) | CCR1; CCR3; D6 |
| HCC-1 (Hemofiltrate CC chemokine 1) | CCR1; CCR5; D6 |
| RANTES (Regulated upon activation, normal | CCR1; CCR3; CCR5; |
| T-cell expressed and secreted) | Duffy; D6 |
| Eotaxin-2 (Eosinophil chemotactic protein 2) | CCR3 |
| TARC (Thymus and activation-regulated | CCR4 |
| chemokine) | |
| T lymphocyte-secreted protein I-309 | CCR8; Duffy |
| Lymphotactin | XCR1 |
| Lungkine | Unknown |
| C10 | CCR1 |
| MIP-1γ (Macrophage inflammatory protein 1- | CCR1 |
| gamma) | |
| MCP-5 (Monocyte chemotactic protein 5) | CCR2; CCR3 |
| LEC (Liver-expressed chemokine) | CCR1; CCR2; CCR5; |
| | CCR8 |
| Exodus-2 | CCR7; CCR11 |
| MIP-3 (Macrophage inflammatory protein 3) | CCR1 |
| TECK (Thymus expressed chemokine) | CCR9; CCR11 |
| Eotaxin-3 | CCR3 |
| CTACK (Cutaneous T-cell-attracting | CCR10 |
| chemokine) | |
| MEC (Mucosae-associated epithelial | CCR10 |
| chemokine) | |
| SCM-1β (Single C motif-1 beta) | XCR1 |
| I-TAC (Interferon-inducible T-cell alpha | CXCR3A; CXCR3B |
| chemoattractant) | |
| BRAK (Breast and kidney-expressed | Unknown |
| chemokine) | |
| SR-PSOX (Scavenger receptor for | CXCR6 |
| phosphatidylserine and oxidized low density | |
| lipoprotein) | |
| Fractalkine | CX3CR1 |
| LD78-β | CCR5; D6 |
| MIP-1b2 (Macrophage inflammatory protein- | CCR5 |
| 1b2) | |

iii. Chemokine/Chemokine Receptor Cellular Profile

Each chemokine receptor has a distinct leukocyte specificity, although the various chemokine receptor-leukocyte specificities can overlap considerably (see e.g. Table 6). For example, distinct receptor subtypes specific for the same chemokine and the same function can be coexpressed on the same cell. Additionally, distinct chemokine ligands acting at separate receptors on the same cell can induce the same cellular response. Further, different chemokine ligands can bind to a common receptor and induce different cellular responses on the target cell. Most chemokines bind to receptors expressed on leukocytes, particularly activated leukocytes, although some chemokine receptors can be expressed on other cell types, such as various tissue residential cells, for example, red blood cells, platelets, astrocytes, endothelial cells, neurons, epithelial cells, adipose cells, and microglial cells of the brain. Table 6 includes a non-exhaustive exemplary list of chemokine receptors and sets forth an exemplary set of leukocyte subtypes and other cell types that are known to express each chemokine receptor under various disease and non-disease circumstances.

TABLE 6
Exemplary Chemokine Receptor-Leukocyte Specificities

| Chemokine Receptor | Leukocyte Subtype(s) |
|---|---|
| CCR1 | NK cell; T cell; IDC; MNP; TAM; Basophil; |
| | Eosinophil; PMN; Platelet |
| CCR2 | NK cell; B cell; T cell; IDC; MNP; Basophil; |
| | PMN |
| CCR3 | T cell; Th2; MDC; Basophil; Eosinophil; Platelet; |
| | MaC |
| CCR4 | Thymocyte; NK cell; T cell; Th2; IDC; MDC; |
| | MNP; Basophil; Platelet |
| CCR5 | Thymocyte; NK cell; B cell; T cell; Th1; IDC; |
| | MDC; MNP; GC; TAM; Adipocyte |
| CCR6 | B cell; T cell; IDC |
| CCR7 | B cell; T cell; MDC |
| CCR8 | Thymocyte; B cell; T cell; Th2; IDC; MDC; MNP |
| CCR9 | Thymocyte; T cell; MDC; MNP |
| CCR10 | T cell |
| CXCR1 | MNP; PMN; MaC; Astrocyte |
| CXCR2 | MNP; Eosinophil; PMN; MaC |
| CXCR3A | NK cell; B cell; T cell; Th1; MaC |
| CXCR3B | NK cell; B cell; T cell |
| CXCR4 | Thymocyte; B cell; T cell; IDC; MDC; MNP; |
| | GC; PMN; Platelet; Adipocyte; Astrocyte |
| CXCR5 | B cell; T cell; Astrocyte |
| CXCR6 | NK cell; T cell |
| XCR1 | NK cell; T cell |
| CX3CR1 | NK cell; T cell; MNP; PMN; Astrocyte |

Key:
NK = natural killer;
Th1 = type 1 helper T cell;
Th2 = type 2 helper T cell;
IDC = immature dendritic cell;
MDC = mature dendritic cell;
MNP = mononuclear phagocytes (monocytes, macrophages and microglia);
GC = giant cell (multinucleated fused macrophage);
TAM = tumor associated macrophage;
PMN = polymononuclear neutrophil;
MaC = mast cell.
Note:
The Table above represents an exemplary, non-exhaustive list of cell types that express particular chemokine receptors.

Each cell type has a chemokine receptor profile that is akin to a fingerprint or “chemoprint” that is dependent on the specific cell type, function type, tissue type, disease state and type of disease, developmental state of the cell type, activation state of cellular receptors, and the extracellular environment, including surrounding cell types and molecules. For example, cells of monocytic lineage tend to be associated with CXCR4 and CCR1-3 and 5 receptors; eosinophils and basophils with CCR1-3 and CXCR3 and 4; PMN with CXCR1, 2 and CCR1; B-cells with CCR1-7 and CXCR3-5; Th1 cells with CXCR3 and CCR5; and finally, Th2 cells with CCR2, 3, 4 and 8 (e.g., Baggiolini, J. Intern. Med., 250: 91-104, 2001).

In general, the binding affinities, specificities, and the differential distribution of receptor subtypes across target cells determine the contribution that a given chemokine will make to the inflammatory process. The biological profile of a given chemokine determined in one setting may not hold true in another, most especially if the ratio and activation status of target cells changes during trauma or disease. Hence the biological profile of a given chemokine, if necessary, can be established on a case by case basis. For example, the effects of monocyte chemotactic protein-3 (MCP-3) are similar to those of MCP-1, but the former binds to a broader range of cells and receptors. In addition to different receptor expression on different cells, receptor numbers expressed on cell surfaces can vary. For example, CCR1 and CCR2 are expressed at the rate of 3,000 receptors per monocyte and lymphocyte, whereas there are about 50,000 CCR3 receptors on eosinophils (Borish and Steinke, J Allergy Clin Immunol., 111: S460-75, 2003). Such differences can have implications on migration direction and response times. For example, the high density of CXCR4 on T cells correlates with faster death induced by HIV, and a higher density of receptors including CCR2 and CCR4 is associated with the recruitment of alveolar T cells in allergic asthma patients (Kallinich et al. (2005) Clin. Exp. Allergy 35, 26-33; Lelievre et al. (2004) AIDS Res. Hum. Retroviruses 20: 1230-43).

Likewise, chemokine receptor profiles often change during trauma or disease. Chemokine ligand/receptor axes are classified as constitutive/homeostatic, inducible/inflammatory or both (see e.g., Table 7). Therefore, the inflammatory chemokine ligands and their receptors are not necessarily expressed until disease or trauma ensues. For example, quiescent cells will quickly change and upregulate receptor expression once activated (e.g., Ghirnikar, et al. (2000) Neurosci. Res. 59:63-73: Henneken et al. (2005) Arthritis Res. Ther. 7: R1001-13; Klitgaard et al. (2004) Acta. Opthalmol. Scand. 82: 179-83; McDonald et al. (2001) IDrugs 4: 427-42). The identity of a chemoprint also depends on the types and abundance of inflammatory and non-inflammatory mediators in the milieu (e.g., Porcheray et al. (2006) Virology 349: 112-20; Stout and Suttles (2004) J Leukoc. Biol. 76: 509-13; Sozzani (2005) Cytokine Growth Factor Res. 16: 581-92; Mantovanni et al., Trends Immunol., 25: 677-86, 2004; Ben-Baruch, Cancer Metastasis Rev., 2006, published ahead of print). Table 7 sets forth exemplary expression profiles of chemokine/receptor axes as a consequence of function under homeostatic or inflammatory conditions.

TABLE 7
Members of the Chemokine Superfamily of Ligands and Receptors

| Systemic | | | Exemplary |
|---|---|---|---|
| Name | Ligand | Receptor(s) | Function |
| CC | | | |
| Chemokines | | | |
| CCL1 | I-309 | CCR8 | Inflammatory |
| CCL2 | MCP-1 | CCR2 | Inflammatory |
| CCL3 | MIP-1 α | CCR1, CCR5 | Inflammatory |
| CCL4 | MIP-1 β | CCR5 | Inflammatory |
| CCL5 | RANTES | CCR1, CCR3, CCR5 | Inflammatory |
| CCL6 | Unknown | Unknown | Unknown |
| CCL7 | MCP-3 | CCR1, CCR2, CCR3 | Inflammatory |
| CCL8 | MCP-2 | CCR3, CCR2 | Inflammatory |
| CCL9 | Unknown | Unknown | Unknown |
| CCL10 | Unknown | Unknown | Unknown |
| CCL11 | Eotaxin | CCR3 | Inflammatory |
| CCL12 | Unknown | CCR2, CCR3 | Unknown |
| CCL13 | MCP-4 | CCR2, CCR3 | Inflammatory |
| CCL14 | HCC-1 | CCR1 | Unknown |
| CCL15 | HCC-2 | CCR1, CCR3 | Unknown |
| CCL16 | HCC-4, LEC | CCR1, CCR2, CCR5 | Unknown |
| CCL17 | TARC | CCR4 | Inflamm/ |
| | | | Homeostatic |
| CCL18 | DC-CK1 | Unknown | Homeostatic |
| CCL19 | MIP-3 β | CCR7 | Homeostatic |
| CCL20 | MIP-3 α | CCR6 | Inflamm/ |
| | | | Homeostatic |
| CCL21 | SCL | CCR7 | Homeostatic |
| CCL22 | MDC | CCR4 | Inflamm/ |
| | | | Homeostatic |
| CCL23 | MPIF-1 | CCR1 | Unknown |
| CCL24 | MPIF-2 | CCR3 | Inflammatory |
| CCL25 | TECK | CCR9 | Homeostatic |
| CCL26 | Eotaxin-3 | CCR3 | Inflammatory |
| CCL27 | CTACK | CCR10 | Homeostatic |
| CCL28 | MEC | CCR10 | Inflamm/ |
| | | | Homeostatic |
| C | | | |
| Chemokines | | | |
| XCL1 | Lymphotactin | XCR1 | Unknown |
| XCL2 | SCM1-α | XCR1 | Unknown |
| CXC | | | |
| Chemokines | | | |
| CXCL1 | GROα | CXCR2 | Inflammatory |
| CXCL2 | GROβ | CXCR2 | Inflammatory |
| CXCL3 | GROγ | CXCR2 | Inflammatory |
| CXCL4 | PF4 | CXCR3A | Unknown |
| CXCL5 | ENA-78 | CXCR2 | Unknown |
| CXCL6 | GCP-2 | CXCR1, CXCR2 | Unknown |
| CXCL7 | NAP-2 | CXCR2 | Unknown |
| CXCL8 | IL-8 | CXCR1, CXCR2 | Inflammatory |
| CXCL9 | MIG | CXCR3B | Inflammatory |
| CXCL10 | IP-10 | CXCR3B | Inflammatory |
| CXCL11 | I-TAC | CXCR3B | Inflammatory |
| CXCL12 | SDF-1α, SDF-1β | CXCR4,CXCR7 | Unknown |
| CXCL13 | BCA-1 | CXCR5 | Homeostatic |
| CXCL14 | BRAK | Unknown | Homeostatic |
| CXCL15 | Unknown | Unknown | Unknown |
| CXCL16 | Unknown | CXCR6 | Inflammatory |
| CX3CL1 | Fractalkine | CX3CR1 | Inflammatory |

It is understood that Table 7 is exemplary only, and the expression of different chemokine/receptor pairs is dependent on a number of factors, such as, but not limited to, the stage or severity of a disease. For example, certain leukocyte subtypes may not be present until a clinical condition has reached a particular stage. Also, receptor expression can change. For example, chemokine receptors prior to spinal cord contusion injury in rats are not detected. The expression of CCR2, CCR3, CCR5, CCR10 and CXCR4 were differentially upregulated in a time dependent manner from one day post injury to 14 days post injury (Ghirnikar, et al. (2000) Neurosci. Res., 59:63-73).

Certain ligand/receptor axes play prominent roles in specific diseases. For example, the MCP-1/CCR2 axis is important in a wide range of diseases which include, but are not limited to, arthritis, asthma, atherosclerosis, restenosis, multiple sclerosis, spinal cord injury (SCI), cancers, and several classes of chronic kidney disease (CKD). In another example, SDF-1β/CXCR4 is relevant in arthritis and a number of cancers including ovarian, prostate, breast and brain cancers. In another example, the MIG, IP-10, I-TAC/CXCR3A axes are relevant in organ transplant rejection, type-1 diabetes, proliferative glomerulonephritis (GN) and multiple sclerosis. In another example, Eotaxin, Eotaxin-2 and Eotaxin-3/CCR3 are important axes in asthma, eosinophilic pneumonia, esophagitis and inflammatory skin diseases.

In most cases, multiple chemokine ligands and chemokine receptors are expressed in particular disease states (e.g., Mantovani (1999) Immunol. Today 20: 254-7; Borish and Steinke (2003) J. Allergy Clin. Immunol., 111: S460-75; Charo and Ransohoff, N Engl J Med., 354: 610-21, 2006). For example, in an experimental autoimmune encephalomyelitis (EAE) model of multiple sclerosis (MS) T cells can express CCR5, CCR2, and CXCR3 (Matsui et al. (2002) J. Neuroimmunol., 128: 16-22). In another example, in in vitro blood-brain-barrier (BBB) transmigration studies, MCP-1/CCR2 axes is important for CNS extravasation of CCR2 expressing MNP and T cells, though T cells can express CCR2, CCR5 and CXCR3 (Mahad et al. (2006) Brain 129: 212-23; Callahan et al. (2004) J. Neuroimmunol., 153: 150-7). Thus, when studying specific diseases or traumas the spatial, temporal, biological and clinical profiles of any given ligand/receptor axis or axes can be established in choosing the targeting agent or agents for the toxin conjugate.

Adding to this complexity, in pathological conditions immune cells and contributing tissue resident cells (TRC) can undergo profound changes in phenotype and can express chemokine receptors that are not normally associated with the specific cell type. For example, despite a CXC chemokine preference for PMN, profound PMN chemoattraction by the CC chemokines MCP-1 and MIP-1α occurred in a rat model of vasculitis sepsis and a murine model of sepsis (Johnston et al. (1999) J. Clin. Invest. 103:1269-76; Speyer et al. (2004) Am. J. Pathol. 165: 2187-96). Receptor changes also occur in disease on MNP, T lymphocytes and MaC. They can be induced to express CXCR1 and CXCR2 in specific inflammatory microenvironments (Smith et al. (2005) Am. J. Physiol. Heart Circ. Physiol. 289: H1976-84; Lippert et al. (2004) Exp. Dermatol. 13: 520-5). Eosinophils often express functional CCR2 the cognate receptor for MCP-1 (Dunzendorfer (2001) J. Allergy Clin. Immunol. 108: 581-7).

iv. Exemplary Chemokine Targeting Agents

Chemokine ligands used in the ligand-toxin conjugates provided herein typically are any chemokine with specificity to at least one chemokine receptor, but typically more than one chemokine receptor, expressed on one or more immune effector cell, including leukocytes or other contributing effector cells, involved in immunomodulatory or inflammatory processes such as pathological inflammation that promote secondary tissue damage. Such receptors are generally members of the superfamily of G-protein coupled, seven transmembrane-domain, rhodopsin-like receptors, including but are not limited to, for example, one or more of the receptors known in the art as the Duffy antigen receptor for chemokines (DARC), D6, CXCR-1, CXCR-2, CXCR-3A, CXCR3B, CXCR-4, CXCR-5, CXCR-6, CXCR-7, CCR-1, CCR-2A, CCR-2B, CCR-3, CCR-4, CCR-5, CCR-6, CCR-7, CCR-8, CCR-9, CCR10, CX3CR-1, XCR1 and other chemokine receptors. In some examples, the chemokine selected for use as a targeting agent in a conjugate provided herein can bind to a specific receptor, whereas in other examples, the chemokine selected can bind to more than one receptor. In addition, a selected chemokine for use as a targeting agent in a conjugate can exhibit overlapping and differential receptor specificities with other chemokines (see e.g., Table 5).

Included among such chemokine ligands are any set forth in Table 4 above, including any of the alpha and beta chemokines, and other similar sub-groups of chemokines. More particularly, chemokines presently preferred for use as the proteinaceous ligand moiety in the ligand-toxin conjugates include, but are not limited to, the alpha-chemokines known in the art as IL-8; granulocyte chemotactic protein-2 (GCP-2); growth-related oncogene-α (GRO-α) GRO-β, and GRO-γ; epithelial cell-derived neutrophil activating peptide-78 (ENA-78); connective tissue activating peptide III (CTAP III; neutrophil activating peptide-2 (NAP-2); monokine induced by interferon-γ (MIG); interferon inducible protein 10 (IP-10, which possesses potent chemoattractant actions primarily but not exclusively for neutrophils and T cells); the stromal cell derived factors SDF-1α, SDF-1β, and SDF-2; the beta-chemokines known in the art as the monocyte chemotactic proteins MCP-1, MCP-2, MCP-3, MCP-4, and MCP-5; the macrophage inflammatory proteins MIP-1α, MIP-1β, MIP-1γ, MIP-2, MIP-2α, MIP-2β, MIP-3α, MIP-3β, MIP-4, and MIP-5; macrophage-derived chemokine (MDC); human chemokine 1 (HCC-1); RANTES; Eotaxin 1; Eotaxin 2; Eotaxin-3; TARC; SCYA17 and I-309; dendritic cell chemokine-1 (DC-CK-1); the γ-chemokine, lymphotactin; the soluble form of the CX3C chemokine fractalkine (which are chemoattractant primarily but not exclusively for monocytes, macrophages, eosinophils and T cells); any others known to those of skill in the art; and any synthetic or modified proteins designed to bind to the chemokine receptors. Chemokines can be isolated from natural sources using routine methods, or expressed using nucleic acid encoding the chemokine. Biologically active chemokines have been recombinantly expressed in E. coli (e.g., those commercially available from R&D Systems, Minneapolis, Minn.).

Examples of other chemokine targeting agents include any that bind to and/or activate one or more immune cells such as any secondary tissue damage-promoting cells, such as for example, the acylated LDL scavenger receptors 1 and 2, and the receptors for LDL, very low density lipoprotein-1 (VLDL-1), VLDL-2, glycoprotein 330/megalin, lipoprotein receptor-related protein (LRP), alpha-2-macroglobulin, sorLA-1. A particularly useful receptor associated protein, as yet unnamed, has a molecular weight of about 39,000 daltons and binds to and modulates the activity of proteins, such as members of the low density lipoprotein (LDL)-receptor family.

It is understood that other chemokines are known and that such chemokines and receptors specific therefor can be identified, and where necessary produced and used to produce conjugates as described herein. As described in detail below, the diseases for which the resulting conjugates can be used can be determined by the specificity and cell populations upon which receptors therefor are expressed, and also can be determined empirically using in vitro and in vivo models known to those of skill in the art, including those exemplified, described and/or referenced herein.

b. Non-Chemokine Cytokines

Conjugates that include classic cytokines that are non-chemokine cytokines that bind to specific cytokine receptors on cell types involved in secondary tissue damage, including any that also express chemokine receptors, also can be used in the conjugate provided herein and in the methods of generating the conjugates provided herein. Conjugates that include such classic cytokines have been used for therapies, such as cancers treatments by targeting the tumor cells. It is intended herein, that cytokines are selected for their ability to bind to chemokine-receptor bearing cells, such as leukocytes that infiltrate tumors, and other cells associated with undesirable inflammatory responses.

Although chemokines are ostensibly classified as cytokines, they are a distinct class of proteins. Their classification as cytokines is more historical than actual. When new proteins are discovered they are named for example, after their apparent activity or their cellular source. Thus the early cytokines were thought to be hormones or were called growth factors. Because cytokines share many properties with hormones and growth factors, the distinction has been and still is a grey area. For example, in a review article (see, e.g., Wells et al. (1996) Ann Rev Biochem 65:609-34) the phrase “hematopoietic hormones/cytokines” is used (a reference to the similarity of biological activities with the various colony-stimulating factors) to describe cytokines. Some cytokine activities originally were isolated from lymphocytes and monocytic cells and were termed lymphokines and monokines, respectively. When it was realized that these molecules represent a broad spectrum of activities and were derived from numerous cell types the term “cytokine” was coined.

Classic cytokines (12-40 kDa proteins) include interferons (IFNs), tumor necrosis factors (TNFs) and interleukins (so-called because their activity includes communication between leukocytes), hematopoietic growth factors, growth hormone, ciliary neurotrophic factor and others. These cytokines regulate the proliferation and differentiation of many different cell types via structurally homologous class I cytokine receptors. The Class I receptors are typically composed of two polypeptide chains, an “ligand-specific subunit and a β signal transducing subunit. This class of receptors can be subdivided on the basis of an identical a subunit and the utility of a third subunit. The interferons act via a structurally distinct set of (α, β, and γ) Class II receptors. There is now an emerging family of distinct TNF receptors.

Cytokine receptors usually signal via the JAK/STAT intracellular signal pathway, but also can signal through other signaling cascades. Significantly, none of the cytokines, such as the interleukins, that bind to these receptors bind to any of the structurally distinct chemokine receptors (described above) and no chemokine ligand binds to any of the above described cytokine receptors.

Hence, reference to non-chemokine cytokines is meant to encompass classic cytokines. The non-chemokine cytokines that are useful as ligand moieties for targeting conjugates to receptors on cells, for example, cells that also bear chemokine receptors, include, but are not limited to, endothelial monocyte activating polypeptide II (EMAP-II), colony stimulating factor (CSF), granulocyte-macrophage-CSF (GM-CSF), granulocyte-CSF (G-CSF), macrophage-CSF (M-CSF), interleukin 1 (IL-1), IL-1a, IL-1b, interleukin 2 (IL-2), interleukin-3 (IL-3), interleukin 4 (IL-4), interleukin 5 (IL-5), interleukin 6 (IL-6), interleukin 7 (IL-7), interleukin 8 (IL-8), interleukin 10 (IL-10), interleukin 12 (IL-12), interleukin-13 (IL-13), interleukin 15 (IL-15), interleukin 18 (IL-18), interferon alpha (IFNα), interferon beta (IFNβ), interferon gamma (IFNγ), interferon omega (IFNω), interferon tau (IFNτ), interferon gamma inducing factor I (IGIF), Flt-3 ligand, erythropoietin (EPO), tumor necrosis factor (TNF), a proliferation-inducing ligand (APRIL), CD40 ligand, CD30 ligand, CD27 ligand, fas ligand, 4-1BB ligand, LIGHT, HVEM, TWEAK, GITRL, TNF-related apoptosis-inducing ligand (TRAIL), TNF-related activation-induced cytokine (TRANCE), TNF and apoptosis ligand-related leukocyte-expressed ligand 1 (TALL-1), which bind to families of cytokine receptors on cells involved in an inflammatory response, such as on secondary tissue damage-promoting cells.

Exemplary of cytokine receptors for targeting by any non-chemokine cytokine provided herein include, but are not limited to hematopoietin family receptors (e.g., receptors for IL-2 through IL-7 and GM-CSF), interferon family receptors (e.g., receptors for IFNα, IFNβ and IFNγ), and Tumor Necrosis Factor family receptors (e.g., receptors for TNFα, lymphotoxin, Fas ligand, LIGHT, BTLA, CD40 ligand, 4-1BB ligand, OX-40 ligand and others including, but not limited to any of TNF receptor (TNFR) such as, but not limited to, TNFR1, TNFR2, LtβR, Fas, CD40, CD27, D30, 4-1BB, OX40, DR3, DR5, and HVEM).

c. Antibody Ligand Moieties

The targeting agent in the ligand-toxin conjugate also can be an antibody, particularly a monoclonal antibody, or a functional fragment of thereof, that is specific for a receptor expressed on the surface of cells involved in the inflammatory response, particularly a chemokine receptor, cytokine receptor and other receptors expressed on cells that express chemokine receptors. It is preferred that the monoclonal antibody be specific for a chemokine receptor, for example, CCR-1, CCR-2A, CCR-2B, CCR-3, CCR-4, CCR-5, CCR-6, CCR-7, CCR-8, CCR-9, CCR-10, CXCR-1, CXCR-2, CXCR-3A, CXCR3B, CXCR-4, CXCR-5, CXCR-6, DARC, XCR1, CX3CR-1, and other such receptors.

In some instances, the antibody can be specific for a non-chemokine cytokine receptor, such as, for example, a receptor for any one or more of cytokines EMAPII, GM-CSF, G-CSF, M-CSF, IL-1, IL-2, IL-3, IL-4, IL-5, IL-6, IL-12, IL-13. Conjugates containing these antibodies can be used for targeting to cells that express the targeted cytokine receptors. Such cells include cells involved in secondary tissue damage. The targeted cells also can express one or more chemokine receptors.

Non-limiting examples of monoclonal antibodies that can be used in the conjugates include, but are not limited to, MAC-1, MAC-3, ED-1, ED-2, ED-3, and monoclonal antibodies against the following antigens CD5, 14, 15, 19, 22, 34, 35, 54 and 68; OX4, 6, 7, 19 and 42; Ber-H2, BR96, Fib75, EMB-11, HLA-DR, LN-1, and Ricinus communis agglutinin-1.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see, e.g., U.S. Pat. Nos. 4,036,945 and 4,331,647, and references contained therein, which reference also are hereby incorporated in their entireties by reference; see, also Porter, R. R., (1959) Biochem. J., 73: 119-126). Other methods of cleaving antibodies, such as separation of heavy chains to form monovalent light-heavy chain fragments, further cleavage of fragments, or other enzymatic, chemical, or genetic techniques also can be used, as long as the fragments bind to the antigen that is recognized by the intact antibody.

Fv fragments contain an association of VH and VL chains. This association can be noncovalent, as described in Inbar et al. (1972) Proc. Nat'l Acad. Sci. U.S.A. 69:2659-62. Alternatively, the variable chains can be linked by an intermolecular disulfide bond or cross-linked by chemicals such as glutaraldehyde. Typically, the Fv fragments contain VH and VL chains connected by a peptide linker. These single-chain antigen binding proteins (sFv) are prepared by constructing a nucleic acid molecule encoding the VH and VL domains connected by an oligonucleotide. The resulting construct is inserted into an expression vector, which is introduced into a host cell, such as E. coli. The recombinant host cells synthesize a single polypeptide chain with a linker peptide bridging the two V domains. Methods for producing sFvs are described, for example, by Whitlow and Filpula (1991) Methods, 2: 97-105; Bird et al. (1988) Science 242:423-426; Pack et al. (1993) Bio/Technology 11:1271-77; and Ladner et al., U.S. Pat. No. 4,946,778).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, using the polymerase chain reaction to synthesize the variable region from RNA of antibody-producing cells (see, e.g., Larrick et al. (1991) Methods, 2: 106-10; and Orlandi et al. (1989) Proc. Natl. Acad. Sci. U.S.A. 86:3833-3837).

Antibodies that bind to a chemokine receptor or non-chemokine cytokine receptor on a secondary tissue damage-promoting cell can be prepared using an intact polypeptide or biologically functional fragment containing small peptides of interest as the immunizing antigen. The polypeptide or a peptide used to immunize an animal (derived, for example, from translated cDNA or chemical synthesis) can be conjugated to a carrier protein, if desired. Commonly used carriers that are chemically coupled to the peptide include, but are not limited to, keyhole limpet hemocyanin (KLH), thyroglobulin, bovine serum albumin (BSA), and tetanus toxoid. The coupled peptide is then used to immunize the animal (e.g., a mouse, a rat, or a rabbit).

The preparation of monoclonal antibodies is well known in the art (see e.g., Kohler et al. (1975) Nature 256:495-7; and Harlow et al., in: Antibodies: a Laboratory Manual, (Cold Spring Harbor Pub., 1988). Briefly, monoclonal antibodies can be obtained by injecting mice with a composition containing an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography and are well known to those of skill in the art (see e.g., Pharmacia Monoclonal Antibody Purification Handbook (e.g., Cat. # 18-1037-46)).

Antibodies also can be derived from subhuman primate antibodies. Such method for raising therapeutically useful antibodies in baboons are known to those of skill in the art (see, e.g., Goldenberg et al. (1991) Published International PCT application No. WO 91/11465 and Losman et al. (1990) Int. J. Cancer, 46:310-314). Therapeutically useful antibodies can be derived from a “humanized” monoclonal antibody. Such methods and antibodies are known. For example, humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al. (1989) Proc. Nat'l Acad. Sci. USA 86:3833-7, which is hereby incorporated in its entirety by reference. Techniques for producing humanized monoclonal antibodies are described, for example, by Jones et al. (1986) Nature 321:522-5; Riechmann et al. (1988) Nature 332:323-7; Verhoeyen et al. (1988) Science 239:1534-6; Carter et al. (1992) Proc. Nat'l Acad. Sci. USA 89:4285-9; Sandhu (1992) Crit. Rev. Biotech. 12:437-62; and Singer et al. (1993) J. Immunol. 150:2844-67).

Anti-idiotype technology can be used to produce monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

d. Other Targeting Agents and Receptor Targets

Conjugates provided herein can contain any targeting agent that targets the conjugate to a cell surface receptor. In addition to the targeting agents mentioned above including chemokines, cytokines, and antibodies, such targeting agents also include, for example, but are not limited to, growth factors, hormones, and other ligands or allelic variants, muteins, or fragments thereof, so long as the targeting agent is internalized by a cell surface receptor to which it binds. Such targeting agents can be used to generate a ligand-toxin conjugate using the methods provided herein. Further, such targeting agents can be used to construct a ligand-toxin conjugate containing a targeting agent linked directly or indirectly to modified toxins or toxin variants, including the modified SA1 variants provided herein.

Exemplary targeting agents include, but are not limited to, transforming growth factor beta (TGF-β), Leishmania elongation initiating factor (LEIF), platelet derived growth factor (PDGF), epidermal growth factor (EGF), amphiregulin, neuregulin-1, neuregulin-2, neuregulin-3 or neuregulin-4, growth factors including vascular endothelial growth factor (VEGF), fibroblast growth factor, (FGF), hepatocyte growth factor (HGF), nerve growth factor (NGF), placental growth factor (P1GF), brain derived neurotrophic factor (BDNF), betacellulin (BTC), midkine, inhibin, endothelial growth factor, insulin, insulin-like growth factor (IGF) neurotrophin-2 (NT-2), neurotrophin-3 (NT-3), neurotrophin-4 (NT-4), neurotrophin-5 (NT-5), glial cell line-derived neurotrophic factor (GDNF), ciliary neurotrophic factor (CNTF), pleiotrophin, stem cell factor (SCF), oncostatin M, sensory and motor neuron-derived factor (SMDF), leukemia inhibitory factor (LIF), Müllerian-inhibiting substance (MIS), cardiotrophin-1, thrombopoietin, angiopoietin, activin, bone morphogenic protein (BMP), APM1, growth hormone (GH), leptin, and prolactin or allelic variants, muteins, or fragments thereof. Also included as a targeting agent are pathogen recognition receptors (PRRs) that participate in the phagocytosis or endocytosis of pathogens. For example, other exemplary targeted agents include molecules that target to the mannose receptor (MR), Dectin-1, and receptors for collectins or collectin-like proteins including, but not limited to, receptors for surfactant protein A (SP-A), surfactant protein D, mannose binding lectin (MBL), or complement protein 1q (C1q). Preferred targeting agents are polypeptides that when bound to receptors are internalized into the cell.

The ligand-toxin conjugates generated using such targeting agents can be used to treat any disease or disorder having a cellular component involved in its pathology. Preferred among such diseases or disorders are any having a pathological cellular component, which cellular component expresses one or more cell surface receptors that can be targeted. Other diseases and disorders also are contemplated particularly angiogenic diseases including, but not limited to cancer, and retinopathies such as ocular or diabetic retinopathies, such as via targeting of endothelial cells involved in angiogenesis.

Growth Factors

Growth factors that bind to endocytic cell surface receptors on cell types involved in inflammation and/or secondary tissue damage, also can be used as targeting agents for the conjugate provided herein and can be used in the methods provided herein. Such growth factors also can be used as targeting agents to target cells involved in angiogenic diseases, including cancers and other diseases such as eye diseases or various chronic inflammatory states, via targeting a ligand-toxin conjugate to endothelial cells. Growth factors such as, for example vascular endothelial growth factor (VEGF), or any modified version thereof including those having amino acid substitutions, deletions, insertions or additions, can be used to target toxin moieties to specific cell types, so long as they retain the ability to bind to receptors and be internalized (see, e.g., U.S. Patent Application Nos. 2004/0166565 and US20010031485). Hence, such growth factors can be used to construct a ligand-toxin conjugate containing a growth factor linked directly or indirectly to modified toxins or toxin variants, including the modified Shiga toxin A1 variants provided herein. Targeted cell types can include, for example, endothelial cells involved in angiogenesis, which is a process of growing new blood vessels often associated with tumor growth and chronic inflammatory states.

Angiogenesis is a tightly controlled process of growing new blood vessels (see e.g., Folkman & Shing (1992) J. Biol. Chem., 267: 10931-4; Hanahan (1997) Science, 277:48-50, for reviews). Under normal circumstances angiogenesis occurs only during embryonic development, wound healing and development of the corpus luteum. Angiogenesis occurs in a large number of pathologies, such as solid tumor and metastasis growth, various eye diseases, chronic inflammatory states, and ischemic injuries (see, Folkman (1995) Nat. Med., 1:27-31, for review). Thus, growing endothelial cells present unique targets for treatment of several major pathologies.

VEGF proteins are a family of secreted dimeric glycoproteins that are positive regulators of angiogenesis (e.g., Cross and Claesson-Welsh, Trends Pharmacol Sci., 22: 201-7, 2001). Exemplary VEGF proteins include, but are not limited to, VEGF-A (UniProt NO:P15692), VEGF-B (UniProt NO:P49765), VEGF-C (UniProt NO:P49767), VEGF-D (UniProt NO:043915), and PGF (placental growth factor, VEGF-related protein; UniProt NO:Q53XY6), and splice variants, allelic variants or species variants thereof. Exemplary of VEGF-A precursor polypeptides are set forth in SEQ ID NOS:204-210 and include a 26 amino acid signal peptide corresponding to amino acids 1-26 of SEQ ID NOS:204-206 and, as a result of alternative splicing, mature polypeptides of varying length. For example, mature VEGF-A polypeptides can be 206, 189, 183, 165, 148, 145, or 121 amino acids in length. VEGF-B precursor polypeptides are set forth in SEQ ID NOS:211 and 212 and include a 21 amino acid signal peptide corresponding to amino acids 1-21 of SEQ ID NOS:211 and 212 and mature polypeptides that are 186 and 167 amino acids in length and correspond to amino acids 22-207 of SEQ ID NO:211 and 22-188 of SEQ ID NO:212, respectively. The precursor polypeptide for VEGF-C is set forth in SEQ ID NO:213 and includes a 31 amino acid signal peptide corresponding to amino acids 1-31 of SEQ ID NO:213, two propeptide sequences corresponding to amino acids 32-111 and 228-419 of SEQ ID NO:213, and a mature 116 amino acid polypeptide corresponding to amino acids 112-227 of SEQ ID NO:213. The precursor polypeptide for VEGF-D is set forth in SEQ ID NO:214 and includes a 21 amino acid signal peptide corresponding to amino acids 1-21 of SEQ ID NO:214, two propeptide sequences corresponding to amino acids 22-88 and 206-354 of SEQ ID NO:214, and a mature 117 amino acid polypeptide corresponding to amino acids 89-205 of SEQ ID NO:214. The precursor polypeptide for PGF is set forth in SEQ ID NO:215.

The action of VEGF on endothelial cells is mediated by tyrosine kinase receptors, VEGFR-1 (flt-1), VEGFR-2 (KDR/flk-1) and VEGFR-1-related. These receptors are preferentially expressed on endothelial cells. The receptors are single span transmembrane protein tyrosine kinases that belong to the immunoglobulin superfamily and contain seven Ig-like loops in the extracellular domain and share homology with the receptor for platelet-derived growth factor. VEGF binding to these receptors induces receptor dimerization followed by tyrosine phosphorylation of the SH2 and SH3 domains in the dimer. The KDR/flk-1-VEGF complex is then internalized via receptor-mediated endocytosis. Thus, because it can be internalized by cells expressing its receptor or receptors, any VEGF protein, such as any described herein, can serve as a targeting agent in a conjugate containing a modified RIP polypeptide such a modified SA1 polypeptide, or active fragment thereof.

2. Linker Moieties

In preparing the conjugates provided herein, the RIP toxin, such as for example, a modified SA1 toxin provided herein, is linked directly or indirectly to a targeting agent. For example, conjugates provided herein include the following components: (targeting agent)n, (L)q and (targeted agent)m, where L is a linker for linking the targeting agent to the toxin; the targeting agent is any moiety that binds to and is internalized by a receptor expressed on a cell surface; m and n, which are selected independently, are at least 1; and q is 0 or more as long as the resulting conjugate binds to the targeted receptor, is internalized and delivers the targeted agent. The linkage of the components in the conjugate can be by any method presently known in the art for attaching two moieties, so long as the attachment of the linker moiety to the proteinaceous ligand does not substantially impede binding of the proteinaceous ligand to the target cell, that is, to a receptor on the target cell, or substantially impede the internalization or metabolism of the ligand-toxin so as to lower the toxicity of the modified RIP toxin for the target cell. The linkage can be any type of linkage, including, but are not limited to, ionic and covalent bonds, and any other sufficiently stable associate, whereby the targeted agent (e.g., a modified RIP toxin) will be internalized by a cell to which the conjugate is targeted.

The targeting agent, such as a chemokine, is optionally linked to the modified RIP toxin, or active fragment thereof, via one or more linkers. The linker moiety is selected depending upon the properties desired. For example, the length of the linker moiety can be chosen to optimize the kinetics and specificity of ligand binding, including any conformational changes induced by binding of the ligand to a target receptor. The linker moiety should be long enough and flexible enough to allow the proteinaceous ligand moiety and the target cell receptor to freely interact. If the linker is too short or too stiff, there can be steric hindrance between the proteinaceous ligand moiety and the cell toxin. If the linker moiety is too long, the cell toxin can be proteolysed in the process of production, or can not deliver its toxic effect to the target cell effectively. Linkers, such as chemical linkers can be attached to purified ligands using numerous protocols known in the art (see Pierce Chemicals “Solutions, Cross-linking of Proteins: Basic Concepts and Strategies,” Seminar #12, Rockford, Ill.).

a. Exemplary Linkers

Any linker known to those of skill in the art can be used herein. Generally a different set of linkers will be used in conjugates that are fusion proteins from linkers in chemically-produced conjugates. Linkers and linkages that are suitable for chemically linked conjugates include, but are not limited to, disulfide bonds, thioether bonds, hindered disulfide bonds, and covalent bonds between free reactive groups, such as amine and thiol groups. These bonds are produced using heterobifunctional reagents to produce reactive thiol groups on one or both of the polypeptides and then reacting the thiol groups on one polypeptide with reactive thiol groups or amine groups to which reactive maleimido groups or thiol groups can be attached on the other. Other linkers include, acid cleavable linkers, such as bismaleimideothoxy propane, acid labile-transferrin conjugates and adipic acid diihydrazide, that would be cleaved in more acidic intracellular compartments; cross linkers that are cleaved upon exposure to UV or visible light and linkers, such as the various domains, such as CH1, CH2, and CH3, from the constant region of human IgG1 (see, Batra et al. (1993) Molecular Immunol. 30:379-386). In some embodiments, several linkers can be included in order to take advantage of desired properties of each linker. Chemical linkers and peptide linkers can be inserted by covalently coupling the linker to the chemokine receptor targeting agent and the modified RIP toxin. The heterobifunctional agents, described below, can be used to effect such covalent coupling. Peptide linkers also can be linked by expressing DNA encoding the linker and targeting agent, linker and modified RIP toxin, or linker, modified RIP toxin and targeting agent as a fusion protein. Flexible linkers and linkers that increase solubility of the conjugates are contemplated for use; either alone or with other linkers also is contemplated herein.

Linkers can be any moiety suitable to associate a modified RIP toxin and a targeting agent. Such moieties include, but are not limited to, peptidic linkages; amino acid and peptide linkages, typically containing between one and about 60 amino acids; chemical linkers, such as heterobifunctional cleavable cross-linkers. Other linkers include, but are not limited to peptides and other moieties that reduce steric hindrance between the modified RIP toxin and targeting agent, intracellular enzyme substrates, linkers that increase the flexibility of the conjugate, linkers that increase the solubility of the conjugate, linkers that increase the serum stability of the conjugate, photocleavable linkers and acid cleavable linkers.

i. Heterobifunctional Cross-Linking Reagents

Numerous heterobifunctional cross-linking reagents that are used to form covalent bonds between amino groups and thiol groups or to introduce thiol groups into proteins, are known to those of skill in this art (see, e.g., the PIERCE CATALOG, ImmunoTechnology Catalog & Handbook, 1992-1993, which describes the preparation of and use of such reagents and provides a commercial source for such reagents; see, also, e.g., Cumber et al. (1992) Bioconjugate Chem. 3:397-401; Thorpe et al. (1987) Cancer Res. 47:5924-5931; Gordon et al. (1987) Proc. Natl. Acad. Sci. 84:308-312; Walden et al. (1986) J. Mol. Cell. Immunol. 2:191-197; Carlsson et al. (1978) Biochem. J. 173: 723-737; Mahan et al. (1987) Anal. Biochem. 162:163-170; Wawryznaczak et al. (1992) Br. J. Cancer 66:361-366; Fattom et al. (1992) Infection & Immun. 60:584-589; reagents for crosslinking are available: Pierce Chemical Company, Rockford, Ill.; Sigma Chemical Company, St. Louis, Mo.; Molecular Probes, Inc., Eugene, Oreg.). Functional groups that can be used for crosslinking include primary amines, sulfhydryls, carbonyls, carbohydrates and carboxylic acids. Exemplary groups for use in heterobifunctional cross-linking reagents include, but are not limited to, aryl azides, maleimides, carbodiimides, N-hydroxysuccinimide (NHS)-esters, hydrazides, PFP-esters, hydroxymethyl phosphines, psoralens, imidoesters, pyridyl disulfides, isocyanates, and vinyl sulfones. Heterobifunctional cross-linking reagents can be used to form covalent bonds between the targeting agents, such as for example, a chemokine, and a modified RIP toxin. An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker can react with the lysine residue(s) of one polypeptide and through the thiol reactive group, the cross-linker, already tied up to the first protein, reacts with the cysteine residue (free sulfhydryl group) of the other polypeptide. Exemplary heterobifunctional cross-linking reagents include, but are not limited to: N-succinimidyl-3-(2-pyridyldithio)propionate (SPDP; disulfide linker); sulfosuccinimidyl 6-[3-(2-pyridyldithio)propionamido]hexanoate (sulfo-LC-SPDP); succinimidyloxycarbonyl-methyl benzyl thiosulfate (SMBT, hindered disulfate linker); succinimidyl 6-[3-(2-pyridyldithio)propionamido]hexanoate (LC-SPDP); sulfosuccinimidyl 4-(N-maleimidomethyl)cyclohexane-1-carboxylate (sulfo-SMCC); succinimidyl 3-(2-pyridyldithio)butyrate (SPDB; hindered disulfide bond linker); sulfosuccinimidyl 2-(7-azido-4-methylcoumarin-3-acetamide) ethyl-1,3′-dithiopropionate (SAED); sulfo-succinimidyl 7-azido-4-methylcoumarin-3-acetate (SAMCA); sulfosuccinimidyl 6-[alpha-methyl-alpha-(2-pyridyldithio)toluamido]hexanoate (sulfo-LC-SMPT); 1,4-di-[3′-(2′-pyridyldithio)propionamido]butane (DPDPB); 4-succinimidyl-oxycarbonyl-methyl-(2-pyridylthio)toluene (SMPT, hindered disulfate linker); 4-succinimidyl-oxycarbonyl-a-(2-pyridyldithio)toluene; sulfosuccinimidyl6[-methyl-(2-pyridyldithio)toluamido]hexanoate (sulfo-LC-SMPT); m-maleimidobenzoyl-N-hydroxysuccinimide ester (MBS); m-maleimidobenzoyl-N-hydroxysulfosuccinimide ester (sulfo-MBS); N-succinimidyl(4-iodoacetyl)aminobenzoate (SLAB; thioether linker); sulfosuccinimidyl(4-iodoacetyl)amino benzoate (sulfo-SLAB); succinimidyl-4(p-maleimidophenyl)butyrate (SMPB); sulfosuccinimidyl4-(p-maleimidophenyl)butyrate (sulfo-SMPB); azidobenzoyl hydrazide (ABH); 3-(2-pyridyldithio)-propionyl hydrazide; Ellman's reagent; dichlorotriazinic acid, S-(2-thiopyridyl)-L-cysteine. Further exemplary bifunctional linking compounds are disclosed, for example, in U.S. Pat. Nos. 5,349,066, 5,618,528, 4,569,789, 4,952,394, and 5,137,877.

ii. Acid Cleavable, Photocleavable and Heat Sensitive Linkers

Acid cleavable linkers, photocleavable and heat sensitive linkers also can be used, particularly where it is necessary to cleave the modified RIP toxin to permit it to be more readily accessible to reaction. Many cleavable groups are known in the art (see, for example, Jung et al. (1983) Biochem. Biophys. Acta 761: 152 162; Joshi et al. (1990) J. Biol. Chem. 265: 14518 14525; Zarling et al. (1980) J. Immunol. 124: 913 920; Bouizar et al. (1986) Eur. J. Biochem. 155: 141 147; Park et al. (1986) J. Biol. Chem. 261: 205 210; Browning et al. (1989) J. Immunol. 143: 1859-1867). Moreover a broad range of cleavable, bifunctional linker groups is commercially available from suppliers such as Pierce.

Acid cleavable linkers include, but are not limited to, bismaleimideothoxy propane; and adipic acid dihydrazide linkers (see, e.g., Fattom et al. (1992) Infection & Immun. 60:584-589) and acid labile transferrin conjugates that contain a sufficient portion of transferrin to permit entry into the intracellular transferrin cycling pathway (see, e.g., Welhöner et al. (1991) J. Biol. Chem. 266:4309-4314).

Photocleavable linkers are linkers that are cleaved upon exposure to light (see, e.g., Goldmacher et al. (1992) Bioconj. Chem. 3:104-107), thereby releasing the targeted agent upon exposure to light. Photocleavable linkers that are cleaved upon exposure to light are well known (see, e.g., Hazum et al. (1981) in Pept., Proc. Eur. Pept. Symp., 16th, Brunfeldt, K (Ed), pp. 105-110, which describes the use of a nitrobenzyl group as a photocleavable protective group for cysteine; Yen et al. (1989) Makromol. Chem. 190:69-82, which describes water soluble photocleavable copolymers, including hydroxypropylmethacrylamide copolymer, glycine copolymer, fluorescein copolymer and methylrhodamine copolymer; Goldmacher et al. (1992) Bioconj. Chem. 3:104-107, which describes a cross-linker and reagent that undergoes photolytic degradation upon exposure to near UV light (350 nm); and Senter et al. (1985) Photochem. Photobiol 42:231-237, which describes nitrobenzyloxycarbonyl chloride cross linking reagents that produce photocleavable linkages). Such linkers would have particular use in treating dermatological or ophthalmic conditions that can be exposed to light using fiber optics. After administration of the conjugate, the eye or skin or other body part can be exposed to light, resulting in release of the modified RIP toxin from the conjugate. Such photocleavable linkers are useful in connection with diagnostic protocols in which it is desirable to remove the targeting agent to permit rapid clearance from the body of the animal.

iii. Other Linkers for Chemical Conjugation

Other linkers, include trityl linkers, particularly, derivatized trityl groups to generate a genus of conjugates that provide for release of therapeutic agents at various degrees of acidity or alkalinity. The flexibility thus afforded by the ability to pre-select the pH range at which the therapeutic agent will be released allows selection of a linker based on the known physiological differences between tissues in need of delivery of a therapeutic agent (see, e.g., U.S. Pat. No. 5,612,474). For example, the acidity of tumor tissues appears to be lower than that of normal tissues.

iv. Peptide Linkers

The linker moieties can be peptides. Peptide linkers can be employed in fusion proteins and also in chemically linked conjugates. The peptide typically has from about 2 to about 60 amino acid residues, for example from about 5 to about 40, or from about 10 to about 30 amino acid residues. The length selected will depend upon factors, such as the use for which the linker is included.

The proteinaceous ligand binds with specificity to a receptor(s) on one or more of the target cell(s) and is taken up by the target cell(s). In order to facilitate passage of the ligand-toxin conjugate into the target cell, it is presently preferred that the size of the ligand-toxin conjugate be no larger than can be taken up by the target cell of interest. Generally, the size of the ligand-toxin conjugate will depend upon its composition. In the case where the ligand toxin conjugate contains a chemical linker and a chemical toxin (i.e., rather than proteinaceous one), the size of the ligand-toxin is generally smaller than when the ligand-toxin conjugate is a fusion protein. Peptidic linkers can conveniently be encoded by nucleic acid and incorporated in fusion proteins upon expressed in a host cell, such as E. coli.

Peptide linkers are advantageous when the targeting agent is proteinaceous. For example, the linker moiety can be a flexible spacer amino acid sequence, such as those known in single-chain antibody research. Examples of such known linker moieties include, but are not limited to, GGGGS (SEQ ID NO: 192), (GGGGS)ₙ (SEQ. ID NO:193), GKSSGSGSESKS (SEQ ID NO:194), GSTSGSGKSSEGKG (SEQ. ID NO: 195), GSTSGSGKSSEGSGSTKG (SEQ ID NO: 196), GSTSGSGKSSEGKG (SEQ ID NO:197), GSTSGSGKPGSGEGSTKG (SEQ ID NO: 198), EGKSSGSGSESKEF (SEQ ID NO: 199), SRSSG (SEQ. ID NO:200), SGSSC (SEQ ID NO:201). A Diphtheria toxin trypsin sensitive linker having the sequence AMGRSGGGCAGNRVGSSLSCGGLNLQAM (SEQ ID NO:202) also is useful.

Alternatively, the peptide linker moiety can be VM or AM (SEQ ID NO:34), or have the structure described by the formula: AM(G_{2 to 4}S)ₓAM wherein X is an integer from 1 to 11 (SEQ ID NO:203). Additional linking moieties are described, for example, in Huston et al. (1988) Proc. Natl. Acad. Sci. U.S.A. 85:5879-5883; Whitlow, M., et al. (1993) Protein Engineering 6:989-995; Newton et al. (1996) Biochemistry 35:545-553; A. J. Cumber et al. (1992) Bioconj. Chem. 3:397-401; Ladurner et al. (1997) J. Mol. Biol. 273:330-337; and U.S. Pat. No. 4,894,443.

Other linkers include, but are not limited to: enzyme substrates, such as cathepsin B substrate, cathepsin D substrate, trypsin substrate, thrombin substrate, subtilisin substrate, Factor Xa substrate, and enterokinase substrate; linkers that increase solubility, flexibility, and/or intracellular cleavability include linkers, such as (glyₘser)ₙ and (serₘgly)ₙ, in which m is 1 to 6, generally 1 to 4, and typically 2 to 4, and n is 1 to 30, or 1 to 10, and typically 1 to 4 (see, e.g., International PCT application No. WO 96/06641, which provides exemplary linkers for use in conjugates). In some embodiments, several linkers can be included in order to take advantage of desired properties of each linker.

3. Exemplary Leukocyte Population Modulator (LPM) Conjugates

Exemplary ligand-toxin conjugates include LPM conjugates, which contain a chemokine linked directly or indirectly to a Shiga toxin A1 (SA1) variant, such as, for example, any of the SA1 variants described herein. Typically, such conjugates contain the mature portion of the chemokine polypeptide or a portion of the polypeptide that can bind to the receptor. Optionally, where linkage is indirect, the nucleic acid molecule that encodes the conjugate can contain a sequence encoding a linker polypeptide between the chemokine targeting agent polypeptide and the SA1 variant targeted moiety, such as for example, an Ala-Met linker (SEQ ID NO:34; technically, the Met is the Shiga toxin start codon for bacterial expression). In some examples, additional nucleic acid molecules can be added to the nucleic acid that encodes the ligand-toxin conjugate or can be linked to the ligand-toxin conjugate by other means, such as by a chemical linker, to facilitate purification, expression, cloning, or detection. For example, restriction enzyme sites can be engineered at one or both of the 3′ and 5′ ends of the nucleic acid molecule to facilitate cloning. In one such example, an NdeI restriction site at positions 1-6 and a BamHI restriction site at positions 967-972 were engineered in the nucleic acid molecule as set forth in SEQ ID NOS:37 and 39.

Among the LPM conjugates provided herein is a conjugate of a mature MCP-1 chemokine polypeptide linked directly or indirectly to a variant 1 Shiga toxin A1 (SA1) subunit as the targeted agent. For example, as described in the Examples provided, the LPM1a conjugate contains a mature MCP-1 polypeptide (set forth in SEQ ID NO:69) linked to residues 23-268 of the SA1 subunit polypeptide, containing the ribosome inactivating (RIP) domain (referred to herein as SA1 variant 1; corresponding to the sequence of amino acids set forth in SEQ ID NO:22). The MCP-1 polypeptide and the SA1 polypeptide are linked indirectly via an Ala-Met linker (SEQ ID NO:34) to produce a ligand:linker:toxin fusion polypeptide. An exemplary nucleic acid that encodes the LPM1a polypeptide is set forth in SEQ ID NO:37. The encoded LPM1a polypeptide is set forth in SEQ ID NO:38.

Also among the LPM conjugates provided herein is a conjugate of a mature MCP-1 chemokine polypeptide linked directly or indirectly to a variant 2 Shiga toxin A1 (SA1) subunit as the targeted agent. For example, as described in the Examples provided, the LPM1b conjugate contains a mature MCP-1 polypeptide (set forth in SEQ ID NO:69) linked to a truncated Shiga toxin A1 subunit polypeptide (referred to herein as SA1 variant 2, corresponding to the sequence of amino acids set forth in SEQ ID NO:24). The MCP-1 polypeptide and the variant 2 SA1 polypeptide are linked indirectly via an Ala-Met linker (SEQ ID NO:34) to produce a ligand:linker:toxin fusion polypeptide. An exemplary nucleic acid that encodes the LPM1b polypeptide is set forth in SEQ ID NO:39, where nucleotides 7-966 encode the ligand-toxin conjugate polypeptide, and nucleotides 964-966 encode an engineered stop codon. The encoded LPM1b polypeptide set forth in SEQ ID NO:40 is 320 amino acids in length and contains a 5′ start methionine residue (at amino acid position 1) followed by a mature MCP-1 (amino acids 2-77), an Ala-Met linker (amino acids 78-79), and an SA1 variant 2 subunit (amino acids 80-320).

Another exemplary LPM provided herein is a conjugate of a mature MCP-1 chemokine polypeptide linked directly or indirectly to a mutant variant 1 (also called variant 3) Shiga toxin A1 (SA1) subunit as the targeted agent, which is a modified SA1 polypeptide identified in the selection methods herein. For example, LPM1c conjugate contains a mature MCP-1 polypeptide (set forth in SEQ ID NO:69) linked to a mutant Shiga toxin A1 subunit polypeptide (referred to herein as SA1 variant 3, corresponding to the sequence of amino acids set forth in SEQ ID NO:26). The MCP-1 polypeptide and the SA1 polypeptide variant are linked indirectly via an Ala-Met linker (SEQ ID NO:34) to produce a ligand:linker:toxin fusion polypeptide. The SA1 variant 3 has a L to R mutation at position 38 with respect to the mature wild-type SA1 polypeptide set forth in SEQ ID NO:22. As described in the Examples, LPM1c was generated in a screen for modified forms of the SA1 portion of the LPM1a conjugate (SEQ ID NO:38). An exemplary nucleic acid that encodes the LPM1c polypeptide is set forth in SEQ ID NO:41. The encoded LPM1c polypeptide is set forth in SEQ ID NO:42.

Another exemplary LPM provided herein is a conjugate of a mature MCP-1 chemokine polypeptide linked directly or indirectly to a mutant variant 2 (also called variant 4) Shiga toxin A1 (SA1) subunit as the targeted agent, which is a modified SA1 polypeptide identified in the selection methods herein. For example, LPM1d conjugate contains a mature MCP-1 chemokine polypeptide (set forth in SEQ ID NO:69) linked to a mutant Shiga toxin A1 subunit polypeptide (referred to herein as SA1 variant 4, corresponding to the sequence of amino acids set forth in SEQ ID NO:28). The MCP-1 polypeptide and the SA1 polypeptide variant are linked indirectly via an Ala-Met linker (SEQ ID NO:34) to produce a ligand:linker:toxin fusion polypeptide. The SA1 variant 4 has a V to A mutation at position 219 with respect to the mature truncated SA1 variant 2 polypeptide set forth in SEQ ID NO:24. As described in the Examples, LPM1d was generated in a screen for variants of the SA1 portion of the LPM1b conjugate (SEQ ID NO:40). An exemplary nucleic acid that encodes the LPM1d polypeptide is set forth in SEQ ID NO:43. The encoded LPM1d polypeptide is set forth in SEQ ID NO:44.

Another exemplary LPM provided herein is a conjugate of the chemokine Eotaxin linked directly or indirectly to a modified Shiga toxin A1 (SA1) subunit as the targeted agent. For example, LPM2 conjugate contains a mature Eotaxin polypeptide (corresponding to amino acids 24-97 of the sequence set forth in SEQ ID NO: 113) linked to an SA1 variant 4 polypeptide (corresponding to the sequence of amino acids set forth in SEQ ID NO:28) via an Ala-Met linker (SEQ ID NO:34). Exemplary of a nucleic acid molecule that encodes LPM2 is set forth in nucleotides 7-960 of SEQ ID NO:45, including an engineered stop codon at nucleotides 958-960. The encoded LPM2 polypeptide set forth in SEQ ID NO:46 is 318 amino acids in length which contains a 5′ start methionine residue (at amino acid position 1) followed by a mature Eotaxin (amino acids 2-75), an Ala-Met linker (amino acids 76-77), and an SA1 variant 4 subunit (amino acids 78-318).

Another exemplary LPM provided herein that is a conjugate of the chemokine Eotaxin linked to a modified Shiga toxin A1 (SA1) subunit as the targeted agent is LPM12. The Eotaxin polypeptide used in LPM12 has the same amino acid sequence as that of the Eotaxin in LPM2, however due to differences in the way they were synthesized (see Example 3), their nucleic acid sequences differ. Exemplary of a nucleic acid molecule that encodes LPM12 is set forth in nucleotides 7-960 of SEQ ID NO:65, including an engineered stop codon at nucleotides 958-960. The encoded LPM12 polypeptide set forth in SEQ ID NO:46 is 318 amino acids in length which contains a 5′ start methionine residue (at amino acid position 1) followed by a mature Eotaxin (amino acids 2-75), an Ala-Met linker (amino acids 76-77), and an SA1 variant 4 subunit (amino acids 78-318).

Another exemplary LPM provided herein is a conjugate of the chemokine SDF-1β linked directly or indirectly to a modified Shiga toxin A1 (SA1) subunit as the targeted agent. In one example, the modified SA1 subunit is the variant 4 SA1 polypeptide identified in the selection methods herein. For example, the LPM3 conjugate contains a mature SDF-1β polypeptide (corresponding to amino acids 22-93 of the sequence set forth in SEQ ID NO:114) linked to an SA1 variant 4 polypeptide (corresponding to the sequence of amino acids set forth in SEQ ID NO:28) via an Ala-Met linker (SEQ ID NO:34). Exemplary of a nucleic acid molecule that encodes LPM3 is set forth in nucleotides 7-954 of SEQ ID NO:47, including an engineered stop codon at nucleotides 952-954. The encoded LPM3 polypeptide set forth in SEQ ID NO:48 is 316 amino acids in length which contains a 5′ start methionine residue (at amino acid position 1) followed by a mature SDF-1β (amino acids 2-73), an Ala-Met linker (amino acids 74-75), and an SA1 variant 4 subunit (amino acids 76-316).

Another exemplary LPM provided herein is a conjugate of the chemokine GRO-α linked directly or indirectly to a modified Shiga toxin A1 (SA1) subunit as the targeted agent. In one example, the modified SA1 subunit is the variant 4 SA1 polypeptide identified in the selection methods herein. For example, the LPM4 conjugate contains a mature GRO-α polypeptide (corresponding to amino acids 35-107 of the polypeptide set forth in SEQ ID NO: 115) linked to an SA1 variant 4 polypeptide (corresponding to the sequence of amino acids set forth in SEQ ID NO:28) via an Ala-Met linker (SEQ ID NO:34). Exemplary of a nucleic acid molecule that encodes LPM4 is set forth in nucleotides 7-957 of SEQ ID NO:49, including an engineered stop codon at nucleotides 955-957. The encoded LPM4 polypeptide set forth in SEQ ID NO:50 is 317 amino acids in length which contains a 5′ start methionine residue (at amino acid position 1) followed by a mature GRO-α (amino acids 2-74), an Ala-Met linker (amino acids 75-76), and an SA1 variant 4 subunit (amino acids 77-317).

Another exemplary LPM provided herein is a conjugate of the chemokine MIP-1β linked directly or indirectly to a modified Shiga toxin A1 (SA1) subunit as the targeted agent. In one example, the modified SA1 subunit is the variant 4 SA1 polypeptide identified in the selection methods herein. For example, the LPM5 conjugate contains a mature MIP-1 polypeptide (corresponding to amino acids 24-92 of the polypeptide set forth in SEQ ID NO: 116) linked to an SA1 variant 4 polypeptide (corresponding to the sequence of amino acids set forth in SEQ ID NO:28) via an Ala-Met linker (SEQ ID NO:34). Exemplary of such an LPM5 sequence are nucleotides 7-945, including an engineered stop codon at nucleotides 943-945, of the nucleic acid sequence set forth in SEQ ID NO:51. The encoded LPM5 polypeptide set forth in SEQ ID NO:52 is 313 amino acids in length which contains a 5′ start methionine residue (at amino acid position 1) followed by a mature MIP-1β (amino acids 2-70), an Ala-Met linker (amino acids 71-72), and an SA1 variant 4 subunit (amino acids 73-313).

Another exemplary LPM provided herein is a conjugate of the chemokine IL-8 linked directly or indirectly to a modified Shiga toxin A1 (SA1) subunit as the targeted agent. In one example, the modified SA1 subunit is the variant 4 SA1 polypeptide identified in the selection methods herein. For example, the LPM6 conjugate contains a mature IL-8 polypeptide (corresponding to amino acids 21-99 of the polypeptide set forth in SEQ ID NO: 117) linked to an SA1 variant 4 sequence (corresponding to the sequence of amino acids set forth in SEQ ID NO:28) via an Ala-Met linker (SEQ ID NO:34). Exemplary of a nucleic acid molecule that encodes LPM6 is set forth in nucleotides 7-969 of SEQ ID NO:453, including an engineered stop codon at nucleotides 967-969. The encoded LPM6 polypeptide set forth in SEQ ID NO:54 is 321 amino acids in length which contains a 5′ start methionine residue (at amino acid position 1) followed by a mature IL-8 (amino acids 2-78), an Ala-Met linker (amino acids 79-80), and an SA1 variant 4 subunit (amino acids 81-321).

Another exemplary LPM provided herein is a conjugate of the chemokine IP-10 linked directly or indirectly to a modified Shiga toxin A1 (SA1) subunit as the targeted agent. In one example, the modified SA1 subunit is the variant 4 SA1 polypeptide identified in the selection methods herein. For example, the LPM7 conjugate contains a mature IP-10 polypeptide (corresponding to amino acids 22-98 of the polypeptide set forth in SEQ ID NO:118) linked to an SA1 variant 4 polypeptide (corresponding to the sequence of amino acids set forth in SEQ ID NO:28) via an Ala-Met linker (SEQ ID NO:34). Exemplary of a nucleic acid molecule that encodes LPM7 is set forth in nucleotides 7-969 of SEQ ID NO:55, including an engineered stop codon at nucleotides 967-969. The encoded LPM7 polypeptide set forth in SEQ ID NO:56 is 321 amino acids in length which contains a 5′ start methionine residue (at amino acid position 1) followed by a mature IP-10 (amino acids 2-78), an Ala-Met linker (amino acids 79-80), and an SA1 variant 4 subunit (amino acids 81-321).

Another exemplary LPM provided herein is a conjugate of the chemokine MCP-3 linked directly or indirectly to a modified Shiga toxin A1 (SA1) subunit as the targeted agent. In one example, the modified SA1 subunit is the variant 4 SA1 polypeptide identified in the selection methods herein. For example, the LPM8 conjugate contains a mature MCP-3 polypeptide (corresponding to amino acids 24-99 of the polypeptide set forth in SEQ ID NO: 119) linked to an SA1 variant 4 polypeptide (corresponding to the sequence of amino acids set forth in SEQ ID NO:28) via an Ala-Met linker (SEQ ID NO:34). Exemplary of a nucleic acid molecule that encodes LPM8 is set forth in nucleotides 7-966 of SEQ ID NO:57, including an engineered stop codon at nucleotides 964-966. The encoded LPM8 polypeptide set forth in SEQ ID NO:58 is 320 amino acids in length which contains a 5′ start methionine residue (at amino acid position 1) followed by a mature MCP-3 (amino acids 2-77), an Ala-Met linker (amino acids 78-79), and an SA1 variant 4 subunit (amino acids 80-320).

Another exemplary LPM provided herein is a conjugate of the chemokine MIP-3α linked directly or indirectly to a modified Shiga toxin A1 (SA1) subunit as the targeted agent. In one example, the modified SA1 subunit is the variant 4 SA1 polypeptide identified in the selection methods herein. For example, the LPM9 conjugate contains a mature MIP-3α polypeptide (corresponding to amino acids 27-96 of the polypeptide set forth in SEQ ID NO: 120) linked an SA1 variant 4 polypeptide (corresponding to the sequence of amino acids set forth in SEQ ID NO:28) via an Ala-Met linker (SEQ ID NO:34). Exemplary of a nucleic acid molecule that encodes LPM9 is set forth in nucleotides 7-948 of SEQ ID NO:59, including an engineered stop codon at nucleotides 946-948. The encoded LPM9 polypeptide set forth in SEQ ID NO:60 is 314 amino acids in length which contains a 5′ start methionine residue (at amino acid position 1) followed by a mature MIP-3α (amino acids 2-71), an Ala-Met linker (amino acids 72-73), and an SA1 variant 4 subunit (amino acids 74-314).

Another exemplary LPM provided herein is a conjugate of the chemokine MDC linked directly or indirectly to a modified Shiga toxin A1 (SA1) subunit as the targeted agent. In one example, the modified SA1 subunit is the variant 4 SA1 polypeptide identified in the selection methods herein. For example, the LPM10 conjugate contains a mature MDC polypeptide (corresponding to amino acids 25-93 of the polypeptide set forth in SEQ ID NO: 121) linked to an SA1 variant 4 polypeptide (corresponding to the sequence of amino acids set forth in SEQ ID NO:28) via an Ala-Met linker (SEQ ID NO:34). Exemplary of a nucleic acid molecule that encodes LPM10 is set forth in nucleotides 7-945 of SEQ ID NO:61, including an engineered stop codon at amino nucleotides 943-945. The encoded LPM10 polypeptide set forth in SEQ ID NO:62 is 313 amino acids in length which contains a 5′ start methionine residue (at amino acid position 1) followed by a mature MDC (amino acids 2-70), an Ala-Met linker (amino acids 71-72), and an SA1 variant 4 subunit (amino acids 73-313).

Another exemplary LPM provided herein is a conjugate of the chemokine MIP-1α linked directly or indirectly to a modified Shiga toxin A1 (SA1) subunit as the targeted agent. In one example, the modified SA1 subunit is the variant 4 SA1 polypeptide identified in the selection methods herein. For example, the LPM11 conjugate contains a mature MIP-1α polypeptide (corresponding to amino acids 24-92 of the polypeptide set forth in SEQ ID NO:122) linked to an SA1 variant 4 polypeptide (corresponding to the sequence of amino acids set forth in SEQ ID NO:28) via an Ala-Met linker (SEQ ID NO:34). Exemplary of a nucleic acid molecule that encodes LPM11 is set forth in nucleotides 7-945 of SEQ ID NO:63, including an engineered stop codon at a nucleotides 943-945. The encoded LPM11 polypeptide set forth in SEQ ID NO:64 is 313 amino acids in length which contains a 5′ start methionine residue (at amino acid position 1) followed by a mature MIP-1α (amino acids 2-70), an Ala-Met linker (amino acids 71-72), and an SA1 variant 4 subunit (amino acids 73-313).

Another exemplary LPM provided herein is a conjugate of the chemokine BCA-1 linked directly or indirectly to a modified Shiga toxin A1 (SA1) subunit as the targeted agent. In one example, the modified SA1 subunit is the variant 4 SA1 polypeptide identified in the selection methods herein. For example, the LPM13 conjugate contains a mature BCA-1 polypeptide (corresponding to amino acids 23-109 of the polypeptide set forth in SEQ ID NO: 123) linked to an SA1 variant 4 polypeptide (corresponding to the sequence of amino acids set forth in SEQ ID NO:28) via an Ala-Met linker (SEQ ID NO:34). Exemplary of a nucleic acid molecule that encodes LPM13 is set forth in nucleotides 7-999 of SEQ ID NO:66, including an engineered stop codon at nucleotides 997-999. The encoded LPM13 polypeptide set forth in SEQ ID NO:67 is 331 amino acids in length which contains a 5′ start methionine residue (at amino acid position 1) followed by a mature BCA-1 (amino acids 2-88), an Ala-Met linker (amino acids 89-90), and an SA1 variant 4 subunit (amino acids 91-331).

### G. PREPARATION OF MODIFIED RIP TOXINS AND CONJUGATES THEREOF

Conjugates of targeting moieties linked to targeted agents can be prepared either by chemical conjugation, recombinant DNA technology, or combinations of recombinant expression and chemical conjugation. The methods herein can be used to prepare and use conjugates of any targeting agent with any targeted agent, such as a RIP toxin, either directly or via linkers as described herein. The targeting agent and targeted agent can be linked in any orientation and more than one targeting agent and/or targeted agent can be present in a conjugate. The methods herein are exemplified with particular reference to conjugates containing a targeting agent, such as a chemokine, and a targeted agent, such as a modified Shiga-toxin A1 polypeptide.

Further, methods are provided herein for expression and production of recombinant polypeptides. Such methods can be used to express modified toxins, or toxin variants, provided herein either alone or as a conjugate fusion protein (e.g., ligand-RIP toxin conjugate) with a selected targeting agent, such as a chemokine. In examples, where the targeted agent, such as modified toxin provided herein, and the targeting agent are expressed as individual peptides, conjugates of the modified targeted agent with the targeting agent can be generated via chemical means as discussed elsewhere herein.

1. Methods of Generating and Cloning Toxin Polypeptides, or Conjugates Containing Toxin Polypeptides

Nucleic acids encoding a modified toxin, or a conjugate containing a modified toxin, including ligand-toxin conjugates, can be cloned or isolated using any available methods known in the art for cloning and isolating nucleic acid molecules. For example, conjugates containing chimeric fusion proteins of a targeting agent, or ligand, and one or more targeted agents can be produced by well known techniques of protein synthesis if the nucleic acid sequence of the targeting agent or targeted agent are known, or chemical synthesis of DNA molecules that encode the selected targeting agent or targeted agent. Alternatively, if the nucleic acid sequence of the targeting agent or targeted agent are unknown, the sequence can first be determined using well known methods, such as, but not limited to screening of libraries, including nucleic acid hybridization screening, antibody-based screening and activity based screening. Such methods of screening also can be used to obtain nucleic acid sequences that encode a particular protein when only a portion of the amino acid sequence is known.

Methods for amplification of nucleic acids can be used to isolate nucleic acid molecules encoding a targeting agent and/or a targeted agent, including for example, polymerase chain reaction (PCR) methods. A nucleic acid containing material can be used as a starting material from which a targeting agent- or targeted agent-encoding nucleic acid molecule can be isolated. For example, DNA and mRNA preparation, cell extracts, tissue extracts, fluid samples (e.g., blood, serum, saliva), samples from healthy and/or diseased subjects can be used in amplification methods. Nucleic acid libraries also can be used as a source of starting material. Primers can be designed to amplify the desired molecule. For example, primers can be designed based on expressed sequences from which a toxin or ligand molecule (i.e. chemokine) is generated. Primers can be designed based on back-translation of a particular known amino acid sequence. Nucleic acid molecules generated be amplification can be sequenced and confirmed to encode the molecule.

Some of the genes that encode a targeting agent or targeted agent are commercially available. For example, nucleic acid molecules encoding chemokines or cell toxins are available. An advantage of obtaining commercially available genes is that the sequences have generally been optimized for expression in hosts, such as E. coli. A polynucleotide encoding a protein, peptide or polynucleotide of interest can be produced using nucleic acid synthesis technology. Methods of manipulating a DNA molecule including, but not limited to, cloning into vectors, mutagenesis of nucleic acid residues, and addition or deletion of nucleic acid residues, are well known in the art, and can be used to generate modified RIP toxins or ligand-RIP toxin conjugates provided herein.

In one embodiment, the chimeric ligand-RIP toxin is produced as a fusion protein. The fusion protein can be produced by recombinant nucleic acid technology in which a single polypeptide includes a targeting moiety, such as a chemokine, is linked directly to a proteinaceous targeted agent, such as a cell toxin. Alternatively, the proteins can be separated by a distance to ensure that the protein forms proper secondary and tertiary structures. Suitable linker sequences (1) will adopt a flexible extended conformation, (2) will not exhibit propensity for developing an ordered secondary structure which could interact with the functional domains of the fusion polypeptide, and (3) will have minimal hydrophobic or charged character with could promote interaction with the functional protein domains. The targeting moiety can be positioned at the amino-terminus relative to the cell toxin moiety in the polypeptide. An example of such a fusion protein has the generalized structure: (amino terminus) Targeting agent:Peptide linker:Toxin (carboxy terminus). Alternatively, the targeting moiety can be positioned at the carboxy-terminus relative to the cell toxin moiety within the fusion protein, for example, having the generalized structure: (amino terminus) Toxin:Peptide linker:Targeting agent (carboxy terminus).

Also contemplated herein are fusion proteins that contain additional amino acid sequences at the amino and/or carboxy termini, such as sequences for epitope tags or other moieties that facilitate protein purification. For example, polyhistidine tags, that can facilitate processes, such as cloning, expression, post-translational modification, purification, detection, and administration can be employed. In some cases, where there is more than one RIP toxin, more than one linker, or more than one ligand, the genes can be arranged in any order provided that the desired activity of the targeting agent or targeted agent is not eliminated.

Fusion proteins can be prepared using conventional techniques of enzyme cutting and ligation of fragments from desired sequences. For example, desired sequences can be synthesized using an oligonucleotide synthesizer, isolated from the DNA of a parent cell which produces the protein by appropriate restriction enzyme digestion, or obtained from a target source, such as a cell, tissue, vector or other target source, by PCR of genomic DNA with appropriate primers. In one example, toxin conjugates, such as any ligand-toxin conjugate provided herein containing a modified toxin moiety, can be generated by successive rounds of ligating DNA target sequences, into a vector at engineered recombination site. The digested products can be subcloned into a vector for further recombinant manipulation of a sequence, such as to create a fusion with another nucleic acid sequence already contained within a vector, or for the expression of a target molecule.

In some cases, PCR amplification can be employed as a means to obtain sufficient quantities of digested product. PCR primers used in the PCR amplification also can be engineered to facilitate the operative linkage of nucleic acid sequences. For example, non-template complementary 5′ extension can be added to primers to allow for a variety of post-amplification manipulations of the PCR product without significant effect on the amplification itself. For example, these 5′ extension can include restriction sites, promoter sequences, restriction enzyme linker sequences, a protease cleavage site sequence or sequences for epitope tags. In one example, for the purpose of creating a fusion sequence, sequences that can be incorporated into a primer include, for example, a sequence encoding a myc tag, his tag, or other small epitope tag, such that the amplified PCR product effectively contains a fusion of a nucleic acid sequence of interest with an epitope tag.

In another example, incorporation of restriction enzyme sites into a primer can facilitate subcloning of the amplification product into a vector that contains a compatible restriction site, such as by providing sticky ends for ligation of a nucleic acid sequence. Subcloning of multiple PCR amplified products into a single vector can be used as a strategy to operatively link or fuse different nucleic acid sequences. Other methods for subcloning of PCR products into vectors include blunt end cloning, TA cloning, ligation independent cloning, and in vivo cloning.

Prior to subcloning of a PCR product containing exposed restriction enzyme sites into a vector, such as for creating a fusion with a sequence of interest, it is sometimes necessary to resolve a digested PCR product from those that remain uncut. In such examples, the addition of fluorescent tags at the 5′ end of a primer can be added prior to PCR. This allows for identification of digested products since those that have been digested successfully will have lost the fluorescent label upon digestion. In some instances, the use of amplified PCR products containing restriction sites for subsequent subcloning into a vector for the generation of a fusion sequence can result in the incorporation of restriction enzyme linker sequences in the fusion protein product. Generally such linker sequences are short and do not impair the function of a polypeptide so long as the sequences are operatively linked.

2. Production of Conjugates Containing Fusion Proteins and Expression Systems

The nucleic acid molecule encoding a toxin or a conjugate thereof, such as any ligand-toxin conjugates provided herein, can be provided in the form of a vector, which contains the nucleic acid molecule. One example of such a vector is a plasmid. Many expression vectors are available and known to those of skill in the art and can be used for expression of an toxin polypeptide, including toxin conjugates. The choice of expression vector can be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector. In addition, many expression vectors offer either an N-terminal or C-terminal epitope tag adjacent to the multiple cloning site so that any resulting protein expressed from the vector will have an epitope tag inserted in frame with the polypeptide sequence.

The fusion protein can be produced using well known techniques, wherein a host cell is transfected with an expression vector containing expression control sequences operably linked to a nucleic acid molecule coding for the fusion protein to be expressed (Molecular Cloning A Laboratory Manual, Sambrook et al., eds., 2nd Ed., Cold Spring Harbor Laboratory, N.Y., 1989). DNA encoding a toxin, generally in the form of a fusion protein containing a ligand linked directly or indirectly to a modified toxin, such as any of the ligand-toxin conjugates provided herein, is transfected into a host cell for expressions. Toxin polypeptides, including ligand-toxin conjugates, can be expressed in any organism suitable to produce the required amounts and form of polypeptide needed for administration and treatment. Generally, any cell type that can be engineered to express heterologous DNA and has a secretory pathway is suitable. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

a. Plasmids and Host Cells for Expression

The construction of expression vectors that contain a nucleic acid molecule that encodes the RIP toxin variant or a ligand-RIP toxin variant conjugate provided herein and the expression of the nucleic acid in transfected cells involves the use of molecular cloning techniques well known in the art. Such methods include construction of expression vectors containing a nucleic acid molecule encoding a polypeptide operably linked to appropriate transcriptional/translational control signals. These methods also include in vitro recombinant nucleic acid (e.g. DNA or RNA) techniques, synthetic techniques and in vivo recombination/genetic recombination (see, e.g., techniques described in Molecular Cloning: A Laboratory Manual, Sambrook et al., eds., 2nd ed., Cold Spring Harbor Laboratory, N.Y., 1989; Current Protocols in Molecular Biology, Vols. 1 and 2, Ausubel, et al. eds., Current Protocols, 1987-1994; John Wiley and Sons, Inc., 1994-1999; and Cloning Vectors: A Laboratory Manual, Vols I-IV, Pouwels, et al., eds., and Supplements therein, Elsevier, N.Y., 1995-1998).

Recombinant nucleic acid molecules for expression of the polypeptide of interest in host cells generally will be in the form of an expression vector, which includes expression control sequences operatively linked to a nucleic acid molecule encoding the polypeptide. Methods of obtaining stable transfer so that the foreign nucleic acid is continuously maintained in the host also are known in the art. Transformation of a host cell with recombinant nucleic acid can be carried out by conventional techniques as are well known to those skilled in the art.

A variety of host-expression vector systems can be used to express the RIP toxin variant or ligand-RIP toxin variant conjugate protein. These include, but are not limited to, microorganisms, such as bacteria, transformed with recombinant plasmid DNA, bacteriophage DNA, or cosmid DNA expression vectors containing the nucleic acid molecule that encodes the RIP toxin variant or ligand-RIP toxin variant conjugate; yeast transformed with recombinant yeast expression vectors containing the nucleic acid molecule that encodes the RIP toxin variant or ligand-RIP toxin variant conjugate; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the nucleic acid molecule that encodes the RIP toxin variant or ligand-RIP toxin variant conjugate; insect cell systems infected with recombinant virus expression vectors (e.g., baculovirus) containing the nucleic acid molecule that encodes the RIP toxin variant or ligand-RIP toxin variant conjugate; or animal cell systems transformed with recombinant plasmid expression vectors containing the nucleic acid molecule that encodes the RIP toxin variant or ligand-RIP toxin variant conjugate or infected with recombinant virus expression vectors (e.g., DNA or RNA viruses, such as, but not limited to retroviruses, adenoviruses, and vaccinia viruses) containing the nucleic acid molecule that encodes the RIP toxin variant or ligand-RIP toxin variant conjugate, or transformed animal cell systems engineered for stable expression of the RIP toxin variant or ligand-RIP toxin variant conjugate.

Depending on the host/vector system used, any of a number of suitable transcription and translation elements, including constitutive and inducible promoters, transcription enhancer elements, transcription terminators, etc. can be operably linked to the nucleic acid encoding the REP toxin variant or ligand-RIP toxin variant conjugate in the expression vector (see, e.g., Bitter et al., Methods in Enzymology 153: 516-544, 1987). For example, when a bacterial system is used, inducible promoters such as, but not limited to, P_{L} of bacteriophage S, P_{LAC}, P_{TRP}, P_{TAC} (P_{TRP-LAC} hybrid promoter), or T7, can be used. In another example, when a mammalian cell system is used, promoters derived from the genome of mammalian cells (e.g., metallothionein promoter) or from mammalian viruses (e.g., the retrovirus long terminal repeat, the adenovirus late promoter, or the vaccinia virus 7.5K promoter) can be used. Promoters produced by recombinant nucleic acid or synthetic techniques also can be used to provide for transcription of the inserted nucleic acid molecule encoding the RIP toxin variant or ligand-RIP toxin variant conjugate.

When the host is prokaryotic, such as E. coli, competent cells that are capable of DNA uptake (i.e. transformation) can be prepared from cells by procedures well known in the art. For example, cells can be harvested after exponential growth phase and subsequently treated by a CaCl₂ method. Alternatively, MgCl₂ or RbCl can be used. Transformation also can be performed after forming a protoplast of the host cell or by electroporation. Generally a prokaryotic host is used as the host cell.

When the host is a eukaryotic cell, methods of transfection of recombinant nucleic acid molecules include formation of calcium phosphate co-precipitates and conventional mechanical procedures, such as microinjection, electroporation, and insertion of plasmid encased in liposomes. Another method of nucleic acid transfer involves the use of a eukaryotic viral vector, such as simian virus 40 (SV40), adenovirus, vaccinia virus, bovine papilloma virus, or recombinant autonomous parvovirus vector (e.g., as described in U.S. Pat. No. 5,585,254) to transiently infect, or transform, eukaryotic cells and express the protein (Eukaryotic Viral Vectors, Cold Spring Harbor Laboratory, Gluzman ed., 1982). Eukaryotic cells also can be cotransfected with a nucleic acid molecule encoding the RIP toxin variant or ligand-RIP toxin variant conjugate polypeptide and a second nucleic acid molecule encoding a selectable phenotype, such as the Herpes simplex thymidine kinase gene. Alternatively, the nucleic acid molecule encoding the RIP toxin variant or ligand-RIP toxin variant conjugate polypeptide and the nucleic acid molecule encoding a selectable phenotype are present on the same vector or plasmid.

Eukaryotic expression systems can allow for further post-translational modifications of expressed mammalian proteins to occur. Such cells possess the cellular machinery for post-translational processing of the primary transcript, if so desired. Such modifications include, but are not limited to, glycosylation, phosphorylation, and farnesylation. Such host cell lines can include but are not limited to CHO, VERO, BHK, HeLa, COS, MDCK, Jurkat, HEK-293, and WI38.

i. Bacterial Cell Expression Systems

In bacterial systems, a number of expression vectors can be advantageously selected depending upon the desired attributes of the system. For example, when large quantities of the RIP toxin variant or ligand-RIP toxin variant conjugate protein are to be produced, vectors which direct the expression of high levels of the RIP toxin variant or ligand-RIP toxin variant conjugate protein products that are readily purified can be desirable. Those which are engineered to contain a cleavage site to aid in recovering the expressed polypeptide are preferred. Excellent results can and have been obtained using several commercially available vectors, including pET 11a, b, c, or d (Novagen, Madison, Wis.).

Particularly preferred plasmids for transformation of E. coli cells include the pET expression vectors (see, e.g., U.S. Pat. No. 4,952,496; available from NOVAGEN, Madison, Wis.; see, also literature published by Novagen describing the system). Such plasmids include pET 11c and/or pET 11a, which contains the T7lac promoter, T7 terminator, the inducible E. coli lac operator, and the lac repressor gene; pET 12a-c, which contains the T7 promoter, T7 terminator, and the E. coli ompT secretion signal; and pET 15b (Novagen, Madison, Wis.), which contains a His-Tag™ leader sequence (Seq. ID NO. 40) for use in purification with a His column and a thrombin cleavage site that permits cleavage following purification over the column; the T7-lac promoter region and the T7 terminator.

Nucleic acid encoding a targeting agent, such as a chemokine, linked to a targeted agent with and without linkers, and other such constructs, can be inserted into the pET vectors, such as pET11c, pET-11a, and pET-15b expression vectors (NOVAGEN, Madison, Wis.), for intracellular or periplasmic expression of the RIP toxin variant or ligand-RIP toxin variant conjugate proteins. Alternatively, the targeted agent or targeting agents can be inserted in the pET vectors and expressed individually.

Other plasmids include the pKK plasmids, particularly pKK 223-3, which contains the tac promoter, (available from Pharmacia; see also, Brosius et al. (1984) Proc. Natl. Acad. Sci. 81: 6929; Ausubel et al. Current Protocols in Molecular Biology; and U.S. Pat. Nos. 5,122,463, 5,173,403, 5,187,153, 5,204,254, 5,212,058, 5,212,286, 5,215,907, 5,220,013, 5,223,483, and 5,229,279), which contain the tac promoter. Plasmid pKK has been modified by insertion of a kanamycin resistance cassette with EcoRI sticky ends (purchased from Pharmacia; obtained from pUC4K (see, e.g., Vieira et al. (1982) Gene 19:259-268; and U.S. Pat. No. 4,719,179) into the ampicillin resistance marker gene.

Other preferred vectors include, but are not limited to, the PPL-lambda inducible expression vector and the tac promoter vector pDR450 (see, e.g., U.S. Pat. Nos. 5,281,525, 5,262,309, 5,240,831, 5,231,008, 5,227,469, 5,227,293; available from Pharmacia P.L. Biochemicals, see; also Mott, et al. (1985) Proc. Natl. Acad. Sci. U.S.A. 82:88; and De Boer et al. (1983) Proc. Natl. Acad. Sci. U.S.A. 80:21); and baculovirus vectors, such as a pBlueBac vector (also called pJVETL and derivatives thereof; see, e.g., U.S. Pat. Nos. 5,278,050, 5,244,805, 5,243,041, 5,242,687, 5,266,317, 4,745,051, and 5,169,784), including pBlueBac III.

Other vectors include, but are not limited to, the pIN-IIIompA plasmids, such as pIN-IIIompA2 (see, e.g., U.S. Pat. No. 4,575,013 and Duffaud et al. (1987) Meth. Enzymology 153: 492-507). The pIN-IIIompA plasmids include an insertion site for heterologous DNA linked in transcriptional reading frame with functional fragments derived from the lipoprotein gene of E. coli. The plasmids also include a DNA fragment that encodes the signal peptide of the ompA protein of E. coli, positioned such that the desired polypeptide is expressed with the ompA signal peptide at its amino terminus, thereby allowing efficient secretion across the cytoplasmic membrane. The plasmids further include DNA encoding a specific segment of the E. coli lac promoter-operator, which is positioned in the proper orientation for transcriptional expression of the desired polypeptide, as well as a separate functional E. coli lacI gene encoding the associated repressor molecule that, in the absence of lac operon inducer, interacts with the lac promoter-operator to prevent transcription therefrom. Expression of the desired polypeptide is under the control of the lipoprotein (lpp) promoter and the lac promoter-operator, although transcription from either promoter is normally blocked by the repressor molecule. The repressor is selectively inactivated by means of an inducer molecule thereby inducing transcriptional expression of the desired polypeptide from both promoters.

The repressor protein can be encoded by the plasmid containing the construct or a second plasmid that contains a gene encoding for a repressor-protein. The repressor-protein is capable of repressing the transcription of a promoter that contains sequences of nucleotides to which the repressor-protein binds. The promoter can be derepressed by altering the physiological conditions of the cell. The alteration can be accomplished by the addition to the growth medium of a molecule that inhibits, for example, the ability to interact with the operator or with regulatory proteins or other regions of the DNA or by altering the temperature of the growth media. Preferred repressor-proteins include, but are not limited to, the E. coli lacI repressor responsive to IPTG induction, the temperature sensitive c1857 repressor. The E. coli lacI repressor is preferred.

In certain embodiments, the constructs also include a transcription terminator sequence. The promoter regions and transcription terminators are each independently selected from the same or different genes. In some embodiments, the DNA fragment is replicated in bacterial cells, such as in E. coli. The DNA fragment also typically includes a bacterial origin of replication, to ensure the maintenance of the DNA fragment from generation to generation of the bacteria. In this way, large quantities of the DNA fragment can be produced by replication in bacteria. Preferred bacterial origins of replication include, but are not limited to, the f1-ori and colE1 origins of replication.

Exemplary bacterial hosts contain chromosomal copies of DNA encoding T7 RNA polymerase operably linked to an inducible promoter, such as the lacUV promoter (see, U.S. Pat. No. 4,952,496). Such hosts include, but are not limited to, lysogens E. coli strains HMS174(DE3)pLysS, BL21(DE3)pLysS, HMS174(DE3) and BL21(DE3). Strain BL21(DE3) is preferred. The pLys strains provide low levels of T7 lysozyme, a natural inhibitor of T7 RNA polymerase. Preferred bacterial hosts are the insect cells Spodoptera frugiperda (sf9 cells; see, e.g., Luckow et al. (1988) Biotechnology 6:47-55 and U.S. Pat. No. 4,745,051).

Expression systems employing bacterial hosts are easily scaleable for small or large scale protein production. For large scale protein production, methods, such as batch fermentation, can be used to express recombinant proteins, such as the modified RIP toxins or ligand-RIP toxin conjugates provided herein. Exemplary methods of batch fermentation are known in the art and also can be found, for example, in the Examples provided herein. For example, bacterial host cells that contain expression vectors, such as a pET vector carrying a nucleic acid molecule that encodes a RIP toxin or ligand-RIP toxin conjugate provided herein, can be grown in vessels, such as fermentors, for batch fermentation. Typically such fermentors are used for growth of bacteria in 5 to 100 liters or more of liquid culture. The liquid culture used for growth is typically a standard enriched media culture, which can optionally contain additional components that enhance growth of the bacteria and/or production of the expressed protein. For example, RIP toxin inhibitors, such as 4-APP, can be added to the culture to enhance the growth of bacteria that express RIP toxins or ligand-RIP toxin conjugate proteins. As described elsewhere herein, addition of 4-APP inhibits the toxic activities of the expressed RIP toxin on the host bacterial cells, thereby allowing for higher protein production. For protein expression when using inducible vectors, such as a pET vector, an inducing agent (e.g., IPTG) is typically added to the culture for a period of time once the culture reaches a particular density of growth. The concentration of inducing agent and length of induction time can be empirically determined or experimentally determined using methods well known in the art for determining optimal growth conditions for protein expression. Methods for purification of expressed proteins from bacterial host cells is well known in the art and can include, for example, solubilization with a homogenizer in a suitable solubilization buffer, such as a strong denaturing solution (e.g., guanidine hydrochloride/urea solution) that optionally includes a detergent, followed by column purification. An exemplary method for purification of RIP toxins and ligand-RIP toxin conjugates is provided in the Examples herein.

ii. Insect Cell Expression Systems

An alternative expression system that can be used to express the RIP toxin variant or ligand-RIP toxin variant conjugate protein is an insect system. In one such system, Autographa californica nuclear polyhedrosis virus (AcNPV) is used as a vector to express foreign genes. The virus grows in Spodoptera frugiperda cells. The nucleic acid encoding the RIP toxin variant or ligand-RIP toxin variant conjugate can be cloned into non-essential regions (for example, the polyhedrin gene) of the virus and placed under control of an AcNPV promoter (for example the polyhedrin promoter). Successful insertion of nucleic acid encoding the RIP toxin variant or ligand-RIP toxin variant conjugate will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (see e.g., U.S. Pat. No. 4,215,051).

For insect hosts, baculovirus vectors, such as a pBlueBac (also called pJVETL and derivatives thereof) vector, particularly pBlueBac III, (see, e.g., U.S. Pat. Nos. 4,745,051, 5,242,687, 5,243,041, 5,244,805, 5,266,317, 5,270,458, 5,278,050, and 5,169,784; and published International PCT Application WO 93/10139; available from Invitrogen, San Diego) also can be used for expression of the polypeptides. The pBlueBacIII vector is a dual promoter vector and provides for the selection of recombinants by blue/white screening as this plasmid contains the β-galactosidase gene (lacZ) under the control of the insect recognizable ETL promoter and is inducible with IPTG. The DNA construct introduced into the pBlueBac III baculovirus vector is operably linked to the polyhedrin promoter to generate the expression plasmid, which is then co-transfected with wild type virus into insect cells Spodoptera frugiperda (sf9 cells; see, e.g., Luckow et al. (1988) Biotechnology 6: 47-55 and U.S. Pat. No. 4,745,051). Blue occlusion minus viral plaques are selected and plaque purified and screened for the presence of the DNA molecule encoding the conjugate protein by any standard methodology, such as western blots using appropriate anti-sera or Southern blots using an appropriate probe. Selected purified recombinant virus is then co-transfected, such as by CaPO₄ transfection or liposomes, into Spodoptera frugiperda cells (sf9 cells) with wild type baculovirus and grown in tissue culture flasks or in suspension cultures.

iii. Yeast Cell Expression Systems

Another expression system that can be used to express the REP toxin variant or ligand-RIP toxin variant conjugate protein is yeast. In yeast, a number of vectors containing constitutive or inducible promoters can be used. Such vectors are well known (see, e.g., techniques described in Molecular Cloning: A Laboratory Manual, Sambrook et al., eds., 2nd ed., Cold Spring Harbor Laboratory, N.Y., 1989; Bitter, et al. (1987) Methods in Enzymol. 153: 516-544; Bitter et al. (1987) Methods in Enzymol., 152: 673-684; Rothstein, DNA Cloning, Vol. II, Glover, D. M., ed., IRL Press, Wash., D.C., Ch. 3, 1986; and The Molecular Biology of the Yeast Saccharomyces, Strathern et al., eds., Cold Spring Harbor Press, Vols. I and II, 1982). A constitutive yeast promoter such as ADH or LEU2 or an inducible promoter such as GAL can be used (see e.g., Rothstein, DNA Cloning, Vol. II, Glover, D. M., ed., IRL Press, Wash., D.C., Ch. 3, 1986). Alternatively, vectors that promote integration of foreign DNA sequences into the yeast chromosome can be used.

iv. Plant Cell Expression Systems

Another expression system that can be used to express the REP toxin variant or ligand-RIP toxin variant conjugate protein is a plant cell system. In cases where plant expression vectors are used, the expression of a DNA molecule encoding a conjugate protein can be driven by any of a number of promoters. For example, viral promoters such as the 35S RNA and 19S RNA promoters of CaMV (see e.g., Brisson et al., (1984) Nature 310: 511-514), or the coat protein promoter to TMV (see e.g., Takamatsu et al. (1987) EMBO J. 6: 307-311) can be used; alternatively, plant promoters such as the small subunit of RuBisCO (see e.g., Coruzzi et al. (1984) EMBO J. 3: 1671-1680 and Broglie et al. (1984) Science 224: 838-843); or heat shock promoters, e.g., soybean hsp17.5-E or hsp17.3-B (see e.g., Gurley, et al. (1986) Mol. Cell. Biol. 6: 559-565) can be used. These constructs can be introduced into plant cells using Ti plasmids, Ri plasmids, plant virus vectors, direct DNA transformation, microinjection, electroporation, among other techniques. For reviews of such techniques see, for example, Weissbach and Weissbach (1988) Methods for Plant Molecular Biology, Academic Press, NY, Section VIII, pp. 421-463 and Plant Molecular Biology, 2d Ed., Covet, S, N., Ed., Ch. 7-9, Blackie, London 1988.

v. Mammalian Cell Expression Systems

Another expression system that can be used to express the RIP toxin variant or ligand-RIP toxin variant conjugate protein is a mammalian cell system. Expression constructs can be transferred to mammalian cells by viral infection, such as adenovirus or vaccinia virus, or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high level expression, for example the SV40 promoter-enhancer, the human cytomegalovirus (CMV) promoter, and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha-fetoprotein, alpha 1-antitrypsin, beta-globin, myelin basic protein, myosin light chain-2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ζ and Fc_{ε}RI-γ can direct expression of the proteins in an active state on the cell surface.

Many cell lines are available for mammalian expression including mouse, rat human, monkey, and chicken and hamster cells. Exemplary cell lines include, but are not limited to, CHO, VERO, BHK, HT1080, MDCK, W138, Balb/3T3, HeLa, MT2, mouse NS0 (non-secreting) and other myeloma cell lines, hybridoma and heterohybridoma cell lines, lymphocytes, RPMI 1788 cells, fibroblasts, Sp2/0, COS, NIH3T3, HEK293, 293S, 2B8, EBNA-1, and HKB cells (see e.g. U.S. Pat. Nos. 5,618,698, 6,777,205). Cell lines also are available adapted to serum-free media which facilitates purification of secreted proteins from the cell culture media (e.g., EBNA-1, Pham et al., (2003) Biotechnol. Bioeng. 84:332-42).

Mammalian cell systems that use recombinant viruses or viral elements to direct expression can be engineered. For example, when using adenovirus expression vectors, nucleic acid encoding the RIP toxin variant or ligand-RIP toxin variant conjugate can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing nucleic acid encoding the RIP toxin variant or ligand-RIP toxin variant conjugate in infected hosts (e.g., see Logan and Shenk (1984) Proc. Natl. Acad. Sci. USA, 81: 3655-3659). Alternatively, the vaccinia virus 7.5K promoter can be used (see e.g., Mackett et al. (1982) Proc. Natl. Acad. Sci. USA, 79: 7415-7419; Mackett et al. (1984) J. Virol. 49: 857-864, 1984; and Panicali et al. (1982) Proc. Natl. Acad. Sci. USA, 79: 4927-4931). Of particular interest are vectors based on bovine papilloma virus which have the ability to replicate as extrachromosomal elements (Sarver, et al., Mol. Cell. Biol. 1: 486-96, 1981). Shortly after entry of this DNA into mouse cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as the neo gene. Alternatively, the retroviral genome can be modified for use as a vector capable of introducing and directing the expression of the REP toxin variant or ligand-RIP toxin variant conjugate in host cells (Cone and Mulligan, Proc. Natl. Acad. Sci. USA, 81:6349-6353, 1984). High level expression also can be achieved using inducible promoters, including, but not limited to, the metallothionein IIA promoter and heat shock promoters.

For long-term, high-yield production of recombinant proteins, stable expression is preferred. Rather than using expression vectors which contain viral origins of replication, host cells can be transformed with cDNA encoding the RIP toxin variant or ligand-RIP toxin variant conjugate protein controlled by appropriate expression control elements (e.g., promoter, enhancer, sequences, transcription terminators, polyadenylation sites, etc.), and a selectable marker. The selectable marker in the recombinant plasmid confers resistance to the selection and allows cells to stably integrate the plasmid into their chromosomes and grow to form foci which in turn can be cloned and expanded into cell lines. For example, following the introduction of foreign DNA, engineered cells can be allowed to grow for 1-2 days in an enriched media, and then are switched to a selective media. A number of selection systems can be used, including but not limited to the Herpes simplex virus thymidine kinase (Wigler et al., Cell, 11: 223-32, 1977), hypoxanthine-guanine phosphoribosyltransferase (Szybalska and Szybalski (1982) Proc. Natl. Acad. Sci. USA, 48: 2026-30), and adenine phospho-ribosyltransferase (Lowy et al. (1980) Cell 22: 817-31) genes can be employed in tk⁻, hgprt⁻ or aprt⁻ cells respectively. Also, antimetabolite resistance can be used as the basis of selection for dhfr, which confers resistance to methotrexate (Wigler et al. (1980) Proc. Natl. Acad. Sci. USA 78: 3567-70; O'Hare et al. (1981) Proc. Natl. Acad. Sci. USA, 8: 1527-31, 1981); gpt, which confers resistance to mycophenolic acid (Mulligan and Berg (1981) Proc. Natl. Acad. Sci. USA 78: 2072-6; neo, which confers resistance to the aminoglycoside G-418 (Colberre-Garapin et al. (1981) J. Mol. Biol. 150:1-14); and hygro, which confers resistance to hygromycin (Santerre et al. (1984) Gene 30: 147-56) genes. Recently, additional selectable genes have been described, namely trpB, which allows cells to use indole in place of tryptophan; hisD, which allows cells to use histinol in place of histidine (Hartman and Mulligan (1988) Proc. Natl. Acad. Sci. USA 85: 8047-51); and ODC (ornithine decarboxylase) which confers resistance to the ornithine decarboxylase inhibitor, 2-(difluoromethyl)-DL-ornithine, DFMO (McConlogue and Coffino (1983) J. Biol. Chem. 258: 8384-8388).

b. Purification

Techniques for the isolation and purification of expressed modified RIP toxins or ligand-RIP toxin conjugates from host cells depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary further the proteins can be extracted and further purified using standard methods in the art.

In some instances, prior to purification, conditioned media containing the secreted fusion polypeptide, including ligand-toxin conjugates, can be obtained. The conditioned media can be tested in neat form. In other examples, the conditioned media can be clarified and/or concentrated. Clarification can be by centrifugation followed by filtration. Concentration can be by any method known to one of skill in the art, such as for example, using tangential flow membranes or using stirred cell system filters. Various molecular weight (MW) separation cut offs can be used for the concentration process. For example, a 10,000 MW separation cutoff can be used.

Modified RIP toxins or ligand-RIP toxin conjugates produced either by prokaryotes or eukaryotes can be effected using standard protein purification techniques known in the art including but not limited to, SDS-PAGE, differential precipitation, diafiltration, ultrafiltration, column electrofocusing, flat-bed electrofocusing, gel filtration, isotachophoresis, size fractionation, ammonium sulfate precipitation, high performance liquid chromatography, chelate chromatography, adsorption chromatography, ionic exchange chromatography (e.g., cationic, anionic), hydrophobic interaction chromatography, and molecular exclusion chromatography. Affinity purification techniques also can be used to improve the efficiency and purity of the preparations. For example, use of monoclonal or polyclonal antibodies, receptors and other molecules that bind modified RIP toxins or ligand-RIP toxin conjugates can be used in affinity purification. Expression constructs also can be engineered to add an affinity tag such as a myc epitope, GST fusion or His₆ and affinity purified with myc antibody, glutathione resin, and Ni-resin, respectively, to a protein. Purity can be assessed by any method known in the art including gel electrophoresis and staining and spectrophotometric techniques.

Following transformation, large amounts of the protein can be isolated and purified in accordance with conventional methods. For example, a lysate can be prepared from the expression host and the desired protein (e.g., a ligand-RIP toxin variant) purified using HPLC, exclusion chromatography, gel electrophoresis, affinity chromatography, or other purification techniques. The purified protein will generally be about 80% to about 90% pure, and can be up to and including 100% pure. Pure is intended to mean free of other proteins, as well as cellular debris.

In some examples, a selected purification method can affect the protein structure and thus additional preparation steps are needed following purification to generate the desired recombinant protein. For example, some expressed proteins require refolding following purification techniques that employ strong denaturation conditions. Methods for refolding proteins are known in the art and can include, for example, dialysis, in the presence of low levels of reducing agent (see e.g., Example 4).

3. Production of Chemical Conjugates

To effect chemical conjugation herein, the targeting agent is linked via one or more selected linkers or directly to the targeted agent. Chemical conjugation can be used if the targeted agent and the targeting agent are expressed as separate polypeptides and must be used if the targeted agent is other than a peptide or protein, such a nucleic acid or a non-peptide drug. Any means known to those of skill in the art for chemically conjugating selected moieties can be used. Several methods are described elsewhere herein and include, but are not limited to, crosslinking agents such homo- and heterobifunctional linking compounds.

The nucleic acid molecules encoding the RIP toxin variants or targeting agents also can be modified to facilitate post-translational chemical conjugation of the targeted agent, such as a modified RIP toxin variant provided herein, to the targeting agent. For example the nucleic acid molecules that encode the RIP toxin variant or targeting agent can be fused to nucleic acid molecules that encode linker polypeptides that can link the RIP toxin to the targeting agent following expression and, optionally, purification of the REP toxin and the targeting agent. In another example, the nucleic acid molecules that encode RIP toxin variants or targeting agents can be modified to mutate particular codons to generate amino acids in the polypeptides which can be used as sites for chemical modification and attachment of polypeptides, such as linkers, for conjugation. More specifically, by removing and/or introducing an amino acid residue containing an attachment group for the linker moiety it is possible to specifically adapt the polypeptide so as to make the molecule more susceptible to conjugation to linker moiety of choice (see e.g., U.S. Patent Publication No. 20060252690)

### H. METHODS TO INCREASE PRODUCTION OF RIP POLYPEPTIDES, OR CONJUGATES THEREOF

Provided herein is a method for increasing the production of a recombinantly expressed RIP toxin or ligand-RIP toxin conjugate or variants thereof by reducing the toxic activity of the RIP toxin in order to allow the host to produce increased quantities of the toxic polypeptide. In such methods, a RIP toxin, or conjugate thereof, such as any provided herein, can be produced as described, for example, in Section G above, and in the presence of one or more RIP inhibitor. Any RIP toxin inhibitor known to one of skill in the art, or subsequently identified hereto, which can inactivate a REP toxin can be used in the methods provided herein. As described elsewhere herein, exemplary RIP toxin inhibitors include, for example, REP-specific oligonucleotide inhibitors, such as RNA aptamers, RIP-specific antibodies, and/or adenine isomers including, for example, adenine, 4-aminopyrazolo[3,4-d]pyrimidine (4-APP), and other similar isomers. For the methods provided herein, typically, such RIP toxin inhibitors are any that inhibit toxic activity by targeting the conserved N-glycosidase activity of RIP toxins. For purposes herein, any RIP inhibitor, such as adenine or any analog thereof can be used in the methods herein so long as the inhibitor exhibits an inhibitory activity against the RIP toxin, or conjugate thereof. Accordingly, a REP inhibitor, such as 4-APP, can be used in the methods herein for protein production of a RIP toxin, a ligand-RIP toxin conjugate, or variants thereof, including, but not limited to, a modified SA1, saporin, momordin, or bryodin.

The choice of RIP inhibitor used in the method of improving production provided herein are dependent on a number of factors including, but not limited to, the choice of host cell employed for recombinant protein expression and the specific RIP polypeptide to be expressed. The specificity of RIP inhibitors for a given RIP polypeptide is known, or can be determined based on routine assays to assess toxicity of a RIP polypeptide. A discussion of RIP inhibitor specificity is described elsewhere herein. For example, based on specificity of known and tested adenine analogs, 4-APP is a candidate for use in the methods herein for expression and improved production of Shiga toxin, including the SA1 portion, active fragments thereof, and conjugates thereof. In particular, 4-APP can be used in methods of improved production herein to increase the yield of any modified SA1 polypeptide provided herein, or any conjugates thereof, such as, for example, any LPM conjugate provided herein.

The amount of RIP inhibitor used in the methods of polypeptide expression can be empirically determined based on its known effects on the toxic activity of a RIP toxin, a ligand-RIP toxin conjugate, or variant thereof. It is important that the RIP inhibitor used in the methods herein is itself not toxic to the specific host cell, which toxicity is known or can be determined by one of skill the art depending on the host cell chosen. Hence, typically in the methods of expression herein, a RIP inhibitor, such as for example 4-APP, is added at about or at 0.1 mM, 0.2 mM, 0.3 mM, 0.4 mM, 0.5 mM, 0.6 mM, 0.7 mM, 0.8 mM, 0.9 mM, 1.0 mM, 1.5 mM, 2.0 mM, 3.0 mM, 4.0 mM, 5.0 mM, 10 mM, 15 mM, 20 mM, 30 mM, 40 mM, 50 mM or more so long as the inhibitor is itself not toxic to the host cell chosen. It is understood that the concentration of the RIP inhibitor chosen can vary depending on the host cell chosen, the conditions used for recombinant expression, the time of duration of incubation with the inhibitor, the particular RIP inhibitor chosen, and/or the particular RIP toxin or ligand-toxin conjugate that is being produced.

In one example, the concentration of RIP inhibitor can be empirically determined by performing a dose-response experiment and assaying for the amount of protein expressed at each concentration of inhibitor. For example, Example 4 herein describes such an exemplary experiment for the determination of an optimal concentration of 4-APP to be used in the production of various LPMs by assessing expression profiles of the various LPMs by Coomassie Blue Staining following their expression in the presence of increased concentrations of 4-APP. As exemplified in Example 4, the results show some differences in the level of polypeptide expressed between different LPM conjugates in the presence of increasing concentrations of 4-APP. To determine the concentration of RIP inhibitor to use in the method of production, one could perform a similar experiment using any RIP polypeptide, or conjugate thereof, and any RIP inhibitor such as, for example, 4-APP, to determine the concentration of the RIP inhibitor that provides maximal protein expression. For example, LPM1d is expressed at maximal levels at or about 10 mM or more of 4-APP, while LPM7 is expressed at maximal levels in the presence of at or about 2.0 mM or more of 4-APP. Generally, LPM conjugates, such as those containing a chemokine ligand linked directly or indirectly to a variant 4 modified SA1 moiety, are produced in the methods herein in the presence of at or about 2.0 mM, 3.0 mM, 4.0 mM, 5.0 mM, 10.0 mM or 20 mM of 4-APP.

The RIP inhibitor can be added before, during, or after transformation of the host cells with the nucleic acid that encodes the RIP toxin, a ligand-toxin conjugate, or variant thereof. In the case where an inducing agent is used to induce protein expression, the RIP inhibitor can be added before, during, and/or after introduction of the inducing agent to the host cells. For example, a RIP inhibitor can be added at a single concentration before an inducing agent is added. In another example, a RIP inhibitor can be added at a single concentration before an inducing agent is added, and the medium can be supplemented with additional RIP inhibitor during or after the incubation with the inducing agent. In some cases, the concentrations of RIP inhibitor added to the expression system can vary at different stages according to the specific expression system used. For example, as exemplified in Example 4, the RIP inhibitor 4-APP was added to E. coli cells transformed with nucleic acid encoding an LPM at a concentration of 2.0 mM during the initial overnight and growth of the ligand-toxin conjugate, however, additional 4-APP was added during the incubation with the inducing agent at up to 10-fold higher concentration. As exemplified in Example 4, the concentrations of RIP inhibitor used, and the timing of administration of the RIP inhibitor, can be optimized depending on the specific expression system used.

One or more than one RIP inhibitor can be used in the methods herein for improving production of a RIP toxin, or conjugate thereof. In addition, other methods of improving recombinant protein expression and production, such as any described above, also can be used in the methods herein. For example, any method known in the art that have been used to increase the expression and production of a RIP polypeptide, or conjugate thereof, can be performed in the presence or absence of a RIP inhibitor, such as 4-APP.

Additional Methods to Increase Protein Production

In addition to increasing production of a RIP polypeptide or ligand-toxin conjugate by using a RIP inhibitor, such as 4-APP, during expression and production of the protein, other methods also can be used. Methods to improve expression and production of polypeptides, such as any RIP polypeptide or conjugate thereof provided herein, include any method known in art. Use of such methods depends on the expression system employed to generate the polypeptides (e.g., bacterial, yeast, mammalian, insect, plant etc.) and can involve modification of factors such as choice of expression vectors (e.g. for regulated or constitutive expression), growth conditions of the host cells, or protein induction parameters. The methods of purification as mentioned above for isolation of the expressed polypeptides from host cells (e.g., methods of host cell lysis and protein separation) also can be optimized by variations known in the art to improve the amount of protein generated. Exemplary of additional methods to improve production are discussed below.

Growth conditions of the host cells can be altered, for example, by a variety of methods including, but not limited to, changes in pH, temperature, atmospheric content (e.g. oxygen or carbon dioxide concentration), media content, including osmolarity, nutrient concentrations (e.g. glucose and other sugars, minerals, and phosphates or other ions), or presence of other molecules that affect host growth (e.g., antibiotics, antiviral or antimicrobial compounds, protein inhibitors etc.). Modifications to the growth conditions also can be made to decrease the production of inhibitor molecules, such as sulfates, that can affect protein expression (see e.g., U.S. Pat. No. 6,686,180).

Induction parameters can be altered, for example, by changes in the concentration of inducing agent (e.g. IPTG or other inducer molecule, temperature, oxygen content etc), length of induction time, temperature of induction, concentration of host cells at time of induction, and influence of host background on levels of expression.

Choice of host cells also can affect levels of protein production. For example, bacterial strains are available that differ in genetic backgrounds which can affect protein production. Such differences include, but are not limited to, mutations in proteases (e.g. lon and ompT), recombinases (e.g., recA), or endonucleases (e.g., endA), mutations that improve disulfide bond formation and protein folding (e.g., trxB/gor), presence of DE3 lysogens for T7 promoter-driven expression (e.g., LysE or LysS), and mutations that affect the control of protein induction (e.g. lacZY or lacI^{q}) or sugar usation of the host cell. Host cells also can contain copies of rare tRNA genes to improve recognition of rare codons in the nucleic acid sequence encoding the polypeptide.

Another method to alter levels of expression of a polypeptide provided herein is to modify the nucleic acid that encodes the polypeptide or to alter the expression vector that contains the nucleic acid molecule that encodes the polypeptide. As described elsewhere herein and known in the art, many vectors are available for the expression of polypeptides provided herein, including the RIP toxin variants and ligand-RIP toxin variants provided herein. Methods for improving the production of the polypeptides provided herein include selection of a vector with properties, such as, but not limited to, a strong promoter for high level of expression, a regulatable promoter to control to timing of expression, a constitutive promoter for continuous expression, or a stable promoter for long term expression. Use of a vector that allows for high levels of protein expression and tight regulation is preferred for expression of toxic proteins, such as the RIP toxin variants and the ligand RIP toxin variants provided herein. Examples of such vectors are known in the art and include, for example, pET vectors, as described elsewhere herein and in the Examples, vectors with anaerobically regulated promoters (e.g. nirB) and L-rhamnose inducible vectors, which are repressed by D-glucose (pET vectors are commercially available from Novagen; Debinski et al., (1991) Mol. Cell. Biol. 11:3:1751-1753; Debinski and Pastan (1992) Cancer Res. 52: 5379 5385; Debinski et al. (1992) J. Clin. Invest. 90:405-411; Oxer et al. (1991) Nucl. Acids Res. 19(11) 2889-2892; Giacalone et al. (2006) Biotechniques 40 (3): 355-363).

The nucleic acid encoding the polypeptide also can be modified to contain mutations in codons that encode the amino acids of the polypeptide such that codons that are rare in the host in which the polypeptide is to be expressed are mutated to codons that are more common in the host, without altering the encoded amino acid. Use of a higher usage codon for a particular host can improve the production of the polypeptide by improving the rate of translation of the polypeptide. Codon usage frequencies for particular hosts, such as bacterial hosts, are known in the art and can be used to generate optimized nucleic acids that encode the polypeptides provided herein.

### I. IN VITRO AND IN VIVO ASSAYS TO MEASURE ACTIVITY OF TOXINS CONJUGATES

Generally, the ligand-toxin conjugates provided herein exhibit toxic activity against one or more host cells and/or exhibit one or more other activities such as via virtue of their ability to target and bind to a cell surface receptor. As such, the conjugates are candidate therapeutics. If needed, conjugates can be screened using in vitro and in vivo assays to monitor or identify an activity of a toxin conjugate and to select conjugates that exhibit such activity. In vitro assays for testing any conjugate provided herein include any assay to determine if the conjugate displays activity towards particular host cell targeted populations. Such activities include, but are not limited to, toxicity assays, including cell-based toxicity assays, receptor binding assays, cell internalization assay, and chemotaxis assays. Further, a variety of in vivo animal models is known or can be designed to assess the effects of a particular toxin in a specific disease model.

1. In Vitro Activity Assays

a. Cell-Based Toxicity Assays

Conjugates provided herein can be tested for their toxic activity to host cells, such as due to their N-glycosidase activity. Assays to test toxic activity are described in detail in Section D above and include, but are not limited to, assays to assess protein synthesis, depurination of ribosomes, and cell growth or viability of the host cell. For example, the host cell chosen for toxic activity assessment can be one known to express the targeted receptor. Such cells can include those obtained directly from a subject, i.e. from the blood, serum or other tissue source, or any cell line known to express a cell surface receptor. Such cells include activated cells. The cells can be activated in vitro by any number of stimuli and/or can be obtained directly from a subject having a disease or disorder, in particular any inflammatory disease or disorder characterized by activated leukocytes or other cell type. Examples of cell types that can be tested in toxic activity assays include, but are not limited to, any immune cell including, but not limited to, monocytes, macrophages (including alveolar macrophages, microglia, kupffer cells), dendritic cells (including immature or mature dendritic cells or langerhans cells), T cells (including CD4 positive such as, but not limited to, Th1 and/or Th2 cells, or CD8 positive), B cells, eosinophils, basophils, mast cells, natural killer (NK) cells, neutrophils, and endothelial cells, or activated forms thereof. Other cells that can be tested for toxicity to a ligand toxin conjugate include, for example, cancer cells or cancer cell lines such as U251, HT-29, or THP-1 cells. As described above, cell survival (or cell death) of cells can be determined, for example, by the ability to release ATP into the culture medium, by the ability of cells to reduce the vital dye MTT, and/or via the ability to exclude the dye trypan blue.

b. Receptor Binding Assays and Internalization

Ligand-toxin conjugates, such as any chemokine toxin conjugate, for example, any LPM provided herein containing a modified SA1 moiety, are designed to target a cell surface receptor on one or more targeted host cells. Toxin conjugate binding to such cell surface receptors can be assessed directly by assessing binding of a toxin conjugate to cells. In some examples, binding of toxin conjugates to monocytes, macrophages (including alveolar macrophages, microglia), T cells (including Th1 and Th2 cells), B cells, eosinophils, basophiles, dendritic cells, kupffer cells, mast cells, natural killer (NK) cells, neutrophils, and endothelial cells can be determined. If desired, the cells can be activated first with any known activating agent in order to induce the expression of a receptor, such as often occurs under inflammatory and pathogenic conditions observed in various diseases and disorders, prior to performing the binding experiments. The cells tested can be cell lines or primary cells derived from any suitable donor isolated directly from the donor or cultured long term under conditions to induce the appropriate cellular phenotype. In some examples, competitive assays can be employed with the cognate non-conjugated ligand to assess the activity of the toxin conjugate compared to the ligand. For example, if the toxin conjugate LPM1d is tested (containing the chemokine MCP-1 conjugated to a modified SA1), MCP-1 alone can be used in competition assays.

In one example, the ability of toxin conjugates to bind to a host cell known to express a specific cell surface receptor can be assessed by labeling the conjugate with any known detectable agent, such as but not limited to, a fluorescent moiety, a radioactivity moiety, or a tag polypeptide (i.e. Flag, His tag, myc tag). For example, toxin conjugates can be labeled with a fluorescent moiety such as fluorescein isothiocynate (FITC). Increasing concentrations of the FITC-labeled toxin conjugate can be added to any desired cell type and incubated at 4° C. for a designated time, for example, 30 minutes or 1 hour. Upon washing of the cells to remove any unbound toxin conjugate, cell bound fluorescence can be measured by flow cytometry. In some cases, the binding affinity of the toxin conjugate can be determined by comparing the binding affinity of a ligand to the ligand toxin conjugate by dividing the concentration of the toxin conjugate by the concentration of the ligand that give equal mean fluorescent values in the flow cytometry measurements (see e.g., Thompson et al. (2001) Protein Engineering, 14: 1035-1041). Additionally, if desired, the ability of the toxin conjugate to be internalized by cells can be assessed by comparing the fluorescence at 4° C. versus 37° C. The incubation time can be adjusted to ensure that the toxin conjugates are not toxic to the cells during the 37° C. incubation. Other methods of assessing binding and internalization are known to those of skill in the art and include, but are not limited to, use of radioactivity, cell-based ELISAs, and other such assays.

c. Chemotaxis Assays

Toxin conjugates; in particular any one or more of chemokine toxin conjugates such as any LPM conjugate provided herein containing a modified SA1 moiety, can be tested for their ability to modulate the chemotaxis of cells using conventional chemotaxis assays. Such a determination correlates with the ability of the chemokine to bind a cognate chemokine receptor. In such assays, the migration of leukocytes, including activated leukocytes, can be induced by chemokines and measured by counting cells that migrate through a filter using a routine Boyden chamber set up (see e.g., McDonald et al. (2001) IDrugs, 4: 427-442). For example, any desired cell including but not limited to monocytes, macrophages (including alveolar macrophages, microglia), T cells (including Th1 and Th2 cells), B cells, eosinophils, basophiles, dendritic cells, kupffer cells, mast cells, natural killer (NK) cells, neutrophils, and endothelial cells can be plated into the top well of a modified Boyden chamber. Such cells can be cell lines or can be primary cells from any suitable donor isolated directly from the donor or cultured long term under conditions to induce the appropriate cellular phenotype. The lower chambers of the Boyden chamber typically contain culture medium containing the ligand chemokine. In some cases, certain cells are constitutively active and can migrate without any specific exogenous stimulus. Such cells include, for example, THP-1 cells. Hence, if THP-1 cells are used in chemotaxis assays, no exogenous chemokine is required, and the effects of the chemokine conjugate can be compared to active cells present in the bottom chamber via migration and inactive cells remaining in the top chamber (McDonald et al. (2001) IDrugs, 4: 427-442). One or both of the top and bottom wells of the Boyden chamber can be treated with various concentrations of the chemokine toxin conjugate. Following incubation over time from 30 minutes, 1 hour, 2 hours, 5 hours, 10 hours, 15 hours, 24 hours, or more, the number of cells in each well of the chamber (or present on the filter) can be determined. The effects of the chemokine toxin conjugate on the cells in each of the respective chambers can be determined and compared to control wells not containing the toxin conjugate. The absence of cells in one or both of the chambers and/or the absence of migrating active cells in the bottom chamber indicates that the chemokine toxin conjugate is active against the target cell population.

2. In Vivo Animal Models for Testing of Conjugates

The conjugates provided herein, such as any polypeptide conjugated to a modified SA1 or active portion thereof including, for example, any LPM conjugate provided herein containing a modified SA1 moiety, can be used to treat diseases in which chemokines and/or receptors therefor are involved or implicated. Particular conjugates for particular diseases are described herein. One of skill in the art can, if needed, test conjugates in well known models to confirm or identify conjugates for use for particular indications. Animal models of disease are well known. Such models include any animal model of inflammatory diseases, particularly diseases involving activated leukocytes or cells that express chemokine receptors in certain disease states, are contemplated herein to be treated with a ligand-toxin conjugate. Assaying the activity of the toxin conjugates in such animal models can confirm activity and/or to identify those toxin conjugates suitable for treatment of a particular disease or condition contemplated herein.

Ligand-toxin conjugates provided herein, including any LPM conjugate containing a modified SA1 moiety, also can be tested in models of diseases for which other conjugates have been used, such as for example, the mouse xenograft model to identify anti-tumor activity (see, e.g., Beitz et al. (1992) Cancer Research 52:227-230; Houghton et al. (1982) Cancer Res. 42:535-539; Bogden et al. (1981) Cancer (Philadelphia) 48:10-20; Hoogenhout et al. (1983) Int. J. Radiat. Oncol., Biol. Phys. 9:871-879; Stastny et al. (1993) Cancer Res. 53:5740-5744).

Animal models for selecting candidates for treatment of mammals are well known and there are numerous recognized models. In addition, the roles of activated immune cells in these disease states have been demonstrated. Exemplary models for such diseases and conditions include, but are not limited to, those in the following discussion.

a. Spinal Cord Injury (SCI)

Models for testing and demonstrating activity of the conjugates herein for treatment of SCI are known to those of skill in the art. Exemplary references that provide and use animal models of SCI which can be used to test ligand-toxin conjugates, such as LPM conjugates containing a modified SA1 moiety, include, but are not limited to, the following references set forth herein.

Bennett et al. (1999), Spasticity in rats with sacral spinal cord injury, J. Neurotrauma 16:69-84, provides a rat model of muscular spasticity that is minimally disruptive, not interfering with bladder, bowel, or hindlimb locomotor function. Spinal transections were made at the S2 sacral level and, thus, only affected the tail musculature. After spinal transection, the muscles of the tail were inactive for 2 weeks. Following this initial period, hypertonia, hyperreflexia, and clonus developed in the tail, and grew more pronounced with time. These changes were assessed in the alert rat, since the tail is readily accessible and easy to manipulate. Muscle stretch or cutaneous stimulation of the tail produced muscle spasms and marked increases in muscle tone, as measured with force and electromyographic recordings. When the tail was unconstrained, spontaneous or reflex induced flexor and extensor spasms coiled the tail. Movement during the spasms often triggered clonus in the end of the tail. The tail hair and skin were extremely hyperreflexive to light touch, withdrawing quickly at contact, and at times clonus could be entrained by repeated contact of the tail on a surface. Segmental tail muscle reflexes, e.g., Hoffman reflexes (H-reflexes), were measured before and after spinalization, and increased significantly 2 weeks after transection. These results indicate that sacral spinal rats develop symptoms of spasticity in tail muscles with similar characteristics to those seen in limb muscles of humans with spinal cord injury, and thus provide a convenient preparation for studying this condition.

Taoka et al. (1998), Spinal cord injury in the rat, Prog Neurobiol 56:341-58, provides a review of the pathologic mechanisms of trauma-induced spinal cord injury in rats to further development of new therapeutic strategies. Spinal cord injury induced by trauma is a consequence of an initial physical insult and a subsequent progressive injury process that involves various pathochemical events leading to tissue destruction; the latter process should therefore be a target of pharmacological treatment. Recently, activated neutrophils have been shown to be implicated in the latter process of the spinal cord injury in rats. Activated neutrophils damage the endothelial cells by releasing inflammatory mediators such as neutrophil elastase and oxygen free radicals. Adhesion of activated neutrophils to the endothelial cell also could play a role in endothelial cell injury. This endothelial cell injury could in turn induce microcirculatory disturbances leading to spinal cord ischemia. Some therapeutic agents that inhibit neutrophil activation alleviate the motor disturbances observed in the rat model of spinal cord injury. Methylprednisolone (MPS) and GM1 ganglioside, which are the only two pharmacological agents currently clinically available for treatment of acute spinal cord injury, do not inhibit neutrophil activation in this rat model. Taken together, these observations raise a possibility that other pharmacological agents that inhibit neutrophil activation used in conjunction with MPS or GM1 ganglioside can have a synergistic effect in the treatment of traumatic spinal cord injury in humans.

Carlson et al. (1998), Acute inflammatory response in spinal cord following impact injury, Exp Neurol 151:77-88, examines the rostral-caudal distribution of neutrophils and macrophages/microglia at 4, 6, 24, and 48 h after contusion injury to the T10 spinal cord of rat (10 g weight, 50 mm drop). Neutrophils were located predominantly in necrotic regions, with a time course that peaked at 24 h as measured with assays of myeloperoxidase activity (MPO). The sharpest peak of MPO activity was localized between 4 mm rostral and caudal to the injury. Macrophages/microglia were visualized with antibodies against ED1 and OX-42. Numerous cells with a phagocytic morphology were present by 24 h, with a higher number by 48 h. These cells were predominantly located within the gray matter and dorsal funiculus white matter. The number of cells gradually declined through 6 mm rostral and caudal to the lesion. OX-42 staining also revealed reactive microglia with blunt processes, particularly at levels distant to the lesion. The number of macrophages/microglia was significantly correlated with the amount of tissue damage at each level.

Expression of pro-inflammatory cytokine and chemokine mRNA upon experimental spinal cord injury in mouse: an in situ hybridization study, Bartholdi et al. (1997) Eur J Neurosci 9:1422-38, describes a study of the expression pattern of proinflammatory and chemoattractant cytokines in an experimental spinal cord injury model in mouse. In situ hybridization shows that transcripts for the proinflammatory cytokines TNF alpha and IL-1 as well as the chemokines MIP-1α and MIP-1β are upregulated within the first hour following injury. In this early phase, the expression of the pro-inflammatory cytokines is restricted to cells in the surroundings of the lesion area probably resident CNS cells. While TNF alpha is expressed in a very narrow time window, IL-1 can be detected in a second phase in a subset of polymorphonuclear granulocytes which immigrate into the spinal cord around 6 h. Message for the chemokines MIP-1α and -β is expressed in a generalized way in the grey matter of the entire spinal cord around 24 h and gets again restricted to the cellular infiltrate at the lesion site at 4 days following injury. The data indicate that resident CNS cells, most probably microglial cells, and not peripheral inflammatory cells, are the main source for cytokine and chemokine mRNAs. The defined cytokine pattern observed indicates that the inflammatory events upon lesioning the CNS are tightly controlled. The very early expression of pro-inflammatory cytokine and chemokine messages can represent an important element of the recruitment of inflammatory cells.

Morphometric analysis of blood vessels in chronic experimental spinal cord injury: hypervascularity and recovery of function, Blight et al. (1991), J Neurol Sci 106:158-74, provides a model of spinal cord trauma in guinea pigs, based on compression to a set thickness, which was described previously. Compression injuries of the lower thoracic cord were produced in 11 anesthetized, adult guinea pigs, and the outcome monitored, using successive behavioral tests and morphometry of the lesion at 2-3 months. This report describes changes in the vascularity of the spinal cord, based on light microscopic analysis of 1 micron plastic transverse sections through the center of the lesion. Mean blood vessel density in these lesions was approximately twice that found in equivalent regions of normal, uninjured spinal cords, and hypervascularity of the white matter extended at least four spinal cord segments cranially and caudally from the lesion center. Capillary diameter distribution was significantly shifted to larger values and large perivascular spaces surrounded most capillaries and pre- and post-capillary vessels. Extent of hypervascularity was not correlated with the overall severity of the injury, but there was a significant positive correlation between the density of blood vessels in the outer 400 microns of the white matter and secondary loss of neurological function below the lesion, seen between one day and eight weeks after injury. These data indicate that hypervascularization of the lesion is related to secondary pathological mechanisms in spinal cord injury, possibly inflammatory responses, that are relatively independent of the primary mechanical injury but more closely connected with loss and recovery of function.

Increased levels of the excitotoxin quinolinic acid in spinal cord following contusion injury, Blight et al. (1993), Brain Res 632:314-6, shows that products of inflammatory phagocytes are potential contributors to secondary pathology following spinal cord trauma, and presents a study quantifying the levels of the neurotoxin and product of activated macrophages, quinolinic acid (QUIN), in the lower thoracic spinal cord of adult guinea pigs 5 days after brief compression injury. At the injured site (T13), elevations in tissue QUIN levels (>10-fold) accompanied proportional increases in the activity of indoleamine-2,3 dioxygenase (>2-fold) and the concentrations of L-kynurenine (>2.5-fold). In contrast, no significant changes occurred in two uninjured regions examined compared to controls, namely cervical spinal cord (C2) and the somatosensory cortex.

Forbes et al. (1994), Inhibition of neutrophil adhesion does not prevent ischemic spinal cord injury, Ann Thorac Surg 58:1064-8, relies on animal models to show that paraplegia can occur after transient aortic occlusion as a consequence of primary ischemia to the spinal cord or injury during the reperfusion period. In animal models of ischemia/reperfusion there is evidence that reperfusion injury can be modulated partially by neutrophils. The efficacy of the neutrophil adherence blocking murine monoclonal antibody (MAb 60.3) was assessed in spinal cord ischemia/reperfusion in rabbits. Spinal cord ischemia was accomplished by balloon catheter occlusion of the infrarenal aorta. Neurologic assessment was graded as normal, partial neurologic deficit, or complete paralysis. Electrophysiologic monitoring with somatosensory evoked potentials was used to determine the optimal length of time of occlusion. Animals were treated randomly with 2 mg/kg of intravenous Mab 60.3 (n=8) or saline solution (n=9) with the investigator unaware of treatment. Mean occlusion times were no different between groups (control, 32.7+/−3.6 minutes versus MAb, 32.4+/−6.0 minutes). Five (55%) saline-treated and four (50%) MAb 60.3-treated animals became paraplegic. Animals with initial paraparesis all progressed to flaccid paraplegia within 24 hours. The study concludes that spinal cord injury after transient aortic occlusion is independent of the CD11/CD18 glycoprotein complex of the neutrophil. Injury in this setting can occur during ischemia and thus may not be dependent on neutrophils or reperfusion.

Liu et al. (1997), Neuronal and glial apoptosis after traumatic spinal cord injury, J Neurosci 17:5395-406, examines the spinal cords of rats subjected to traumatic insults of mild to moderate severity. Within minutes after mild weight drop impact (a 10 gm weight falling 6.25 mm), neurons in the immediate impact area showed a loss of cytoplasmic Nissl substances. Over the next 7 d, this lesion area expanded and cavitated. Terminal deoxynucleotidyl transferase (TdT)-mediated deoxyuridine triphosphate-biotin nick end labeling (TUNEL)-positive neurons were noted primarily restricted to the gross lesion area 4-24 hr after injury, with a maximum presence at 8 hr after injury. TUNEL-positive glia were present at all stages studied between 4 hr and 14 d, with a maximum presence within the lesion area 24 hr after injury. Seven days after injury, a second wave of TUNEL-positive glial cells was noted in the white matter peripheral to the lesion and extending at least several millimeters away from the lesion center. Apoptosis as a mechanism was evidenced by electron microscopy, as well as by nuclear staining with Hoechst 33342 dye, and by examination of DNA prepared from the lesion site. Furthermore, repeated intraperitoneal injections of cycloheximide, beginning immediately after a 12.5 mm weight drop insult, produced a substantial reduction in histological evidence of cord damage and in motor dysfunction assessed 4 weeks later. The data support the hypothesis that apoptosis dependent on active protein synthesis contributes to the neuronal and glial cell death, as well as to the neurological dysfunction, induced by mild-to-moderate severity traumatic insults to the rat spinal cord.

Exemplary results of an LPM conjugate in a model of spinal cord injury is set forth in Example 8, which shows exemplary results of LPM1d in a spinal cord injury model. Other LPMs, such as any provided herein containing a chemokine conjugated to a modified SA1, also can be tested in similar assays. Such results demonstrate that LPMs can be used as candidate therapeutics for treatment of spinal cord injury.

b. Neurodegenerative Diseases, Including Traumatic Brain Injury and Stroke

Models for testing and demonstrating activity of the conjugates herein for treatment of neurodegenerative diseases, such as traumatic brain injury and stroke are known to those of skill in the art. Exemplary references that provide and use animal models of neurodegenerative diseases, such traumatic brain injury and stroke, are provided. Such models can be used to confirm or identify ligand-toxin conjugates, such as LPM conjugates containing a modified SA1 moiety, include, but are not limited to, the following references set forth herein.

Ghirnikar et al. (1996), Chemokine expression in rat stab wound brain injury, J Neurosci Res 46:727-33, describes that traumatic injury to the adult mammalian central nervous system (CNS) results in reactive astrogliosis and the migration of hematogenous cells into the damaged neural tissue. Chemokines are recognized as mediators of the inflammatory changes that occur following injury. The expression of MCP-1 had been demonstrated in trauma in the rat brain (Berman et al. (1996) J Immunol 156:3017-3023). Using a stab wound model for mechanical injury, expression of two other chemokines: RANTES and MIP-1β in the rat brain was studied. The stab wound injury was characterized by widespread gliosis and infiltration of hematogenous cells. Immunohistochemical staining revealed the presence of RANTES and MIP-1β in the injured brain. RANTES and MIP-1β were both diffusely expressed in the necrotic tissue and were detected as early as 1 day post-injury (dpi). Double-labeling studies showed that MIP-1β, but not RANTES, was expressed by reactive astrocytes near the lesion site. In addition, MIP-1β staining was also detected on macrophages at the site of injury. The initial expression of the chemokines closely correlated with the appearance of inflammatory cells in the injured CNS, indicating that RANTES and MIP-1β could play a role in the inflammatory events of traumatic brain injury. This study also demonstrated MIP-1β expression in reactive astrocytes following trauma to the rat CNS.

Wang et al. (1998), Prolonged expression of interferon-inducible protein-10 in ischemic cortex after permanent occlusion of the middle cerebral artery in rat, J Neurochem 71:1194-204, investigates the role of IP-10 in focal stroke, and studies temporal expression of IP-10 mRNA after occlusion of the middle cerebral artery in rat by means of Northern analysis. IP-10 mRNA expression after focal stroke demonstrated a unique biphasic profile, with a marked increase early at 3 h (4.9-fold over control; p 0.01), a peak level at 6 h (14.5-fold; p 0.001) after occlusion of the middle cerebral artery, and a second wave induction 10-15 days after ischemic injury (7.2- and 9.3-fold increase for 10 and 15 days, respectively; p 0.001). In situ hybridization confirmed the induced expression of IP-10 mRNA and revealed its spatial distribution after focal stroke. Immunohistochemical studies demonstrated the expression of IP-10 peptide in neurons (3-12 h) and astroglial cells (6 h to 15 days) of the ischemic zone. A dose-dependent chemotactic action of IP-10 on C6 glial cells and enhanced attachment of rat cerebellar granule neurons was demonstrated. The data indicated that ischemia induces IP-10, which plays a pleiotropic role in prolonged leukocyte recruitment, astrocyte migration/activation, and neuron attachment/sprouting after focal stroke.

Galasso et al. (1998), Excitotoxic brain injury stimulates expression of the chemokine receptor CCR5 in neonatal rats, Am J Pathol 153:1631-40, evaluates the impact of intrahippocampal injections of NMDA on CCR5 expression in postnatal day 7 rats. Reverse transcription polymerase chain reaction revealed an increase in hippocampal CCR5 mRNA expression 24 hours after lesioning, and in situ hybridization analysis demonstrated that CCR5 mRNA was expressed in the lesioned hippocampus and adjacent regions. Western blot analysis demonstrated increased CCR5 protein in hippocampal tissue extracts 32 hours after lesioning. Complementary immunocytochemistry studies identified infiltrating microglia/monocytes and injured neurons as the principal CCR5-immunoreactive cells. These results provide evidence that acute excitotoxic injury regulates CCR5 expression.

Vannucci et al. (1999), Rat model of perinatal hypoxic-ischemic brain damage, J Neurosci Res 55:158-63, uses an immature rat model to gain insights into the pathogenesis and management of perinatal hypoxic-ischemic brain damage. The model entails ligation of one common carotid artery followed thereafter by systemic hypoxia. The insult produces permanent hypoxic-ischemic brain damage limited to the cerebral hemisphere ipsilateral to the carotid artery occlusion. This model is used in investigations to identify therapeutic strategies to prevent or minimize hypoxic-ischemic brain damage.

c. Neurodegenerative Diseases—Alzheimer's Disease (AD)

Models for testing and demonstrating activity of the conjugates herein for treatment of neurodegenerative diseases, such as (AD) are known to those of skill in the art. Exemplary animal models of such diseases are provided. These models can be used to confirm or identify conjugates for use for treatment of neurodegenerative diseases, such as Alzheimer's disease which can be used to test ligand-toxin conjugates, such as LPM conjugates containing a modified SA1 moiety, includes, but is not limited to, the following reference set forth herein.

Hauss-Wegrzyniak et al. (1998), Chronic neuroinflammation in rats reproduces components of the neurobiology of Alzheimer's disease, Brain Res 780:294-303, describes that inflammatory processes play a role in the pathogenesis of the degenerative changes and cognitive impairments associated with Alzheimer's disease and describes use of lipopolysaccharide (LPS) from the cell wall of gram-negative bacteria to produce chronic, global inflammation within the brain of young rats. Chronic infusion of LPS (0.25 μg/h) into the 4th ventricle for four weeks produced (1) an increase in the number of glial fibrillary acidic protein-positive activated astrocytes and OX-6-positive reactive microglia distributed throughout the brain, with the greatest increase occurring within the temporal lobe, particularly the hippocampus, (2) an induction in interleukin-1 beta, tumor necrosis factor-alpha and beta-amyloid precursor protein mRNA levels within the basal forebrain region and hippocampus, (3) the degeneration of hippocampal CA3 pyramidal neurons, and (4) a significant impairment in spatial memory as determined by decreased spontaneous alternation behavior on a T-maze.

Numerous other Alzheimer disease models, including rodents genetically engineered to express the mutated form of a human gene involved in production of Aβ in families with early onset Alzheimer's disease, are known and available to those of skill in this art.

d. Multiple Sclerosis

Multiple sclerosis (MS) is an inflammatory disease of the central nervous system (CNS) characterized by localized areas of demyelination. Immune responses to myelin antigens contribute to the disease process. MS is a heterogeneous chronic autoimmune disease characterized by marked inflammation, loss of oligodendrocyte myelin sheath, neurodegeneration, gliosis and axon loss. (see, e.g., Bruck, W. & Stadelmann, C. Neurol Sci 24 Suppl 5, S265-7 (2003); Bruck, W. & Stadelmann, C., Curr Opin Neurol 18, 221-4 (2005); Fox, E. J., Neurology 63, S3-7 (2004); Fox, R. J. & Ransohoff, R. M, Trends Immunol 25, 632-6 (2004); Hendriks, J. J., Teunissen, C. E., de Vries, H. E. & Dijkstra, C. D. Brain Res Brain Res Rev 48, 185-95 (2005); Prat, A. & Antel, J., Curr Opin Neurol 18, 225-30 (2005); Liu, L., Callahan, M. K., Huang, D. & Ransohoff, R. M., Curr Top Dev Biol 68, 149-81 (2005); Mahad, D. et al., Ernst Schering Res Found Workshop, 59-68 (2004)). The disease affects approximately 400,000 people in North America and 2.5 million worldwide (see, e.g., Cross, A. H. & Stark, J. L., Immunol Res 32, 85-98 (2005); Hafler, D. A., J Clin Invest 113, 788-94 (2004); Sindern, E., Front Biosci 9, 457-63 (2004); and Steinman, L., Neuron 24, 511-4 (1999)). The onset is normally between 20-40 years of age, but there are forms of atypical MS including cases of early (under 18 years) and late (over 50 years) onset that present differential prognostic and diagnostic challenges (see, e.g., Krupp, L. B. & Macallister, W. S., Curr Treat Options Neurol 7, 191-199 (2005); Martinelli, V., Rodegher, M., Moiola, L. & Comi, G., Neurol Sci 25 Suppl 4, S350-5 (2004); Stadelmann, C. & Bruck, W., Neurol Sci 25 Suppl 4, S319-22 (2004); Stadelmann, C. et al., Brain 128, 979-87 (2005)).

Approximately 85% of usual cases begin as relapsing-remitting episodes of impaired sensory modalities including impaired vision, temporary blindness and motor co-ordination. This can give way to secondary progressive disease. Another form of MS is called primary progressive MS. Relapses continue to occur until the neurodegenerative phase takes over (see, e.g., Bruck, W. & Stadelmann, C., Neurol Sci 24 Suppl 5, S265-7 (2003); Bruck, W. & Stadelmann, C., Curr Opin Neurol 18, 221-4 (2005); Fox, E. J., Neurology 63, S3-7 (2004); Fox, R. J. & Ransohoff, R. M., Trends Immunol 25, 632-6 (2004); Steinman, L., Curr Opin Immunol 13, 597-600 (2001); and Zaffaroni, M., Neurol Sci 24 Suppl 5, S279-82 (2003)). Immune, genetic, and environmental (such as, viruses, bacteria) components are implicated in the etiology of MS (see, e.g., Zaffaroni, M., Neurol Sci 24 Suppl 5, S279-82 (2003)).

A hallmark of MS pathology is white matter plaques or lesions throughout the CNS including the spinal cord (see, e.g., Bruck, W. & Stadelmann, C., Neurol Sci 24 Suppl 5, S265-7 (2003); Mahad, D. et al., Ernst Schering Res Found Workshop, 59-68 (2004); Fawcett, J. W. & Asher, R. A., Brain Res Bull 49, 377-91 (1999); and Zhang, Y. et al., J Clin Immunol 25, 254-64 (2005)). The most populous leukocyte groups in chronic active lesions are activated CCR2⁺/CCR3⁺/CCR5⁺/CXCR3⁺ macrophages. Other cells include B cells, T cells and microglia with a similar receptor expression pattern. The cognate ligands for these receptors are produced in lesion surrounding astrocytes and the participating leukocyte groups (see, e.g., Banisor, I., Leist, T. P. & Kalman, B., J Neuroinflammation 2, 7 (2005); Cartier, L., Hartley, O., Dubois-Dauphin, M. & Krause, K. H., Brain Res Brain Res Rev 48, 16-42 (2005); Galimberti, D., Bresolin, N. & Scarpini, E., Expert Rev Neurother 4, 439-53 (2004); and Putheti, P. et al., Eur J Neurol 10, 529-35 (2003)). Once in the CNS leukocyte groups cause immune damage via an armament of noxious substances including reactive oxygen and nitrogen species; MMP; leukotrienes; production of autoantibodies and release of proinflammatory cytokines and chemokines. This in turn causes axonal damage, lesion formation and oligodendrocyte and neuronal cell death.

1) EAE Model

In the EAE model, the demyelinating disease is induced in mice. Activated monocytes, macrophages microglia and T cells are responsible for the damage to tissue. While the model in this case is acute (like chronic progressive MS as opposed to relapsing-remitting), it is in essence prior to exacerbations there is an upregulation of CCR2 (the receptor for example, for LPM1d; the sequence of amino acids of LPM1d polypeptide is set forth in SEQ ID NO:44) on those leukocyte groups, infiltration of the CNS and disease. Hence the model evidences treatments applicable to all types of MS. An exemplary reference that provides and uses animal models of multiple sclerosis that can be used to test ligand-toxin conjugates, such as LPM conjugates containing a modified SA1 moiety, includes, but is not limited to, Liu et al. (1998), Nat Med 4:78-83, which describes use of a rodent model, experimental autoimmune encephalomyelitis (EAE) for studying MS. Data showing effectiveness of conjugates provided herein in the EAE model are provided in Example 10.

2) Leukocytes Involved in MS

In numerous studies macrophages and microglia are essential in the pathology of human MS and in the EAE model. DC, MaC and B cells also play a role (Cross, A. H. & Stark, J. L., Immunol Res 32, 85-98 (2005); Zhang, Y. et al., J Clin Immunol 25, 254-64 (2005); Mouzaki, A., Tselios, T., Papathanassopoulos, P., Matsoukas, I. & Chatzantoni, K., Curr Neurovasc Res 1, 325-40 (2004); Heppner, F. L. et al., Nat Med 11, 146-52 (2005); Huiting a, I. et al., Clin Exp Immunol 100, 344-51 (1995); Polfliet, M. M. et al., J Neuroimmunol 122, 1-8 (2002); Behi, M. E. et al., Immunol Lett 96, 11-26 (2005); Theoharides, T. C. & Cochrane, D. E., J Neuroimmunol 146, 1-12 (2004); Chavarria, A. & Alcocer-Varela, J., Autoimmun Rev 3, 251-60 (2004); Kouwenhoven, M. et al., J Neuroimmunol 126, 161-71 (2002); Greter, M. et al., Nat Med 11, 328-34 (2005)). Microglial cells are activated and proliferate prior to the onset of EAE (see, e.g., Ponomarev, E. D., Shriver, L. P., Maresz, K. & Dittel, B. N., J Neurosci Res 81, 374-89 (2005)). Microglia and macrophage deactivation and T and MNP cell depletion ameliorates the severity of EAE (see, e.g., Heppner, F. L. et al., Nat Med 11, 146-52 (2005); Huiting a, I. et al., Clin Exp Immunol 100, 344-51 (1995); Polfliet, M. M. et al., J Neuroimmunol 122, 1-8 (2002); Raj an, A. J., Asensio, V. C., Campbell, I. L. & Brosnan, C. F., J Immunol 164, 2120-30 (2000); Rajan, A. J., Klein, J. D. & Brosnan, C. F. J Immunol 160, 5955-62 (1998); Bauer, J. et al., Glia 15, 437-46 (1995); Kotter, M. R., Zhao, C., van Rooijen, N. & Franklin, R. J., Neurobiol Dis 18, 166-75 (2005); and Tran, E. H., Hoekstra, K., van Rooijen, N., Dijkstra, C. D. & Owens, T., J Immunol 161, 3767-75 (1998)). Studies have shown that peripheral macrophages are pivotal for their activation of T cells and development of EAE (see, e.g., Polfliet, M. M. et al., J Neuroimmunol 122, 1-8 (2002); Deloire, M. S. et al., Mult Scler 10, 540-8 (2004); Inrich, H. & Harzer, K., J Neural Transm 108, 379-95 (2001); Raivich, G. & Banati, R., Brain Res Brain Res Rev 46, 261-81 (2004)). The depletion of B cells with Rituxan (anti-CD₂O mAb) in MS patients resulted in significant clinical improvement (see, Stuve, O. et al., Arch Neurol 62, 1620-3 (2005)). Depletion of PMN in EAE inhibit the effector phase of the disease. PMN in MS patients express high levels of several cell-surface antigens which is linked to exacerbation of the disease (see, e.g., McColl, S. R. et al., J Immunol 161, 6421-6 (1998); Ziaber, J. et al., Mediators Inflamm 7, 335-8 (1998)). Mouse studies indicate that CNS PMN are potent suppressors of T cell responses to myelin and adjuvant antigens and a recent case of autoimmune neutropenia was reported in an MS male patient (Kozuka, T. et al., Intern Med 42, 102-4 (2003); and Zehntner, S. P. et al., J Immunol 174, 5124-31 (2005)).

Highly activated microglia, perivascular MNP and infiltrating MNP play several roles in MS. Apart from their inflammatory destructive roles by releasing noxious substances, they present antigen to infiltrating T cells to promote myelin specific T cell responses and further recruitment of T cells and macrophages via chemokines (see, e.g., Deng, X. & Sriram, S., Curr Neurol Neurosci Rep 5, 239-44 (2005); Behi, M. E. et al., Immunol Lett 96, 11-26 (2005); Raivich, G. & Banati, R., Brain Res Brain Res Rev 46, 261-81 (2004); Nelson, P. T., Soma, L. A. & Lavi, E., Ann Med 34, 491-500 (2002); Zhang, S. C., Goetz, B. D., Carre, J. L. & Duncan, I. D., Glia 34, 101-9 (2001); Izikson, L., Klein, R. S., Luster, A. D. & Weiner, H. L., Clin hnmunol 103, 125-31 (2002)). Macrophages also are involved in the pathology of degeneration in MS. Cellular infiltrates are associated with axonal loss in MS lesions (see, e.g., Hendriks, J. J., Teunissen, C. E., de Vries, H. E. & Dijkstra, C. D., Brain Res Brain Res Rev 48, 185-95 (2005)). Phagocytotic MNP and microglia destroy the axonal myelin sheaths, which lead to patient dysfunctions (see, e.g., Cartier, L., Hartley, O., Dubois-Dauphin, M. & Krause, K. H., Brain Res Brain Res Rev 48, 16-42 (2005); Raivich, G. & Banati, R., Brain Res Brain Res Rev 46, 261-81 (2004); and Smith, M. E., van der Maesen, K. & Somera, F. P., J Neurosci Res 54, 68-78 (1998)). Microglia can induce neuronal cell death and inhibit neurite outgrowth as well as phagocytosing neuronal apoptotic bodies (see, e.g., Munch, G. et al., Exp Brain Res 150, 1-8 (2003); and Stolzing, A. & Grune, T., Faseb J 18, 743-5 (2004)).

Monocyte-derived DC, B-cells, MaC and activated astrocytes also are involved in the pathology of MS (see, e.g., Zhang, Y. et al., J Clin Immunol 25, 254-64 (2005); Behi, M. E. et al., Immunol Lett 96, 11-26 (2005); Chavarria, A. & Alcocer-Varela, J., Autoimmun Rev 3, 251-60 (2004); Corcione, A. et al., Autoimmun Rev 4, 549-54 (2005); and Zang, Y. C. et al., Mult Scler 10, 499-506 (2004)). Activated astrocytes release several chemokines and other mediators to attract leukocytes to the sites of inflammation (see, e.g., Ambrosini, E. et al., J Neuropathol Exp Neurol 64, 706-15 (2005); Andjelkovic, A. V., Kerkovich, D. & Pachter, J. S., J Leukoc Biol 68, 545-52 (2000); Krumbholz, M. et al., J Exp Med 201, 195-200 (2005)). MaC release proteases that cause vascular permeability and facilitate fibrin deposition in lesions (see, e.g., Theoharides, T. C. & Cochrane, D. E., J Neuroimmunol 146, 1-12 (2004); Pedotti, R., De Voss, J. J., Steinman, L. & Galli, S. J., Trends Immunol 24, 479-84 (2003)). DC present antigens facilitating activation of T cell and the progression of disease (see, e.g., Greter, M. et al., Nat Med 11, 328-34 (2005)). Ectopic lymphoid tissue is evident at the sites of inflammation in the meninges of MS patients. The meninges of such patients contain B, T, plasma, and DC cells, which represent a step in maintaining humoral autoimmunity and disease exacerbation (see, e.g., Serafini, B., Rosicarelli, B., Magliozzi, R., Stigliano, E. & Aloisi, F., Brain Pathol 14, 164-74 (2004)).

In addition to MNP, T cells, Ig and immune complexes, B cells also occur in MS lesions. Over 70% of active lesions contain complement and antibodies (see, e.g., Cross, A. H. & Stark, J. L. Immunol Res 32, 85-98 (2005)). Clonally expanded antibody-secreting B cells and centroblasts are found in the CSF of MS patients (see, e.g., Zhang, Y. et al., J Clin Immunol 25, 254-64 (2005); Ziemssen, T. & Ziemssen, F., Autoimmun Rev 4, 460-7 (2005); Corcione, A. et al., Autoimmun Rev 4, 549-54 (2005); and Corcione, A. et al., Proc Natl Acad Sci USA 101, 11064-9 (2004)). Over 90% of MS patients have intrathecal oligoclonal Ig and increased amounts of antibodies in the CSF, which correlate with episodes of MS worsening (see, e.g., Cross, A. H. & Stark, J. L., Immunol Res 32, 85-98 (2005)). The B cells are thought to be derived from a CNS germinal center; the brain provides a favorable microenvironment for long term survival, proliferation and the formation of ectopic lymphoid structures. B cells make antibodies to myelin proteins (increasing myelin opsonization), present antigen and costimulatory molecules to T cells and increase leukocyte recruitment. A study has found that a proportion of circulating B cells are not permanently inactivated, but are continually activated and become the cause of autoimmune attacks (see, e.g., Gauld, S. B., Benschop, R. J., Merrell, K. T. & Cambier, J. C., Nat Immunol 6, 1160-7 (2005)). The B cells in MS can be activated as they differentiate within the CNS.

3) Chemokines in MS

Chemokine-messaging system of ligands and receptors play pivotal roles in the pathology of EAE and MS. The system orchestrates the trafficking, CNS infiltration and aberrant inflammatory functions of a range of leukocyte subtypes in these autoimmune diseases. Numerous chemokines and their receptors have been identified in multiple sclerosis lesions including CCL-1-8, CXCL8-13, CCR1-3,5 and CXCR1-3, 4 (see, e.g. Banisor, I., Leist, T. P. & Kalman, B., J Neuroinflammation 2, 7 (2005); Cartier, L., Hartley, O., Dubois-Dauphin, M. & Krause, K. H., Brain Res Brain Res Rev 48, 16-42 (2005); Galimberti, D., Bresolin, N. & Scarpini, E., Expert Rev Neurother 4, 439-53 (2004); Putheti, P. et al., Eur J Neurol 10, 529-35 (2003); and Raivich, G. & Banati, R., Brain Res Brain Res Rev 46, 261-81 (2004)). For example, CCR1, 2, 5 and 6 and CXCR3 occur on CD3+ T cells and CCR1, 2, 3 and 5 and CXCR3 on foamy macrophages and activated microglia in MS lesions (see, e.g., Banisor, I., Leist, T. P. & Kalman, B., J Neuroinflammation 2, 7 (2005); Cartier, L., Hartley, O., Dubois-Dauphin, M. & Krause, K. H., Brain Res Brain Res Rev 48, 16-42 (2005); Galimberti, D., Bresolin, N. & Scarpini, E., Expert Rev Neurother 4, 439-53 (2004); Putheti, P. et al., Eur J Neurol 10, 529-35 (2003); Raivich, G. & Banati, R., Brain Res Brain Res Rev 46, 261-81 (2004); Malamud, V. et al., J Neuroimmunol 138, 115-22 (2003); Pedotti, R., De Voss, J. J., Steinman, L. & Galli, S. J., Trends Immunol 24, 479-84 (2003); Serafini, B., Rosicarelli, B., Magliozzi, R., Stigliano, E. & Aloisi, F., Brain Pathol 14, 164-74 (2004); Corcione, A. et al., Proc Natl Acad Sci USA 101, 11064-9 (2004); Gauld, S. B., Benschop, R. J., Merrell, K. T. & Cambier, J. C., Nat Immunol 6, 1160-7 (2005); Bartosik-Psujek, H. & Stelmasiak, Z., Eur J Neurol 12, 49-54 (2005)).

Astrocyte-derived CCL2 and CXCL10 were demonstrated in EAE studies. These chemokines trigger further neural immune responses and contribute to the recruitment of leukocytes from the periphery (see, e.g., Galimberti, D., Bresolin, N. & Scarpini, E., Expert Rev Neurother 4, 439-53 (2004); Huang, D. et al., Immunol Rev 177, 52-67 (2000); Jee, Y., Yoon, W. K., Okura, Y., Tanuma, N. & Matsumoto, Y., J Neuroimmunol 128, 49-57 (2002)). CCL2/CCR2 and CXCL9/10/11/CXCR3 are among the targets for therapeutic intervention because of their distribution on several specific pathological leukocyte cell types and their frequent detection in MS and EAE studies. The chemokine axis CCL2/CCR2 plays a role in transendothelial migration of MNP and T cells into the CNS, and is implicated in blood-brain barrier (BBB) damage and collapse (see, e.g., Chavarria, A. & Alcocer-Varela, J Autoimmun Rev 3, 251-60 (2004); Mahad, D. et al., Brain (2005); Dzenko, K. A., Andjelkovic, A. V., Kuziel, W. A. & Pachter, J. S., J Neurosci 21, 9214-23 (2001); Dzenko, K. A., Song, L., Ge, S., Kuziel, W. A. & Pachter, J. S., Microvasc Res (2005); Stamatovic, S. M. et al., J Cereb Blood Flow Metab 25, 593-606 (2005); Minagar, A. & Alexander, J. S., Mult Scler 9, 540-9 (2003)). CCL2 increases BBB permeability by altering the tight junctions between endothelial cells via CCR2 (Stamatovic, S. M. et al., J Cereb Blood Flow Metab 25, 593-606 (2005)). Incoming MNP also alter the permeability by secreting CCL2 and then migrate into the CNS. MNP- and T cell-derived MMP also are associated with the breakdown and collapse of the BBB and aids cellular transmigration (see, e.g., Abraham, M., Shapiro, S., Karni, A., Weiner, H. L. & Miller, A., J Neuroimmunol 163, 157-64 (2005); Avolio, C. et al., J Neuroimmunol 136, 46-53 (2003); Karabudak, R. et al., J Neurol 251, 279-83 (2004); Uccelli, A., Pedemonte, E., Narciso, E. & Mancardi, G., Neurol Sci 24 Suppl 5, S271-4 (2003); Brundula, V., Rewcastle, N. B., Metz, L. M., Bernard, C. C. & Yong, V. W., Brain 125, 1297-308 (2002); Sellebjerg, F. & Sorensen, T. L., Brain Res Bull 61, 347-55 (2003); Vos, C. M., van Haastert, E. S., de Groot, C. J., van der Valk, P. & de Vries, H. E., J Neuroimmunol 138, 106-14 (2003)). CXCR3⁺ marks T cells for trafficking to the BBB, but it is the expression of CCR2 on these cells that allows diapedesis (see, Mahad, D. et al., Brain (2005); Callahan, M. K. et al., J Neuroimmunol 153, 150-7 (2004), which describes down regulation of CCR2 on T cells and monocytes after crossing the BBB). The CCL2/CCR2 chemokine pair is involved in BBB permeability and a significant increase in the CCL2/CCR2 axis on several leukocyte types in the CNS parenchyma and within lesions is observed (Mahad, D. J. & Ransohoff, R. M., Semin Immunol 15, 23-32 (2003). In addition, the CCL2/CCR2 axis is noted in lesions of MS brains, the blood and the CSF of patients (see, Banisor, I., Leist, T. P. & Kalman, B., J Neuroinflammation 2, 7 (2005); Cartier, L., Hartley, O., Dubois-Dauphin, M. & Krause, K. H., Brain Res Brain Res Rev 48, 16-42 (2005); Putheti, P. et al., Eur J Neurol 10, 529-35 (2003); and Mahad, D. J. & Ransohoff, R. M., Semin Immunol 15, 23-32 (2003))). Receptor expression varies over time because chemoprints are temporal and spatial and change according to the prevailing microenvironment (Karpus, W. J. & Ransohoff, R. M. J Immunol 161, 2667-71 (1998)). As monocytes cross the BBB they down-regulate then re-express CCR2 as they mature into differentiate into macrophages. This is evidenced in studies with post-mortem MS biopsies showing low levels of CCR2, CCR3 and CCR5 expressed by microglial cells throughout control CNS tissue. In chronic active MS lesions, CCR2, CCR3 and CCR5 occur on foamy macrophages and activated microglia. CCR2 and CCR5 also are present on large numbers of infiltrating lymphocytes and there is a smaller number of CCR3-positive lymphocytes (see, e.g., Simpson, J. et al., J Neuroimmunol 108, 192-200 (2000)). Similarly, CXCR3 and CCR5 are preferentially expressed on Th1 cells (proinflammatory cytokine producers) and significantly upregulated in the peripheral blood during MS relapses. The levels of receptors drop as patients go into remission (Mahad, D. J., Lawry, J., Howell, S. J. & Woodroofe, M. N., Mult Scler 9, 189-98 (2003)). Expression of CXCL10 is upregulated in the CSF from MS patients and is spatially associated with demyelination in CNS tissue sections correlating tightly with the expression of its receptor, CXCR3 (see, e.g., Sorensen, T. L. et al., J Neuroimmunol 127, 59-68 (2002)). Further evidence of the participation of CCL2/CCR2 in autoimmune demyelination comes from EAE studies. It was found that CCL2 is highly expressed in the CNS, and anti-CCL2 treatment blocks relapses of disease in mice. Studies with CCR2^{-/-} mice show that CCL2/CCR2 is important for the development of EAE (Fife, B. T., Huffnagle, G. B., Kuziel, W. A. & Karpus, W. J., J Exp Med 192, 899-905 (2000); Izikson, L., Klein, R. S., Charo, I. F., Weiner, H. L. & Luster, A. D., J Exp Med 192, 1075-80 (2000)). A CCL2 DNA vaccine protected the animals from developing EAE, and upregulation of CCL2 and CCR2 was closely associated with the relapse phase of the disease (Jee, Y., Yoon, W. K., Okura, Y., Tanuma, N. & Matsumoto, Y., J Neuroimmunol 128, 49-57 (2002); Youssef, S. et al., J Immunol 161, 3870-9 (1998)). CCL2 was shown to cause encephalopathy when chronically expressed in mice showing that the chemokine can induce lesion formation (Huang, D. et al., Faseb J 19, 761-72 (2005)).

The CXCL9/10/11 chemokines and their cognate CXCR3 receptor also play a role in EAE and MS (see, e.g., Liu, L., Callahan, M. K., Huang, D. & Ransohoff, R. M., Curr Top Dev Biol 68, 149-81 (2005); Cartier, L., Hartley, O., Dubois-Dauphin, M. & Krause, K. H., Brain Res Brain Res Rev 48, 16-42 (2005); Sorensen, T. L. et al., J Neuroimmunol 127, 59-68 (2002); Mahad, D. J., Lawry, J., Howell, S. J. & Woodroofe, M. N., Mult Scler 9, 189-98 (2003); and Lazzeri, E. & Romagnani, P., Curr Drug Targets Immune Endocr Metabol Disord 5, 109-118 (2005). In MS, the inflammatory balance is in favor of Th1 cells (proinflammatory cytokine producers), which are associated with the expression of CXC3 and CCR5 as opposed to a Th2 cell environment (anti-inflammatory cytokine producers), and that characteristically express CCR3, 4 and 8 (see, e.g., Mouzaki, A., Tselios, T., Papathanassopoulos, P., Matsoukas, I. & Chatzantoni, K., Curr Neurovasc Res 1, 325-40 (2004); Teleshova, N. et al., J Neurol 249, 723-9 (2002); and Misu, T. et al., J Neuroimmunol 114, 207-12 (2001)). CXCR3 and CCR5 expressing T cells are significantly enriched in the MS CSF compared with blood. CCR5⁺/CCR3⁻ cells are absent from the CSF indicating that CCR5 is not responsible for T cell trafficking to the CSF alone (see, e.g., Kivisakk, P. et al., Clin Exp Immunol 129, 510-8 (2002); and Sorensen, T. L. et al., J Clin Invest 103, 807-15 (1999)). In a model of inflammation, anti-CXCR3 treatment ameliorated Th1 cell migration to inflamed tissue demonstrating that CXCR3 is a receptor required for trafficking (see, e.g., Xie, J. H. et al., J Leukoc Biol 73, 771-80 (2003); and Sindem, E., Patzold, T., Ossege, L. M., Gisevius, A. & Malin, J. P., J Neuroimmunol 131, 186-90 (2002)). In active MS lesions, CXCL9 and CXCL10 are expressed by macrophages and by astrocytes surrounding the lesion. CXCR3 is expressed on T cells and astrocytes within the lesion. Th1 cell-derived IFN-γ stimulates cells to express the chemoattractants to continue the recruitment of T cells to the CNS (Simpson, J. et al., J Neuroimmunol 108, 192-200 (2000)). CXCR3 and cognate ligands were studied in several EAE models. This axis plays a role in specific EAE models and species of rodent. In one study, CXCL10-null mice displayed the expression, severity and histopathology as the control group. The study concluded that CXCL10 was not required for trafficking, but did determine the decreased threshold of disease susceptibility in the periphery compared to controls (Klein, R. S. et al., J Immunol 172, 550-9 (2004); Oppenheim, J. J. et al., J Leukoc Biol 77, 854-61 (2005)). CXCR3 also are expressed in at least a percentage of the leukocyte groups discussed above (Sorensen, T. L. et al., J Clin Invest 103, 807-15 (1999); Oppenheim, J. J. et al., J Leukoc Biol 77, 854-61 (2005); Kuipers, H. F. et al. Glia (2005); Foley, J. F. et al., J Immunol 174, 4892-900 (2005)). For example, patients had higher expression of CXCR3 and CCR5 on B cells in the CSF and blood, respectively in active MS than controls did (Sorensen, T. L., Roed, H. & Sellebjerg, F., J Neuroimmunol 122, 125-31 (2002)). Mouse and human astrocytes and microglia express the receptor and undergo chemotaxis in response to the cognate ligands in vitro (see, e.g., Biber, K. et al., Neuroscience 112, 487-97 (2002)). Hence these receptors, among others, can be targeted by appropriate selection of an conjugate provided herein by selecting one that targets one or more of these receptors (see Tables, Examples and description herein).

4) Therapeutics in MS

Methylprednisolone, Interferons and Copaxone slow the progression of the relapsing remitting disease. Immunosuppressive drugs including novantrone, azathioprine, methotrexate and cyclophosphamide were used in primary and secondary progressive MS (Table 5). No drug (except for the ill-fated Campath) has definitely modified the course of the disease (see, e.g., Galimberti, D., Bresolin, N. & Scarpini, E., Expert Rev Neurother 4, 439-53 (2004); and Leary, S. M. & Thompson, A. J., CNS Drugs 19, 369-76 (2005)). Leukocyte depletion studies and the good pathology and remission induced by Campath leukocyte depletion (despite the toxicity) bodes well for chemokine-mediated leukocyte depopulation (see, e.g., Coles, A. J. et al., Ann Neurol 46, 296-304 (1999); Moreau, T. et al., Lancet 344, 298-301 (1994)). The chemokine messaging system can serve as a robust therapeutic target for MS. As many leukocyte subtypes active in MS express CCR2 and/or CXCR3, conjugates that target such receptors, such as LPM7 or LPM1d and others exemplified herein, can be used for or in the treatment of MS. Other conjugates that target any of or combinations of two or more of CCL1-8, CXCL8-13, CCR1-3,5, 6 and CXCR1-3, 4 can be used.

e. Arthritis and Autoimmune Disease

Models for testing and demonstrating activity of the conjugates herein for treatment of autoimmune diseases, such as arthritis, lupus, and MS, discussed above, are known to those of skill in the art. Exemplary references that provide and use animal models of arthritis and autoimmune disease, which models can be used to test ligand-toxin conjugates, such as LPM conjugates containing a modified SA1 moiety, include, but are not limited to, the following references set forth herein.

Barnes et al. (1998), Polyclonal antibody directed against human RANTES ameliorates disease in the Lewis rat adjuvant-induced arthritis model, J Clin Invest 101:2910-9, describes that adjuvant-induced arthritis (AIA) is one of many animal models of rheumatoid arthritis, a disease characterized by a T-lymphocyte and macrophage cellular infiltrate. Barnes et al. characterizes the development of this disease model with respect to chemokine expression, and shows that increased levels of two chemokines, RANTES, a T-lymphocyte and monocyte chemo-attractant, and KC (the mouse homolog of human GRO-α), a chemoattractant for neutrophils, were found in whole blood and in the joint. Levels of MIP-1α, another T-lymphocyte and monocyte chemoattractant were unchanged throughout the course of the disease in whole blood and only slightly elevated in the joint. RANTES expression plays an important role in the disease since a polyclonal antibody to RANTES greatly ameliorated symptoms in animals induced for AIA and was found to be as efficacious as treatment with indomethacin, a non-steroidal anti inflammatory. Polyclonal antibodies to either MIP-1α or KC were ineffective.

Weinberg, A. D. (1998), Antibodies to OX-40 (CD134) can identify and eliminate autoreactive T cells: implications for human autoimmune disease, Mol Med Today 4:76-83, describes that autoantigen-specific CD4+ T cells have been implicated as the causative cell type in: multiple sclerosis, rheumatoid arthritis, autoimmune uveitis, diabetes mellitus, inflammatory bowel disease and graft-versus-host disease. Weinberg also describes the use of experimentally induced autoimmune diseases to develop an effective therapy that deletes the autoreactive T cells at the site of autoimmune tissue destruction.

Schrier et al. (1998), Role of chemokines and cytokines in a reactivation model of arthritis in rats induced by injection with streptococcal cell walls, J Leukoc Biol 63:359-63, provides a study of the role of chemokines in an animal model of arthritis. Intraarticular injection of streptococcal cell wall (SCW) antigen followed by intravenous challenge results in a T cell-mediated monoarticular arthritis in female Lewis rats. Initial studies showed that this reactivation response to intravenous SCW antigen is dependent on the presence of interleukin-1 (IL-1) and tumor necrosis factor alpha (TNF-alpha) and that the early phase of swelling is neutrophil dependent. Neutrophil depletion or passive immunization with antibodies to P-selectin or MIP-2 reduced the intensity of ankle edema and the influx of neutrophils. After the first few days, however, the arthritic response is mediated primarily by mononuclear cells. Joint tissues showed up-regulation of mRNA for MCP-1, which could be inhibited in part by anti-IL-4; treatment of rats with antibodies to IL-4 or MCP-1 significantly suppressed development of ankle edema and histopathological evidence of inflammation. Antibodies to interferon-gamma or IL-10 had no effect. Treatment with anti-MCP-1 also suppressed influx of labeled T cells into the ankle joint. These data indicated that the late, mononuclear-dependent phase of SCW-induced arthritis in female Lewis rats requires cytokines that up-regulate MCP-1, which in turn could facilitate recruitment and extravasation of mononuclear cells into the joint.

Oppenheimer-Marks et al. (1998), Interleukin 15 is produced by endothelial cells and increases the transendothelial migration of T cells in vitro and in the SCID mouse-human rheumatoid arthritis model in vivo, J Clin Invest 101:1261-72, examines the capacity of endothelial cells (EC) to produce IL-15 and the capacity of IL-15 to influence transendothelial migration of T cells. Human umbilical vein endothelial cells express IL-15 mRNA and protein. Endothelial-derived IL-15 enhanced transendothelial migration of T cells as evidenced by the inhibition of this process by blocking monoclonal antibodies to IL-15. IL-15 enhanced transendothelial migration of T cells by activating the binding capacity of the integrin adhesion molecule LFA-1 (CD11a/CD18) and also increased T cell motility. In addition, IL-15 induced expression of the early activation molecule CD69. The importance of IL-15 in regulating migration of T cells in vivo was documented by its capacity to enhance accumulation of adoptively transferred human T cells in rheumatoid arthritis synovial tissue engrafted into immune deficient SCID mice. These results demonstrate that EC produce IL-15, which plays a role in stimulation of T cells to extravasate into inflammatory tissue.

Kasama et al. (1995), Interleukin-10 expression and chemokine regulation during the evolution of murine type II collagen-induced arthritis J Clin Invest 95:2868-76, studies the expression and contribution of specific chemokines, MIP-1α and MIP-2, and interleukin 10 (IL-10) during the evolution of type II collagen-induced arthritis (CIA). Detectable levels of chemotactic cytokine protein for MIP-1α and MIP-2 were first observed between days 32 and 36, after initial type II collagen challenge, while increases in IL-10 were found between days 36 and 44. CIA mice passively immunized with antibodies directed against either MIP-1α or MIP-2 demonstrated a delay in the onset of arthritis and a reduction of the severity of arthritis. CIA mice receiving neutralizing anti-IL-10 antibodies demonstrated an acceleration of the onset and an increase in the severity of arthritis. Anti-IL-10 treatment increased the expression of MIP-1α and MIP-2, as well as increased myeloperoxidase (MPO) activity and leukocyte infiltration in the inflamed joints. These data indicate that MIP-1α and MIP-2 play a role in the initiation and maintenance, while IL-10 appears to play a regulatory role during the development of experimental arthritis.

Keffer et al. (1991), Transgenic mice expressing human tumor necrosis factor: a predictive genetic model of arthritis, Embo J 10:4025-31, provides transgenic mouse lines carrying and expressing wildtype and 3′-modified human tumor necrosis factor (hTNF-alpha, cachectin) transgenes, shows correct, endotoxin-responsive and macrophage-specific hTNF gene expression can be established in transgenic mice and presents evidence that the 3′-region of the hTNF gene could be involved in macrophage-specific transcription. Transgenic mice carrying 3′-modified hTNF transgenes shows deregulated patterns of expression and develop chronic inflammatory polyarthritis. Keffer et al. shows transgenic mice that predictably develop arthritis represent a genetic model by which the pathogenesis and treatment of this disease in humans can be further investigated.

Sakai et al. (1998), Potential withdrawal of rheumatoid synovium by the induction of apoptosis using a novel in vivo model of rheumatoid arthritis, Arthritis Rheum 41:1251-7, investigates whether Fas-mediated apoptosis has potential as a therapeutic strategy in rheumatoid arthritis (RA) by use of a model of RA in which human RA tissue is grafted into SCID mice. Fresh rheumatoid synovial tissue including joint cartilage was grafted subcutaneously into the backs of SCID mice. Six weeks after engraftment, anti-Fas monoclonal antibody was injected intraperitoneally. Time-related apoptotic changes caused by anti-Fas monoclonal antibody in grafted synovium were evaluated by nick end-labeling histochemistry. Thirty-six hours after the injection, diffuse apoptotic changes were observed in the grafted synovia. Four weeks after the injection, rheumatoid synovial tissue diminished.

Smith et al. (1999), Diacerhein treatment reduces the severity of osteoarthritis in the canine cruciate-deficiency model of osteoarthritis, Arthritis Rheum 42:545-54, describes a canine model of osteoarthritis (OA). OA was induced in 20 adult mongrel dogs by transection of the anterior cruciate ligament of the left knee. The model was used to test treatments for OA.

f. Inflammatory Lung Diseases

Models for testing and demonstrating activity of the conjugates herein for treatment of inflammatory lung diseases are known to those of skill in the art. Exemplary references that provide and use animal models of inflammatory lung diseases which can be used to test ligand-toxin conjugates, such as LPM conjugates containing a modified SA1 moiety, include, but are not limited to, the following references set forth herein.

Kumagai et al. (1999), Inhibition of Matrix Metalloproteinases Prevents Allergen-Induced Airway Inflammation in a Murine Model of Asthma, J Immunol 162:4212-4219, investigates the role of MMPs in the pathogenesis of bronchial asthma, using a murine model of allergic asthma. Using this model, an increased release of MMP-2 and MMP-9 in bronchoalveolar lavage fluids after Ag inhalation in the mice sensitized with OVA, which was accompanied by the infiltration of lymphocytes and eosinophils, is reported. Administration of tissue inhibitor of metalloproteinase-2 to airways inhibited the Ag-induced infiltration of lymphocytes and eosinophils to airway wall and lumen, reduced Ag-induced airway hyperresponsiveness, and increased the numbers of eosinophils and lymphocytes in peripheral blood. The inhibition of cellular infiltration to airway lumen was observed also with tissue inhibitor of metalloproteinase-1 and a synthetic matrix metalloproteinase inhibitor. The data indicate that MMPs, especially MMP-2 and MMP-9, are crucial for the infiltration of inflammatory cells and the induction of airway hyperresponsiveness, which are pathophysiologic features of bronchial asthma.

Griffiths-Johnson et al. (1997), Animal models of asthma: role of chemokines, Methods Enzymol 288:241-66, describes numerous chemokines that have been discovered through the use of (1) bioassay of in vitro cell culture supernatants and in vivo exudates from animal models of inflammation and (2) molecular biology techniques. There is compelling evidence from animal and clinical studies that eosinophils are important effector cells in asthma. Griffiths-Johnson et al. identify two targets to prevent eosinophil recruitment to the lung: IL-5 and its receptor, which are important in several aspects of eosinophil biology; and eotaxin and its receptor, CCR3. The eotaxin receptor is expressed in high numbers on eosinophils, but not on other leukocytes, and appears to be the major detector of the eosinophil for eotaxin and other chemokines such as MCP-4. Eotaxin and CCR3 knockout mice will allow the evaluation of mediators involved in asthma, as well as the testing of specific therapeutic modalities.

Campbell et al. (1998), Temporal role of chemokines in a murine model of cockroach allergen-induced airway hyperreactivity and eosinophilia, J Immunol 161:7047-53, provides a murine model of cockroach allergen-induced airway disease and assesses specific mechanisms of the response, which resembles atopic human asthma. The allergic responses in this model include allergen-specific airway eosinophilia and significantly altered airway physiology, which directly correlates with inflammation. Specific roles for CC chemokines during these stages, with MIP-1α being an important eosinophil attractant during the primary stage and eotaxin during the secondary re-challenge stage are identified. These models allow the evaluation of mediators involved in both stages of cockroach allergen challenge, as well as the testing of specific therapeutic modalities.

Piguet et al. (1989), Tumor necrosis factor/cachectin plays a role in bleomycin-induced pneumopathy and fibrosis, J Exp Med 170:655-63 and Schrier et al. (1983), The effects of the nude (nu/nu) mutation on bleomycin-induced pulmonary fibrosis. A biochemical evaluation, Am Rev Respir Dis 127:614-617, describe a mouse model of pulmonary fibrosis.

Steinhauser et al. (1999), IL-10 is a major mediator of sepsis-induced impairment in lung antibacterial host defense, J Immunol 162:392-399, describes a murine model of sepsis-induced Pseudomonas aeruginosa pneumonia to explore the mechanism of immunosuppression associated with sepsis. CD-1 mice underwent either cecal ligation using a 26-gauge needle puncture (CLP) or sham surgery, followed by the intratracheal (i.t.) administration of P. aeruginosa or saline. Survival in mice undergoing CLP followed 24 h later by the i.t. administration of saline or P. aeruginosa was 58% and 10%, respectively, whereas 95% of animals undergoing sham surgery followed by P. aeruginosa administration survived. Increased mortality in the CLP/P aeruginosa group was attributable to markedly impaired lung bacterial clearance and the early development of P. aeruginosa bacteremia. The i.t. administration of bacteria to CLP-, but not sham-, operated mice resulted in an intrapulmonary accumulation of neutrophils. Furthermore, P. aeruginosa challenge in septic mice resulted in a relative shift toward enhanced lung IL-10 production concomitant with a trend toward decreased IL-112. The i.p., but not i.t., administration of IL-10 Abs given just before P. aeruginosa challenge in septic mice significantly improved survival and clearance of bacteria from the lungs of septic animals administered P. aeruginosa. Finally, alveolar macrophages isolated from animals undergoing CLP displayed a marked impairment in the ability to ingest and kill P. aeruginosa ex vivo, and this defect was partially reversed by the in vivo neutralization of IL-10. Collectively, these observations indicate that the septic response substantially impairs lung innate immunity to P. aeruginosa, and this effect is mediated by endogenously produced IL-10.

g. Inflammation After Gene Therapy

Models for confirming or identifying conjugates herein for treatment of inflammation, including inflammation after gene therapy are known. For example, Muruve et al. (1999), Adenoviral gene therapy leads to rapid induction of multiple chemokines and acute neutrophil-dependent hepatic injury in vivo, Hum Gene Ther 10:965-76, studies the molecular mechanisms by which replication-deficient adenoviruses induce acute injury and inflammation of infected tissues, which limits their use for human gene therapy. To characterize this response, chemokine expression was evaluated in DBA/2 mice following the intravenous administration of various adenoviral vectors. Administration of adCMVbeta gal, adCMV-GFP, or FG140 intravenously rapidly induced a consistent pattern of C—X—C and C—C chemokine expression in mouse liver in a dose-dependent fashion. One hour following infection with 10(10) PFU of adCMVbeta gal, hepatic levels of MIP-2 mRNA were increased >60-fold over baseline. MCP-1 and IP-10 mRNA levels also were increased immediately following infection with various adenoviral vectors, peaking at 6 hr with >25- and >100-fold expression, respectively. Early induction of RANTES and MIP-1β mRNA by adenoviral vectors also occurred, but to a lesser degree. The induction of chemokines occurred independently of viral gene expression since psoralen-inactivated adenoviral particles produced an identical pattern of chemokine gene transcription within the first 16 hr of administration. The expression of chemokines correlated as expected with the influx of neutrophils and CD11b+ cells into the livers of infected animals. At high titers, all adenoviral vectors caused significant hepatic necrosis and apoptosis following systemic administration to DBA/2 mice. To investigate the role of neutrophils in this adenovirus-induced hepatic injury, animals were pretreated with neutralizing anti-MIP-2 antibodies or depleted of neutrophils. MIP-2 antagonism and neutrophil depletion each and both resulted in reduced serum ALT/AST levels and attenuation of the adenovirus-induced hepatic injury histologically, confirming that this early injury is largely due to chemokine production and neutrophil recruitment. The results clarify the early immune response against replication deficient adenoviral vectors and indicate a strategy to prevent adenovirus-mediated inflammation and tissue injury by interfering with chemokine or neutrophil function.

h. Angiogenesis

Conjugates provided herein can target cells that are upregulated in angiogenesis and processes involved therein. Exemplary references that provide and use animal models of angiogenesis for confirming or identifying ligand-toxin conjugates, such as LPM conjugates containing a modified SA1 moiety, include, but are not limited to, the following references.

Folkman et al. (1987), Angiogenic factors, Science 235:442-7, establishes the role of angiogenesis and factors, such as acidic and basic fibroblast growth factor, angiogenin, and transforming growth factors alpha and beta, and their significance in understanding growth regulation of the vascular system. When evaluated according to the targets, the factors fall into two groups: those that act directly on vascular endothelial cells to stimulate locomotion or mitosis, and those that act indirectly by mobilizing host cells (for example, macrophages) to release endothelial growth factors. In addition to their presence in tumors undergoing neovascularization, the same angiogenic peptides are found in many normal tissues where neovascularization is not occurring. This indicates that physiological expression of angiogenic factors is tightly regulated. In addition to the persistent angiogenesis induced by tumors, it now appears that a variety of nonneoplastic diseases, previously thought to be unrelated, can be considered as “angiogenic diseases” because they are dominated by the pathologic growth of capillary blood vessels.

Leibovich et al. (1987), Macrophage-induced angiogenesis is mediated by tumor necrosis factor-alpha, Nature 329:630-632, describes that macrophages are important in the induction of new blood vessel growth during wound repair, inflammation and tumor growth and investigate this by studying capillary blood vessel formation in the rat cornea and the developing chick chorioallantoic membrane.

Koch et al. (1992), Interleukin-8 as a macrophage-derived mediator of angiogenesis, Science 258:1798-1801, describes that angiogenic factors produced by monocytes/macrophages are involved in the pathogenesis of chronic inflammatory disorders characterized by persistent angiogenesis. The role of interleukin-8 (IL-8), which is chemotactic for lymphocytes and neutrophils, was shown to be potently angiogenic when implanted in the rat cornea and induces proliferation and chemotaxis of human umbilical vein endothelial cells. The data indicate a role for macrophage-derived IL-8 in angiogenesis-dependent disorders, such as rheumatoid arthritis, tumor growth, and wound repair.

i. Tumor Growth

Conjugates provided herein (such as growth factor-toxin conjugates, ErbB receptor conjugates and others) can be used for treatment of tumors, such as by targeting tumor receptors and/or cells involved in tumorigenesis, including angiogenesis. Recruitment of cells involved in angiogenesis and inflammation are associated with tumor growth and development. The following references describe these relationships and that animal models for identifying therapies for tumor, angiogenesis and inflammatory response inhibitors are known to those of skill in the art. These references evidence the availability of animal models for the study of therapeutics for inhibition of tumor growth and cells associated therewith. Ligand-toxin conjugates provided herein, including LPM conjugates containing a modified SA1 moiety, can be used in such models to assess effects on tumor growth.

Phillips et al. (1994), Transforming growth factor-alpha-Pseudomonas exotoxin fusion protein (TGF-alpha-PE38) treatment of subcutaneous and intracranial human glioma and medulloblastoma xenografts in athymic mice, Cancer Res 54:1008-15, exploits the differential expression of epidermal growth factor receptor (EGFR), which is amplified or overexpressed in many malignant gliomas and other primary brain tumors, but is low or undetectable in normal brain, for targeted brain tumor therapy using a TGF-alpha-Pseudomonas exotoxin recombinant toxin, TGF-alpha-PE38 using nude mice bearing glioblastoma or medulloblastoma subcutaneous xenografts. The xenograft model can be useful for studying chemokine receptor-targeting conjugates for treatment of inflammatory responses and targeting of cells involved in tumor development.

Debinski et al. (1994), Interleukin-4 receptors expressed on tumor cells can serve as a target for anticancer therapy using chimeric Pseudomonas exotoxin, Int J Cancer 58:744-748, reports the use of chimeric proteins composed of human IL4 (hIL4) and 2 different mutant forms of a powerful bacterial toxin, Pseudomonas exotoxin A (PE) in a human solid tumor xenograft model. The 2 chimeric toxins, termed hIL4-PE4E and hIL4-PE38QQR, showed specific, hIL4R-dependent and dose-dependent antitumor activities.

Husain et al. (1998), Complete regression of established human glioblastoma tumor xenograft by interleukin-4 toxin therapy, Cancer Res 58:3649-53, shows use of an IL-4 toxin conjugate for targeted treatment of glioblastoma flank tumors in nude mice. Kreitman et al. (1998), Accumulation of a recombinant immunotoxin in a tumor in vivo: fewer than 1000 molecules per cell are sufficient for complete responses, Cancer Res 58:968-975, also demonstrate use of this model.

McDonald et al. (2001), The therapeutic potential of chemokine-toxin fusion proteins, I Drugs 4:427-442, reports that SDF-1β-SA1 (wildtype SA1) retards the growth of HT-29 colon carcinoma tumors in two separate mouse xenograft models. SDF-1β-SA1 also eradicated newly forming intratumoral blood vessels as evidenced by the lack of cross sectioned vessels in treated tumors versus control tumors.

Exemplary results of an LPM conjugate in a model of tumor growth is set forth in Example 9, which shows the results of experiments testing LPM1d in a xenograft model of tumor growth. Other LPMs, such as any provided herein containing a chemokine conjugated to a modified SA1, also can be tested in similar assays. Such results demonstrate that LPMs can be used as candidate therapeutics for treatment of cancer and angiogenesis.

j. Human Immunodeficiency Virus (HIV) and Other Viruses

Conjugates with toxin moieties can target cells infected with viruses, such as, but are not limited to, HIV, hepatitis A, B, and/or C, and other viruses that chronically infect cells. The mode of action can be via the effects of toxins on cellular metabolism. In addition, toxins, such as Shiga toxin, and the modified Shiga toxin and active fragments provided herein, are polynucleotide adenosine glycosidases that depurinate polynucleotides, including RNA and DNA, including viral nucleic acids. Hence conjugates that are targeted to receptors expressed on virally infected cells can treat viral infection.

For example, conjugates provided herein can be used, to target HIV infected cells and destroy viral nucleic acid and/or inhibit or kill the cells. Some exemplary references that provide and use animal models of HIV that can be used to test ligand-toxin conjugates, such as LPM conjugates containing a modified SA1, include, but are not limited to, the following references. Westmoreland et al. (1998), Chemokine receptor expression on resident and inflammatory cells in the brain of macaques with simian immunodeficiency virus encephalitis, Am J Pathol 152:659-665, describes that a correlation between monocyte/macrophage infiltrates in the brain and neurological disease exists, and that chemokines and chemokine receptors could play roles in HIV neuropathogenesis and describes their pattern of expression in the SIV-infected rhesus macaque model of HIV encephalitis. Elevated expression of the chemokines MIP-1α, MIP-1β, RANTES, and IP-10 in the brains of macaque monkeys with SIV encephalitis had been demonstrated and in this study the corresponding chemokine receptors CCR3, CCR5, CXCR3, and CXCR4 were shown to be expressed in perivascular infiltrates in these same tissues. In addition, CCR3, CCR5, and CXCR4 were detected on subpopulations of large hippocampal and neocortical pyramidal neurons and on glial cells in normal and encephalitic brain. The data and results indicate that multiple chemokines and their receptors contribute to monocyte and lymphocyte recruitment to the brain in SIV encephalitis. Furthermore, the expression of known HIV/SIV co-receptors on neurons indicates a possible mechanism whereby HIV or SIV can directly interact with these cells, disrupting their normal physiological function and contributing to the pathogenesis of AIDS dementia complex.

Tyor et al. (1993), A model of human immunodeficiency virus encephalitis in SCID mice, Proc Natl Acad Sci USA 90:8658-62, provides an animal model of HIV-associated dementia complex to aid in development of treatments therefor. Mice with severe combined immunodeficiency (SCID mice), which accept xenografts without rejection, were intracerebrally inoculated with human peripheral blood mononuclear cells and HIV. One to 4 weeks after inoculation, the brains of these mice contained human macrophages (some of which were HIV p24 antigen positive), occasional multinucleated cells, and striking gliosis by immunocytochemical staining. Human macrophages also were frequently positive for tumor necrosis factor type alpha and occasionally for interleukin 1 and VLA-4. Cultures of these brains for HIV were positive. Generally, human macrophages were not present in the brains of control mice, nor was significant gliosis. HIV was not recovered from mice that received HIV only intracerebrally. Pathologically, this model of HIV encephalitis in SCID mice resembles HIV encephalitis in humans and the data indicate that the activation of macrophages by infection with HIV results in their accumulation and persistence in brain and in the development of gliosis. This model of HIV encephalitis provides insights into the pathogenesis and treatment of this disorder.

Toggas et al. (1994), Central nervous system damage produced by expression of the HIV-1 coat protein gp120 in transgenic mice, Nature 367:188-193, provides transgenic mice that express gp120 in their brains and used these mice to study the role of gp120 in the neuronal and glial observed in humans. The changes observed in brains of the transgenic mice resemble abnormalities in brains of HIV-1-infected humans. The severity of damage correlated positively with the brain level of gp120 expression. These results provide in vivo evidence that gp120 plays a role part in HIV-1-associated nervous system impairment. This facilitates the evaluation and development of therapeutic strategies aimed at HIV-brain interactions.

Wykrzykowska et al. (1998), Early regeneration of thymic progenitors in rhesus macaques infected with simian immunodeficiency virus, J Exp Med 187:1767-1778, using the SIV/macaque model of AIDS, examines the early effects of SIV on the thymus.

Krucker et al. (1998) Transgenic mice with cerebral expression of human immunodeficiency virus type-1 coat protein gp120 show divergent changes in short- and long-term potentiation in CA1 hippocampus, Neuroscience 83:691-700, studies transgenic mice constitutively expressing glial fibrillary acidic protein-driven gp120 from brain astrocytes which display neuronal and glial changes resembling abnormalities in human immunodeficiency virus type-1-infected human brains.

Power et al. (1998), Neurovirulence in feline immunodeficiency virus-infected neonatal cats is viral strain specific and dependent on systemic immune suppression, J Virol 72:9109-15, provides an animal model of HIV and its role in immune suppression. Feline immunodeficiency virus (FIV) is a lentivirus that causes immune suppression and neurological disease in cats. To determine the extent to which different FIV strains caused neurological disease, FIV V1CSF and Petaluma were compared in ex vivo assays and in vivo. Both viruses infected and replicated in macrophage and mixed glial cell cultures at similar levels, but V1CSF induced significantly greater neuronal death than Petaluma in a neurotoxicity assay. V1CSF-infected animals showed significant neurodevelopmental delay compared to the Petaluma-infected and uninfected animals. Magnetic resonance spectroscopy studies of frontal cortex revealed significantly reduced N-acetyl aspartate/creatine ratios in the V1CSF group compared to the other groups. Cyclosporin A treatment of Petaluma-infected animals caused neurodevelopmental delay and reduced N-acetyl aspartate/creatine ratios in the brain. Reduced CD4(+) and CD8(+) cell counts were observed in the V1CSF-infected group compared to the uninfected and Petaluma-infected groups. These findings indicate that neurodevelopmental delay and neuronal injury is FIV strain specific but that systemic immune suppression also is an important determinant of FIV-induced neurovirulence.

Models for other viral infections are known and can be used to confirm anti-viral activity for other viruses.

k. Kidney Disease

Conjugates provided herein can be used for treatment of kidney disease. Animal models of kidney disease can be used to test ligand-toxin conjugates, such as LPM conjugates containing a modified SA1. Such animal models include those that mimic different human chronic kidney diseases (CKDs), which are well characterized. An exemplary reference that reviews several well characterized CKD models. including anti-GBM disease, and their relevance to human disease is Durvasula and Shankland (Methods Mol. Med., 86: 47-66, 2003).

For example, anti-Thy-1 induced glomerulonephritis in the rat as a model of human mesangioproliferative glomerulonephritis has been fully described (see Jefferson and Johnson (1999) J. Nephrol. 12:297-307; Westerhuis et al. (2000) Am. J. Pathol., 156: 303-10). Briefly, rats are injected with anti-thymocyte antibody which binds to glomerular mesangial cells (MGCs) and leads to complement-dependent mesangiolysis. Mesangiolysis ends by day 2 which is followed by MGCs proliferation and hypercellularity peaking around days 5-7. Thereafter the MGCs undergo apoptosis until the model resolves itself. During the proliferative phase there is an alteration in MGC phenotype which is associated with the deposition of extra cellular matrix proteins (ECM) an early indicator of fibrosis. In the first minutes there is an upregulation of soluble inflammatory mediators including the chemokine MCP-1 which correlates with an influx of leukocytes, most importantly macrophages. Macrophages are thought to contribute to mesangiolysis in the early phase by producing reactive nitrogen and oxygen species. In the later phase they are thought to contribute to MGC proliferation and ECM production via the production of cytokines and growth factors including the profibrotic TGF-β. The numbers of macrophages peak between around 2 and 4 and gradually decrease thereafter. It has been shown that MCP-1 neutralization in this model ameliorates macrophage infiltration, TGF-β production, and synthesis of ECM proteins. In another study, depletion of macrophages with clodronate liposomes resulted in a marked reduction of mesangial matrix expansion.

Exemplary results of an LPM conjugate in a model of kidney disease is set forth in Example 6, which shows the results of experiments testing LPM1d in an anti-Thy-1 induced glomerulonephritis model. The results show that LPM1d provides renal protection in a number of tested physiological parameters. Other LPMs, such as any provided herein containing a chemokine conjugated to a modified SA1, also can be tested in similar assays. Such results demonstrate that LPMs can be used as candidate therapeutics for treatment of kidney disease.

l. Hypersensitivity

Some exemplary references that provide and use animal models of hypersensitivity, which can be used to test ligand-toxin conjugates, such as LPM conjugates containing a modified SA1, include, but are not limited to, the following references set forth herein. The mouse delayed-type hypersensitivity (MDTH) was initially developed to provide a test for contact hypersensitivity. It has been adapted to screen for suppression of T-cell modulated immune response and is commonly used as a model of chronic inflammatory disease (Staite et al., (1996) Blood 88: 2973-2979). For example, in several models and in particular, the oxazalone (OXA)-induced allergic contact dermatitis mouse model has been used to identify potential anti-inflammatory and immunomodulating drugs (Chapman et al., (1986) Am. J. Dermatopathol. 130-8). Mice sensitized to oxazolone, undergo a reproducible and measureable inflammatory response when a solution of the oxazolone is applied directly to the ear. Hapten-specific dermal T lymphocytes (a mixture of Th1 and Th2 cells) and macrophages are triggered to release proinflammatory cytokines and chemokines. There also is neutrophil activation and infiltration although there numbers are in the minority. Other DTH model studies have shown that neutrophils can indirectly or directly regulate the recruitment of T cells by releasing cytokines and chemokines. Within hours, the ear swells and leukocytes begin to infiltrate the extravascular tissue. Ear thickness and cellular infiltration peak at 24 hours and gradually decline to baseline levels over several days. The mouse ears increase in weight with the influx of leukocytes and production of exudate.

Exemplary results of an LPM conjugate in a model of hypersensitivity is set forth in Example 7, which shows the results of experiments testing LPM1c and LPM1d in a model of hypersensitivity. Most of the leukocyte subtypes involved in ear swelling in the MDTH model express CCR2, the targeted receptor for MCP-1, among other chemokine receptors. Thus, MCP-1-SA1 (LPM1) conjugates containing a modified SA1 were selected to target these cells for elimination. The results exemplify that MCP-1-SA1 (LPM1) variants LPM1c and LPM1d were efficacious in the treatment of hypersensitivity, and that LPM1c and LPM1d have differing potencies consistent with their toxic activity as set forth (see, e.g., in Example 3). Other LPMs, such as any provided herein containing a chemokine conjugated to a modified SA1, also can be tested in similar assays. Any LPM conjugates can be tested, particularly any LPM conjugate known to target a cell surface receptor, such as any cell surface receptor expressed on one or more leukocytes involved in hypersensitivity. Hence, such results demonstrate that LPMs can be used as candidate therapeutics for treatment of hypersensitivity.

### J. FORMULATION AND ADMINISTRATION OF COMPOSITIONS CONTAINING TOXINS AND CONJUGATES THEREOF

Compositions for use in treatment of disorders associated with pathophysiological inflammatory responses, including secondary tissue damage and associated disease states. as well as other diseases are provided herein. Such compositions contain a therapeutically effective amount of a ligand-toxin conjugate that contain a targeting agent, such as for example, a chemokine or active fragment thereof, and a RIP toxin, as described herein. Other conjugates known to those of skill in the art also are contemplated can be modified such that the toxin portion is replaced with the toxins provided herein, and compositions containing such conjugates also are contemplated.

Effective concentrations for treatment of a condition or disease of one or more ligand-toxin conjugates, such as for example the LPMs provided herein, or pharmaceutically acceptable derivatives thereof are mixed with a suitable pharmaceutical carrier or vehicle for systemic, topical or local administration. Compounds are included in an amount effective for treating the selected disorder. The concentration of active compound in the composition will depend on absorption, inactivation, excretion rates of the active compound, the dosage schedule, and amount administered as well as other factors known to those of skill in the art.

Pharmaceutical carriers or vehicles suitable for administration of the conjugates and for the methods provided herein include any such carriers known to those skilled in the art to be suitable for the particular mode of administration. In addition, the compounds can be formulated as the sole pharmaceutically active ingredient in the composition or can be combined with other active ingredients.

The precise amount or dose of the therapeutic agent administered depends on the particular conjugate, the route of administration, and other such considerations. It can be administered in a slow release delivery vehicle, such as, but are not limited to, microspheres, liposomes, microparticles, nanoparticles, and colloidal carbon. Typically a therapeutically effective dosage should produce a serum concentration of active ingredient of from about 0.1 ng/ml to about 50-100 μg/ml. The pharmaceutical compositions typically should provide a dosage of from about 0.01 mg to about 100-2000 mg of conjugate, depending upon the conjugate selected, per kilogram of body weight per day. Typically, for intravenous or systemic treatment a daily dosage of about between 0.05 and 0.5 mg/kg should be sufficient. Local application should provide about 1 ng up to 100 μg, typically about 1 μg to about 10 μg, per single dosage administration. It is understood that the amount to administer will be a function of the conjugate selected, the indication treated, and possibly the side effects that will be tolerated. Dosages can be empirically determined using recognized models for each disorder.

The active ingredient can be administered at once, or can be divided into a number of smaller doses to be administered at intervals of time. It is understood that the precise dosage and duration of treatment is a function of the tissue being treated and can be determined empirically using known testing protocols or by extrapolation from in vivo or in vitro test data. It is to be noted that concentrations and dosage values also can vary with the age of the individual treated. It is to be further understood that for any particular subject, specific dosage regimens should be adjusted over time according to the individual need and the professional judgment of the person administering or supervising the administration of the compositions, and that the concentration ranges set forth herein are exemplary.

The compound can be suspended in micronized or other suitable form or can be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the targeted condition and can be empirically determined. To formulate a composition, the weight fraction of compound is dissolved, suspended, dispersed, or otherwise mixed in a selected vehicle at an effective concentration such that the targeted condition is relieved or ameliorated.

For local internal administration, such as, intramuscular, parenteral or intra-articular administration, the compounds are generally formulated as a solution or suspension in an aqueous-based medium, such as isotonically buffered saline or are combined with a biocompatible support or bioadhesive intended for internal administration.

The resulting mixtures can be solutions, suspensions, emulsions or other such mixtures, and can be formulated as an aqueous mixture, creams, gels, ointments, emulsions, solutions, elixirs, lotions, suspensions, tinctures, pastes, foams, aerosols, irrigations, sprays, suppositories, bandages, or any other formulation suitable for systemic, topical or local administration.

Pharmaceutical and cosmetic carriers or vehicles suitable for administration of the compounds provided herein include any such carriers known to those skilled in the art to be suitable for the particular mode of administration. In addition, the compounds can be formulated as the sole pharmaceutically active ingredient in the composition or can be combined with other active ingredients. The active compound is included in the carrier in an amount sufficient to exert a therapeutically useful effect in the absence of serious toxic effects on the treated individual. The effective concentration can be determined empirically by testing the compounds using in vitro and in vivo systems, including the animal models described herein.

Solutions or suspensions used for local application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid [EDTA]; buffers, such as acetates, citrates and phosphates; and agents for the adjustment of tonicity such as sodium chloride or dextrose. Liquid preparations can be enclosed in ampules, disposable syringes or multiple dose vials made of glass, plastic or other suitable material. Suitable carriers can include physiological saline or phosphate buffered saline [PBS], and the suspensions and solutions can contain thickening and solubilizing agents, such as glucose, polyethylene glycol, and polypropylene glycol and mixtures thereof. Liposomal suspensions also can be suitable as pharmaceutically acceptable carriers. These can be prepared according to methods known to those skilled in the art.

The therapeutic agents for use in the methods can be administered by any route known to those of skill in the art, such as, but are not limited to, topically, intraarticularly, intracistemally, intraocularly, intraventricularly, intrathecally, intravenously, intramuscularly, intraperitoneally, intradermally, intratracheally, as well as by any combination of any two or more thereof.

The most suitable route for administration will vary depending upon the disease state to be treated, for example the location of the inflammatory condition. Modes of administration include, but are not limited to, topically, locally, intraarticularly, intracistemally, intraocularly, intraventricularly, intrathecally, intravenously, intramuscularly, intratracheally, intraperitoneally, intradermally, sterotactically and by a combination of any two or more thereof. For example, for treatment of SCI and other CNS inflammatory conditions, local administration, including administration to the CNS fluid or into the brain (e.g., intrathecally, intraventricularly, or intracisternally) provides the advantage that the therapeutic agent can be administered in a high concentration without risk of the complications that can accompany systemic administration of a therapeutic agent. Alternatively, administration can be by sterotactic inoculation into the brain such as, for example, in treatment of tumors. Similarly, for treatment of inflammatory joint diseases, local administration by injection of the therapeutic agent into the inflamed joint (i.e., intraarticularly, intravenous or subcutaneous means) can be employed. As another example, a disease state associated with an inflammatory skin condition advantageously can be treated by topical administration of the therapeutic agent, for example formulated as a cream, gel, or ointment. For treatment of a disease state associated with an inflammatory lung condition, the preferred route for administration of the therapeutic agent can be by inhalation in an aerosol, or intratracheally.

Hence, the conjugates can be administered by any appropriate route, for example, orally, parenterally (e.g., intravenously, intraperitoneally, intramuscularly, intradermally, via subcutaneous injection or infusion or implant), nasally, or via pulmonary, vagina, rectal, sublingual or topical route, in liquid, semi-liquid or solid form and are formulated in a manner suitable for each route of administration. Preferred modes of administration depend upon the indication treated. Dermatological and opthalmologic indications will typically be treated locally; whereas, tumors and SCI and other such disorders, will typically be treated by systemic, intradermal, intramuscular, stereotactic or other modes of administration. The administration can be by injection (using e.g., intravenous or subcutaneous means), but could also be by continuous infusion for slow or timed-administration (using e.g., slow-release devices or minipumps such as osmotic pumps, or skin patches.)

The therapeutic agent is administered in an effective amount. Amounts effective for therapeutic use will, of course, depend on the severity of the disease and the weight and general state of the subject as well as the route of administration. Local administration of the therapeutic agent will typically require a smaller dosage than any mode of systemic administration, although the local concentration of the therapeutic agent can, in some cases, be higher following local administration than can be achieved with safety upon systemic administration.

Since individual subjects can present a wide variation in severity of symptoms and each therapeutic agent has its unique therapeutic characteristics, it is up to the practitioner to determine a subject's response to treatment and vary the dosages accordingly. Dosages used in vitro can provide useful guidance in the amounts useful for in situ administration of the pharmaceutical composition, and animal models can in some cases be used to determine effective dosages for treatment of particular disorders. In general, however, for local administration, it is contemplated that an effective amount of the therapeutic agent will be an amount within the range from about 0.1 picograms (pg) up to about 1 ng per kg body weight. Various considerations in arriving at an effective amount are described in, e.g., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990; and in the studies of Mantyh et al., (1997) Science 278: 275-79, involving the intrathecal injection of a neuronal specific ligand-toxin.

In one embodiment of the compositions and methods provided herein, the therapeutic agent is administered locally in a slow release delivery vehicle, for example, encapsulated in a colloidal dispersion system or in polymer stabilized crystals. Useful colloidal dispersion systems include nanocapsules, microspheres, beads, and lipid-based systems, including oil-in-water emulsions, micelles, mixed micelles, and liposomes. The colloidal system presently preferred is a liposome or microsphere. Liposomes are artificial membrane vesicles which are useful as slow release delivery vehicles when injected or implanted. Some examples of lipid-polymer conjugates and liposomes are disclosed in U.S. Pat. No. 5,631,018, which is incorporated herein by reference in its entirety. Other examples of slow release delivery vehicles are biodegradable hydrogel matrices (U.S. Pat. No. 5,041,292), dendritic polymer conjugates (U.S. Pat. No. 5,714,166), and multivesicular liposomes (Depofoam®, Depotech, San Diego, Calif.) (U.S. Pat. Nos. 5,723,147 and 5,766,627). One type of microsphere suitable for encapsulating therapeutic agents for local injection (e.g., into subdermal tissue) is poly(D,L)lactide microspheres, as described in D. Fletcher (1997) Anesth. Analg. 84:90-94.

Besides delivering an effective therapeutic dose to the site of trauma and decreasing the chance of systemic toxicity, local administration also decreases the exposure of the therapeutic to degradative processes, such as proteolytic degradation and immunological intervention via antigenic and immunogenic responses. Drug derivatization with, for example, monomethoxypoly(ethyleneglycol) also can decrease the likelihood of the above mentioned drawbacks. Pegylation of therapeutics has been reported to increase resistance to proteolysis; increase plasma half-life, and decrease antigenicity and immunogencity. Examples of pegylation methodologies are given by Lu and Felix (1994) Int. J. Peptide Protein Res., 43:127-138; Lu and Felix (1993) Peptide Res., 6:142-6; Felix et al. (1995) Int. J. Peptide Res., 46:253-64; Benhar et al. (1994) J. Biol. Chem., 269:13398-404; Brumeanu et al. (1995) J. Immunol., 154:3088-95.

The compositions provided herein further can contain one or more adjuvants that facilitate delivery, such as, but are not limited to, inert carriers, or colloidal dispersion systems. Representative and non-limiting examples of such inert carriers can be selected from water, isopropyl alcohol, gaseous fluorocarbons, ethyl alcohol, polyvinyl pyrrolidone, propylene glycol, a gel-producing material, stearyl alcohol, stearic acid, spermaceti, sorbitan monooleate, methylcellulose, as well as suitable combinations of two or more thereof.

A composition provided herein also can be formulated as a sterile injectable suspension according to known methods using suitable dispersing or wetting agents and suspending agents. The sterile injectable preparation also can be a sterile injectable solution or suspension in a non-toxic parenterally-acceptable diluent or solvent, for example, as a solution in 1-4, butanediol. Sterile, fixed oils are conventionally employed as a solvent or suspending medium. For this purpose any bland fixed oil can be employed, including, but are not limited to, synthetic mono- or diglycerides, fatty acids (including oleic acid), naturally occurring vegetable oils like sesame oil, coconut oil, peanut oil, cottonseed oil, and other oils, or synthetic fatty vehicles like ethyl oleate. Buffers, preservatives, antioxidants, and the suitable ingredients, can be incorporated as required, can be formulated as a composition.

Oral compositions generally include an inert diluent or an edible carrier and can be compressed into tablets or enclosed in gelatin capsules. For the purpose of oral therapeutic administration, the active compound or compounds can be incorporated with excipients and used in the form of tablets, capsules or troches. Pharmaceutically compatible binding agents and adjuvant materials can be included as part of the composition.

The tablets, pills, capsules, troches and the like can contain any of the following ingredients, or compounds of a similar nature: a binder, such as microcrystalline cellulose, gum tragacanth and gelatin; an excipient such as starch and lactose, a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, and fruit flavoring.

When the dosage unit form is a capsule, it can contain, in addition to material of the above type, a liquid carrier such as fatty oil. In addition, dosage unit forms can contain various other materials which modify the physical form of the dosage unit, for example, coatings of sugar and other enteric agents. The conjugates also can be administered as a component of an elixir, suspension, syrup, wafer, chewing gum or the like. Syrup can contain, in addition to the active compounds, sucrose as a sweetening agent and certain preservatives, dyes and colorings and flavors.

The active materials also can be mixed with other active materials that do not impair the desired action, or with materials that supplement the desired action, such as cis-platin for treatment of tumors.

Finally, the compounds can be packaged as articles of manufacture containing packaging material, one or more conjugates or compositions as provided herein within the packaging material, and a label that indicates the indication for which the conjugate is provided.

### K. METHODS OF TREATMENT OF DISEASES AND DISORDERS USING TOXINS OR CONJUGATES THEREOF

Provided herein are methods of using any one or more of the ligand-toxin conjugates provided herein, including those containing a modified RIP moiety such as a modified SA1, for the treatment of disease or disorders for which the ligand-toxin conjugate is designed to target. By virtue of receptor-specific targeting of the conjugates to cells expressing the targeted receptor, the compositions and methods provided herein permit the selective, deliberate, and surreptitious delivery of therapeutic agent to cells that orchestrate the response to injury or disease. Targeting of the conjugates to cells involved in the pathophysiological processes of immunomodulatory and inflammatory diseases and other traumas permits receptor-mediated internalization of the conjugates thereby facilitating toxin-mediated cell toxicity and elimination of pathological cell components.

Hence, provided herein are methods of using conjugates to treat inflammatory or immune diseases and conditions. To appreciate the use of such conjugates to treat such diseases or conditions, an understanding of the immune system and the participation of pathological cells to the exacerbation of such disease is required, as is a discussion of the limitations of current candidate therapeutics. The following discussion provides such background prefatory to a discussion of the selection and use of ligand-toxin conjugates, such as those containing a modified toxin, in the treatment of exemplary diseases.

1. The Immune Host Defense System and Inflammation

The immune system can be divided into the innate and adaptive arms which together confer an intact immunosurveillance and host defense system. The system includes several heterogeneous populations of leukocytes which include but are not limited to monocytes or macrophages (collectively referred to as mononuclear phagocytes (MNPs), neutrophils (polymorphonuclear neutrophils, PMN), T cells, B cells, eosinophils, basophils, natural killer (NK) cells, dendritic cells (DCs) and mast cells (MaCs)). The innate immune system relies on cells immediately reactive toward invading entities such as microbes and includes MNPs, dendritic cells, neutrophils and NK cells. The adaptive immune system includes T and B cells, which require activation by antigen presenting cells, principally dendritic cells, in order to target specific host invaders. Cells of the innate and adaptive immune responses work in concert with tissue residential cells (TRC; e.g., epithelial cells) in order to maintain a homeostatic balance in many organ specific processes including embryogenesis, angiogenesis, lymphocyte trafficking, wound healing, tissue repair, removal of cellular debris and other unwanted agents such as microbes, viruses or cancer cell clones (e.g., Esche et al., J. Invest. Dermatol., 125: 615-28, 2005; Chaturvedi et al., Indian J. Med. Res., 124: 23-40, 2006; Bunde, J. Exp. Med. 201: 1031-6, 2005; Krishnaswamy et al., Methods Mol. Biol. 315: 13-34, 2005; Martin and Leibovich, Trends Cell Biol., 15: 599-607, 2005; Kim, Curr. Drug Targets Immune Endocr. Metabol. Disord., 4: 343-61, 2004; Moser and Willimann, Ann. Rheum. Dis. 63(suppl 2): 84-9, 2004; Hoebe et al., Nat. Immunol., 5: 971-4, 2004; Schaerli et al., J. Exp. Med., 199: 1265-75, 2004; Olson and Miller, J. Immunol. 173: 3916-24, 2004; Middleton et al., Blood, 100: 3853-60, 2002; Beyer et al., Glia 31: 262-66, 2000).

a. Homeostatic Inflammation

Homeostatic inflammation is a multi-factorial biochemical process that is orchestrated and perpetuated by activated TRCs and activated cells of leukocyte lineage with a pivotal role of the chemokine-messaging system. Soluble factors released from injured and dying cells, immune complexes or complex charged antigens like bacterial lipopolysaccharides (LPS) and viral envelope proteins working via the complement and toll receptor system are common triggers of leukocyte activation and recruitment. In response, leukocytes undergo profound phenotypic changes including the upregulation of cell adhesion molecules (CAMs) and proinflammatory cytokines and chemokines for trafficking and communication with other leukocyte groups. Once at the site of invasion leukocytes produce an armament of cytotoxic mediators. For example, reactive oxygen and nitrogen species, proteolytic enzymes and eicosanoids kill off invading microbes and fungi which are phagocytosed principally by macrophages and PMN. At wound sites for example leukocyte (especially macrophage)-derived growth factors (GFs) including vascular endothelial growth factor (VEGF) and fibroblast GF (FGF) facilitate angiogenesis. Profibrotic factors such as transforming growth factor-beta TGF-β facilitate scarring and wound healing (Krishnaswamy et al. (2005) Methods Mol. Biol. 315: 13-34; Puneet et al. (2005) Am. J. Physiol. Lung Cell Mol. Physiol. 288: L3-15; Taylor et al. (2005) Annu. Rev. Immunol. 23: 901-44; Byrne et al. (2005) J. Cell Mol. Med. 9: 777-94; Carroll (2004) Nat. Immunol. 5: 981-6; Iwasaki and Medzhitov (2004) Nat. Immunol. 5: 987-95; Martin and Leibovich (2005) Trends Cell Biol. 15: 599-607; Liu and Pope (2004) Rheum. Dis. Clin. North. Am., 30: 19-39; Stark et al. (2005) Immunity, 22: 285-94; Gordon (2003) Nat. Rev. Immunol., 3: 23-5; Borish and Steinke (2003) J. Allergy Clin. Immunol., 111: S460-75; Cross and Claesson-Welsh (2001) Trends Pharmacol. Sci., 22, 201-7; Trautmann, et al. (2000) J. Pathol., 190: 100-6). Generally, such immune mediators generated by activated leukocytes are protective, but in certain pathological situations they can become harmful and perpetuate disease.

b. Pathological Inflammation

Inflammatory responses are mediated by immune defense cells that accumulate at the site of tissue injury or trauma to rid the body of unwanted exogenous agents (e.g., microbes) or endogenous agents (e.g., cancer cell clones); to clean up cellular debris, and to participate in tissue and wound healing. Unfortunately, the molecular mechanisms involved in these reparatory (inflammatory) processes can initiate secondary tissue damage, which, in turn, contributes to the pathogenesis and persistent pathology of a number of inflammatory diseases. The molecular mechanisms and the cellular and chemical mediators involved in secondary tissue damage are similar, if not identical, in most inflammatory diseases of man. For example, in pathological situations induced by activation of a great number of stimuli including, but not limited to, viruses, bacteria, parasites, proinflammatory cytokines, chemokines, hypoxia, ischemia, proteinuria (protein in the urine), advanced glycation end products (AGE), autoantibodies, systemic nucleotides, complement, immune complexes, immunoglobulins, and environmental pollutants such as cigarette smoke can lead to the activation of cells including, but not limited to, a variety of leukocytes and TRC including glial cells of the CNS, mesangial cells (MC) of the kidney, and endothelial cells of many organs. The stimuli can be the initiating factor(s) of disease, but the TRC and inflammatory leukocytes are the soldiers of disease pathology. Activated TRC and resident leukocytes express and secrete among other things members of the cytokine, chemokine, and growth factor superfamilies, which facilitate leukocyte activation, infiltration and proliferation at the sites of inflammation. The specific chemokines and other proinflammatory molecules released by the TRC of any given tissue defines the specific leukocyte infiltrate in any given disease or trauma (Lindemans et al. (2006) Clin. Exp. Immunol., 144:409-17; Puneet et al. (2005) Am. J. Physiol. Lung Cell Mol. Physiol. 288: L3-15; Boyle, J. J. (2005) Curr. Vasc. Pharmacol. 3: 63-8; Liu and Pope (2004) Rheum. Dis. Clin. North. Am., 30: 19-39; Tetley (2002) Chest 121: 156S-159S; de Leeuw et al. (2005) Ann. N.Y. Acad. Sci. 1051: 362-71; Drinda, et al. (2005) Rheumatol. Int. 25: 411-3; Raivich and Banati (2004) Brain Res. Brain Res. Rev., 46: 261-81; Tokarska-Rodak et al. (2004) Ann. Agric. Environ. Med. 11: 227-31; Hou et al. (2004) J. Am. Soc. Nephrol., 15: 1889-96; Hayashida et al. (2001) Arthritis Res. 3: 118-26; Garcia-Ramallo et al., (2002) J. Immunol. 169: 6467-73; Kim et al. (2002) Blood 100: 11-6; Perez de Lema (2001) J. Am. Soc. Nephrol., 12:1369-82; Barnes et al., Eur. Respir. J, 22: 672-88, 2003; Luster et al., Nat. Immunol., 6: 1182-90, 2005; Charo and Ransohoff, N. Eng. J. Med., 354: 610-21, 2006).

Precisely which inflammatory mediators, such as cytokines, chemokines and cognate receptors, employed in pathological inflammation depends upon the exact leukocyte subtype involved, the tissue or organ in question and the stage of injury or disease. In addition, the release of inflammatory mediators can lead to pathological cycles that become perpetuated. For example, cytokines and chemokines perpetuate their own production and are released from leukocytes via autocrine and paracrine mechanisms. They also induce the synthesis and release of cytotoxic compounds from the cells that they target. In addition to neurotoxins, resident and infiltrating leukocytes release the same molecules used for homeostatic purposes in order to mediate tissue damage. Cytokines and chemokines induce the expression of cell adhesion molecules (CAMs) and cell surface antigens (including cytokine and chemokine receptors) on various cell types including leukocytes, endothelial, glial and cancer cells. CAMs and glycosaminoglycans (GAGs) are essential for cell trafficking (or migration) in not only homeostatic circumstances but also in pathological inflammatory conditions including cancer metastasis. The upregulation of cell surface antigens contribute to cellular activation which contributes to further production of inflammatory mediators. In addition, the composition of the microenvironmental milieu of inflammatory factors affects the phenotypes of different cells. For example, neutrophils are known to express CXC receptors but in certain cases like septic acute lung injury and reperfusion injury they express CC receptors including CCR2.

An over-zealous infiltration, (chronic) activation and proliferation (increased numbers) of relatively disease-specific subtypes of leukocytes have been categorically demonstrated to underlie the immunopathology of a wide range of hundreds of different clinical conditions, diseases and traumas (see e.g. Table 6; Table 7). Tissue-specific variations are principally a matter of different leukocyte subgroups occupying the lead role, for example, microglia in the early stages of CNS inflammation; eosinophils, Th2 cells and mast cells (MaCs) in allergic inflammation of the lung; and macrophages, Th1 cells and MaCs in chronic kidney diseases (CKDs). In addition, leukocyte derived soluble mediators such as platelet derived growth factor (PDGF) and transforming growth factor-β (TGF-β) are regulators of other pathological processes such as angiogenesis and fibrosis, respectively. The importance of absolute numbers of leukocytes in disease/chronic inflammation process is underscored by recent studies showing that postmenopausal women with high leukocyte cell counts have a 40-50% higher risk of heart attacks, strokes and death than those women with low counts (Cushman, Arch. Intern. Med. 165: 487-8, 2005; Margolis, et al., Arch. Intern. Med. 165: 500-8, 2005). Alveolar macrophages play a role in the pathogenesis of chronic obstructive pulmonary disease (COPD). Patients with COPD have up to a 10-fold increase in MNP numbers in airways, lung parenchyma, bronchoalveolar lavage fluid and sputum compared to controls. Similarly patients with emphysema showed a 25-fold increase in MNP number in the tissue and alveolar space (Tetley, Chest 121:156S-159S, 2002). Adoptive transfer studies showed that the increased numbers of glomerular macrophages correlated with macrophage induced proteinuria (a marker of kidney injury), glomerular cell proliferation and hypercellularity. Further there is a correlation between leukocyte subtype numbers and the severity and progression of a diverse number of diseases (e.g., Ikezumi, et al., Kidney Int., 63: 83-95, 2003; Brightling et al., N. Engl. J. Med. 346: 1699-705, 2002; Panzer et al., Transplantation 78: 1341-50, 2004). Table 8 below sets forth references supporting the role of various leukocytes in the pathology of a variety of diseases and disorders. Table 9 sets forth exemplary leukocyte populations and other immune cells or tissue resident cells involved in the pathology of a number of diseases.

TABLE 8
Leukocytes in the Pathology of Disease

| Disease/Trauma | Exemplary References |
|---|---|
| Arthritic Diseases | Haringman et al., Ann. Rheum. Dis., 65: 294-300, 2006; |
| | Adamopoulos et al., J. Pathol., 208: 35-43, 2006: Ma and Pope, Curr. |
| | Pharm. Des., 11: 569-580, 2005; Haringman et al., Ann. Rheum. Dis., |
| | 63: 1186-94, 2004; Koch, Arthritis. Rheum., 52: 710-21, 2005. |
| Cancer, Angiogenesis & | Lewis and Pollard, Cancer Res., 66: 605-12, 2006; Kakinumama and |
| Metastasis | Hwang, J Leukoc. Biol., 79: 639-51, 2006; Allavena et al., Curr. Cancer |
| | Ther. Rev., 1: 81-92, 2005; Wang et al., J. Transl. Med., 4: 30, 2006. |
| | Mantovani et al., Semin Cancer Biol., 14: 155-60, 2004; Ben-Baruch, |
| | Cancer Metastasis Rev, Published ahead of print, 2006. |
| Cardiovascular Diseases | Hansson et al., Annu. Rev. Pathol. Mech. Dis. 1: 297-329, 2006, Boyle, |
| | Curr. Vasc. Pharmacol., 3; 63-8, 2005; Charo and Taubman, Circ. Res., |
| | 95: 858-66, 2004; Usui et al., Faseb J., 16: 1838-40, 2002. |
| Chronic Kidney Diseases | Galkina and Ley, J.Am.Soc.Nephrol., 17: 368-77, 2006; Eddy, Adv. |
| | Chronic Kidney Dis., 12: 353-65, 2005; Segerer and Nelson, |
| | WorldScientificJournal 5: 835-44, 2005; Segerer et al., |
| | J.Am.Soc.Nephrol., 11: 152-76, 2000; Tipping and Kitching, Clin. Exp. |
| | Immunol., 142: 207-15, 2005. |
| CNS Diseases and Traumas | Minami et al., J. Pharmacol. Sci., 100: 461-470, 2006; Jones et al., |
| | Curr. Pharm. Des. 11: 1223-36, 2005; Sindern, Front. Biosci., 9: 457-63, |
| | 2004; Kim and de Vellis, J. Neurosci. Res., 81: 302-13, 2005; |
| | Offner et al., J. Cereb. Blood Flow Metab., 26: 654-65, 2006; Kaul and |
| | Lipton, Neurotox. Res., 8: 167-86, 2005; Eugenin et al., J. Neurosci., |
| | 26: 1098-106, 2006; Kaul et al., Cell Death Differ., 12(Suppl 1): 878-92, |
| | 2005. Ubogu et al., Trends Pharmacol. Sci., 27: 48-55, 2006. |
| Eye Diseases | Maruyama et al., J. Clin. Invest., 115: 2363-72, 2005; Klitgaard et al., |
| | Acta. Ophthalmol. Scand. 82: 179-83, 2004; Wallace et al., Prog. |
| | Retin. Eye Res., 23: 435-48, 2004; Yoshida et al., J. Leukoc. Biol., 73: |
| | 137-44, 2003. |
| Inflammatory Bowel | Hanauer, Inflamm. Bowel Dis., 12: S1, S3-9, 2006; Oki et al., Lab |
| Diseases | Invest., 85: 137-45, 2005; Gijsbers et al., Eur, J. Immunol., 34: 1992-2000, |
| | 2004; Middel et al., Gut 55: 220-7, 2006. |
| Liver Diseases | Jaeschke and Haseqawa, Liver Int., 26: 912-9, 2006; Simpson et al., |
| | Clin. Sci. (Lond), 104: 47-63, 2003; Wald et al., Eur. J. Immunol. |
| | 34: 1164-74, 2004; Srazzabosco et al., J. Clin. Gastroenterol., 39: S90-S102, |
| | 2005. Duffield et al., J Clin Invest., 115: 56-65, 2005 |
| Pulmonary Diseases | Puneet et al., Am. J. Physiol. Lung Cell Mol. Physiol., 288: L3-15, |
| | 2005; Scott and Wardlaw, Semin. Respir. Crit. Care Med., 27: 128-33, |
| | 2006; Pawankar, Clin. Exp. Allergy 36: 1-4, 2006; Barnes, Pharmacol. |
| | Rev., 56: 515:-48, 2004; Manabe et al., J. Med. Invest., 52: 85-92, 2005; |
| | Razzaque and Taguchi, Pathol. Int. 53: 133-45, 2003. |
| Skin Diseases | Homey, Adv. Dermatol., 21: 251-77, 2005; Ottaviani et al., Eur. J. |
| | Immunol., 36: 118-28, 2006; Fischer et al., J. Clin. Invest., 116: 2748-56, |
| | 2006; Wang et al., J. Clin. Invest., 116: 2105-14, 2006, Kim et al., |
| | J. Clin. Invest., 115: 798-812, 2005.; Stratis et al., J. Clin. Invest., |
| | 116: 2094-2104, 2006; Pastore et al., Eur. J. Dermatol., 14: 203-8, |
| | 2004. |
| Systemic Diseases | Hussein et al., J. Clin. Pathol., 58: 178-84, 2005; Carulli et al., |
| | Arthritis Rheum., 52: 3772-82, 2005; Cancello and Clement, BJOG, |
| | 113: 1141-7, 2006; Tsiligianni et al., BMC Pulm. Med., 5: 8, 2005; |
| | Hansen et al., Arthritis Rheum., 52: 2109-19, 2005; Zampieri et al., |
| | Ann. N.Y. Acad. Sci., 1051: 351-61, 2005; Uzun, Chest 127: 2243-53, |
| | 2005. |
| Transplantation Rejection | Hoffmann et al., Nephrol. Dial. Transplant., 21: 1373-81, 2006; Nicod, |
| | Proc. Am. Thorac. Soc., 3: 444-9, 2006; Wyburn et al., |
| | Transplantation 80: 1641-7, 2005; Perez-Simon et al., Drugs 66: |
| | 1041-57, 2006; Ruster et al., Clin. Nephrol., 61: 30-9, 2004; Belperio |
| | et al., Semin. Crit. Care. Med., 24: 499-530, 2003. |
| Vascular Diseases | Aries et al., IMAJ., 7: 768-73, 2005; Foell et al., J. Pathol., 204: 311-6, |
| | 2004; Ishibashi, et al, Circ. Res., 94: 1203-10, 2004; Wagner et al., |
| | Clin. Exp. Rheumatol., 21: 185-92, 2003; Falk and Jennette, J. Nephrol., |
| | 17(Suppl 8): S3-9, 2004. |
| Obesity | Neels and Olefsky, J. Clin. Invest., 116: 33-5, 2006; Weisberg et al., J. |
| | Clin. Invest., 116: 115-24, 2006; Fantuzzi, J. Allergy Clin. Immunol., |
| | 115: 911-9, 2005; Martinovic et al., Circ. J., 69: 1484-9, 2005; |
| | Weisberg et al., J. Clin. Invest., 112: 1796-808, 2003) |

TABLE 9
Exemplary Leukocyte Cell Types in Human Diseases

| Disease/Trauma | Exemplary Leukocyte Subtypes |
|---|---|
| Cancers (all organs) | |
| General Growth, Angiogenesis & | TAM, T, Eosinophils, B, MaC, PMN, DC, |
| Metastasis | Basophils |
| Breast Cancer | TAM, DC, T, PMN, B |
| Glioma | TAM, PMN, DC |
| Kidney Cancer | TAM, PMN |
| Ovarian Cancer | MNP, T, NK, MaC |
| Cardiovascular Diseases | |
| Atherosclerosis | MNP, T, PMN |
| Myocardial Infarction | MNP, PMN, T, MaC |
| Restenosis | MNP, T, Eosinophils |
| Chronic Kidney Diseases | |
| Diabetic Nephropathy | MNP, T, PMN, MaC |
| Glomerulonephritides | MNP, PMN, T, MaC, DC |
| IgA Nephropathy | MNP, T, PMN, MaC, DC, B |
| Lupus Nephritis | MNP, T, PMN, B, DC, MaC |
| CNS Diseases and Trauma | |
| Alzheimer's Disease | MNP, T, PMN |
| Multiple Sclerosis | MNP, T, Th1, PMN, B |
| Traumatic Brain Injury | MNP, T, PMN |
| Spinal Cord Injury | MNP, T, PMN |
| Spongiform Encephalopathies | MNP, T, B, DC |
| Stroke | MNP, T, PMN, DC, MaC |
| Eye Diseases | |
| Conjunctivitis | MNP, T, MaC, Eosinophils, B |
| Proliferative Vitreoretinopathy | MNP, PMN, T |
| Retinitis and Iritis | MNP, PMN, B, T |
| Uveitis | MNP, T, PMN, DC |
| HIV and AIDS | MNP, T, MaC, DC |
| Inflammatory Bowel Diseases | |
| Crohn's Disease | DC, T, MNP, B, MaC, Eosinophils, PMN |
| Ulcerative Colitis | MNP, T, B, DC, Eosinophils, MaC, PMN |
| Eosinophilic Gastroenteritis | Eosinophils, Th2, MaC, B, PMN |
| Joint Diseases | |
| Gout | MNP, PMN, T, Eosinophils |
| Osteoarthritis | MNP, B, T, PMN, DC |
| Osteoporosis | MNP, T |
| Rheumatoid Arthritis | MNP, DC, PMN, B, T |
| Liver Diseases | MNP, Th1, K, NK, MaC, B, GC |
| Pulmonary Diseases | |
| Acute Lung Injury | PMN, MNP, T, MaC |
| Acute Respiratory Distress Syndrome | PMN, MNP, T, GC, MaC |
| Asthma | Eosinophils, MNP, B, Th2, MaC, NK |
| Chronic Obstructive Pulmonary Disease | MNP, T, PMN, DC, MaC, Eosinophils |
| Cystic Fibrosis | PMN, MNP, Eosinophils, MaC, T, B |
| Emphysema | MNP, PMN, T, MaC, Eosinophils |
| Eosinophilic Pneumonia | Eosinophils, MNP, MNP, T, GC |
| Pulmonary Fibrosis | PMN, T, Eosinophils, MNP, MaC |
| Skin Diseases | |
| Dermatitis | MNP, DC, T, MaC, Eosinophils, B, PMN |
| Eczema | MNP, T, DC, MaC, Basophils |
| Psoriasis | T, MNP, DC, MaC, Basophils, Eosinophils, |
| | PMN |
| Systemic Diseases | |
| Behcet's Disease | PMN, T, B, MNP, Basophils, MaC |
| Sarcoidosis | MNP, PMN, T, Eosinophils, NK, GC |
| Scleroderma | MNP, T, Eosinophils, MNP, DC, B, Basophils, |
| | NK |
| Sepsis | PMN, MNP, T |
| Sjogren's Syndrome | T, B, MNP, DC, MaC, PMN |
| Systemic Lupus Erythematosus | PMN, T, MaC, B, MNP, DC, Basophils |
| Obesity | MNP, T, MaC, Adipocytes |
| Transplantation | |
| Graft Versus Host Disease | MNP, T, DC, MaC, Eosinophils, PMN, B |
| Graft/Organ Rejection | MNP, T, DC, MaC, Eosinophils, NK, B |
| Vascular Diseases | |
| Giant Cell Arteritis | GC, MNP, T, DC |
| Hypertension | MNP, PMN, T, Basophils |
| Varicose Veins | MaC, MNP, DC, T |
| Vasculitides | T, PMN, MNP, Eosinophils, GC |
| Obesity | MNP, T, PMN |

Key:
B = B cell;
T = T cell;
NK = natural killer cell;
Th2 = type 2 helper T cell;
DC = dendritic cell;
MNP = mononuclear phagocytes (monocytes, macrophages and microglia);
GC = giant cell (multinucleated fused macrophage);
TAM = tumor associated macrophage;
PMN = polymononuclear neutrophil;
MaC = mast cell.

2. Candidate Therapeutics

Several approaches aimed at interfering with cellular activities, including for example, pathological leukocyte and cancer cell activities, have and are being explored. A frequently encountered problem with these many agents is a lack of specificity. For example, immunosuppressive agents such as corticosteroids, cyclophosphamide and azathioprine have been used to treat inflammatory diseases however the nonspecific immunosuppressive effects of these drugs have several drawbacks. First, host defense is compromised and can cause life threatening infections and an increased incidence of malignancies due to a lack of immunosurveillance. Second, direct organ toxicity and disruption of metabolic processes is common (see e.g., Ingelfinger and Schwartz, N. Engl. J. Med. 353: 836-9, 2005; Siegal and Sands, Ailment Pharmacol. Ther., 22: 1-16, 2005; Duncan and Wilkes, Proc. Am. Thorac. Soc., 2: 449-55, 2005; Perez-Simon et al., Drugs 66:1041-57, 2006). Other approaches also are being employed to increase the specificity and hence decrease the side effects of drugs. For example, biological response modifiers (BRMs) including cytokine and chemokine receptor antagonists; cytokine and chemokine anti-ligand antibodies; anti-cell adhesion molecules (CAMs), anti-GAG reagents and molecules which interfere with intracellular signal transduction pathways have been developed (e.g., Johnson et al. (2004) Biochem. Soc. Trans., 32: 366-77; Johnson et al. (2004) J. Immunol., 173: 5776-85; E is et al. (2004) Arch. Immunol. Ther. Exp. (Warsz) 52:164-72; McDonald et al. (2001) IDrugs., 4: 427-42; Ribeiro and Horuk (2005) Pharmacol. Ther. 107: 44-58; Wong (2005) Curr. Opin. Pharmacol. 5: 264-71; de Boer (2005) Drug Discov. Today 10: 93 105; Haringman and Tak (2004) Arthritis Res. Ther., 6:93-7; Barber et al. (2005) Nat Med 11: 933-5; Camps et al. (2005) Nat Med 11: 936-43; Schon et al. (2003) J Invest Dermatol., 121: 951-962).

BRMs, however, have limitations in disease treatment because of the compensatory, pleiotropic and heterogeneous nature of the various networks and cascades employed in homeostatic and inflammatory immune responses. Accordingly, one of the reasons for the limitations of the use of BRMs in the treatment of disease is due to the redundancy and crosstalk of cell signaling machinery, including redundancy among cellular receptors and soluble mediators involved in diseases. For example, there is a great deal of redundancy in mediators involved in inflammation, such as by, for example, members of the cytokine, chemokine, and growth factor systems.

Typically, immune cells can express several receptors for soluble ligand mediators, and each receptor can respond to more than one soluble ligand. For example, the chemokines MIP-1α, RANTES, and LEC bind to CCR5, but also bind to CCR1; CCR1 and CCR3; and CCR1 and CCR2, respectively (see Table 5). Hence, antagonists to CCR5 do not interfere with the binding of MIP-1α, RANTES, and LEC to CCR1, CCR2, and/or CCR3, and continue to exert inflammatory effects (see, e.g., Matsui et al., (2002) J. Neuroimmunol. 128: 16-22). In another example, inhibition of the chemokine MCP-1 to reduce macrophage infiltration via CCR2 in disease is not an optimal therapeutic since other chemokines also use CCR2 including, for example, MCP-3, MCP-2, MCP-5, MCP-4, and LEC and macrophages express other chemokine receptors besides CCR2 (see e.g., Table 5). For example, Fujinaka et al. (J. Am. Soc. Nephrol., 8: 1174-8 (1997)) showed that a neutralizing antibody to MCP-1 decreased the numbers of monocytes and macrophages and proteinuria in the glomeruli when treating the subject at 4 days, however, after 8 days anti-MCP-1 treatment did not decrease cell infiltration, urinary protein excretion, or crescent formation. Thus, in this system, the macrophages were activated not only by MCP-1, but also by other factors that contributed to glomerular injury. For example, apart from other CCR2 ligands, macrophages also express CCR1, CCR3, CCR5, and CCR8 and in some cases CXCR1 and 2, of which some or all could have been factors associated with the observed pathology. Anti-MCP-1 treatment also has been observed to have no effect on clinical or immunohistologic improvement in an arthritis trial (see e.g., Haringman et al., (2006) Arthritis Rheum., 54:2387-92).

Hence, most candidate therapeutics target one but not all of the biochemical mediators released or activated by leukocytes, or they kill one particular leukocyte subtype on the false premise that a single cell type is solely responsible for a given disease, which is rarely if ever the case. A more comprehensive approach to the treatment of disease, disorders, or trauma is to eliminate cellular components, such as leukocytes including pathological leukocytes and/or TRCs, involved in the pathology of the disease. There is a correlation between numbers and increased activity of leukocytes and the severity of disease and measured pathological parameters (see e.g., Wada et al. (1996); Zoja et al. (1996); and Chiang et al. (1996; Nikolic-Paterson and Atkins, Nephrol Dial Transplant., 16 (Suppl 5): 3-7, 2001). For example, elimination of pathological leukocytes, such as activated leukocytes, abolishes the production of inflammatory mediators and toxic molecules, and reduces leukocyte trafficking which is responsible for the exacerbation of many diseases. Exemplary of such candidate therapeutics are ligand-toxin conjugates, particularly the chemokine-receptor targeting conjugates that target activated leukocytes. Hence such conjugates are candidate therapeutics for diseases with an inflammatory component or that share an underlying inflammatory pathology.

3. Ligand-Toxin Conjugates

Ligand-toxin conjugates have been generated and are known that specifically target one or more than one cell population involved in the pathology of a disease. Included among these are chemokine toxin conjugates, such as are described in U.S. application Ser. Nos. 09/360,242; 09/453,851; and 09/792,793, now U.S. Pat. Nos. 7,166,702, 7,157,418 and 7,192,736. Such conjugates target to one, and typically more than one cell type, via recognition by one or more than one specific cell surface receptor and are internalized leading to killing of the cell via the toxin moiety. Using such toxin conjugates, it has been demonstrated that specific and judicious eradication of leukocytes and other cells, including pathological leukocytes, can be efficacious in disease treatment (see e.g., McCarron et al. (2005), Mol. Interv., 5: 368-80; Pastan et al. (2006), Nat. Rev. Cancer 6: 559-65; Frankel et al. (2003), Semin. Oncol., 30: 545-57; Pastan (2003), Immunol. Ther. 52: 338-41; Kreitman, (2006) AAPS. J., 8: E532-51; Carter (2006), Nat. Rev. Immunol., 6: 343-57; Cohen (2005) MedGenMed., 7: 72; Edwards et al. (2004) N. Engl. J. Med. 350: 2572-81; Zeisberger et al. (2006) Br. J Cancer, 95: 272-81; Cross et al., J. Neuroimmunol. Aug. 10, 2006, (published online); Cailhier et al. (2005), J. Immunol., 174: 2336-42; van Roon et al. (2005) Ann. Rheum. Dis. 64: 865-70; Sfikakis et al. (2005) Arthritis Rheum., 52: 501-13; Nikolic-Paterson and Atkins (2001) Nephrol. Dial. Transplant., 16: Suppl 5, 3-7; Rajan et al. (1998), J. Immunol. 160: 5955-62; Hu et al. (1997) Cell Immunol., 177: 26-34; Schuh et al. (2003) Eur. J. Immunol., 33: 3080-90; Taoka et al. (1997), Neuroscience 79: 1177-82; Wolff et al., (2004) J. Vasc. Surg., 39: 878-88; Duffield et al., Am. J. Pathol., 167: 1207-19, 2005).

Provided herein are ligand-toxin conjugates containing a modified RIP toxin polypeptide. The conjugates can be used to treat a variety of diseases and disorders for which the conjugate that contains the unmodified RIP toxins is designed. As discussed above, these modified ligand-toxin conjugates exhibit reduced toxicity to host cells, thereby enabling the high yield production of the toxin. The increased production of such modified ligand-toxin conjugates is advantageous for their use as candidate therapeutics and as therapeutics for treatment of targeted diseases and disorders. Modified ligand-toxin conjugates, including those containing a modified SA1, can be used to eliminate cells or otherwise inhibit growth thereof or alter the metabolism thereof. The targeted cells are those involved in the pathology of diseases or disorders, for example cells involved in inflammation, angiogenesis, or cancers.

Among the conjugates that contain the modified toxins, are chemokine ligand-toxin conjugates, designated leukocyte population modulators (LPMs). As described below, LPMs are designed to eradicate activated pathological (inflammatory) leukocytes and other cells or alter the metabolism thereof through the exploitation of the highly regulated chemokine receptors expressed on these cells. The ligand moiety of the LPM is responsible for gaining entry into the cells via expression of a cognate chemokine receptor. Cells expressing the appropriate chemokine receptor will uptake the LPM molecule, which includes a toxin that inhibits growth of the cells, kills the cells or otherwise alters the metabolism thereof, such as by degrading viral nucleic acid or by interfering with protein synthesis. As the pathological cells are removed or inhibited or killed, there is less and less communication among cells as involved in the disease process and proinflammatory mediators are no longer synthesized. Hence, the multi-stimuli involved in the different processes of inflammation or other disease processes (angiogenesis where the targeted cells are endothelial cells, such as those that express VEGFR) are concomitantly shut down.

The methods provided herein permit generation of and isolation of modified toxins, such as RIPs, or conjugates containing such toxins, that are less toxic to the host cell(s) in which they are produced for use in conjugates or produced as conjugates. Hence, higher quantities can be produced. Since the toxins are so potent, a reduction in toxicity of 10-fold, 100-folled, even a 1000-fold or more does not impact on their use in the therapeutic conjugates. Any conjugate known to those of skill in the art or prepared by those of skill in the art that contains a toxin, particularly, an RIP toxin, can be modified by the methods herein or by replacing the toxin with a modified toxin provided herein. Many such conjugates are known. These include those in U.S. Pat. Nos. 7,166,702, 7,157,418 and 7,192,736 as well as cytokine conjugates, such as conjugates of growth factors and antibodies and other polypeptides targeting agents.

Included among the ligand-toxin conjugates are those having a ligand linked, such as a chemokine or active fragment thereof, directly or indirectly to truncated forms of SA1 such as, for example, a variant 1 or variant 2 SA1 as described herein. Exemplary of such conjugates are LPM1a and LPM1b, set forth in SEQ ID NOS: 38 and 40, respectively. In particular, conjugates containing linkage of a chemokine ligand to a modified SA1, include but not limited to, any modified SA1 identified in the methods herein, such as a mutant variant 1 SA1 (i.e. variant 3) or a mutant variant 2 (i.e. variant 4) SA1 moiety, or any other modified SA1 known or discovered to exhibit reduced toxicity. Exemplary of such LPM for use in the methods of treatment herein are any of the LPM conjugates set forth in any of SEQ ID NOS: 42, 44, 46, 48, 50, 52, 54, 56, 58, 60, 62, 64, or 66.

Any cell or cells can be targeted by the ligand-conjugates provided herein so long as the cell(s) expresses one or more than one cell surface receptor that interacts with the ligand-toxin conjugate thereby resulting in the internalization of the conjugate. For example, any such cell that expresses one or chemokine receptors can be targeted by linking chemokine receptor targeting agent to the modified toxin. Exemplary conjugates are provided herein, and include any one or more of the LPM molecules provided herein. Included among targeted cells are leukocytes or other immune cells, particularly, activated leukocytes and immune cells, such as but not limited to, monocytes, macrophages (including alveolar macrophages, microglia, kupffer cells), dendritic cells (including immature or mature dendritic cells and Langerhan cells), T cells (including CD4 positive such as, but not limited to, Th1 and/or Th2 cells, or CD8 positive cells), B cells, eosinophils, basophils, mast cells, natural killer (NK) cells and neutrophils. Also, included are any tissue residential cell (TRC) such as mesangial cells, glial cells, endothelial cells, epithelial cells, tumor cells, fibroblasts, adipocytes, astrocytes, and/or synoviocytes. The expression of the chemokine receptors on the cells can be constitutive or can be inducible, such as due to activation of the cells. Typically, quiescent leukocytes or leukocytes engaged in other functions are not the target of LPMs, such as any provided herein (McDonald et al, IDrugs, 4: 427-42, 2001). Generally, the LPMs are specific to inducible chemokines, such as those chemokines that are upregulated on activated cells due to inflammatory or other conditions that can become pathological and exacerbate the manifestations of various diseases or disorders. Hence, only activated leukocytes or other activated TRC bearing the targeted chemokine receptors are depleted.

For example, in many cases, in order to initiate and sustain a disease process (e.g., cancer) or an inflammatory response, the cells involved are activated and upregulate their expression of cell surface receptors for a variety of ligands. Because receptors involved in trauma and disease are often upregulated, the likelihood of the therapeutic agent being internalized by the correct cells, is increased. Thus, targeting of cellular receptors upregulated in disease processes increases the specificity of a given toxin conjugate for treating a particular disease or disorder.

Exemplary of disease or disorders treated herein are those having an immune or inflammatory cellular component associated with disease pathology, such as discussed in Tables 8 and 9 above. These include, for example, conditions such as trauma and any disease that has an allergic, angiogenic, autoimmune, inflammatory or tumorigenic component. Hence, targeting activated cells, such as but not limited to any activated leukocyte, such as any activated immune effector cell and/or any activated tissue residential cell or other cell involved in a disease or disorder that expresses one or more than one chemokine receptor is contemplated herein for the treatment of a disease or disorder with a ligand-toxin conjugate, such as any LPM conjugate provided herein.

Any disease or disorder, however, treated by toxin conjugates, including those that target cells involved in angiogenesis and cancer and other diseases, can be modified by replacing the toxin polypeptide portion with a modified toxin provided herein or can be modified by the methods provided herein. Exemplary FDA approved therapeutics that can be modified include, for example, Gemtuzumab-ozogamicin is a ligand-toxin fusion protein composed of a humanized monoclonal antibody against CD33; Denileukin diftitox is a ligand-toxin fusion protein composed of the human IL-2 ligand.

Selection of Ligand-Toxin Conjugate for Treatment of Selected Diseases or Disorders

As discussed above and exemplified in Tables 8 and 9, various cells types exacerbate and/or contribute to the pathology of a number of diseases, disorders, and other conditions. In a given disease or disorder it is possible to assemble a profile of the leukocyte subtype(s) or other cell types involved, and the type and distribution of the associated cell surface receptors expressed. Accordingly, ligand-toxin conjugates can be designed that target a specific cell surface receptor or receptors, thereby providing a modality for entry into the affecting cell and a mechanism to treat the specific disease. As described herein, such ligand-toxin conjugates generally include a modified RIP toxin, or active portion thereof, such as a modified SA1, which upon entry into a target host cell kills the cell as a means to treat disease. Hence, the selection of a chosen ligand portion to target to a host cell is an essential factor for design of a ligand-toxin conjugate. The selection of a specific ligand-toxin conjugates for treating disease requires the following steps: 1) selecting the disease to be treated; 2) determining which cells are present in excess and/or contribute to such disease; 3) determining the expression profile of cell surface receptors on the selected cell types; 4) correlating the expression of the cell surface receptor on other cell types that also can be involved in the disease; 5) choosing a ligand that for the chosen cell surface receptor; and 6) constructing the ligand-toxin conjugate.

Precisely which cytokines, chemokines, growth factors, and/or their cognate receptors to target depends upon the exact cell population(s) involved in a particular disease or disorder, the tissue in question, and/or the stage of injury or disease. For example, it has been shown that specific inflammatory chemokine ligand/receptor axes are expressed and prominent in specific diseases. Therefore it is possible to design drugs for specific diseases by choosing the relevant ligand (i.e. chemokine, cytokine, growth factor) that target its cognate receptor on leukocyte subtypes prominent in specific diseases and traumas.

Table 9 sets forth an exemplary list of diseases, and the leukocyte and other cell populations responsible for the pathology or exacerbation of such disease. One of skill in the art knows or can identify populations of cells, such as any one or more cells set forth in Table 9, which contribute to disease progression. Targeting of any one or more of cells involved in a disease or disorder by a ligand-toxin conjugate such as any provided herein can be used to treat the disease or disorder. The selection of the ligand-toxin conjugate to be used in such treatment depends on the expression of cell surface receptors on the cell or cell populations(s) and the specificity of a ligand for such a receptor(s). One of the skilled in the art knows or could identify receptors expressed on specific cell types including considerations of the tissue in question or the state of injury and/or disease. For example, receptor expression can be determined on a cell or population of cells using routine expression studies such as, but not limited to, flow cytometry or real-time PCR methodologies. The cells tested can be cell lines, cultured primary cells, or cells obtained directly from a patient having the disease or disorder (i.e. cells obtained from the patient's tissue, blood or other source.) Likewise, ligand-receptor specificity can be assessed using routine binding assays known to one of skill in the art such as described herein. Ligand binding can be detected, for example, by directly labeling the ligand with fluorescence or radioactivity for direct measurement of binding to a selected target cell via flow cytometry, fluorimetry or radioactive means. Typically, such binding assays are performed at 4° C., but also can be performed at 37° C. to determine if the targeted cell surface receptor mediates endocytosis and internalization of the specific ligand. For purposes of a ligand-conjugate fusion, internalization is a required consideration since the toxin must gain entry into the cytoplasm of the cell in order to exert its toxic effects.

The discussion below describes the design and selection of ligand-toxin conjugates as exemplified by the selection of leukocyte population modulators based on the known expression profiles of chemokines and their cognate receptors. Similar strategies are known or could be used to design other ligand-toxin conjugates. The discussion is meant to be exemplary only. The design of ligand-toxin conjugates requires disease specific considerations, including, for example the stage and severity of the disease. One of skill in the art could design and test ligand-toxin conjugates in various in vitro assays of toxic activity, such as toxic activity against a specific cell or population of cells, and in vivo assays of disease, such as, but not limited to, any described herein.

Selection and Design of Leukocyte Population Modulators

Design of an LPM aimed at treating a particular disease requires selecting the appropriate targeting agent such as, for example, a chemokine ligand(s). Chemokines for use in the conjugates are selected according to the disease or disorder to be treated. As a first requirement, the leukocytes or other cells associated with a particular disease or condition are identified. As discussed herein, the contributions of various leukocyte populations to disease is known (see e.g., Tables 8 and Table 9) or can be determined. A second step is to choose a particular chemokine ligand that targets one or more than one chemokine receptor expressed on one or more than one of the cell populations to be targeted. Such chemokine ligands are chosen based on the specificity of a chemokine for a receptor, as well as the expression profile of chemokine receptors on various cells. Chemokine receptor expression on leukocyte subtypes and chemokine ligand-receptor interactions are known in the art (see e.g., Tables 5 and 6) or can be determined experimentally by one of skilled in the art.

In particular, selection of a preferred chemokine for use in the ligand-toxin conjugates is one that targets a chemokine receptor that is induced under inflammatory and pathological conditions, but is not expressed on cells during immune homeostasis. For example, Table 7 sets forth the chemokine receptor profiles under inflammatory (i.e. pathological) and homoestatic conditions. Such a Table is exemplary only and it is understood that the induced expression of chemokine receptors is context dependent and influenced by various factors, for example, on the stimuli, disease, state or severity of disease, and particular cell populations tested. One of skill in the art knows or can experimentally determine the chemokine profile expression (i.e. chemoprint) on a cell or a population of cells during various conditions or disease states. Selection of a targeting agent that has activity against pathological cells, but not other bystander or quiescent leukocytes, ensures that the activated cells that contribute to disease progression are targeted for killing.

Certain chemokines appear to have more influence in specific disease states than do others. For example, MCP-1 expression appears to regulate acute experimental autoimmune encephalomyelitis (EAE) whereas MIP-1α expression correlates with the severity of relapsing EAE. In another example, immunohistochemical staining of Alzheimer's disease (AD) brain specimens indicates a predominance of MIP-1β expression over several other chemokines. Thus, for example, MIP-1α and MIP-1β would be the ligands of choice for a LPM conjugate to treat MS and Alzheimer's disease, respectively. Ligands, such as MCP-1, IP-10 and RANTES, would be used for the treatment of human MS as their cognate receptors CCR2, CXCR3 and CCR5, respectively are upregulated in the disease. Eotaxins 1, 2 and 3 show high specificity for CCR3 which is preferentially expressed by eosinophils. Therefore, Eotaxin LPMs can be used for eosinophilic (allergic) diseases including various pulmonary and skin diseases including asthma, eosinophylia-myalgia syndrome, nasal allergy, atopic dermatitis and polyposis. In an additional example, PF-4 is a chemokine used to target endothelial cells and can be used for the treatment of angiogenesis or other associated angiogenic diseases such as ocular disorders or diabetes (see e.g., WO 95/12414).

Hence, consideration of other factors, such as for example, the stage of the disease, the severity of the disease, and the time and duration of treatment also influence the choice of chemokine ligand. For example, a particular chemokine LPM exhibiting a higher degree of receptor specificity can be desirable at an early stage of secondary tissue damage, where, for example, microglia and/or macrophages are initiating inflammation. Removing these cells with a very specific agent can reduce the potential for activation of surrounding, and as yet benign cells. When other leukocyte sub-groups are recruited, at intermediate or late stages of disease, a broader spectrum of cell specificity can be desirable. In addition, an appropriate broad spectrum chemokine LPM would deliver a very strong blow to those restricted populations of leukocytes that express multiple types of chemokine receptors.

For example, MCP-1, Eotaxin and SDF-10 are examples of chemokine ligands that exhibit a restricted and very specific receptor binding profile. Such ligands target very specific cell types through a restricted subset of available receptors. MCP-3 and RANTES are examples of ligands having broad cell and receptor binding profiles. Such chemokine ligands can be relevant to a single or broad range of clinical conditions. A ligand that targets a broad range of cell-types using receptor subtypes can be expressed on all the cells or only certain cells. This is largely a function of the cell types that are specific to a given condition or common to a range of conditions.

Based on the above considerations, LPMs can be designed. For example, if a pulmonary disease, such as acute lung injury (ALI), acute respiratory distress syndrome (ARDS), or chronic obstructive pulmonary disease (COPD) is contemplated for treatment, one of skill in the art knows (i.e. such as set forth in Table 9 above), or could determine, that any one or more of the cell types expressed in such diseases including PMN, MNP, T cells, mast cells, immature or mature DCs, and/or eosinophils express one or more of, for example, CCR1, CCR2 and CCR3. Hence, selecting a ligand that is specific for one or more such chemokine receptors (e.g., MCP1, MCP-3 or Eotaxin) is the first step in designing a ligand-toxin conjugate for the treatment of any one or more of ALI, ARDS, or COPD. A second step is to understand the expression of specific chemokine receptors on the different pathological leukocyte subtypes implicated in the disease. A ligand-toxin conjugate having MCP-1, MCP-3 or Eotaxin as a ligand moiety linked directly or indirectly to a modified RIP such as any discovered by the methods herein and/or described herein could be contemplated for use in treatment of pulmonary diseases. Included among such a ligand-toxin conjugate is LPM1d.

The following table summarizes some exemplary ligands for use in the design of LPMs for treatment of selected diseases and conditions.

TABLE 10
Exemplary Ligand(s) and Disease Treated

Ligand(s)

| | |
|---|---|
| | Disease/Condition |
| MCP-1 and 3, RANTES, IP-10, IL-8, GROα | Atherosclerosis and Restenosis |
| MCP-1 and 3, RANTES, SDF-1β | SCI, Traumatic Brain Injury, Stroke, |
| | AD |
| MCP-1and 3, RANTES, IP-10 | Multiple Sclerosis |
| Eotaxin, RANTES, MDC, MCP-1, SDF-1β | HIV |
| Eotaxin, MCP-1 and 4, MDC, IL-8, ENA-78 | Inflammatory Bowel Diseases |
| MCP-1-4, RANTES, IP-10, MIG, IL-8, ENA-78, GROα, | Inflammatory Joint Diseases (e.g., |
| I-TAC | arthritis) |
| | Inflammatory Lung Diseases |
| MIP-1α, MIP-1β, MCP-1, 2, 3, 4, RANTES, IP-10, IL-8, | Acute lung Injuries and Fibroses |
| ENA-78 | |
| Eotaxin, MCP-4, MDC | Allergic and Eosinophil-associated |
| | Diseases |
| MCP-1, IL-8 | Inflammatory Eye Diseases |
| | Cancers |
| SDF-1β, IP-10, MIG, IL-8, ENA-78, GROα | Glioma |
| MCP-1, 3, and 4, RANTES, SDF-1β | Breast |
| MCP-1, IL-8, ENA-78 | Lung |
| MCP-1, RANTES, IP-10 | Inflammatory Kidney Diseases, |
| | Vasculitis and Transplant rejection |

To that end, a number of chemokine-ligand toxin fusion proteins (i.e. LPMs) have been designed to treat diseases according to the predominant cell types involved in the pathology or aggravation of the disease. Exemplary LPMs for the treatment of specific diseases are set forth in Table 11.

TABLE 11
Exemplary Disease Applications for Leukocyte Population
Modulators

| Chemokine Ligand-Toxin | Exemplary Clinical Applications |
|---|---|
| MCP-1-SA1Var4 (LPM1d) | Kidney, CNS, Pulmonary, Heart and Joint |
| | Diseases, Transplantation |
| Eotaxin-SA1Var4 (LPM2) | Allergic Lung, Nasal and Skin Diseases, |
| | Eosinophilic Gastroenteritis |
| SDF-1β-SA1Var4 (LPM3) | Cancer, Joint Diseases and HIV |
| IP-10-SA1Var4 (LPM 7) | Cancer, CNS, Joint, Kidney, |
| | Transplantation |
| MCP-3-SA1Var4 (LPM8) | CNS, Heart, Joint, Kidney |
| Gro-α-SA1Var4 (LPM4) | Cancer and Joint Diseases |
| IL-8-SA1Var4 (LPM 6) | Cancer, Pulmonary, Kidney, Joint |

4. Exemplary Diseases

Ligand-toxin conjugates that target cells involved in pathologies, such those associated with aberrant angiogenesis, those with an underlying inflammatory component, tumor cells and other aberrant cells, virally infected cells, are known or can be prepared. The particular disease to be treated dictates the ligand (targeting agent) or fragment thereof that is selected. Any such conjugate can include the modified toxins provided and described herein (all such description is incorporated by reference in this section as well as all others).

Exemplary of diseases and disease states, are those associated with the proliferation, activation, and migration of various types of inflammatory immune cells including leukocytes and other contributing cells of epithelial or endothelial origin. These events combine to produce a very aggressive and inhospitable environment at the site of an injury or disease. The cell biology of hundreds of diseases and conditions, involving most organ systems, involve pathophysiological inflammatory responses. The cellular components of many of these pathophysiological diseases are exemplified in Table 9 above. Such diseases and disorders can be treated with any of the ligand-toxin conjugates, including any containing a modified RIP such as a modified SA1 moiety, provided herein and/or produced as described herein. Exemplary of such ligand-toxin conjugates used in the methods herein are LPMs, in particular any LPM provided herein that has been designed and selected to treat the particular disease. Hence, the methods and compositions provided herein are designed to transiently inhibit or suppress the activity of leukocyte subtypes (and/or other cells such as adipocytes, astrocytes, and others) and remove sources that fuel inflammatory mechanisms and secondary damage.

Exemplary disorders and conditions include, but are not limited to, any set forth in Table 9 above such as, for example, cardiovascular disease including stroke, atherosclerosis, and hypertension; liver disease; lung disease such as asthma, chronic obstructive pulmonary disease (COPD), acute lung injury and acute respiratory distress syndrome (ARDS); inflammatory joint disease such as Rheumatoid Arthritis and osteoarthritis; acute hypersensitivity, chronic kidney diseases including diabetic neuropathy and glomerulonephritis; systemic diseases such as systemic lupus erythematosus and obesity; HIV infection and associated diseases including dementia, encephalitis, and nephropathy; growth, neovascularization (angiogenesis) and metastases of several forms of cancer including, cancers of all organs such as brain, breast, lung cancers, and ovarian cancer; central nervous system diseases including Alzheimer's disease; Down's syndrome; multiple sclerosis; spinal cord injury; spongiform encephalopathies; inflammatory bowel disease such as sepsis; ulcerative colitis and Crohn's disease; skin diseases such as eczema and psoriasis; eye diseases including uveitis and retinitis and iritis, and proliferative vitreoretinopathy; and transplantation such as graft versus host disease (GVHD) and graft/organ rejection.

Descriptions of the involvement of leukocytes and other cell types in the pathology of some of these diseases are described below. Such descriptions are meant to be exemplary only and are not limited to a particular LPM conjugate toxin or to a particular ligand-toxin conjugate. One of skill in the art can design and select a ligand-toxin conjugate to be used in the treatment of any desired disease, based on the known cellular components. The particular treatment and dosage can be determined by one of skill in the art. Considerations in assessing treatment include; the disease to be treated, the cellular components involved in the disease, the severity and course of the disease, whether the molecule is administered for preventive or therapeutic purposes, previous therapy, the patient's clinical history and response to therapy, and the discretion of the attending physician.

a. Cancer

Cancers can be viewed as inflammatory diseases even if the cells are not of hematological origin. Cancer cells display many of the phenotypes ascribed to leukocyte subgroups and by definition can be regarded as inflammatory cells. They have the capacity to secrete proteases and proinflammatory mediators (including chemokines) and to perform phagocytosis. In addition, cancer cells express various receptors including cytokine, chemokine, growth factor (GF) receptors; CAMs to facilitate metastasis; and undergo transdifferentiation. As an example of the latter, colon carcinoma cells undergo epithelial-mesenchymal transition with the concomitant increase in expression of CXCR1 and CXCL8 which enhance motility and invasiveness (Bates et al. (2004) Exp. Cell Res., 299:315-24). Quantitative examination of leukocyte infiltrates have revealed, for example, that tumor associated macrophages (TAM) and lymphocytes make up to 50% of the cell mass in breast carcinomas and could arguably be regarded as tumor (Elkak et al. (2005) J. Carcinog., 4:7; Leek et al. (1996) Cancer Res. 56:4625-9; Murdoch et al. (2004) Blood 104:2224-34; Queen et al. (2005) Cancer Res., 65:8896-904). The fact that MNP and recruited monocytes differentiate into TAM that provide growth factors and aid in angiogenesis and metastasis attests to this notion (see e.g., Ueno et al. (2000) Clin. Cancer Res., 6:3282-9; Valkovic et al. (2002) Virchows Arch., 440:583-8; and references in Table 8).

b. Kidney Disease

There are many categories of renal diseases some of which are classified into subgroups. These diseases include, but are not limited to, acute nephritic syndrome; anti-glomerular basement membrane disease; autosomal-dominant polycystic kidney disease; glomerulonephritis (GN), anti-neutrophil cytoplasmic antibody GN; diabetic nephropathy; diabetic glomerulosclerosis; focal segmental glomerulosclerosis; Goodpasture's syndrome; HIV nephropathy; idiopathic crescentic GN; idiopathic rapidly progressive GN; IgA nephropathy IgAN; IgM mesanganoproliferative GN; lupus nephritis; membranoproliferative GN (MPGN, I, II, III); minimal change disease; membranonephropathy; nephritic syndrome; polyomavirus nephropathy; post-streptococcal GN; rapid crescentic GN; renal transplant rejection; renal vasculitides (e.g., Wegener's granulomatosis) and tubulointerstitial nephritis. A small percentage of kidney diseases may resolve, but the most common path is either rapidly or slowly declining to chronic kidney disease (CKD) for which there is no cure. CKD patients eventually decline into kidney failure and end-stage renal disease (ESRD). ESRD requires the patient to rely on dialysis treatment or transplantation.

Leukocytes and chemokines play pivotal roles in renal diseases and in renal allograft rejection. The inflammatory response in CKDs can be initiated by activated leukocytes, autoantibodies, immune complexes immunoglobulins, DNA species, nucleosomes, AGE, complement or a combination of these agents. An important initiating mechanism is antibody mediated tubular and glomerular injury. Antibodies either form complexes with insoluble glomerular antigens and/or immune complexes with circulating antigens which are ultimately deposited in the mesangium of the glomeruli. Several renal and non-renal cells are activated and many different kinds of soluble mediators including cytokines, chemokines and profibrotic growth factors are released into the milieu. Once activated infiltrating leukocytes and all intrinsic renal cells including fibroblasts; mesangial cells (MGCs); tubular epithelial cells (TEC); podocytes; and glomerular parietal epithelial cells (PEC) are capable of expressing these same proinflammatory mediators. The general pathology of CKD involves pyelonephritis (PN), or infection of the kidney; breakdown of the glomerular basement membrane (GBM) and parietal basement membrane (PBM); leukocyte infiltration; crescent formation; fibrosis; destruction of the tubules and nephrons; and collapsing glomeruli. Unless there is resolution or medical intervention, the disease will progress with ongoing inflammation, repeated renal injury, fibrosis and progression to ESRD. Macrophages/monocytes, T cells and MaC are the prominent leukocytes involved in CKD. Several chemokines and their cognate receptors which regulate the activation, migration and proliferation of these leukocyte subtypes in CKDs include MIP-1α/CCR1, ENA-78/CCR5, fractalkine/CX3CR1, MIG/IP-10/1-TAC/CXCR3 and IL-8/CXCR1/2. As demonstrated by animal model and human studies the role of macrophages/monocytes and MCP-1/CCR2 in several different types of CKD is pivotal and makes this chemokine/receptor axis a compelling one for therapeutic intervention (see Table 8 for references). There are no therapeutics that successfully treat all symptoms of CKD and few that do without side effects (e.g., Busauschina et al., Transplant Proc., 36: 229S-233S, 2004; Slattery et al., Am. J. Pathol., 167: 395-407, 2005; Bir et al., J. Rheumatol., 33: 185-7, 2006). Hence there is a need for a different therapeutic approach to CKDs, such as is provided herein.

c. Spinal Cord Injury (SCI)

The outcome of spinal cord injury (SCI) is a result of initial mechanical and ischemic trauma with disruption of cellular ionic homeostasis which is rapidly followed by secondary tissue damage inflicted by the actions of activated leukocyte subtypes including microglia (the resident macrophages of the CNS), leukocyte inflammatory mediator production and robust inflammatory cascades. These cascades are observed within minutes and proceed for several weeks and are followed by a period of partial recovery entailing endogenous repair and regeneration. The secondary damage is detectable as necrotic and apoptotic cell death of neurons and oligodendrocytes; cellular excitotoxicity; blood-brain barrier/blood-spinal barrier disruption; reactive gliosis (which leads to glial scarring); neovascularization; demyelination; loss of sensory and motor function and post-SCI chronic pain (Jones et al. (2005) Curr. Pharm. Des., 11: 1223-6; Klussman and Martin-Villalba (2005) J. Mol. Med., 83:657-71; Lee et al. (2000) Neurochem. Int., 36:417-25; McTigue et al. (1998) J. Neurosci. Res., 53: 368-76; Carlson et al. (1998) Exp. Neurol., 151: 77-88; Bartholdi and Schwab (1997) Eur. J. Neurosci., 9:1422-38; Hains and Waxman (2006) J. Neurosci., 26:4308-17; Abbadie, Trends Immunol., 26: 529-34, 2005). It is a combination of primary necrosis produced by the initial physical injury and apoptotic events initiated by leukocyte and astroglial derived inflammatory mediators that lead to secondary tissue damage in SCI and the pathologic mechanisms are similar in a wide range of CNS traumas and diseases including, for example, traumatic brain injury; stroke; multiple sclerosis (MS), Alzheimer's disease and HIV-associated dementia (see Table 8 for references).

The acute inflammatory injury in SCI lasts several days but is overlapped by CNS reparative mechanisms such as axon sprouting and limited remyelination due in part to differentiating precursor oligodendrocytes. MNPs (microglia and macrophages) have now been identified as facilitators of repair. These cells phagocytose dead cells and debris and provide matrix proteins growth factors, neurotrophins and cytokines that aid CNS repair. The dual role for MNPs in injury and repair is evident in other leukocyte mediated diseases including experimental glomerulonephritis, liver injury; carotid artery injury and MS (Duffield et al. (2005) J. Clin. Invest 115:56-65; Duffield (2003) Clin. Sci. 104:27-38; Danenberg et al. (2002) Circulation 106: 599-605; Raivich and Banati (2004) Brain Res. Brain Res. Rev. 46:261-81). Experimental SCI studies have demonstrated that transient suppression of MNP activity or extravasation and withdrawal of treatment later allows for increased tissue sparing and improved behavioral outcomes. This is due in part to the reparative activities of MNPs and perhaps T cells (Jones et al. (2005) Curr. Pharm. Des., 11:1223-36; Gris et al. (2004) J. Neurosci. 24:4043-51; Wells et al. (2003) Brain 126:1628-37). Depletion of PMN or macrophages in the early stages of experimental SCI has shown similar positive outcomes (Taoka and Okajima, Prog. Neurobiol., 56: 341-58, 1998; Popovich et al. (1999) Exp. Neurol., 158: 351-365). There is a differential expression of chemokine ligands and receptors implicated in the pathology of secondary tissue damage in SCI which allows the identification of target receptors using LPMs (Glaser et al. (2004) J. Neurosci. Res., 77:701-8; Glaser et al., (2006) J. Neurosci. Res. 84: 724-34; Ghimikar et al. (2000) J. Neurosci. Res. 59:63-73; McTigue et al. (1998) J. Neurosci. Res., 53: 368-76; Lee et al., Neurochem. Int. 36:417-25, 2000). It is known that only a few residual axons (10-15%) are needed to effect significant functional recovery after SCI (Jones et al. (2005) Curr. Pharm. Des., 11: 1223-36). Therefore dampening inflammation in the acute phase is a viable approach to therapy. Eradication of activated pathological leukocytes is one avenue.

d. Hypersensitivity

Hypersensitivity reactions have been categorized into four (and sometimes overlapping) main types (I-IV) all of which can be associated with immune-mediated tissue injury. Type I (immediate) hypersensitivity takes place in minutes to hours of exposure to allergens and involves B cell production of IgE antibodies which mediate mast cell and basophil degranulation. Eosinophils also are involved. This reaction is involved in several conditions including asthma, atopic dermatitis, eczema, conjunctivitis and rhinitis. Type II (cytotoxic) hypersensitivity is due to antibodies recognizing either self or extrinsic antigens on cell surfaces and mediating complement-dependent cytotoxicity or antibody-dependent cell mediated cytotoxicity by activated macrophages and natural killer T cells. Conditions associated with this reaction include Goodpasture's syndrome (lungs and kidneys) and thyroiditis. Type III immune complex-mediated hypersensitivity occurs when antibodies bind self or foreign antigens which can be deposited in tissues and results in complement activation and inflammation (activation, proliferation and infiltration of various leukocyte subtypes). This is the classical pathology involved in diseases such as glomerulonephritis, vasculitides, systemic lupus erythematosus and arthritis. Type IV (delayed) cell-mediated hypersensitivity usually takes days to develop and is not antibody dependent. This reaction relies upon different subsets of T cells, cytotoxic T cells and macrophages which aberrantly destroy self target cells complexed with self or extrinsic antigens. Neutrophils, eosinophils and mast cells also are implicated in this type of reaction. This reaction is found in such conditions as contact dermatitis, psoriasis, inflammatory bowel diseases, insulin-dependent diabetes mellitus, multiple sclerosis and rheumatoid arthritis. All the above immune reactions involve the trafficking, activation, and proliferation of leukocytes to the affected tissues and organs (see Table 8 for references).

Contact dermatitis studies have identified several chemokine axes responsible for the recruitment of activated leukocytes include but are not limited to IP-10/CXCR3, IL-8/CXCR2, RANTES/CCR5, MCP-1/CCR2, MIP-1α/CCR1 and 5. Different chemoprints have been identified for allergic contact dermatitis, psoriasis, atopic eczema and atopic dermatitis. Similarly prominent chemokine axes involved in several forms of cutaneous T cell lymphomas, melanomas, scleroderma and systemic sclerosis have been identified. This indicates that treatment with a carefully chosen LPM containing a relevant chemokine receptor targeting agent would be useful in the treatment of inflammatory skin diseases and cancers (see references in Table 6).

e. HIV Infection and AIDS and Infections with Other Pathogens

Activation and infection of CNS microglia and infiltrating macrophages is one hallmark of the pathogenesis of HIV induced diseases Human immunodeficiency viruses (HIV) enter a cells via certain receptors, classically the CD4 receptor that are associated with a specific chemokine co-receptor. The CXCR4, CCR2b, CCR3, CCR5, CCR6, CCR8, CX3CR1 and others can all act in a co-receptor capacity. For example, macrophage-tropic HIV-1 strains generally use CCR5 co-receptors, while T-cell-tropic strains generally use CXCR4. In addition, dual-tropic viruses can use CXCR4 and CCR5 co-receptors for entry, while other subsets of the HIV viral strains use a variety of other chemokine co-receptors (see Rubbert et al., HIV Medicine 2006, Chapter 4, Hoffman et al., eds, Flying Publisher, Paris).

In patients with HIV encephalitis, (HIVE) CXCR-4 is expressed on MNPs, astrocytes, and a sub-population of cholinergic neurons, whereas CCR5 is mainly expressed on MNPs. It should be noted that the majority of infected cells in HIVE patients (children and adults) appear to be MNPs and increased expression of CCR5 appears to correlate with the severity of the disease. This indicates that MNP-mediated events can be important, at least in the late and severe stages of HIVE. The CCR5 receptor also is upregulated following bacterial infection of the CNS and in a rat model of ischemic brain injury.

Increased production of cytokines (e.g., TNF-α) and chemokines (e.g., RANTES, MCP-1, MIP-1α, and MIP-1β) is associated with HIV infection. Increased CNS chemokines in HIV would account for peripheral leukocyte recruitment and cytokine release with direct cytotoxic effects (at least in the case of the cytokine TNF-α on neurons and oligodendrocytes, and precisely mirrors the experience in CNS trauma. Several cytokines including, GM-CSF, macrophage-CSF, IL-1β, IL2, IL-3, IL-6, TNF-α, and TNF-β also can contribute to the pathogenesis of HIV disease by activating and/or augmenting HIV replication.

Secondary damage occurs in HIV-1 positive, asymptomatic, pre-AIDS patients (An et al. (1997) Arch Anat Cytol Pathol 45, 94-105). These investigators were able to detect HIV-1 DNA in 50% of the brains of asymptomatic patients and nearly 90% displayed astrogliosis. These patients also have elevated levels of immunomolecules, and cytokines including, TNF-α, IL-1, IL-4, and IL-6. Neuronal damage was confirmed by the detection of apoptotic neurons.

Direct neurotoxicity and upregulation of the CCR5 co-receptor by MNP-derived excitatory amino acids has also been implicated in the pathology of HIV infection. An increase in inducible nitric oxide synthase activity has been detected in HIV infected microglia from AIDS patients. This indicates that the production of nitric oxide could contribute to lesion formation in HIV infected areas of the nervous system. Once again, the pathology of HIV encephalopathies, and pre- and full blown AIDS affecting the CNS, appear to mimic the secondary tissue damage observed in SCI and other inflammatory diseases.

It has also been found that some chemokines and chemokine receptors also are promicrobial factors and facilitate infectious disease (see, Pease et al. (1998) Semin Immunol 10:169-178). Pathogens exploit the chemokine system. For example, cellular chemokine receptors are used for cell entry by intracellular pathogens, including HIV. In addition viruses use virally-encoded chemokine receptors to promote host cell proliferation. Pathogens also subvert the chemokine system. Virally-encoded chemokine antagonists and virally-encoded chemokine scavengers are known (e.g., Murphy, Nat Immunol., 2: 116-22, 2001: Kotwal, Immunol Today, 21: 242-8, 2000).

f. Inflammatory Joint Disease and Autoimmune Disease

Rheumatoid arthritis (RA) is an inflammatory autoimmune disease characterized by chronic connective tissue damage and bone erosion. The pathogenesis of the disease includes the infiltration of leukocytes into the synovial space, their activation, and the release of inflammatory mediators that ultimately deform and destroy the affected joint. The actual arthritic response appears to be initiated when MNPs release pro-inflammatory cytokines and chemokines. TNFα, IL-1, IL-6, GM-CSF, and the chemokine IL-8, are found in abundance in joint tissue from R^{A} patients and their most likely source includes synovial fibroblasts, in addition to MNPs. The combination of MNPs, neutrophils, and T-cells, with the participation of synovial fibroblasts and synoviocytes, sets up a cascade of inflammation.

IL-1 and TNFα are believed to be responsible for the production of chemokines in the arthritic joint. In one study, increased concentrations of these two cytokines induced the expression of IL-8 (a potent T-cell chemoattractant) and RANTES (a potent neutrophil chemoattractant), in human synovial fibroblasts isolated from RA patients (Rathanaswami et al. (1993) J Biol Chem 268, 5834-9). Other investigators have shown that inflamed synovial tissue from R^{A} and osteoarthritic patients contains high concentrations of MCP-1, and TNFα and IL-1 markedly increased the mRNA expression of this chemokine in cultured synoviocytes derived from these specimens. It appears that chemokines from MNPs and cytokine stimulated synovial fibroblasts and synoviocytes play a role in the pathology of RA by facilitating the recruitment and extravasation of peripheral monocytes, neutrophils and T-cells. In common with other diseases and conditions, activated leukocytes release a range of other tissue damaging mediators. More specifically, leukocyte-derived reactive oxygen species and proteolytic enzymes (e.g. matrix metalloproteinases, cathepsin and neutrophil-derived elastase) have been implicated in the initiation and maintenance of tissue damage in inflammatory joint diseases (see Table 8 for references).

g. Pulmonary Disease

Lung injury covers a wide array of clinical conditions. For purposes herein they are collectively referred to as Inflammatory Diseases of the Lung (ILDs). An ILD is typically the result of specific insult, for example, systemic bacterial infections (e.g., sepsis), trauma (e.g., ischemia-reperfusion injury), and inhalation of antigens (e.g., toxins like cigarette smoke). ILDs also include allergic alveolitis, ARDS (acute or adult respiratory distress syndrome), various forms of asthma, bronchitis, collagen-vascular disease, pulmonary sarcoidosis, eosinophilic lung diseases, pneumonia, and pulmonary fibrosis. In brief, the pathology of these diseases and conditions, involves the activation of macrophages, particularly those located in the alveoli. Neutrophils, eosinophils and T-cells, are activated and recruited to the site of injury subsequent to the release of macrophage, and neighboring endothelial and epithelial cell derived cytokines and chemokines. The specific cytokines and chemokines involved include; GM-CSF, TNF-α, IL-1, IL-3, IL-5, IL-8, MCP-1, MCP-3, MIP-1α, RANTES and Eotaxin.

Leukocytes respond to the pro-inflammatory cytokines and chemokines by releasing the many mediators of secondary tissue damage including; proteases, reactive oxygen species, and biologically active lipids, and by expressing cell surface antigens and cell adhesion molecules. In addition, it appears that specific leukocyte populations play a more prominent role in some ILDs than they do in others. Neutrophils and MNPs are more prominent contributors to secondary damage in acute lung injuries like ARDS and various lung fibroses; whereas T-cells and eosinophils are the chief culprits in eosinophilic lung diseases, which include allergic asthma, fibrosing alveolitis, and sarcoidosis (see Table 8 for references).

h. Other Diseases Mediated by Secondary Tissue Damage

Disease states associated with secondary tissue damage can be treated according to the methods provided herein and using the conjugates provided herein as well as certain non-chemokine cytokines known to those of skill in the art for treatment of other conditions. These disease states, include, but are not limited to, CNS injury, CNS inflammatory diseases, neurodegenerative disorders, heart disease, inflammatory eye diseases, inflammatory bowel diseases, inflammatory joint diseases, inflammatory kidney or renal diseases, inflammatory lung diseases, inflammatory nasal diseases, inflammatory thyroid diseases, cytokine regulated cancers, and other disease states that involve or are associated with secondary tissue damage.

Examples of CNS inflammatory diseases and/or neurodegenerative disorders that can be treated using the methods herein and conjugates provided herein, include, but are not limited to, stroke, closed head injury, leukoencephalopathy, choriomeningitis, meningitis, adrenoleukodystrophy, AIDS dementia complex, Alzheimer's disease, Down's Syndrome, chronic fatigue syndrome, encephalitis, encephalomyelitis, spongiform encephalopathies, multiple sclerosis, Parkinson's disease, spinal cord injury/trauma (SCI), and traumatic brain injury; heart diseases that can be treated using the methods provided herein, include, but are not limited to, atherosclerosis, neointimal hyperplasia and restenosis; inflammatory eye diseases that can be treated using the methods and conjugates provided herein, include, but are not limited to, proliferative diabetes retinopathy, proliferative vitreoretinaopathy, retinitis, scleritis, scleroiritis, choroiditis and uveitis. Examples of inflammatory skin diseases that can be treated using conjugates and methods as provided herein include, but are not limited to, psoriasis, eczema and dermatitis.

Examples of inflammatory bowel diseases that can be treated using the methods and conjugates provided herein, include, but are not limited to, chronic colitis, Crohn's disease and ulcerative colitis. Examples of inflammatory joint diseases that can be treated using the methods and conjugates provided herein include, but are not limited to, juvenile rheumatoid arthritis, osteoarthritis, rheumatoid arthritis, spondylarthropathies, such as ankylosing spondylitis, Reiter's syndrome, reactive arthritis, psoriatic arthritis, spondylitis, undifferentiated spondylarthopathies and Behcet's syndrome; examples of inflammatory kidney or renal diseases that can be treated using the methods and conjugates provided herein include, but are not limited to, glomerulonephritis, lupus nephritis and IgA nephropathy. Examples of inflammatory lung diseases that can be treated using the methods and conjugates provided herein, include, but are not limited to, eosinophilic lung disease, chronic eosinophilic pneumonia, fibrotic lung diseases, acute eosinophilic pneumonia, bronchoconstriction, including asthma, bronchopulmonary dysplasia, bronchoalveolar eosinophilia, allergic bronchopulmonary aspergillosis, pneumonia, acute respiratory distress syndrome, and chronic obstructive pulmonary disease (COPD); examples of inflammatory nasal diseases that can be treated using the methods and conjugates provided herein, include, but are not limited to, polyposis, rhinitis, sinusitus; examples of inflammatory thyroid diseases that can be treated using the methods and conjugates provided herein, include, but are not limited to, thyroiditis; and examples of cytokine-regulated cancers that can be treated using the methods provided herein, include, but are not limited to, gliomas, atheromas carcinomas, adenocarcinomas, granulomas, glioblastomas, granulamatosis, lymphomas, leukemias, melanomas, lung cancers, myelomas, sarcomas, sarcoidosis, microgliomas, meningiomas, astrocytomas, oligodendrogliomas, Hodgkins disease, and breast and prostate cancers. Other inflammatory diseases susceptible to treatment using the methods and conjugates provided herein, include, but are not limited to, vasculitis, autoimmune diabetes, insulin dependent diabetes mellitus, graft versus host disease (GVHD), psoriasis, systemic lupus erythematosus, sepsis, systemic inflammatory response syndrome (SIRS), and injurious inflammation due to burns.

As noted above, these disorders, although diverse, share the common features related to the inflammatory response. Spinal cord injury or trauma, which can be treated by administering to a subject in need thereof an effective amount of a therapeutic agent as described herein, is exemplary of the disorders contemplated. The treatments herein are designed to attack the adverse results of this response involving proliferation and migration of leukocytes. The treatments will eliminate or reduce the leukocyte proliferation and migration and by virtue of this lead to an amelioration of symptoms, a reduction in adverse events or other beneficial results that can enhance the effectiveness of other treatments.

5. Combination Therapies

The ligand-toxin conjugates, such as any LPM provided herein, can be used in combinations for the treatment of the indicated diseases. Combination therapy can be achieved by administering a ligand-toxin conjugate with any other therapeutic agent for treating a particular disease. Such agents are known to those of skill in the art. Combination treatment also can be effected using molecules composed of two or more, such as two different chemokines attached at either end of a toxin moiety. In that case, these dual chemokine fusions can include one ligand from each of α and β chemokines family.

### L. EXAMPLES

The following examples are included for illustrative purposes and are not intended to limit the scope of the invention.

### Example 1

### Selection of Modified Shiga Toxin A1 (SA1) Variants for Construction of LPMs

A. Cloning and Expression of LPMs for selection of SA1 variants

A nucleic acid molecule encoding an MCP-1/Shiga Toxin fusion protein (designated LMP1a) was designed such that the fusion protein starts with a methionine (Met) residue followed by the published sequence of mature MCP-1 (set forth in SEQ ID NO:69, and encoded by a sequence of nucleotides set forth in SEQ ID NO:68), an Ala-Met linker (SEQ ID NO:34), and residues 23-268 of the Shiga-A1 toxin subunit containing the ribosome inactivating (RIP) domain (referred to herein as variant 1 SA1; corresponding to SEQ ID NO:22 and encoded by the nucleic acid sequence set forth in SEQ ID NO:23). To facilitate removal and replacement of the gene sequence into different expression vectors, restriction endonuclease sites were incorporated into the gene sequence at the 3′ and 5′ ends. The sequence of LPM1a was designed to have an NdeI restriction site, which contains the methionine start codon (SEQ ID NO:31) at the 5′ end, and also was designed to have a stop codon followed by a BamHI restriction site (SEQ ID NO:33) at the 3′ end. A nucleic acid molecule encoding LPM1a was synthesized following the principles of codon usage and secondary structure optimization by a DNA synthesis service organization (Blue Heron Biotechnology, Seattle Wash.) and supplied in a pUC plasmid with the multiple cloning site removed (pUC minus M, SEQ ID NO:86). The sequence of the LPM1a nucleic acid molecule and encoded fusion protein are set forth in SEQ ID Nos: 37 and 38, respectively.

Since the variant 1 sequence of SA1 contained within the LMP1a fusion protein contains a cysteine residue corresponding to amino acid 242 of SEQ ID NO: 22, a further truncated SA1 moiety was generated to avoid cysteine-induced dimerization among highly purified LMP fusion proteins. This SA1 moiety (referred to herein as variant 2) lacks the five C-terminal amino acids (CHHHA) corresponding to amino acids 242-246 of the polypeptide sequence set forth in SEQ ID NO:22. The amino acid sequence of the variant 2 SA1 is set forth in SEQ ID NO:24, and encoded by a nucleic acid sequence set forth in SEQ ID NO:25. An MCP-1 fusion protein containing the variant 2 SA1 moiety, termed LPM1b, was generated. A nucleic acid sequence encoding the LPM1b fusion protein (MCP-1-AM-SA1 (variant 2)), containing the variant 2 SA1 sequence was synthesized and supplied as described above for the LPM1a fusion protein. The sequence of the LPM1b nucleic acid molecule and encoded fusion protein are set forth in SEQ ID Nos: 39 and 40, respectively.

The resulting LMP1a and LMP1b constructs in the pUC minus M vector were digested with NdeI and BamHI to produce an ˜1 Kb NdeI/BamHI fragment which was cloned into a T7 expression vector, pET9c (Novagen, SEQ ID NO: 84), at the corresponding NdeI/BamHI sites. The pET9c plasmid containing LPM1a was transformed into the expression host strain HMS174 (DE3) pLyS (F⁻ recA1 hsdR(r_{K12}⁻m_{K12}⁺) (DE3) pLysS (Cam^{R}, Rif^{R}) according to the manufacturer's instructions (Novagen). The pET9c plasmid containing LPM1b was transformed into the expression host strain HMS174 (DE3) (F⁻ recA1 hsdR(r_{K12}⁻m_{K12}⁺) (DE3) (Rif^{R}) according to the manufacturer's instructions (Novagen).

### B. Selection of Mutants

LPM1a and LPM1b produce fusion proteins that contain an SA1 RIP toxin moiety, as described in part A above. The expression of the SA1 moiety is toxic to host cells and disrupts the production of the LPM fusion proteins. To select for mutants in SA1 that exhibit less toxicity, the pET9c plasmid constructs containing LPM1a or LPM1b were used for mutation selection in the presence or absence of varying concentrations of 4APP (4-aminopyrazolo[3,4-d]-pyrimidine). Following transformation of the pET9c-containing LPM construct into the appropriate host strain as described in part A, transformed bacteria were selected on LB kanamycin (km) at 50 μg/ml in the presence or absence of varying concentrations of 4APP. The results set forth below are based on selection of LPM1a transformed bacterial cells on LB kanamycin (km) at 50 μg/ml in the absence of 4APP and selection of LPM1b transformed bacterial cells on LB kanamycin (km) at 50 μg/ml in the presence of 0.5 mM 4APP.

1. LPM1a Mutants

Transformation of HMS 174(DE3) pLyS host cells with the pET9c plasmid construct containing LPM1a in the absence of 4APP yielded 82 transformants. All 82 selected colonies were screened for LPM1a expression and plasmid integrity. Plasmid DNA was isolated from bacterial transformants using a standard miniprep procedure. Expression of the full length protein was confirmed by SDS-PAGE. The LPM1a insert from the pET9c plasmid was purified following digestion with NdeI/BamHI and the insert was sequenced with T7 primer and T7t primer to confirm the sequence. T7: 5′ TAA,TAC,GAC,TCA,CTA,TAG,GG 3′ (SEQ ID NO:35); T7t: 5′GCT,AGT,TAT,TGC,TCA,GCG 3′ (SEQ ID NO:36).

Few of the colonies expressed LPM1. Some of the selected colonies expressed truncated forms of LPM1. One colony expressed an LPM1 containing an L to R mutation at position 117 in the SA1 moiety portion of the fusion protein compared to the LPM1a sequence set forth in SEQ ID NO: 38 (corresponding to L38R in the amino acid sequence for the variant 1 SA1 moiety set forth in SEQ ID NO:22). This mutant LPM1 is referred to herein as LPM1c. The nucleotide and amino acid sequences for LPM1c are set forth in SEQ ID Nos: 41 and 42, respectively, and can be compared to the parent LPM1a molecules set forth in SEQ ID Nos: 37 and 38. The L38R mutation in SA1 is referred to herein as mutant variant 1 (also referred to as variant 3 herein) and is set forth in SEQ ID NO:26, and encoded by a nucleic acid having a sequence set forth in SEQ ID NO:27.

2. LPM1b Mutants

Transformation of HMS 174(DE3) host cells with the pET9c plasmid construct containing LPM1b in the presence of 0.5 mM 4APP yielded 10 transformants. All 10 transformants were selected, plasmid DNA prepared, and analyzed as described above for the LPM1a mutants.

Two selected colonies expressed an LPM1 containing a V to A mutation in the SA1 moiety at position 298 compared to the parent LPM1b sequence set forth in SEQ ID NO:40 (corresponding to V219A in the amino acid sequences for the variant 1 and variant 2 SA1 moieties set forth in SEQ ID NO:22 and SEQ ID NO:24, respectively). This mutant LPM1 is referred to herein as LPM1d. The nucleotide and amino acid sequences for LPM1d are set forth in SEQ ID Nos: 43 and 44, respectively, and can be compared to the parent LPM1b sequence set forth in SEQ ID Nos: 39 and 40. The V219A mutation in SA1 is referred to herein as mutant variant 2 (also referred to as variant 4 herein) and is set forth in SEQ ID NO:28, and encoded by a nucleic acid having a sequence set forth in SEQ ID NO:29.

### Example 2

### Comparison of the Activities of Variant LPM1s

The consequence of mutations in SA1 on LPM1 activity was assessed by measuring the activities of LPM1c (containing the variant 3 SA1 sequence) and LPM1d (containing the variant 4 SA1 sequence) in a rabbit reticulocyte lysate (RIP) assay. LPM1c and LPM1d proteins were expressed and partially purified (see EXAMPLE 4). The activities of these proteins were assessed by measuring inhibition of protein synthesis using a commercially available rabbit reticulocyte lysate system (i.e. RIP assay) designed to assay the translation of luciferase RNA (Promega, Madison, Wis.; all reagents included). Briefly, protein samples were diluted to 1 μg/ml and serially diluted in 10 fold steps in PBS, pH 7.4, containing 1 mg/ml BSA. Diluted protein (10 μl) was added to 5 μl reaction mix (reaction mix: 2 μl of a 1 mg/ml solution of luciferase RNA; 1 μl of a 1:1 ratio 0.1 mM amino acid mixture minus methionine and amino acid mixture minus lysine; 2 μl of ribonuclease inhibitor) and 35 μl rabbit reticulocyte lysate. Samples were incubated at 30° C. for 1.5 hours before the reaction was stopped by incubating the samples on ice. Samples were diluted 1:25 using the reaction mix described above. The reaction mixture (100 μl) was transferred to 96-well white polystyrene plates (Corning Corporation, NY) and 100 μl of the luminescent dye Bright-Glo (Promega) was added to each reaction. Plates were analyzed using a preheated (20-25° C.) FLUOstar luminometer (BMG Lab Technologies, Durham, N.C.). In parallel, reaction mixture only or reagent blank were used as negative controls, and the RIP protein saporin (Sigma, St. Louis, Mo.) was used as a positive control. The saporin positive control consistently had relative activity (RIC₅₀) values in the range of 8-12 pM. The shiga holotoxin has a reported RIC₅₀ value of 9 pM (Skinner and Jackson (1997) J. Bacteriol. 179: 1368-174). Purified Variant 4 SA1 subunit (SEQ ID NO: 28) had an RIC₅₀ value of 50 pM. LPM1c (SEQ ID NO:42) and LPM1d (SEQ ID NO:44) had RIC₅₀ values of 5 nM and 80-100 pM, respectively. Based on the observed RIP activities of the mutant variants tested, new LPMs containing the SA1 sequence from LPM1d, which is the mutant variant 2 (i.e. variant 4) SA1, were constructed as described in Example 3 below.

### Example 3

### Construction of LPM Genes Containing SA1 Variant 4

LPMs 2-13 (Table 12) were constructed to encode fusion proteins of the respective chemokine sequence linked by an alanine-methionine dipeptide to the mutant variant 2 (i.e. variant 4) truncated version of the mature SA1 shiga toxin subunit (set forth in SEQ ID NO:28). The sequences encoding LPMs 2-13 were inserted into the pET9c plasmid (SEQ ID NO:84) by two different methods, which are described below. Each of the methods relied on the presence of an internal EcoRI restriction site within the 5′ sequence of the SA1 shiga toxin subunit sequence (e.g., corresponding to nucleotides 4-9 of the variant 1 sequence set forth in SEQ ID NO:23, or the variant 4 sequence set forth in SEQ ID NO:29), resulting in an SA1 moiety lacking the 5′ lysine residue which was reconstituted by the design of a chemokine linker moiety containing an encoded lysine adjacent to an EcoRI restriction site. All protocols used for plasmid manipulation were from Maniatis et al., Molecular Cloning: A Laboratory Manual, Cold Spring Harbor Laboratory, New York (1982).

TABLE 12
LPM Variants

| | | | SEQ ID NO | SEQ ID NO |
|---|---|---|---|---|
| LPM | Chemokine | SA1 Variant | (nucleotide) | (amino acid) |
| LPM1a | MCP-1 | 1 | 37 | 38 |
| LPM1b | MCP-1 | 2 | 39 | 40 |
| LPM1c | MCP-1 | 3 | 41 | 42 |
| LPM1d | MCP-1 | 4 | 43 | 44 |
| LPM2 | Eotaxin-1 | 4 | 45 | 46 |
| LPM3 | SDF-1β | 4 | 47 | 48 |
| LPM4 | GRO-α | 4 | 49 | 50 |
| LPM5 | MIP-1β | 4 | 51 | 52 |
| LPM6 | IL-8 | 4 | 53 | 54 |
| LPM7 | IP-10 | 4 | 55 | 56 |
| LPM8 | MCP-3 | 4 | 57 | 58 |
| LPM9 | MIP-3α | 4 | 59 | 60 |
| LPM10 | MDC | 4 | 61 | 62 |
| LPM11 | MIP-1α | 4 | 63 | 64 |
| LPM12 | Eotaxin-1 | 4 | 65 | 46 |
| LPM13 | BCA-1 | 4 | 66 | 67 |

Briefly, the sequences of the nucleic acid molecule encoding the chemokines used for the construction of LPMs 2-13 (Table 12) were synthesized, following the principles of codon usage and secondary structure optimization, by a DNA synthesis service organization (Bio S&T, Montreal) and supplied in a pUC plasmid (pUC19, SEQ ID NO: 85). For each chemokine gene, an NdeI restriction site containing a methionine start codon (SEQ ID NO:31) was added to the 5′ end and a linker sequence encoding the amino acids Ala-Met-Lys followed by an EcoRI site (SEQ ID NO:32) was added to the 3′ end. The nucleotide sequence for each chemokine construct is described in Table 13 and set forth in SEQ ID NOS:72-83. A second eotaxin sequence was optimized and supplied by Blue Heron Biotechnology and is set forth in SEQ ID NO:82. Each of the respective nucleic acid molecules encoding the chemokines were used to generate LPM fusion proteins by one of two cloning methods, which are set forth below.

TABLE 13
Nucleotide Positions for Sequence Components
of Chemokine Constructs for LPMs 2-13

| | | SEQ | NdeI I | | | |
|---|---|---|---|---|---|---|
| | Chemokine | ID | restriction | Mature | Linker | EcoRI |
| LPM | construct | NO: | site | chemokine | sequence | site |
| 2 | Eotaxin | 72 | 1-6 | 7-228 | 229-237 | 238-243 |
| 3 | SDF-1β | 73 | 1-6 | 7-222 | 223-231 | 232-237 |
| 4 | GRO-α | 74 | 1-6 | 7-225 | 226-234 | 235-240 |
| 5 | MIP-1β | 75 | 1-6 | 7-213 | 214-222 | 223-228 |
| 6 | IL-8 | 76 | 1-6 | 7-237 | 238-246 | 247-252 |
| 7 | IP-10 | 77 | 1-6 | 7-237 | 238-246 | 247-252 |
| 8 | MCP-3 | 78 | 1-6 | 7-234 | 235-243 | 244-249 |
| 9 | MIP-3α | 79 | 1-6 | 7-216 | 217-225 | 226-231 |
| 10 | MDC | 80 | 1-6 | 7-213 | 214-222 | 223-228 |
| 11 | MIP-1α | 81 | 1-6 | 7-213 | 214-222 | 223-228 |
| 12 | Eotaxin | 82 | 1-6 | 7-228 | 229-237 | 238-243 |
| 13 | BCA-1 | 83 | 1-6 | 7-267 | 268-276 | 277-282 |

### A. Cloning Method 1

The components of LPMs 4-10, LPM12 and LPM13 (see Table 13) were assembled in a pUC19 plasmid (SEQ ID NO:85) and then subcloned into the pET9c vector. Briefly, the SA1 Variant 4 component was generated by digestion of the pET9c vector containing LPM1d (see Example 1) with EcoRI and BamHI to yield a 750 bp EcoRI/BamHI DNA fragment containing the SA1 Variant 4 gene. The digested fragment was gel purified and inserted into the pUC19 plasmid that also had been cut at the corresponding EcoRI/BamHI sites to yield a pUC19BB plasmid. The chemokine sequence component of LPMs 4-10, LPM12 and LPM13 was generated by digestion of the respective chemokine containing pUC19 plasmid, as described above, by digestion with NdeI and EcoRI to yield an ˜250 bp NdeI/EcoRI DNA fragment for each chemokine. To generate a complete LPM sequence (see Table 12), the digested chemokine fragment was gel purified and inserted into the pUC19BB plasmid containing the SA1 variant 4 sequence that also had been digested at the corresponding NdeI and EcoRI restriction sites. pUC19BB containing a complete LPM gene sequence was then digested with NdeI and BamHI to yield an ˜1 kb fragment, which was gel purified and subcloned into the pET9c plasmid (SEQ ID NO:84) that also had been digested with NdeI and BamHI. The plasmids were confirmed for expression of the respective LPM and sequenced to confirm the identity of the insert. Table 12 above sets forth sequence identifiers for the respective nucleic acid and encoded amino acids for the cloned LPM variants LPMs 4-10, LPM12 and LPM13.

### B. Cloning Method 2

To generate LPMs 2, 3, and 11 (see Table 12), the respective chemokine genes were directly inserted into the pET9c expression plasmid (SEQ ID NO:84) using the method described herein. First, to prevent digestion of the vector during subsequent cloning steps, the EcoRI site was removed from the pET9c plasmid to yield the vector pET9DE. Briefly, the pET9c plasmid was digested with EcoRI and the ends were filled in with T4 DNA polymerase. The plasmid DNA was ligated, yielding the pET9DE vector, and transformed into DH5α E. coli cells (Invitrogen, Carlsbad, Calif.). Plasmid DNA was isolated from bacterial transformants using a standard miniprep procedure and the deletion of the EcoRI site in the pET9DE vector was confirmed by restriction digestion.

To clone the genes encoding LPMs 2, 3, and 11, the pUC19BB plasmid from cloning Method 1 above containing the sequence for the complete LPM1d gene was digested with NdeI and BamHI to yield a 1 kb fragment. The fragment was gel purified and subcloned into the pET9DE vector that also had been digested with NdeI and BamHI to generate the pET9DE-BB plasmid. The chemokine sequence component of LPMs 2, 3, and 11 were generated by digestion of the respective chemokine containing pUC19 plasmid, as described above, by digestion with NdeI and EcoRIto yield an ˜250 bp NdeI/EcoRI DNA fragment for each chemokine. To generate a complete LPM sequence (see Table 12), the digested fragment was gel purified and inserted into the pET9DE-BB plasmid that had been digested at the corresponding NdeI/EcoRI site. Table 12 above sets forth sequence identifiers for the respective nucleic acid and encoded amino acids for the cloned LPM variants LPM2, 3, and 11.

### Example 4

### Expression and Purification of LPM Variants

### A. Expression of LPM Variants

Following transformation of HMS174(DE3) with a pET9c/LPM plasmid, the effect of the RIP inhibitor 4APP on expression of the different LPMs were tested by growing the transformants overnight at 37° C. in MTB medium (1xM9 medium with 24 g/L yeast extract, 12 g/L tryptone and 0.4% glycerol) with 50 μg/ml Kanamycin and 2 mM 4APP. Prior to induction of the respective LMP with IPTG, the cells were subcultured (1:10 dilution) in the same medium and grown for an additional 3 hours at 37° C. The cells were induced by induction in the presence or absence of 1 mM IPTG and in the presence of increasing concentrations of 4APP (e.g., 0, 0.1, 2, 5, 10, 15 and 20 mM 4APP) for an additional 3.5 hours at the same temperature. Inclusion bodies containing the LPM fusion proteins were harvested from the cells (see method below). Upon induction in the presence of IPTG, the strains with the desired expression profiles showed an ˜36 kD expression band in a 4APP dose response fashion. Protein identity was verified by Western Blotting using an anti-SA1 antibody. Antibodies to the SA1 subunit were raised in rabbits to a synthesized peptide of SA1 (SEQ ID NO: 30) (Covance Research Laboratories, Denver Pa.), and the sera collected.

Table 14 sets forth the relative expression of the LPM conjugates LPM1d, LPM8, LPM3, LPM6 or LPM7. Following expression and harvesting of the LPM fusion proteins from the cells, the proteins were separated on an SDS-PAGE gel and total protein was visualized by staining with Coomasie Blue. The percent expression along the 4-APP dose curve was estimated by loading identically prepared samples from each of the identical shake flask fermentations (0.1 to 20 mM 4-APP). The 100% expression of any given LPM was based on seeing no more increased levels of protein on the gel at a given concentration of 4-APP. The percent expression was estimated by visually comparing the designated 100% expression to the lanes with samples that had lower expression and lower 4-APP in the fermentation broths. The experiments were performed at least two times. Generally, expression levels increased from little or no detectable amount of the desired protein at 0.1 mM 4APP to high levels at 10-20 mM 4APP.

TABLE 14
LPM Expression Levels with 4-APP

| | 4-APP (mM) | | | | |
|---|---|---|---|---|---|
| LPM | 0.1 | 2.0 | 5.0 | 10.0 | 15.0 |
| LPM1d | >5 | >5 | >5 | 70 | 100 |
| LPM8 | >5 | >5 | 25 | 100 | 100 |
| LPM3 | 15 | 30 | 100 | 100 | 100 |
| LPM6 | >5 | 0 | 100 | 100 | 100 |
| LPM7 | >5 | 100 | 100 | 100 | 100 |

### B. Protein Production

A batch fermentation method was developed for LPM production. Host cells (HMS174(DE3)) carrying a pET9c/LPM plasmid with the selected SA1 variant were grown in liquid enriched media culture (5-100 L in a fermentor or 400 ml in a 2.8 L shake flask) at 37° C. in the presence of 2 mM 4APP. Product expression was induced by 1 mM IPTG for 3-6 hours in the presence of 10 mM 4 APP and the cells were harvested by centrifugation. The cell pellet was homogenized (via sonication or 3-4 passages through a homogenizer) followed by debris removal and recovery of inclusion bodies (Ibs) using centrifugation. Ibs were washed 2-3 times with several volumes of dH₂O. Ibs were solubilized in buffer containing 6M guanidine hydrochloride centrifuged and the supernatant dialyzed against 8 M urea. Typical starting LPM yields from a fermentor or shake flask are estimated to be ˜1 g/L (OD_{600 nm}˜50) and 300 mg/L (OD_{600 nm}˜7), respectively. The noted optical density (OD) at 600 nm were a measurement of E. coli density using an Ultraspec Pro spectrophotometer. Nucleic acids were removed from the IB solution with 0.1% (v/v) polyethyleneimine. After centrifugation, additional DNA was removed by passage through an anion exchange resin filter or column (Q-sepharose-FF), which binds residual DNA and allows proteins with high isoelectric points such as LPMs to pass through. The protein product was captured via cation exchange resin chromatography (S-sepharose-FF or HP) and eluted with an NaCl gradient in the presence of urea. The flow through fraction from this column contains a small amount of free SA1 toxin moiety. Protein fractions were collected throughout the NaCl gradient and analyzed by SDS-PAGE. Fractions containing LPMs were then pooled. Refolding of the product was performed by dialysis against 25 mM Tris-HCL, 1 M urea, 0.5 M L-arginine, 1 mM reduced glutathione and 0.1 mM oxidized glutathione at pH 8.0 for 16-24 h. Refolded material was then dialyzed into formulation buffer (50 mM sodium citrate, 0.05 mM EDTA, and 20% sucrose) and stored at −80° C. This material was over 80% pure as assessed by SDS-PAGE and was further purified using cation exchange or hydrophobic interaction chromatography as a polishing step prior to formulation. Another process employed the same initial steps but the product from the initial anion-exchange was immediately refolded by dilution into 25 mM sodium phosphate, 1 M urea, 200 mM L-arginine, 20% (w/v) sucrose, 1 mM reduced glutathione and 0.1 mM oxidized glutathione at pH 8.0 for 16-24 h. The refolded material was then subjected to cation-exchange followed by hydrophobic interaction chromatography before being dialyzed into the formulation buffer described above.

### Example 5

### Cell Based Cytotoxicity Assay

Cell toxicity of LPM1d and LPM12 were measured in a cell-based cytotoxicity assay. In this assay, cells were grown in the presence or absence of toxin (i.e. LPM protein containing an SA1 moiety) for a period of time. The amount of ATP in the culture upon cell lysis served as a measurable indicator of cell viability.

### A. Cell Culture and Sample Additions

THP-1 monocyte cells were grown according to the manufacturer's instructions (ATCC, Manassas, Va.) in complete media containing RPMI media supplemented with 10% FBS (Invitrogen, Carlsbad, Calif.) and passaged twice a week to keep the cell density below 5×10⁵ cells/mL. Cells were collected by centrifugation and washed with fresh warm media and resuspended in an appropriate volume of media to reach a density of 3-4×10⁴ cells/ml for the cell-based cytotoxicity assay. Cells were seeded by transferring 100 μL aliquots of the cell suspension to each of the internal 60 wells of a 96-well cell culture plate (the outer wells were filled with complete media only). 20 μl of vehicle (buffer only) and LPM1d or LPM12 protein samples (at concentrations ranging from 25 μg/ml to 100 μg/ml) were added to the wells in triplicate and gently mixed with the cells. The cells were then incubated for 24 hours at 37° C. (5% CO₂).

### B. Assessment of Cell-Based Cytotoxicity

The CellTiter-Glo™ Luminescent Cell Viability Assay Kit (Promega, Madison Wis.) was used (as per the manufacturer's instructions) to assay cell viability as a measure of cell-based cytotoxicity. Upon lysis of the cells with the ATP reaction mixture (supplied by the manufacturer as CellTiter-Glo® Reagent), ATP drives the oxygenation of luciferin resulting in a luminescent signal which is proportional to ATP concentrations in the wells. This is directly proportional to the number of viable cells in the culture. Aliquots of 100 μL from the THP-1 cell plate from part A above were transferred to white flat bottomed culture plates (Corning Corporation, NY) to allow luminescent measurement, and allowed to equilibrate for 30 minutes before adding 100 μL of the ATP reaction mixture. After addition of the ATP reaction mixture, the contents of the plates were shaken gently for 30 seconds using a vortex to induce cell lysis and incubated at room temperature for 10 minutes to stabilize the luminescent signal. Luminescence was measured using a FLUOstar luminometer (BMG Lab Technologies, Durham, N.C.). Matched control wells were prepared for each LMP protein containing vehicle only (buffer only). Triplicate values were averaged and background luminescence subtracted for all conditions tested. The ATP content in the presence of an LMP fusion protein was presented as a percentage of the ATP content in the presence of the matched control (which was set at 100%). LPM1d and LPM12 were tested in the cell based cytotoxicity assay and the results are set forth in Table 15 and Table 16, respectively. The results show that the ATP content is dose-dependently decreased in the presence of increasing concentrations of LPM1d, showing that LPM1d is toxic to THP-1 cells. LPM12 also was toxic to THP-1 cells, but only at concentrations of 33 μg/ml or greater and no dose-dependent effect of LPM12 was observed on these cells. The observed effect of LPM12 (a fusion protein containing the eotaxin chemokine) could be due to the lack of expression of the eotaxin receptor, CCR3, on THP-1 cells, and the presence of the MCP-1 receptor, CCR2, on THP-1 cells for which eotaxin binds at high concentrations (Ogilvie et al., (2001) Blood 97: 1920-1924).

TABLE 15
Cell Toxicity of LPM1d on THP-1 Cells

| | Percentage of Matched Control |
|---|---|
| Concentration of LMP | (Buffer only) ATP Content |
| 0 μg/ml | 100% |
| 25 μg/ml | 96.02% |
| 35 μg/ml | 63.52% |
| 50 μg/ml | 52.23% |
| 75 μg/ml | 38.83% |
| 100 μg/ml | 26.47% |

TABLE 16
Cell Toxicity of LPM12 on THP-1 Cells

| | Percentage of Matched Control |
|---|---|
| Concentration of LMP | (Buffer only) ATP Content |
| 0 μg/ml | 100% |
| 25 μg/ml | 127.87% |
| 35 μg/ml | 56.01% |
| 50 μg/ml | 81.36% |
| 75 μg/ml | 55.22% |
| 100 μg/ml | 61.88% |

### Example 6

### Activity of LPM1d in Anti-Thymocyte Serum (ATS)-Induced Mesangioproliferative Glomerulonephritis in Rats

The following example demonstrates the effects of LPM treatment on the progression of anti-thymocyte serum (ATS)-induced mesangioproliferative glomerulonephritis in rats.

### A. ATS Injection and LPM1d Treatment

The effect of LPM1d treatment on the progression of ATS-induced mesangioproliferative glomerulonephritis in rats was assessed. Twenty-four rats were weighed and set up in metabolic cages for 24 hrs for a basal urine collection. Urine volumes were recorded and the urine processed and quantified for creatinine and protein using standard procedures. Rats were anesthetized, and 0.5-1.0 ml of blood was taken from a marginal tail vein. The blood was clotted and serum retained for measurement of blood urea nitrogen (BUN), creatinine, and cholesterol using standard procedures. The rats were injected on Day 0 with 20 mg/100 g body weight of anti-thymocyte (Thyl) IgG fraction (Probotex, San Antonio, Tex.) and returned to their cages upon recovery. The rats were monitored daily and body weights and state of health recorded. The rats were divided into three groups of 8: two groups were injected every other day (Days 2, 4, 6 and 8) with LPM1d at 50 or 100 μg/kg, respectively, and the third group was injected with vehicle only (50 mM sodium citrate buffer pH 6.2 containing 0.05 mM EDTA) as a disease control group starting on Day 2 after antibody administration. On day 4 the rats were returned to the metabolic cage for a midpoint urine collection. On the following day, blood was obtained from the tail vein for a midpoint serum collection. On day 8 the rats were again set up in metabolic cages for terminal 24-hr urine collection. Animals were healthy throughout the experiment. The glomerular filtration rate (as measured by urine creatinine clearance) of LPM treated groups generally did not differ from controls, with only a slight increase observed in the higher dosed animals on days 5 and 9. BUN and cholesterol levels were all in the normal range for all animals. Urine protein was determined at midpoint in the study (24 h urine collection Day 4-5). The low and high dose treated rats were found to have a 34% and 39% decreases in urine protein, respectively compared to control indicating that LPM1d had protective effect on renal function.

### B. Histological Analysis

On day 9, all rats were sacrificed, blood was collected and kidneys were processed for histology. The kidney cortexes from this experiment were sliced in 2-3 mm coronal sections and either flash frozen in liquid nitrogen, placed in formalin or placed in methacam and fixed overnight at 4° C.

1. Immunohistochemical Staining for Fibrotic Process Markers

Frozen sections were processed using antibodies to fibronectin and alpha smooth muscle actin (α-SMA) (clone IST-9, Serotec, Harlan Bioproducts for Science, Indianapolis, Ind. and clone 1A4 from Sigma, St. Louis Mo., respectively). Fibronectin is a marker for extracellular matrix (ECM) deposition and synthesis, and alpha smooth muscle actin (α-SMA) is a marker for hypercellular mesangial cells undergoing phenotypic changes which is a prelude to ECM deposition. Expression of fibronection and α-SMA are indicative of the fibrotic process. For staining for either α-SMA or fibronectin, the results are depicted on a scale of 0-4, which indicates zero, slight, moderate, high, and severe staining, respectively. Table 17 depicts the results of staining frozen sections with α-SMA as an average (AV) score of all 4 rats in a Group. The results show that there is decreased expression of α-SMA in the presence of increased concentrations of LPM1d. Thus, there is decreased activation of mesangial cells in LPM1d treated kidneys.

TABLE 17
α-SMA Levels in Frozen Kidney Sections

| Treatment | AV Score Group 1 | AV Score Group 2 |
|---|---|---|
| Vehicle | 2.18 | 2.10 |
| 50 μg/kg LPM1d | 1.76 | 2.08 |
| 100 μg/kg LPM1d | 1.69 | 1.30 |

Table 18 depicts the results of staining frozen kidney sections for fibronectin upon treatment of rats with LPM1d. The results show a decreased expression of fibronection by immunohistochemical staining, particularly at high concentrations of LPM1d (100 μg/kg). Thus, there is a decreased ECM deposition in LPM1d treated kidneys.

TABLE 18
Fibronectin levels in Frozen Kidney Sections

| Treatment | AV Score Group 1 | AV Score Group 2 |
|---|---|---|
| Vehicle | 1.86 | 1.98 |
| 50 μg/kg LPM1d | 1.48 | 2.0 |
| 100 μg/kg LPM1d | 1.49 | 1.43 |

2. Haematoxylin and Eosin Stain (H&E) of Renal Lesions

Formalin treated samples were processed for haematoxylin and eosin stain (H&E) assessment of renal lesions. H&E staining of frozen sections allowed the visualization and global assessment of renal lesions and glomerular integrity and structure which are scored on a scale of 0-4 from normal appearance to severe damage. The results (Table 19) show a decreased presence of renal lesions in α-Thy1 treated rat kidneys in the presence of LPM1d. There were no distinct lesions observed in the group of rats treated with 100 μg/Kg LPM1d (i.e. equivalent to a score of 1.44). Thus, there is a reduction of renal lesions and structural damage in LPM1d treated kidneys.

TABLE 19
H&E staining of Renal Lesions in Frozen Kidney Sections

| Treatment | Average Score (n = 4) |
|---|---|
| Vehicle | 2.4 |
| 50 μg/kg LPM1d | 2.25 |
| 100 μg/kg LPM1d | 1.44 |

3. Immunohistochemical Staining for Proliferating Cells

Methacam treated samples were used for immunohistochemical assessment of macrophage numbers using the ED-1 antibody (Chemicon Corporation, Temecula, Calif.). In this model, the number of macrophages peaks at about day 5. For assessment of ED-1 positive macrophages, the total number of macrophages (i.e. ED-1 positive cells) was counted from 25 glomeruli at day 9. The results are depicted in Table 20 as raw numbers of counted macrophages in each of the 4 rats in a group. The results show that there is a decreased presence of macrophages in LPM1d treated kidneys.

TABLE 20
Number of Macrophages in Glomeruli at Day = 9

| Treatment | Raw # per Animal |
|---|---|
| Vehicle | 109, 100, 120, 110 |
| 50 μg/kg LPM1d | 79, 71, 63, 90 |
| 100 μg/kg LPM1d | 62, 71, 68, 87 |

### Example 7

### Activity of LPM1c and LPM1d in a Mouse Delayed Type Hypersensitivity Model

The following example demonstrates the effects of LPM1c and LPM1d treatment on the degree of inflammatory response to oxazolone in mouse ears.

Effects of LPM proteins on a cell-based immune response was assessed in a mouse model of delayed type hypersensitivity (MDTH) induced by the administration of the antigen oxazolone. The effects of LPM1c and LPM1d treatment on the degree of the inflammatory response to oxazolone in mouse ears were assessed. 56 female Balb/c mice weighing ˜20-25 grams were divided into seven treatment groups as outlined in Table 21. Mice were sensitized to 2% oxazolone (Sigma, St. Louis, Mo.) on day −7 and day −6 by applying the antigen solution to a shaved area on the body. On day 0, mice were challenged with 2% oxazolone solution applied directly to both ears. On day 0 and day 1, mice were treated with LPMIc (100 μg/kg), LPM1d (10 μg/kg or 25 μg/kg), dexamethazone (an anti-inflammatory corticosteroid, 0.2 mg/kg (Vedco Inc, St. Joseph, Mo.), or vehicle control.

TABLE 21
MDTH Treatment Groups

| Group | Treatment (n = 8) |
|---|---|
| 1 | Non-sensitized, challenged + Vehicle |
| 2 | Sensitized, challenged + Vehicle |
| 3 | Sensitized, challenged + LMP1c 100 μg/kg |
| 4 | Sensitized, challenged + LPM1d 25 μg/kg |
| 5 | Sensitized, challenged + LPM1d 10 μg/kg |
| 6 | Sensitized, challenged + Dexamethasone 0.2 mg/kg |
| 7 | Non-challenged |

To assess the degree of inflammatory response to oxazolone and compare the effects of the treatments outlined in Table 21, the thickness and total weight of the ears were measured. Both ears of the mice were measured with a caliper prior to challenge, at 24 hours post challenge, and at study termination (48 hours post challenge). In addition, the ears were removed and weighed at study termination.

The mean+/−standard error of the final ear weight in grarns was determined. A two-tailed t-test was used to analyze the statistical significance of the results and all LPM treatments gave a statistically significant (*p<0.05) relative decrease in ear width compared to the vehicle-treated/sensitized/challenged group (Group 2 in Table 18), as did the positive control dexamethazone treated group. The percent decrease in ear width relative to the vehicle-treated/sensitized/challenged group was calculated for each Group. The percent decrease was calculated by the formula: 1−[(treated−negative control)/(positive control−negative control)]×100%. The results are set forth in Table 22. Ear thickness measurements in LPM1c (group 3) and LPM1d (group 4 and group 5) treatment groups were nearly as reduced as in the dexamethazone (group 6) treatment group (29%).

TABLE 22
Effects of LPM Treatment on Ear Weight in MDTH

| | Percent Decrease |
|---|---|
| | Ear |
| | Weight relative to |
| | vehicle/sensitized |
| Treatment Group | challenged Group 2 |
| Group 1: Non-sensitized, challenged + Vehicle | 74% |
| Group 2: Sensitized, challenged + Vehicle | 0% |
| Group 3: Sensitized, challenged + LMP1c 100 μg/kg | 20% |
| Group 4: Sensitized, challenged + LPM1d 25 μg/kg | 29% |
| Group 5: Sensitized, challenged + LPM1d 10 μg/kg | 22% |
| Group 6: Sensitized, challenged + Dexamethasone | 28% |
| 0.2 mg/kg | |
| Group 7: Non-challenged | 100% |

### Example 8

### Activity of LPM1d in a Spinal Cord Injury Model

### A. Spinal Cord Injury and LPM Administration

A spinal cord injury (SCI) model experiment was designed in which LPM1d was administered only in the first 1-3 days post injury in order to quantify the decrease in macrophage and neutrophil populations. Briefly, spinal cord injury was induced as follows: Adult 6-8 week old CD-1 mice (Charles River Laboratories, Montreal, Quebec, Canada) were anesthetized with a mixture of ketamine-xylazine (85 mg/kg and 15 mg/kg, intraperitoneal (I.P.)), respectively and were subjected to a moderate (60 kdyne) T9/10 contusion spinal cord injury (SCI) (Infinite Horizons Impactor, Precision Systems Instrumentation, Kentucky, USA). The injury has been well characterized in rodents and produces a moderate lesion in a reproducible manner that mimics the pathophysiology of human SCI (see e.g., Wells et al. (2003) Brain, 126: 1628-37). Following injury the mice were allowed to recover on a warm blanket and received 0.5 ml saline to compensate for loss of blood and dehydration. Bladders were manually expressed 2-3 times daily until spontaneous voiding returned. All experiments were conducted in accordance with the University of Calgary Animal Care Ethics Committee adhering to guidelines of the Canadian Council on Animal Care.

Following injury, LPM1d or vehicle controls were administered to the mice. The mice were randomly assigned into four treatment groups as set forth in Table 23 below.

TABLE 23
Treatment with LPM1d in SCI Models

| Group | Treatment |
|---|---|
| I | Single bolus of LPM1d (100 μg/kg, I.P.) 2 hours post-SCI |
| II | Two injections of LPM1d (100 μg/kg, I.P.) 2 hours and |
| | 24 hours post-SCI |
| III | Three injections of LPM1d (100 μg/kg, I.P.) 2 hours, 24 hours, |
| | and 48 hours post-SCI |
| IV | Vehicle (I.P.) |

### B. Harvesting of Tissue and Blood for Data Analysis

1. Fresh Tissue

Fresh tissue was collected from each of the treatment groups at 24 and 48 hours after SCI injury after mice were anesthetized, and ˜1 ml of whole blood was collected by cardiac puncture into 100 μl of Heparin solution. Immediately following blood collection, the animals were perfused with ice-cold PBS and the spinal cord (2 cm centered around the lesion site) was rapidly isolated and placed into ice-cold PBS. Blood and spinal cord samples were then prepared for flow cytometry.

2. Fixed Tissue

Fixed tissue was collected from each of the treatment groups at 5 days post-SCI injury. Animals were anesthetized with a lethal dose of ketamine-xylazine, perfused with PBS, followed by perfusion-fixation with a solution of 4% paraformaldehyde in PBS. The spinal cords (T6 to T13) were removed and post-fixed in 4% paraformaldehyde overnight and subsequently cryoprotected in 30% sucrose. The spinal cords were then placed into blocks, frozen, and stored at −70° C. until sectioned. Blocks were sectioned in the transverse plane at a thickness of 20 μm and the tissue sections were collected on Superfrost slides (Fisher Scientific, Houston, Tex.) organized into five adjacent section series.

3. Statistical Analysis

Statistical analysis was performed using SigmaStat Software (SPSS, Inc.). Differences between the treatment groups were tested using an analysis of variance (ANOVA) and the Holm-Sidak post-hoc analysis when warranted. In the case of unequal variances, the Kruskal-Wallis one way ANOVA on ranks was used. Differences with a P value less than 0.05 were considered significant.

### C. Data Analysis

1. Flow Cytometry

Spinal cord samples from fresh tissue were mechanically disrupted with a small glass dounce homogenizer, and single cell suspensions were obtained by passing the solution through a wire mesh screen (Sigma-Aldrich, Canada). Samples were then subjected to centrifugation at 4° C. at 1100 RPM (200×g) for 10 minutes with low break. Pellets were resuspended in FBS staining buffer (BD Biosciences) and were subjected to centrifugation (3000 RPM for 7 minutes, slow brake at 4° C.). Pellets were then resuspended in FBS staining buffer.

Spinal cord cells were stained with antibodies to markers used to determine populations of resident microglia (CD45dim:CD11b) and blood-derived leukocytes (granulocytes and monocytes; CD45high:CD11b). To optimize antibody dilutions, cell numbers were first counted using trypan blue staining by diluting the cells 1:1 in trypan blue (10 μl trypan blue to 10 μl of each sample) and counting cell number using a hemacytometer. Samples were first incubated with Fc Block™ (Purified rat anti-mouse CD16/CD32 (FcγIII/II receptor; BD Biosciences; 0.5 mg/ml)) to reduce nonspecific binding due to antibody binding to the Fc receptor. After incubation with Fc block for about 5 minutes, the following monoclonal antibodies (BD Biosciences) were added to the cell samples to assess the presence of resident microglia and blood derived leukocytes: R-Phycoerythrin (R-PE)-conjugated rat anti-mouse CD11b (0.2 mg/ml), FITC anti-mouse Ly-6G and Ly-6C (Gr-1; 0.5 mg/ml), FITC anti-mouse CD3 molecular complex (0.5 mg/ml), and Peridinin chlorophyll-a protein (PerCP)-conjugated rat anti-mouse CD45 (Leukocyte common antigen, Ly-5; 0.2 mg/ml). To control for non-specific binding and autofluorescence, staining also was performed using appropriate isotype control antibodies (i.e. PE labeled rat IgG2a, k isotype control (0.2 mg/ml); FITC labeled rat IgG2b, k isotype control (0.5 mg/ml); and PerCP-conjugated rat IgG2b isotype control (0.2 mg/ml)). A cell only sample also was included in the staining incubation. Cells were incubated for 30 minutes at 4° C. Following the incubation, the cell samples were washed twice in FBS staining buffer and resuspended in 1% buffered formalin. Cell samples were stored at 4° C. and analyzed using a BD FACScan (BD Biosciences).

The results of the flow cytometry was determined from density plots (CD45, y-axis; CD11b, x-axis) using WinMD1 version 2.8 software (Scripps Research Institute, California, USA) and compared between the different treatment groups. Using the WinMD1 version 2.8 software, the mean fluorescence of CD45 and CD11b staining was determined for each treatment group. The ratio of blood-derived leukocytes over resident microglia was determined as a ratio of the mean fluorescence values of CD45:CD11b. Standard errors of the mean also were determined for each of the treatment groups. The result are depicted in Table 24. The results show that 24 hours after SCI, mice in Group I treated with LPM1d 2 hours post-SCI exhibited a reduced ratio of blood derived leukocytes in the spinal cord compared to mice treated with vehicle only. The analysis shows that mice receiving one dose of LPM1d at two hours post-SCI revealed that the blood-derived leukocyte:microglia ratio at 24 hours was significantly (P<0.05) reduced compared to controls. This represents a 30% reduction in blood-derived leukocytes as microglia numbers did not change between the groups. At 48 hours post-SCI, the ratio of spinal cord cells from mice treated with two doses of LPM1d at 2 and 24 hours post-infection (i.e. Group II) showed no significant difference between the test and control ratios.

TABLE 24
The Ratio of Blood-derived Leukocytes vs. Resident Microglia at 24
and 48 hrs Post-SCI

| Treatment | n | Mean | SEM | Timepoint |
|---|---|---|---|---|
| vehicle | 6 | 2.431 | 0.317 | 24 hr |
| Group I | 6 | 1.703 | 0.102 | 24 hr |
| vehicle | 6 | 3.02 | 0.38 | 48 hr |
| Group II | 6 | 3.619 | 0.965 | 48 hr |

2. Immunohistochemistry to Detect Microglia/Macrophages

Fluorescence immunohistochemistry was performed on slides containing fixed tissue sections from 5 day-post injured spinal cords. Slides were thawed, rinsed three times in PBS, and blocked in 10% normal goat serum for 30 minutes at room temperature. To detect microglia/macrophages, slides were incubated with a rabbit anti-Iba1 antibody (1:1000; Wako Chemicals USA, Inc.) for two hours at room temperature. Following three washed in PBS, slides were incubated for one hour at room temperature with the Alexa488 goat anti-rabbit secondary antibody (1:1000, Molecular Probes Inc., USA) to visualize Tba-1. Slides were then washed three times in PBS and submerged in Hoechst 33258 (1 μg/ml, Sigma-Aldrich, Canada).

To quantify microglial/macrophage activation/recruitment within the lesioned spinal cord, an overlay box (1024 by 1024 pixels) was placed onto digitally captured confocal thresholded images of transverse spinal cord sections containing Iba-1 signal using SigmaScan Pro software (SPSS, Chicago, Ill.). The percentage of the area occupied by Iba-1 signal was calculated to determine the density of spinal cord microglia/macrophages using lba-1 tissue staining at 5 days post-SCI. At least two sections from the center of the lesion site of each animal was assessed (n=2-3 animals per group). The mean and standard error of the mean (SEM) lba-1 signal was determined and the results are depicted in Table 25. The results show that animals receiving a single dose of LPM1d at 2 hours (Group I) and mice receiving three doses of LPM1d at 2, 24, and 48 hours post-SCI (Group III) showed a 40% and 60% reduction in cell numbers, respectively, compared to vehicle only controls. This was statistically significant (P<0.05 difference between the treatment groups Kruskal-Wallis one way ANOVA on Ranks). In this experiment, mice in Group II receiving two doses of LPM1d at 2 hours and 24 hours showed no significant difference in the percentage of cells that were lba-1 positive compared to control cells.

TABLE 25
Density of Iba-1 (% area) Positive Microglia/Macrophages 5 days
post-SCI

| Treatment | n | Mean | SEM |
|---|---|---|---|
| vehicle | 2 | 12.148 | 2.822 |
| Group I | 3 | 7.342 | 0.503 |
| Group II | 2 | 13.465 | 1.337 |
| Group III | 2 | 4.89 | 1.297 |

### Example 9

### Activity of LPMs in a Xenograft Model

The effects of LPMs in breast cancer were assessed using an established tumor xenograft model. Female athymic nude mice (nu/nu) were injected with 2.5 million cells (in 0.2 ml of PBS/Matrigel) of the estrogen dependent breast carcinoma cell line MCF-7 (American Type Culture Collection (ATCC), Manassas, Va.) and the effects of two LPM molecules on tumor growth were assessed.

### A. SDF-1β-SA1Var1 LPM

In this study, SDF-1β-SA1Var1 LPM (SEQ ID NO:216) was used in the treatment regime. This LPM contains a mature SDF-1β chemokine linked to a wildtype SA1 moiety. Intraperitoneal dosing of 100 μg/kg SDF-1β-SA1Var1 LPM or vehicle control began on day 7 after MCF-7 injection and continued every day through day 21. Tumors were allowed to continue to grow until Day 31.

1. Tumor Growth

The effects of SDF-1β-SA1Var1 in retarding the progression of MCF-7 breast carcinoma cells in this mouse xenograft model was determined by assessing tumor growth as measured by tumor weight and tumor volume. In the absence of SDF-1β-SA1Var1, tumor growth steadily increased from day 7 (starting at about 100 mm3) through day 31, reaching maximal tumor volume of about 980 mm³ by day 28. Treatment of mice with LPM at a dose of 100 μg/kg also resulted in a steady increase in tumor growth; however, the magnitude of tumor growth was significantly less than in the absence of LPM. For example, in the presence of LPM maximal tumor growth was reached at day 28, however, maximal tumor volume only reached about 500 mm3. The results show treatment of mice with LPM resulted in a statistically significant decrease in the rate of MCF-7 tumor growth. The final tumor weights from test animals decreased by an average of 35% and final tumor volumes by 41.5% that of control (significant using p<0.05 two tail t-test).

2. Inflammatory Infiltrate

Microscopic examination was used to determine the effects of SDF-1-SA1Var1 LPM on the inflammatory infiltrate in this model. Tumors were excised and sectioned ten days after the last dose of SDF-1β-SA1Var1 LPM was given to the mice (i.e. day 31), and examined by microscopy to evaluate the leukocyte infiltrate around the periphery of the tumors. Cells were visualized with Haematoxylin and Eosin (H&E) staining. The results showed that there was 36% less cells in the tissue of LPM treated animals compared to animals treated with vehicle only, which was statistically significant.

3. CD-31 Staining

Histological examination allowed the visualization of the extent of intratumoral necrosis and vacuolization. Anti-CD-31 (goat polyclonal PECAM-1 (clone M-20), Santa Cruz Biotechnology, Santa Cruz, Calif.) was used to visualize PECAM-1, a cell adhesion molecule and glycoprotein expressed on the cell surfaces of monocytes, neutrophils, platelets and a subpopulation of T cells. PECAM-1 also is expressed on the surface of adult and embryonic endothelial cells. Tumors were excised, sectioned and stained with anti-CD-31 antibodies ten days after the last dose of SDF-1β-SA1Var1 LPM was given to the mice (i.e. day 31). Briefly, formalin-fixed paraffin embedded tumor specimen sections were deparaffinized and hydrated. A pretreatment of heat-induced-epitope retrieval in Target Retrieval Solution (pH 9.0, DakoCytomation, Carpenteria, Calif.) was used prior to primary antibody incubation. Endogenous peroxidase activity was inhibited by incubation with 3% H₂O₂, and nonspecific staining was blocked with DAKO Protein Block Serum-Free (DakoCytomation, Carpenteria, Calif.). Sample were incubated with primary (anti-CD-31) antibody diluted 1:800 for 30 min at room temperature. Tissue sections were then incubated with biotinylated rabbit anti-goat immunoglobin (Vector Laboratories, Burlington, Calif.) diluted 1:400 for 30 minutes at room temperature, followed by application of Dako Envision+Rabbit System Labeled Polymer, HRP (DakoCytomation, Carpenteria, Calif.). Staining was developed with Liquid DAB+ (DakoCytomation, Carpenteria, Calif.) and counterstained with Haematoxylin. Some leukocytes (visualized by circular staining under histological analysis) and endothelial cells (visualized by staining of cells with an elongated shape) stained positive for CD-31. These results indicated the presence of angiogenesis in the growing tumor. Conversely, there was no CD-31 staining in the SDF-1β-SA1Var1 LPM treated tumors indicating the absence of macrophages (athymic mice have no T cells) and the absence of intratumoral endothelial cell blood vessels.

4. Ki-67 Staining

Histological examination also was used to assess the effects of SDF-10-SAlVarl LPM on cell proliferation in this model by staining cells with rabbit polyclonal anti-Ki-67 antibody (Santa Cruz Biotechnology, Santa Cruz, Calif.) ten days after the last dose of SDF-1β-SA1Var1 LPM was given to the mice (i.e. day 31). This antigen is expressed during all active phase of the cell cycle (G1, S, G2 and M phases), but is absent in resting cells (G0-phase). The Ki-67 antigen is rapidly degraded as the cell enters the non-proliferative state, and there is no expression of Ki-67 during DNA repair processes. Briefly, formalin-fixed paraffin embedded tumor specimen sections were deparaffinized and hydrated. A pretreatment of heat-induced-epitope retrieval in Target Retrieval Solution (pH 9.0, DakoCytomation, Carpenteria, Calif.) was used prior to primary antibody incubation. Endogenous peroxidase activity was inhibited by incubation with 3% H₂O₂, and nonspecific staining was blocked with DAKO Protein Block Serum-Free (DakoCytomation, Carpenteria, Calif.). Samples were incubated with primary (anti-Ki-67) antibody diluted 1:200 for 30 min at room temperature. Tissue sections were then incubated with biotinylated goat anti-rabbit immunoglobin (Vector Laboratories, Burlington, Calif.) diluted 1:400 for 30 minutes at room temperature, followed by application of Dako Envision+Rabbit System Labeled Polymer, HRP (DakoCytomation, Carpenteria, Calif.). Staining was developed with Liquid DAB+ (DakoCytomation, Carpenteria, Calif.) and counterstained with Haematoxylin. A large number of cells were proliferating in the non-treated tumors as shown by Ki-67 staining. In contrast, SDF-1β-SA1Var1 LPM treated tumors showed little staining with anti-Ki-67 indicating decreased tumor progression. In addition, it appeared that many cancer cells had undergone necrosis as evidenced by the clear vacuoles in the field.

### B. MCP-1-SA1Var4 (LPM1d)

In this study, MCP-1-SA1Var4 (LPM1d) was used in the treatment regime. Intraperitoneal dosing began on day 7 and cohorts received either vehicle; (1) one dose of 2 mg/Kg LPM1d on day 7; (2) 2 mg/Kg LPM1d on days 7, 11, 15 and 21 or (3) 100 μg/Kg LPM1d every day from day 7 through day 21. Tumors were allowed to continue to grow until Day 32. The percent change in body weight of the treated animals between different cohorts including the control did not exceed 0.5%.

In the absence of LPM1d, tumor growth steadily increased from day 7 through day 32, reaching maximal tumor volume of about 1500 mm³ by day 32. Treatment of mice with LPM at all dosing regimes resulted in a steady increase in tumor growth; however, the magnitude of tumor growth was significantly less than in the absence of LPM1d. Treatment with MCP-1-SA1Var4 (LPM1d) induced a statistically significant decrease in the MCF-7 tumor growth as measured by tumor volume and weight. The decrease in tumor growth for all LPM1d treatment groups was similar from day 7 until day 29, although by day 32 there were some differences in the effects of the different LPM1d treatment groups on tumor growth. The final tumor weights from groups 1-3 decreased by 41, 58.6 and 36% that of control (significant using p<0.05 two tail t-test). The final tumor volumes from groups 1-3 decreased by 47, 63 and 40.4% that of control (significant using p<0.05 two tail t-test). This study indicates that a single or minimal repeated dosing is enough to significantly decrease the rate of tumor growth.

A second MCF-7 xenograft experiment was conducted with LPM1d. Dosing regimes from the first experiment yielded similar results. An additional dosing regime was added whereby tumors were allowed to first grow to ˜700 mm³ tumor volume (instead of ˜100 mm3) before treatment with LPM1d in order to test whether treatment could affect large growing tumors (with more prominent vasculature). Thus, the tumors were allowed to grow until about day 27 before administration of LPM1d or vehicle control. The animals were treated with 100 μg/kg LPM1d via intraperitoneal injection every fourth day from day 27 through 43. Treatment with LPM1d significantly reduced tumor volume immediately after the first injection (p<0.05) compared to controls. This trend continued out to day 43.

### Example 10

### Activity of LPMd in Experimental Autoimmune Encephalomyelitis (EAE) an Animal Model of Multiple Sclerosis

Eight- to ten-week old C57BL/6 female mice (Jackson Laboratory, Bar Harbor, Me.) were divided into 4 groups (Gr1-4). To induce EAE Groups 1 and 2 (n=9) were injected subcutaneously at the back of the tail on Day 0 with 100 μg of myelin oligodendrocytes glycoprotein (MOG)33-55 peptide (Bernard et al. (1997) J Mol Med 75:77-88) emulsified in 100 μl of complete Freund's adjuvant (Difco Laboratories, Detroit, Mich.). These mice also received an intraperitoneal (i.p.) dose of 300 ng of reconstituted lyophilized pertussis toxin in 200 μl of phosphate buffered saline on Day 0 and again on Day 2 (Liu et al. (1998) Nat Med 4:78-83). Groups 1 and 2, respectively, received 6 daily injections (Day 3-8) of LPM1d (500 μg/kg) in buffer (50 mM sodium citrate buffer, pH 6.2, 0.05 mM EDTA and 10% v/v glycerol) or buffer alone, Control mice (n=6) in Groups 3 and 4, respectively, received no injections at all and 6 daily (D3-8) injections of only 500 μg/kg LPM1d.

Animals were evaluated daily using a scoring system that makes the assessment of disease on a scale ranging from 0 to 15 (Weaver et al., 2005) The disease score is the sum of the state of the tail and all of the four limbs. For the tail, a score of 0 reflects no signs, 1 represents a half paralyzed tail, while a score of 2 is given to a mouse with a fully paralyzed tail. For each of the hind- or forelimbs, each assessed separately, 0 signifies no signs, a score of 1 is a weak or altered gait, 2 represents paresis, while a score of 3 denotes a fully paralyzed limb. Thus, a fully paralyzed quadriplegic animal would attain a score of 14. Mortality equals a score of 15. The results show that control mice (groups 3 and 4) exhibited no paralysis. Group 2, the EAE model treated with buffer, develop paralysis starting about 9 days post-injection and increasing linearly to a mean Clinical score of greater than 6 by day 14. The treated EAE animals started to develop paralysis, with a mean score of less that about 4 at day 10, which deceased to control levels (close to 0) by day 12 and remained there on day 13.

In the acute experimental autoimmune encephalomyelitis (EAE) model of multiple sclerosis, OPL-CCL2-LPM had a dramatic effect on the disease course. The onset and severity of disease were significantly retarded and reduced, respectively. In this study animals were treated daily with vehicle or LPM on days 3-8. Control and test animals showed an initial onset of disease on day 10 as predicted. Thereafter clinical severity scores of disease returned to zero (no behavioral indications of disease) in treated versus control animals (exhibiting clinical severity scores of 6-10) over the next four days.

Mean Clinical Severity Scores (rounded to nearest 0.5 on scale)

| Day | Control (Vehicle) | LPM Treated Animals |
|---|---|---|
| 1-9 | 0 | 0 |
| 10 | 2.5 | 2.5 |
| 11 | 3.5 | 1.0 |
| 12 | 4.5 | 0.0 |
| 13 | 6.5 | 0.5 |
| 14 | 10.0 | 1.0 |
| 15 | 10.0 | 3.5 |
| 16 | 9.5 | 6.5 |
| 17 | 9.0 | 6.5 |

Animals with no injections or vehicle alone (no disease) scored zero throughout.

### Example 11

OPL-CCL2-LPM was tested in a model of Anti-Thymocyte Serum (ATS)-induced mesangioproliferative glomerulonephritis. Male rats were injected with ATS on day 0 and treated intravenously with vehicle, 50 or 100 μg/kg of the recombinant protein Q2D from day 2 until day 8. Urine and blood collections were made prior to ATS injection and on days 5 and 9. Animals were sacrificed on day 9. No treatment related effects on body weight or signs of clinical toxicity were observed. Urine protein levels were decreased in treated animals. Histopathological analyses of kidney sections revealed maximum reductions of 40, 36, 38, and 28% for glomerular lesions, M/M count, fibronectin and α-smooth muscle actin, respectively. The latter two proteins are markers for extracellular matrix synthesis and mesangial cell activation, respectively. These results indicate a significant renal-protective effect in this model of nephritis and indicate that the chemokine-ligand toxins can be used in the treatment of diseases.

Since modifications will be apparent to those of skill in this art, it is intended that this invention be limited only by the scope of the appended claims.

1. A method of selecting a modified ribosome inactivation protein (RIP) or an active fragment thereof, comprising:
a) introducing a nucleic acid molecule encoding a RIP, or active fragment thereof, into a host cell(s);
b) growing the cells;
c) isolating cells that grow;
d) from among the cells that grow, isolating a cell that expresses a RIP or active fragment thereof, wherein the RIP or fragment contains a modification compared to that encoded by the nucleic acid molecule that is introduced in step a); and, optionally, expanding the isolated cell that expresses a RIP; and
e) identifying or isolating or purifying the modified RIP or active fragment thereof that is expressed in the isolated cell.

2. The method of claim 1, wherein cells are grown in medium that does not contain a selective modulator.

3. The method of claim 1, wherein the medium in which the cells are grown does not contain an adenine analog.

4. The method of claim 3, wherein the adenine analog is 4-amino-pyrazolo[3,4-d]pyrimidine (4-APP).

5. The method of claim 1, further comprising growing the cells at step b) in the presence of a selective modulator.

6. The method of claim 5, wherein the selective modulator is a RIP inhibitor, and is provided at a concentration that is not toxic to the host cells and inhibits or reduces toxicity of the RIP on the host cell, whereby the amount of RIP expressed is increased compared to in the absence of the RIP inhibitor, adenine analog, or 4-APP.

7. The method of claim 6, wherein the RIP inhibitor is an adenine analog.

8. The method of claim 7, wherein the adenine analog is 4-amino-pyrazolo[3,4-d]pyrimidine (4-APP).

9. The method of claim 8, wherein the concentration of 4-APP is about 0.1 mM to about 5.0 mM.

10. The method of claim 1, wherein the host cell is a eukaryotic cell or the method of claim 1, wherein the host cell is a prokaryotic cell.

11. The method of claim 10, wherein the host cell is a prokaryotic cell and is E. coli.

12. The method of claim 1, wherein the RIP encoded by the introduced nucleic acid molecule is a type I RIP, or an active fragment thereof, or wherein the RIP encoded by the introduced nucleic acid molecule is a type II RIP, the catalytic subunit thereof or an active fragment thereof.

13. The method of claim 12, wherein the RIP is selected from among dianthin 30, dianthin 32, lychnin, saporin-1, saporin-2, saporin-3, saporin-4, saporin-5, saporin-6, saporin-7, saporin-8, saporin-9, PAP, PAP II, PAP-R, PAP-S, PAP-C, mapalmin, dodecandrin, bryodin-L, bryodin, bryodin II, clavin, colicin-1, colicin-2, luffin-A, luffin-B, luffin-S, 19K-PSI, 15K-PSI, 9K-PSI, alpha-kirilowin, beta-kirilowin, gelonin, momordin, momordin-II, momordin-Ic, Mirabilis Antiviral Protein (MAP), MAP-30, alpha-momorcharin, beta-momorcharin, trichosanthin, TAP-29, trichokirin, barley RIP I, barley RIP II, tritin, flax RIP, maize RIP 3, maize RIP 9, maize RIP X, asparin-1, and asparin 2.

14. The method of claim 1, wherein the RIP is a type II RIP and is selected from among Shiga toxin (Stx), Shiga-like toxin II (Stx2), volkensin, ricin, nigrin-CIP-29, abrin, vircumin, modeccin, ebulitin-α, ebulitin-β, ebultin-γ, and porrectin.

15. The method of claim 14, wherein the RIP comprises subunit A, or an active fragment thereof.

16. The method of claim 14, wherein the RIP is Shiga toxin, the Shiga toxin comprises subunit A1 (SA1), or an active fragment thereof or consists only of the subunit A1 or an active fragment thereof.

17. The method of claim 16, wherein the SA1 is truncated.

18. The method of claim 16, wherein the SA1 is modified by replacement of a Cys with another amino acid.

19. The method of claim 18, wherein the replacing amino acid is Ser.

20. The method of claim 16, wherein the SA1 comprises or consists of the sequence of amino acid residues set forth in SEQ ID NO: 22 or SEQ ID NO: 24.

21. The method of claim 1, wherein the RIP encoded by the introduced nucleic acid molecule is conjugated to a ligand to form a ligand-toxin conjugate.

22. The method of claim 21, wherein the RIP and ligand in the conjugate are linked directly or indirectly via a covalent or ionic linkage or are joined via a linker.

23. The method of claim 22, wherein the RIP and ligand are joined via a linker and the linker is a peptide, polypeptide or an amino acid.

24. The method of claim 23, wherein the linker is an Ala-Met linker.

25. The method of claim 21, wherein the ligand-toxin conjugate is a fusion protein.

26. The method of claim 21, wherein the ligand is selected from among a chemokine receptor targeting agent, a non-chemokine cytokine, a hormone, a growth factor, an antibody specific for a cell surface receptor, a TNF superfamily ligand, and a pattern recognition receptor (PRR) ligand.

27. The method of claim 26, wherein the ligand is a vascular endothelial growth factor (VEGF).

28. The method of claim 26, wherein:
the chemokine receptor targeting agent is a chemokine, or a fragment of the chemokine, or an antibody that specifically binds to a chemokine receptor, or a fragment of an antibody, wherein the fragment of the chemokine or antibody binds to the chemokine receptor.

29. The method of claim 28, wherein the chemokine, monoclonal antibody or fragment specifically binds to an antigen selected from among (DARC), D6, CXCR-1, CXCR-2, CXCR-3A, CXCR3B, CXCR-4, CXCR-5, CCR-1, CCR-2A, CCR-2B, CCR-3, CCR-4, CCR-5, CCR-6, CCR-7, CCR-8, CCR-9, CCR10, CX3CR-1, and XCR1.

30. The method of claim 28, wherein the chemokine, monoclonal antibody or fragment specifically binds to an antigen selected from among CXCR-6 and CXCR-7.

31. The method of claim 28, wherein the targeting agent is a chemokine or fragment thereof selected from among monocytes chemotactic protein-1 (MCP-1), MCP-2, MCP-3, MCP-4, MCP-5, eosinophils chemotactic protein 1 (Eotaxin-1), Eotaxin-2, Eotaxin-3, stromal derived factor-1β (SDF-1β), SDF-1α, SDF-2, macrophage inhibitory protein 1α (MIP-1α), MIP-1β, MIP-1γ, MIP-2, MIP-2α, MIP-2β, MIP-3, MIP-3β, MIP-3α, MIP-4, MIP-5, Regulated on Activation, Normal T cell Expressed and Secreted (RANTES) protein, interleukin-8 (IL-8), growth regulated protein α (GRO-α), interferon-inducible protein 10 (IP-10), macrophage-derived chemokine (MDC), granulocyte chemotactic protein 2 (GCP-2), epithelial-derived neutrophil-activating protein 78 (ENA-78), platelet basic protein (PBP), gamma interferon-induced monokine (MIG), platelet factor 4 (PF-4), hemofiltrate CC chemokine 1 (HCC-1), thymus and activation-regulated chemokine (TARC), lymphotactin, lungkine, C10, liver-expressed chemokine (LEC), exodus-2 (SLC), thymus expressed chemokine (TECK), cutaneous T-cell attracting chemokine (CTACK), mucosae-associated epithelial chemokine (MEC), single C motif 1-β (SCM-1β), interferon-inducible T-cell alpha chemoattractant (I-TAC), breast and kidney-expressed chemokine (BRAK), fractalkine, and B cell-attracting chemokine 1 (BCA-1).

32. The method of claim 21, wherein the ligand-toxin conjugate comprises the sequence of amino acid residues set forth in SEQ ID NO: 38 or SEQ ID NO:40.

33. The method of claim 21, wherein the ligand-toxin conjugate is encoded by a nucleic acid molecule comprising the sequence set forth as in SEQ ID NO: 37 or SEQ ID NO:39.

34. The method of claim 1, wherein the identified RIP contains a mutation compared to the RIP encoded by the introduced nucleic acid molecule.

35. The method of claim 1, wherein the identified RIP retains toxicity compared to the RIP encoded by the introduced nucleic acid molecule.

36. The method of claim 1, further comprising:
a) introducing a nucleic acid molecule encoding the identified RIP, or active fragment thereof into a host cell(s);
b) incubating the cells in the presence of a RIP inhibitor, wherein the amount of RIP inhibitor is selected to decrease the toxicity of the RIP polypeptide; and
c) growing the cells under conditions, whereby the RIP or active fragment thereof is produced.

37. The method of claim 36, further comprising purifying the RIP of step c), whereby the amount of RIP expressed or purified or both is greater than in the absence of the RIP inhibitor.

38. A method for increasing production of a ribosome inactivating protein (RIP), or active fragment thereof, comprising:
a) introducing a nucleic acid comprising a sequence of nucleotides encoding a RIP, or active fragment thereof, into a host cell(s);
b) incubating the cells in the presence of a RIP inhibitor, wherein the amount of RIP inhibitor is selected to decrease the toxicity of the RIP;
c) growing the cells under conditions, whereby a RIP or active fragment thereof is produced in an amount greater than when grown in the absence of the RIP inhibitor; and
d) purifying the RIP of step c), whereby the amount of RIP expressed or purified or both is greater than in the absence of the RIP inhibitor.

39. The method of claim 38, wherein the RIP encoded by the introduced nucleic acid is a type I RIP, or an active fragment thereof or is a type II RIP or an active fragment thereof.

40. The method of claim 38, wherein the RIP is selected from among dianthin 30, dianthin 32, lychnin, saporin-1, saporin-2, saporin-3, saporin-4, saporin-5, saporin-6, saporin-7, saporin-8, saporin-9, PAP, PAP II, PAP-R, PAP-S, PAP-C, mapalmin, dodecandrin, bryodin-L, bryodin, bryodin II, clavin, colicin-1, colicin-2, luffin-A, luffin-B, luffin-S, 19K-PSI, 15K-PSI, 9K-PSI, alpha-kirilowin, beta-kirilowin, gelonin, momordin, momordin-II, momordin-Ic, Mirabilis Antiviral Protein (MAP), MAP-30, alpha-momorcharin, beta-momorcharin, trichosanthin, TAP-29, trichokirin, barley RIP I, barley RIP II, tritin, flax RIP, maize RIP 3, maize RIP 9, maize RIP X, asparin-1, and asparin 2 or a fragment theref.

41. The method of claim 38, wherein the RIP is selected from among Shiga toxin (Stx), Shiga-like toxin II (Stx2), Shiga-like toxin I, volkensin, ricin, nigrin-CIP-29, abrin, vircumin, modeccin, ebulitin-α; ebulitin-β, ebultin-γ, and porrectin.

42. The method of claim 41, wherein the RIP is Shiga toxin or subunit A1 (SA1) thereof or an active fragment thereof.

43. The method of claim 42, wherein the SA1 is truncated.

44. The method of claim 38, wherein the RIP is modified.

45. The method of claim 44, wherein the RIP is or comprises SA1 that is modified by replacement of one or more amino acids.

46. The method of claim 45, wherein the SA1 is modified by replacement of Cys with another amino acid.

47. The method of claim 46, wherein the replacing amino acid is Ser.

48. The method of claim 45, wherein:
the SA1 is modified by replacement of one or both of positions 38 or position 219; and
the positions are with reference to amino acid positions in an SA1 having a sequence of amino acids set forth in SEQ ID NO:22.

49. The method of claim 48, wherein the amino acid replacement corresponds to L38R and/or V219A.

50. The method of claim 49, wherein the amino acid replacement corresponds to V219A.

51. The method of claim 48, wherein the SA1 has a sequence of amino acids set forth in SEQ ID NO: 26 or 28.

52. The method of claim 48, wherein the SA1 is encoded by a sequence of nucleic acids set forth in SEQ ID NO: 27 or 29.

53. The method of claim 38, wherein the RIP inhibitor is an adenine analog.

54. The method of claim 53, wherein:
the adenine analog is 4-aminopyrazolo[3,4-d]pyrimidine (4-APP); and
the concentration of 4-APP is effective to decrease the toxicity of the RIP by at least about 10%.

55. The method of claim 54, wherein the concentration of 4-APP is about 1 mM to about 40.0 mM.

56. The method of claim 38, wherein the host cells are eukaryotic cells or wherein the host cells are prokaryotic cells.

57. The method of claim 38, wherein the host cells are E. coli.

58. The method of claim 38, wherein the RIP polypeptide is expressed after induction with an induction agent.

59. The method of claim 58, wherein the induction agent is isopropyl-β-D-1-thiogalactopyranoside (IPTG).

60. The method of claim 58, wherein the RIP inhibitor is added before, during and/or after the addition of the induction agent.

61. The method of claim 38, wherein the nucleic acid molecule that encodes the RIP comprises a sequence of nucleotides encoding a ligand, whereby the molecule encodes a ligand-toxin conjugate.

62. The method of claim 61, wherein the RIP and ligand in the conjugate are linked directly via a covalent or ionic linkage or indirectly via a linker.

63. The method of claim 62, wherein the RIP and ligand in the conjugate are linked via a linker that is a peptide, polypeptide or an amino acid.

64. The method of claim 63, wherein the linker is an Ala-Met linker.

65. The method of claim 61, wherein the ligand-toxin conjugate is a fusion protein.

66. The method of claim 61, wherein the ligand in the ligand-toxin conjugate is selected from among a chemokine receptor targeting agent, a non-chemokine cytokine, a hormone, a growth factor, an antibody specific for a cell surface receptor, a TNF superfamily ligand, and a pattern recognition receptor (PRR) ligand.

67. The method of claim 61, wherein the ligand is vascular endotheial growth factor (VEGF).

68. The method of claim 66, wherein the chemokine receptor targeting agent is a an antibody that binds to the receptor or receptor-binding fragment thereof, a chemokine or a fragment of the chemokine that binds to the chemokine receptor, or an antibody that specifically binds to a chemokine receptor, or a fragment of the antibody that binds to the receptor.

69. The method of claim 68, wherein chemokine receptor targeting agent is specific for a receptor selected from among (DARC), D6, CXCR-1, CXCR-2, CXCR-3A, CXCR3B, CXCR-4, CXCR-5, CCR-1, CCR-2A, CCR-2B, CCR-3, CCR-4, CCR-5, CCR-6, CCR-7, CCR-8, CCR-9, CCR10, CX3CR-1, and XCR1.

70. The method of claim 68, wherein chemokine receptor targeting agent is specific for a receptor selected from CXCR-6 and CXCR-7.

71. The method of claim 68, wherein the chemokine receptor targeting agent is a chemokine selected from among monocytes chemotactic protein-1 (MCP-1), MCP-2, MCP-3, MCP-4, MCP-5, eosinophils chemotactic protein 1 (Eotaxin-1), Eotaxin-2, Eotaxin-3, stromal derived factor-1β (SDF-1β), SDF-1α, SDF-2, macrophage inhibitory protein 1α (MIP-1α), MIP-1β, MIP-1γ, MIP-2, MIP-2α, MIP-2β, MIP-3, MIP-3β, MIP-3α, MIP-4, MIP-5, Regulated on Activation, Normal T cell Expressed and Secreted (RANTES) protein, interleukin-8 (IL-8), growth regulated protein α (GRO-α), interferon-inducible protein 10 (IP-10), macrophage-derived chemokine (MDC), granulocyte chemotactic protein 2 (GCP-2), epithelial-derived neutrophil-activating protein 78 (ENA-78), platelet basic protein (PBP), gamma interferon-induced monokine (MIG), platelet factor 4 (PF-4), hemofiltrate CC chemokine 1 (HCC-1), thymus and activation-regulated chemokine (TARC), lymphotactin, lungkine, C10, liver-expressed chemokine (LEC), exodus-2 (SLC), thymus expressed chemokine (TECK), cutaneous T-cell attracting chemokine (CTACK), mucosae-associated epithelial chemokine (MEC), single C motif 1-β (SCM-1β), interferon-inducible T-cell alpha chemoattractant (1-TAC), breast and kidney-expressed chemokine (BRAK), fractalkine, and B cell-attracting chemokine 1 (BCA-1), and allelic or species variants thereof.

72. The method of claim 61, wherein RIP is a Shiga toxin, or active fragment thereof; or is a modified Shiga toxin or active fragment thereof that includes a modification.

73. The method of claim 61, wherein the ligand-toxin conjugate comprises the sequence of amino acid residues set forth in any of SEQ ID NOS: 42, 44, 46, 48, 50, 52, 54, 56, 58, 60, 62, 64, or 67.

74. The method of claim 1, further comprising preparing a conjugate containing the identified RIP.

75. The method of claim 38, further comprising preparing a conjugate containing the identified RIP.

76. A modified Shiga Toxin polypeptide, or active fragment thereof, comprising one or more amino acid modifications in a Shiga Toxin, allelic or species variant thereof, catalytically active fragment thereof, or active fragment thereof, wherein the one or more amino acid modifications are replacements of one or both of positions corresponding to positions 38 and/or 219 with reference to amino acid positions in Shiga Toxin A1 subunit (SA1) comprising a sequence of amino acids set forth in SEQ ID NO:22.

77. The modified Shiga Toxin of claim 76 that has at least about 65% sequence identity to the polypeptide comprising the sequence of amino acids set forth in SEQ ID NO: 22 and that includes modifications at loci corresponding to amino acid positions 38 and/or 219 or is an allelic or species variant of the polypeptide SEQ ID NO: 22 that includes modifications at loci corresponding to amino acid positions 38 and/or 219.

78. The modified Shiga Toxin of claim 76, wherein the modifications correspond to L38R and/or V219A.

79. The modified Shiga Toxin polypeptide of claim 76, that is the Shiga Toxin A1 chain (SA1), or an active fragment thereof.

80. The modified Shiga Toxin of claim 79, wherein the SA1 is truncated.

81. The modified Shiga Toxin of claim 79 that comprises or consists of the sequence of amino acids set forth in SEQ ID NOS: 26 or 28, or is an allelic or species variant thereof.

82. A conjugate, comprising the modified Shiga Toxin, or active fragment thereof of claim 76; and
a targeting agent that binds to a cell surface agent, whereby the conjugate binds to the cell surface receptor resulting in internalization of the targeted agent in cells bearing the receptor.

83. The conjugate of claim 82, comprising the following components: (targeting agent)ₙ, (L)_{q}, and (targeted agent)ₘ, wherein:
L is a linker for linking the targeting agent to the targeted agent;
targeting agent is any moiety that selectively binds to a cell surface receptor;
m and n, which are selected independently, are at least 1;
q is 0 or more as long as the resulting conjugate binds to the targeted receptor, is internalized and delivers the targeted agent;
the resulting conjugate binds to a receptor that interacts with and internalizes a targeting agent, whereby the targeted agent(s) is internalized in a cell bearing the receptor; and
when the conjugate contains a plurality of targeted agents the targeted agents are the same or different, and when the conjugate contains a plurality of targeting agents the targeting agents are the same or different.

84. The conjugate of claim 83, wherein m and n, which are selected independently, are 1-6.

85. The conjugate of claim 83, wherein q is 1, n is 2 and m is 1.

86. The conjugate of claim 82, wherein:
the targeting agent is selected from among a chemokine receptor targeting agent, a non-chemokine cytokine, a hormone, a growth factor, an antibody specific for a cell surface receptor, a TNF superfamily ligand, a pattern recognition receptor (PRR) ligand and fragments thereof that bind to a chemokine receptor to effect internalization of the conjugate.

87. The conjugate of claim 82, wherein the targeting agent is vascular endothelial growth factor (VEGF) or a portion thereof that binds to a VEGF receptor resulting in internalization of the conjugate.

88. The conjugate of claim 86, wherein the targeting agent is a chemokine or fragment thereof selected from among monocytes chemotactic protein-1 (MCP-1), MCP-2, MCP-3, MCP-4, MCP-5, eosinophils chemotactic protein 1 (Eotaxin-1), Eotaxin-2, Eotaxin-3, stromal derived factor-1β (SDF-1β), SDF-1α, SDF-2, macrophage inhibitory protein 1α (MIP-1α), MIP-1β, MIP-1γ, MIP-2, MIP-2α, MIP-2β, MIP-3, MIP-3β, MIP-3α, MIP-4, MIP-5, Regulated on Activation, Normal T cell Expressed and Secreted (RANTES) protein, interleukin-8 (IL-8), growth regulated protein α (GRO-α), interferon-inducible protein 10 (IP-10), macrophage-derived chemokine (MDC), granulocyte chemotactic protein 2 (GCP-2), epithelial-derived neutrophil-activating protein 78 (ENA-78), platelet basic protein (PBP), gamma interferon-induced monokine (MIG), platelet factor 4 (PF-4), hemofiltrate CC chemokine 1 (HCC-1), thymus and activation-regulated chemokine (TARC), lymphotactin, lungkine, C10, liver-expressed chemokine (LEC), exodus-2 (SLC), thymus expressed chemokine (TECK), cutaneous T-cell attracting chemokine (CTACK), mucosae-associated epithelial chemokine (MEC), single C motif 1-β (SCM-1β), interferon-inducible T-cell alpha chemoattractant (1-TAC), breast and kidney-expressed chemokine (BRAK), fractalkine, and B cell-attracting chemokine 1 (BCA-1) and fragments thereof.

89. The conjugate of claim 82, wherein the targeting agent specifically binds to one or more cell surface receptors on one or more immune effector cells, or other cells associated with an immune or inflammatory response.

90. The conjugate of claim 89, wherein the immune effector cell or cells is a leukocyte.

91. The conjugate of claim 89, wherein the cells are selected from among monocytes, macrophages, dendritic cells, T cells, B cells, eosinophils, basophils, mast cells, natural killer (NK) cells and neutrophils.

92. The conjugate of claim 91, wherein the macrophages are tissue macrophages selected from among alveolar macrophages, microglia, and kupfer cells.

93. The conjugate of claim 91, wherein the dendritic cells are selected from among immature dendritic cells, mature dendritic cells, and langerhans cells.

94. The conjugate of claim 91, wherein the T cells are selected from among CD4+ and CD8+ T cells.

95. The conjugate of claim 94, wherein the CD4+ T cells are Th17 cells.

96. The conjugate of claim 94, wherein the CD4+ T cells are Th1 or Th2 cells.

97. The conjugate of claim 89, wherein:
the one or more cells is another cell associated with the immune or inflammatory condition and is a tissue residential cells (TRC); and
the TRC are selected from among mesangial cells, glial cells, endothelial cells, epithelial cells, tumor cells, fibroblasts, and synoviocytes.

98. The conjugate of claim 89, wherein the cells are activated.

99. The conjugate of claim 98, wherein activation induces the expression of one or more cell surface receptors.

100. The conjugate of claim 82, wherein:
the cell surface receptor is a chemokine receptor selected from among CXCR1, CXCR2, CXCR3A, CXCR3B, CXCR4, CXCR5, CXCR6, CCR1, CCR2A, CCR2B, CCR3, CCR4, CCR5, CCR6, CCR7, CCR8, CCR9, XCR1 and CX3CR-1; and
the chemokine effects binding to a receptor, whereby the conjugate is internalized into a cell bearing the receptor.

101. The conjugate of claim 82, comprising or consisting of the sequence of amino acids set forth in any of SEQ ID NOS: 44, 46, 48, 50, 52, 54, 56, 58, 60, 62, 64 or 67.

102. A nucleic acid molecule, comprising a sequence of nucleotides that encodes a conjugate of claim 82.

103. A plasmid, comprising the nucleic acid molecule of claim 102.

104. A host cell, comprising the plasmid of claim 103.

105. A pharmaceutical composition comprising a conjugate of claim 82 in a pharmaceutically acceptable vehicle.

106. A method for inhibiting a disease or disorder, comprising administering a conjugate of claim 82 to an animal wherein:
the disease or disorder is an immune or inflammatory condition associated with inflammatory responses and/or secondary tissue damage associated with activation, proliferation and migration of one or more cells;
the conjugate binds to one or more cell surface receptors expressed on one or more cells resulting in internalization of the targeted agent in cells bearing the receptor; and
the conjugate inhibits the activation, proliferation or migration of one or more cells.

107. A conjugate, comprising a chemokine selected from among I-309, MCP-1, MIP-1β, MIP-1, RANTES, MCP-3, MCP-2, IL-8, MIG, IP-10, I-TAC, SDF-1α, SDF-1β, BCA-1, an Eotaxin, MCP-4, MCP-5, C10, LEC and MIP-1b2 or a fragment thereof linked directly or via a linker to a modified shiga toxin or SA1 subunit thereof or active fragment thereof of claim 76.

108. The method of claim 106, wherein:
the disease is multiple sclerosis (MS); and
the conjugate targets cells involved in the etiology or pathology of MS.

109. The method of claim 108, wherein the cells express receptors selected from among one or more of CCL1-8, CXCL8-13, CCR1-3,5, 6 and CXCR1-3, 4.

110. The method of claim 108, wherein the conjugate targets at least two receptors selected from among CCL1-8, CXCL8-13, CCR1-3,5, 6 and CXCR1-3, 4.

111. The method of claim 108, wherein;
the conjugate comprises a chemokine or fragment thereof sufficient for binding and internalization by a receptor therefor; and
the chemokine is selected from among I-309, MCP-1, MIP-1α, MIP-1β, RANTES, MCP-3, MCP-2, IL-8, MIG, IP-10, I-TAC, SDF-1α, SDF-1β, BCA-1, an Eotaxin, MCP-4, MCP-5, C10, LEC and MIP-1b2.

112. The method of claim 108, wherein the conjugate is LPM7 or LPM1d.
